SEQUENCE LISTING (174 sequences)

SEQ ID NO: 1 - protein, 118 aa, Artificial Sequence
  Synthetic Kv6.1  CD38 (VHa)
QVQLVESGGGLVKPGGSLRLSCAASGFTFSDDYMSWIRQAPGKGLEWVASVSNGRPTTYYADSVRGRFTISRDNAKNSLYLQMNSLRAEDTAVYYCAREGWSGEFTDWGQGTLVTVSS

SEQ ID NO: 2 - protein, 103 aa, Artificial Sequence
  Synthetic Kv6.1  CD38 (CHa)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 3 - protein, 20 aa, Artificial Sequence
  Synthetic Kv6.1  first linker
GGSGSGSGGSSGGGSGGGGS

SEQ ID NO: 4 - protein, 120 aa, Artificial Sequence [sequence omitted]

SEQ ID NO: 5 - protein, 103 aa, Artificial Sequence
  Synthetic Kv6.1  CD3 (CHb)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSS

SEQ ID NO: 6 - protein, 20 aa, Artificial Sequence
  Synthetic Kv6.1  hinge
DKTHTCPPCPAPEAAGGPSV

SEQ ID NO: 7 - protein, 100 aa, Artificial Sequence
  Synthetic Kv6.1  CH2
FLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 8 - protein, 107 aa, Artificial Sequence
  Synthetic Kv6.1  CH3
GQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 9 - protein, 110 aa, Artificial Sequence
  Synthetic Kv6.1  CD38 (VLa)
QSALTQPPSASGTSGQRVTISCSGSSSNIGFHFVYWYQHLPGTAPKLLIYKNNQRPSGVPDRFSGSKSGNSASLAISGLRSEDEADYYCAAWDDSLSGYVFGSGTKVTVL

SEQ ID NO: 10 - protein, 106 aa, Artificial Sequence
  Synthetic Kv6.1  CD38 (CLa)
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - protein, 20 aa, Artificial Sequence
  Synthetic Kv6.1  second linker (DP linker)
GGSGSGSGGSSGGGSGGGGS

SEQ ID NO: 12 - protein, 11 aa, Artificial Sequence
  Synthetic Kv6.1  second linker (EG linker)
TSGSGGSGGSV

SEQ ID NO: 13 - protein, 17 aa, Artificial Sequence
  Synthetic Kv6.1  second linker (EI linker)
TSGSGGSPLGMGGSGSV

SEQ ID NO: 14 - protein, 17 aa, Artificial Sequence
  Synthetic Kv6.1  second linker (EJ linker)
TSGSGGSPLGVGGSGSV

SEQ ID NO: 15 - protein, 17 aa, Artificial Sequence
  Synthetic Kv6.1  second linker (EK linker)
TSGSGGSPAALGGSGSV

SEQ ID NO: 16 - protein, 17 aa, Artificial Sequence
  Synthetic Kv6.1  second linker (EL linker)
TSGSGGSPAGLGGSGSV

SEQ ID NO: 17 - protein, 14 aa, Artificial Sequence
  Synthetic Kv6.1  second linker (EM linker)
TSGSGGSPLGMVGV

SEQ ID NO: 18 - protein, 14 aa, Artificial Sequence
  Synthetic Kv6.1  second linker (EN linker)
TSGSGGSPLGVVGV

SEQ ID NO: 19 - protein, 14 aa, Artificial Sequence
  Synthetic Kv6.1  second linker (EO linker)
TSGSGGSPAALVGV

SEQ ID NO: 20 - protein, 14 aa, Artificial Sequence
  Synthetic Kv6.1  second linker (EP linker)
TSGSGGSPAGLVGV

SEQ ID NO: 21 - protein, 14 aa, Artificial Sequence
  Synthetic Kv6.1  second linker (EQ linker)
TSGSGGSPLGMVLV

SEQ ID NO: 22 - protein, 14 aa, Artificial Sequence
  Synthetic Kv6.1  second linker (ER linker)
TSGSGGSPLGVVLV

SEQ ID NO: 23 - protein, 14 aa, Artificial Sequence
  Synthetic Kv6.1  second linker (ES linker)
TSGSGGSPAALVLV

SEQ ID NO: 24 - protein, 14 aa, Artificial Sequence
  Synthetic Kv6.1  second linker (ST linker)
TSGSGGSPAGLVLV

SEQ ID NO: 25 - protein, 106 aa, Artificial Sequence [sequence omitted]

SEQ ID NO: 26 - protein, 107 aa, Artificial Sequence
  Synthetic Kv6.1  CD3 (CLb)
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 27 - protein, 20 aa, Artificial Sequence
  Synthetic Kv6.1  hinge
DKTHTCPPCPAPEAAGGPSV

SEQ ID NO: 28 - protein, 100 aa, Artificial Sequence; identical to SEQ ID NO: 7
  Synthetic Kv6.1  CH2

SEQ ID NO: 29 - protein, 107 aa, Artificial Sequence
  Synthetic Kv6.1  CH3
GQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 118 aa, Artificial Sequence; identical to SEQ ID NO: 1
  Synthetic Kv6.2  CD38 (VHa)

SEQ ID NO: 31 - protein, 103 aa, Artificial Sequence; identical to SEQ ID NO: 5
  Synthetic Kv6.2  CD38 (CHa)

SEQ ID NO: 32 - protein, 20 aa, Artificial Sequence
  Synthetic Kv6.2  1st hinge
DKTHTCPPCPAPEAAGGPSV

SEQ ID NO: 33 - protein, 100 aa, Artificial Sequence; identical to SEQ ID NO: 7
  Synthetic Kv6.2  CH2

SEQ ID NO: 34 - protein, 107 aa, Artificial Sequence; identical to SEQ ID NO: 8
  Synthetic Kv6.2  CH3

SEQ ID NO: 35 - protein, 15 aa, Artificial Sequence
  Synthetic Kv6.2  first linker
GSGGSSGGGSGGGGS

SEQ ID NO: 36 - protein, 120 aa, Artificial Sequence [sequence omitted]

SEQ ID NO: 37 - protein, 103 aa, Artificial Sequence; identical to SEQ ID NO: 2
  Synthetic Kv6.2  CD3 (CHb)

SEQ ID NO: 38 - protein, 110 aa, Artificial Sequence; identical to SEQ ID NO: 9
  Synthetic Kv6.2  CD38 (VLa)

SEQ ID NO: 39 - protein, 106 aa, Artificial Sequence
  Synthetic Kv6.2  CD38 (CLa)
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTESS

SEQ ID NO: 40 - protein, 25 aa, Artificial Sequence
  Synthetic Kv6.2  2nd hinge
GGSGGDKTHTCPPCPAPEAAGGPSV

SEQ ID NO: 41 - protein, 100 aa, Artificial Sequence; identical to SEQ ID NO: 7
  Synthetic Kv6.2  CH2

SEQ ID NO: 42 - protein, 107 aa, Artificial Sequence; identical to SEQ ID NO: 29
  Synthetic Kv6.2  CH3

SEQ ID NO: 43 - protein, 15 aa, Artificial Sequence
  Synthetic Kv6.2  second linker (DX linker)
GSGGSSGGGSGGGGS

SEQ ID NO: 44 - protein, 11 aa, Artificial Sequence
  Synthetic Kv6.2  second linker (EH linker)
TSGSGGSGGSV

SEQ ID NO: 45 - protein, 17 aa, Artificial Sequence
  Synthetic Kv6.2  second linker (EU linker)
TSGSGGSPLGMGGSGSV

SEQ ID NO: 46 - protein, 17 aa, Artificial Sequence
  Synthetic Kv6.2  second linker (EV linker)
TSGSGGSPLGVGGSGSV

SEQ ID NO: 47 - protein, 17 aa, Artificial Sequence
  Synthetic Kv6.2  second linker (EW linker)
TSGSGGSPAALGGSGSV

SEQ ID NO: 48 - protein, 17 aa, Artificial Sequence
  Synthetic Kv6.2  second linker (EX linker)
TSGSGGSPAGLGGSGSV

SEQ ID NO: 49 - protein, 14 aa, Artificial Sequence
  Synthetic Kv6.2  second linker (EY linker)
TSGSGGSPLGMVGV

SEQ ID NO: 50 - protein, 14 aa, Artificial Sequence
  Synthetic Kv6.2  second linker (EZ linker)
TSGSGGSPLGVVGV

SEQ ID NO: 51 - protein, 14 aa, Artificial Sequence
  Synthetic Kv6.2  second linker (FA linker)
TSGSGGSPAALVGV

SEQ ID NO: 52 - protein, 14 aa, Artificial Sequence
  Synthetic Kv6.2  second linker (FB linker)
TSGSGGSPAGLVGV

SEQ ID NO: 53 - protein, 14 aa, Artificial Sequence
  Synthetic Kv6.2  second linker (FC linker)
TSGSGGSPLGMVLV

SEQ ID NO: 54 - protein, 14 aa, Artificial Sequence
  Synthetic Kv6.2  second linker (FD linker)
TSGSGGSPLGVVLV

SEQ ID NO: 55 - protein, 14 aa, Artificial Sequence
  Synthetic Kv6.2  second linker (FE linker)
TSGSGGSPAALVLV

SEQ ID NO: 56 - protein, 14 aa, Artificial Sequence
  Synthetic Kv6.2  second linker (FF linker)
TSGSGGSPAGLVLV

SEQ ID NO: 57 - protein, 106 aa, Artificial Sequence [sequence omitted]

SEQ ID NO: 58 - protein, 107 aa, Artificial Sequence [sequence omitted]

SEQ ID NO: 59 - protein, 118 aa, Artificial Sequence; identical to SEQ ID NO: 1
  Synthetic Kv5.1  CD38 (VHa)

SEQ ID NO: 60 - protein, 103 aa, Artificial Sequence; identical to SEQ ID NO: 2
  Synthetic Kv5.1  CD38 (CHa)

SEQ ID NO: 61 - protein, 20 aa, Artificial Sequence
  Synthetic Kv5.1  first linker
GGSGSGSGGSSGGGSGGGGS

SEQ ID NO: 62 - protein, 120 aa, Artificial Sequence [sequence omitted]

SEQ ID NO: 63 - protein, 103 aa, Artificial Sequence; identical to SEQ ID NO: 5
  Synthetic Kv5.1  CD3 (CHb)

SEQ ID NO: 64 - protein, 20 aa, Artificial Sequence
  Synthetic Kv5.1  1st hinge
DKTHTCPPCPAPEAAGGPSV

SEQ ID NO: 65 - protein, 100 aa, Artificial Sequence; identical to SEQ ID NO: 7
  Synthetic Kv5.1  CH2

SEQ ID NO: 66 - protein, 107 aa, Artificial Sequence; identical to SEQ ID NO: 8
  Synthetic Kv5.1  CH3

SEQ ID NO: 67 - protein, 110 aa, Artificial Sequence; identical to SEQ ID NO: 9
  Synthetic Kv5.1  CD38 (VLa)

SEQ ID NO: 68 - protein, 106 aa, Artificial Sequence; identical to SEQ ID NO: 10
  Synthetic Kv5.1  CD38 (CLa)

SEQ ID NO: 69 - protein, 106 aa, Artificial Sequence [sequence omitted]

SEQ ID NO: 70 - protein, 107 aa, Artificial Sequence; identical to SEQ ID NO: 26
  Synthetic Kv5.1  CD3 (CLb)

SEQ ID NO: 71 - protein, 25 aa, Artificial Sequence
  Synthetic Kv5.1  2nd hinge
GGSGGDKTHTCPPCPAPEAAGGPSV

SEQ ID NO: 72 - protein, 100 aa, Artificial Sequence; identical to SEQ ID NO: 7
  Synthetic Kv5.1  CH2

SEQ ID NO: 73 - protein, 107 aa, Artificial Sequence; identical to SEQ ID NO: 29
  Synthetic Kv5.1  CH3

SEQ ID NO: 74 - protein, 118 aa, Artificial Sequence; identical to SEQ ID NO: 1
  Synthetic Kv4.33  CD38 (VHa)

SEQ ID NO: 75 - protein, 103 aa, Artificial Sequence; identical to SEQ ID NO: 5
  Synthetic Kv4.33  CD38 (CHa)

SEQ ID NO: 76 - protein, 27 aa, Artificial Sequence
  Synthetic Kv4.33  1st hinge
RVEPKSSDKTHTCPPCPAPEAAGGPSV

SEQ ID NO: 77 - protein, 100 aa, Artificial Sequence; identical to SEQ ID NO: 7
  Synthetic Kv4.33  CH2

SEQ ID NO: 78 - protein, 107 aa, Artificial Sequence; identical to SEQ ID NO: 8
  Synthetic Kv4.33  CH3

SEQ ID NO: 79 - protein, 15 aa, Artificial Sequence
  Synthetic Kv4.33  first linker
GSGGSSGGGSGGGGS

SEQ ID NO: 80 - protein, 20 aa, Artificial Sequence
  Synthetic Linker
GGSGSGSGGSSGGGSGGGGS

SEQ ID NO: 81 - protein, 120 aa, Artificial Sequence [sequence omitted]

SEQ ID NO: 82 - protein, 103 aa, Artificial Sequence; identical to SEQ ID NO: 2
  Synthetic Kv4.33  CD3 (CHb)

SEQ ID NO: 83 - protein, 110 aa, Artificial Sequence; identical to SEQ ID NO: 9
  Synthetic Kv4.33  CD38 (VLa)

SEQ ID NO: 84 - protein, 106 aa, Artificial Sequence; identical to SEQ ID NO: 39
  Synthetic Kv4.33  CD38 (CLa)

SEQ ID NO: 85 - protein, 25 aa, Artificial Sequence
  Synthetic Kv4.33  2nd hinge
GGSGGDKTHTCPPCPAPEAAGGPSV

SEQ ID NO: 86 - protein, 100 aa, Artificial Sequence; identical to SEQ ID NO: 7
  Synthetic Kv4.33  CH2

SEQ ID NO: 87 - protein, 107 aa, Artificial Sequence; identical to SEQ ID NO: 29
  Synthetic Kv4.33  CH3

SEQ ID NO: 88 - protein, 106 aa, Artificial Sequence [sequence omitted]

SEQ ID NO: 89 - protein, 107 aa, Artificial Sequence [sequence omitted]

SEQ ID NO: 90 - protein, 118 aa, Artificial Sequence
  Synthetic Kv4.33  BCMA (VHa)
EVQLVESGGGLVKPGGSLRLSCAASGFTSSTAWMSWVRQAPGKGLEWVGRIKSKSDGGTTDYAAPVKGRFTISRDDSKNTLFLQMNSLKTEDTAVYYCAKGGGTYGYWGQGTTVTVSS

SEQ ID NO: 91 - protein, 96 aa, Artificial Sequence
  Synthetic Kv4.33  BCMA (CHa)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDK

SEQ ID NO: 92 - protein, 27 aa, Artificial Sequence
  Synthetic Kv4.33  hinge
RVEPKSSDKTHTCPPCPAPEAAGGPSV

SEQ ID NO: 93 - protein, 100 aa, Artificial Sequence; identical to SEQ ID NO: 7
  Synthetic Kv4.33  CH2

SEQ ID NO: 94 - protein, 107 aa, Artificial Sequence; identical to SEQ ID NO: 8
  Synthetic Kv4.33  CH3

SEQ ID NO: 95 - protein, 15 aa, Artificial Sequence
  Synthetic Linker
GSGGSSGGGSGGGGS

SEQ ID NO: 96 - protein, 120 aa, Artificial Sequence [sequence omitted]

SEQ ID NO: 97 - protein, 103 aa, Artificial Sequence; identical to SEQ ID NO: 2
  Synthetic Kv4.33 CD3(CHb)

SEQ ID NO: 98 - protein, 110 aa, Artificial Sequence
  Synthetic Kv4.33  BCMA (VLa)
SYVLTQPASVSGSPGQSVTISCTGTSSAHGGHYYVSWYQQHPGKAPKLMIYDVSNRPSGVSNRFSGSKSGNTASLTISGLQAEDEADYYCGSYTSSGSYVFGTGTKLTVL

SEQ ID NO: 99 - protein, 106 aa, Artificial Sequence; identical to SEQ ID NO: 39
  Synthetic Kv4.33  BCMA (CLa)

SEQ ID NO: 100 - protein, 25 aa, Artificial Sequence
  Synthetic Kv4.33  hinge
GGSGGDKTHTCPPCPAPEAAGGPSV

SEQ ID NO: 101 - protein, 100 aa, Artificial Sequence; identical to SEQ ID NO: 7
  Synthetic Kv4.33  CH2

SEQ ID NO: 102 - protein, 107 aa, Artificial Sequence; identical to SEQ ID NO: 29
  Synthetic Kv4.33  CH3

SEQ ID NO: 103 - protein, 106 aa, Artificial Sequence [sequence omitted]

SEQ ID NO: 104 - protein, 107 aa, Artificial Sequence [sequence omitted]

SEQ ID NO: 105 - protein, 120 aa, Artificial Sequence
  Synthetic Kv5.1 EGFR (VHa)
EVQLVESGGGVVQPGRSLRLSCAASGFIFHHYAMHWVRQAPGKGLEWVAHISSDGSSKFYADSVKGRFTVSRDNSKNTLYLQMNSLRAEDTAVYYCARFTEVLYYGADLWGQGTLVTVSS

SEQ ID NO: 106 - protein, 103 aa, Artificial Sequence; identical to SEQ ID NO: 2
  Synthetic Kv5.1 EGFR (CHa)

SEQ ID NO: 107 - protein, 10 aa, Artificial Sequence
  Synthetic Kv5.1  first linker
GGGGSGGGGS

SEQ ID NO: 108 - protein, 119 aa, Artificial Sequence
  Synthetic Kv5.1  PD-L1 (VHb)
EVQLVESGAEVKKPGSSVKVSCKASGGTFSSYAISWVRQAPGQGLEWMGGIIPIFGTANYAQKFQGRVTITADESTSTAYMELSSLRSEDTAVYYCARAPYYYYYMDVWGQGTTVTVSS

SEQ ID NO: 109 - protein, 103 aa, Artificial Sequence
  Synthetic Kv5.1  PD-L1 (CHb)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSS

SEQ ID NO: 110 - protein, 20 aa, Artificial Sequence
  Synthetic Kv5.1  hinge
DKTHTCPPCPAPEAAGGPSV

SEQ ID NO: 111 - protein, 100 aa, Artificial Sequence; identical to SEQ ID NO: 7
  Synthetic Kv5.1  CH2

SEQ ID NO: 112 - protein, 107 aa, Artificial Sequence; identical to SEQ ID NO: 8
  Synthetic Kv5.1  CH3

SEQ ID NO: 113 - protein, 110 aa, Artificial Sequence
  Synthetic Kv5.1 EGFR (VLa)
QSVLTQPPSVSEAPRQRVSISCSGSSSNIGNNAVNWYQQLPGKAPKLLIYFDDLLPLGVSDRFSGSKSGTSASLAISGLQSEDEADYYCAAWDDSLKGYVFGTGTKVTVL

SEQ ID NO: 114 - protein, 106 aa, Artificial Sequence
  Synthetic Kv5.1 EGFR (CLa)
GQPKAAPSVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 115 - protein, 109 aa, Artificial Sequence
  Synthetic Kv5.1  PD-L1 (VLb)
QSALTQPASVSGSLGQSVTISCTGSSSDVGSYNLVSWYQQHPGKAPNLMIYDVSKRSGVSNRFSGSKSGNTASLTISGLQAEDEADYYCSSYTGISTVVFGGGTKLTVL

SEQ ID NO: 116 - protein, 106 aa, Artificial Sequence; identical to SEQ ID NO: 39
  Synthetic Kv5.1  PD-L1 (CLb)

SEQ ID NO: 117 - protein, 20 aa, Artificial Sequence
  Synthetic Kv5.1  hinge
DKTHTCPPCPAPEAAGGPSV

SEQ ID NO: 118 - protein, 100 aa, Artificial Sequence; identical to SEQ ID NO: 7
  Synthetic Kv5.1  CH2

SEQ ID NO: 119 - protein, 107 aa, Artificial Sequence; identical to SEQ ID NO: 29
  Synthetic Kv5.1  CH3

SEQ ID NO: 120 - protein, 119 aa, Artificial Sequence; identical to SEQ ID NO: 108
  Synthetic Kv4.33  PD-L1 (VHa)

SEQ ID NO: 121 - protein, 103 aa, Artificial Sequence; identical to SEQ ID NO: 109
  Synthetic Kv4.33  PD-L1 (CHa)

SEQ ID NO: 122 - protein, 20 aa, Artificial Sequence
  Synthetic Kv4.33  hinge
DKTHTCPPCPAPEAAGGPSV

SEQ ID NO: 123 - protein, 100 aa, Artificial Sequence; identical to SEQ ID NO: 7
  Synthetic Kv4.33  CH2

SEQ ID NO: 124 - protein, 107 aa, Artificial Sequence; identical to SEQ ID NO: 8
  Synthetic Kv4.33  CH3

SEQ ID NO: 125 - protein, 10 aa, Artificial Sequence
  Synthetic Kv4.33  first linker
GGGGSGGGGS

SEQ ID NO: 126 - protein, 120 aa, Artificial Sequence; identical to SEQ ID NO: 105
  Synthetic Kv4.33  EGFR (VHb)

SEQ ID NO: 127 - protein, 103 aa, Artificial Sequence; identical to SEQ ID NO: 2
  Synthetic Kv4.33 EGFR (CHb)

SEQ ID NO: 128 - protein, 109 aa, Artificial Sequence; identical to SEQ ID NO: 115
  Synthetic Kv4.33  PD-L1 (VLa)

SEQ ID NO: 129 - protein, 106 aa, Artificial Sequence; identical to SEQ ID NO: 39
  Synthetic Kv4.33  PD-L1 (CLa)

SEQ ID NO: 130 - protein, 20 aa, Artificial Sequence
  Synthetic Kv4.33  hinge
DKTHTCPPCPAPEAAGGPSV

SEQ ID NO: 131 - protein, 100 aa, Artificial Sequence; identical to SEQ ID NO: 7
  Synthetic Kv4.33  CH2

SEQ ID NO: 132 - protein, 107 aa, Artificial Sequence; identical to SEQ ID NO: 29
  Synthetic Kv4.33  CH3

SEQ ID NO: 133 - protein, 110 aa, Artificial Sequence; identical to SEQ ID NO: 113
  Synthetic Kv4.33  EGFR (VLb)

SEQ ID NO: 134 - protein, 106 aa, Artificial Sequence; identical to SEQ ID NO: 114
  Synthetic Kv4.33 EGFR (CLb)

SEQ ID NO: 135 - protein, 118 aa, Artificial Sequence
  Synthetic Kv5.1 BCMA (VHa)
EVQLVESGGGLVKPGGSLRLSCAASGFTSSTAWMSWVRQAPGKGLEWVGRIKSKSDGGTTDYAAPVKGRFTISRDDSKNTLYLQMNSLKTEDTAVYYCAKGGGTYGYWGQGTTVTVSS

SEQ ID NO: 136 - protein, 103 aa, Artificial Sequence; identical to SEQ ID NO: 2
  Synthetic Kv5.1 BCMA (CHa)

SEQ ID NO: 137 - protein, 20 aa, Artificial Sequence
  Synthetic Kv5.1  first linker
GGSGSGSGGSSGGGSGGGGS

SEQ ID NO: 138 - protein, 120 aa, Artificial Sequence [sequence omitted]

SEQ ID NO: 139 - protein, 103 aa, Artificial Sequence; identical to SEQ ID NO: 5
  Synthetic Kv5.1  CD3 (CHb)

SEQ ID NO: 140 - protein, 20 aa, Artificial Sequence
  Synthetic Kv5.1  hinge
DKTHTCPPCPAPEAAGGPSV

SEQ ID NO: 141 - protein, 100 aa, Artificial Sequence; identical to SEQ ID NO: 7
  Synthetic Kv5.1  CH2

SEQ ID NO: 142 - protein, 107 aa, Artificial Sequence; identical to SEQ ID NO: 8
  Synthetic Kv5.1  CH3

SEQ ID NO: 143 - protein, 110 aa, Artificial Sequence
  Synthetic Kv5.1 BCMA (VLa)
SYVLTQPASVSGSPGQSITISCTGTSSAHGGHYYVSWYQQHPGKAPKLMIYDVSNRPSGVSNRFSGSKSGNTASLTISGLQAEDEADYYCGSYTSSGSYVFGTGTKLTVL

SEQ ID NO: 144 - protein, 106 aa, Artificial Sequence; identical to SEQ ID NO: 10
  Synthetic Kv5.1 BCMA (CLa)

SEQ ID NO: 145 - protein, 106 aa, Artificial Sequence [sequence omitted]

SEQ ID NO: 146 - protein, 107 aa, Artificial Sequence; identical to SEQ ID NO: 26
  Synthetic Kv5.1  CD3 (CLb)

SEQ ID NO: 147 - protein, 25 aa, Artificial Sequence
  Synthetic Kv5.1  hinge
GGSGGDKTHTCPPCPAPEAAGGPSV

SEQ ID NO: 148 - protein, 100 aa, Artificial Sequence; identical to SEQ ID NO: 7
  Synthetic Kv5.1  CH2

SEQ ID NO: 149 - protein, 107 aa, Artificial Sequence; identical to SEQ ID NO: 29
  Synthetic Kv5.1  CH3

SEQ ID NO: 150 - protein, 1210 aa, Artificial Sequence [sequence omitted]

SEQ ID NO: 151 - protein, 290 aa, Artificial Sequence
  Synthetic PD-L1 antigen
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 152 - protein, 300 aa, Artificial Sequence
  Synthetic CD38 antigen
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 153 - protein, 184 aa, Artificial Sequence
  Synthetic BCMA antigen
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 154 - protein, 468 aa, Artificial Sequence
  Synthetic Control anti-EGFR antibody VH
MEWSWVFLFFLSVTTGVHSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 233 aa, Artificial Sequence
  Synthetic Control anti-EGFR antibody VL
MEWSWVFLFFLSVTTGVHSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 156 - protein, 11 aa, Artificial Sequence
  Synthetic Linker
TSGSGGSGGSV

SEQ ID NO: 157 - protein, 12 aa, Artificial Sequence
  Synthetic Linker
  misc_feature (3)..(12): “Ser Gly” may or may not be present
SGSGSGSGSGSG

SEQ ID NO: 158 - protein, 18 aa, Artificial Sequence
  Synthetic Linker
  misc_feature (4)..(18): “Ser Gly Gly” may or may not be present
SGGSGGSGGSGGSGGSGG

SEQ ID NO: 159 - protein, 24 aa, Artificial Sequence
  Synthetic Linker
  misc_feature (5)..(24): “Ser Gly Gly Gly” may or may not be present
SGGGSGGGSGGGSGGGSGGGSGGG

SEQ ID NO: 160 - protein, 18 aa, Artificial Sequence
  Synthetic Linker
  misc_feature (4)..(18): “Ser Ser Gly” may or may not be present
SSGSSGSSGSSGSSGSSG

SEQ ID NO: 161 - protein, 12 aa, Artificial Sequence
  Synthetic Linker
  misc_feature (3)..(12): “Gly Ser” may or may not be present
GSGSGSGSGSGS

SEQ ID NO: 162 - protein, 18 aa, Artificial Sequence
  Synthetic Linker
  misc_feature (4)..(18): “Gly Gly Gly” may or may not be present
GGGGGGGGGGGGGGGGGG

SEQ ID NO: 163 - protein, 30 aa, Artificial Sequence
  Synthetic Linker
  misc_feature (6)..(30): “Gly Ser Gly Gly Ser” may or may not be present
GSGGSGSGGSGSGGSGSGGSGSGGSGSGGS

SEQ ID NO: 164 - protein, 18 aa, Artificial Sequence
  Synthetic Linker
  misc_feature (4)..(18): “Gly Ser Gly” may or may not be present
GSGGSGGSGGSGGSGGSG

SEQ ID NO: 165 - protein, 30 aa, Artificial Sequence
  Synthetic Linker
  misc_feature (6)..(30): “Gly Gly Gly Gly Ser” may or may not be present
GGGGSGGGGSGGGGSGGGGSGGGGSGGGGS

SEQ ID NO: 166 - protein, 24 aa, Artificial Sequence
  Synthetic Linker
  misc_feature (5)..(24): “Gly Gly Gly Ser” may or may not be present
GGGSGGGSGGGSGGGSGGGSGGGS

SEQ ID NO: 167 - protein, 42 aa, Artificial Sequence
  Synthetic Linker
  misc_feature (8)..(42): “Gly Gly Gly Gly Ser Gly Ser” may or may not be present
GGGGSGSGGGGSGSGGGGSGSGGGGSGSGGGGSGSGGGGSGS

SEQ ID NO: 168 - protein, 48 aa, Artificial Sequence
  Synthetic Linker
  misc_feature (9)..(48): “Gly Gly Gly Gly Ser Gly Gly Ser” may or may not be present
GGGGSGGSGGGGSGGSGGGGSGGSGGGGSGGSGGGGSGGSGGGGSGGS

SEQ ID NO: 169 - protein, 18 aa, Artificial Sequence
  Synthetic Linker
  misc_feature (4)..(18): “Gly Gly Ser” may or may not be present
GGSGGSGGSGGSGGSGGS

SEQ ID NO: 170 - protein, 10 aa, Artificial Sequence
  Synthetic IgG1 upper hinge sequence
EPKSCDKTHT

SEQ ID NO: 171 - protein, 4 aa, Artificial Sequence
  Synthetic IgG1 core hinge sequence
  misc_feature (3)..(3): X is P, R or S
CPXC

SEQ ID NO: 172 - protein, 9 aa, Artificial Sequence
  Synthetic lower hinge/CH2 sequence
PAPELLGGP

SEQ ID NO: 173 - protein, 12 aa, Artificial Sequence
  Synthetic Fc region (CH2)
SVFLFPPKPKDT

SEQ ID NO: 174 - protein, 23 aa, Artificial Sequence
  Synthetic hinge sequence
EPKSCDKTHTCPPCPAPELLGGP

(19) United States
(12) Patent Application Publication
(10) Pub. No.: US 2022/0324974 A1
(43) Pub. Date: Oct. 13, 2022

(54) Activatable Multi-Specific Antigen Binding Protein Complexes

(71) Applicant: Sorrento Therapeutics, Inc., San Diego, CA (US)

(72) Inventors: Yanliang Zhang, San Diego, CA (US); Gunnar F. Kaufmann, San Diego, CA (US); Xiao He, Lincolnshire, IL (US); Yun Wei Lai, San Diego, CA (US)

(73) Assignee: Sorrento Therapeutics, Inc., San Diego, CA (US)

(21) Appl. No.: 17/602,908

(22) PCT Filed: Apr. 10, 2020
(86) PCT No.: PCT/US2020/027663

§ 371 Date: Oct. 11, 2021

Related U.S. Application Data
(60) Provisional application No. 62/899,347, filed on Sep. 12, 2019.
(60) Provisional application No. 62/833,360, filed on Apr. 12, 2019.

(51) Int. Cl. C07K 16/28

(52) U.S. Cl. CPC C07K 16/2809; C07K 16/2827; C07K 16/2878; C07K 16/2896; C07K 2317/31; C07K 2317/73; C07K 2317/55; C07K 2317/60; C07K 2317/92

(57) ABSTRACT

The present disclosure provides several embodiments of multi-specific antigen binding protein complexes. In some embodiments, the multi-specific antigen binding protein complex is composed of either two or three polypeptide chains that assemble with each other to form het-erodimeric complexes comprising two different Fab regions each capable of binding two different epitopes and comprising an Fc region which is capable of exhibiting Fc effector function, thus having a relatively simple structure compared to certain other multi-specific antibodies. In one embodiment, the multi-specific antigen binding protein complex is activatable as one of the polypeptide chains that compose the protein complex carries a cleavable linker.

### CROSS-REFERENCE TO RELATED APPLICATIONS

This application claims the benefit of priority under 35 U.S.C. § 119 to U.S. provisional application No. 62/833,360, filed Apr. 12, 2019, and U.S. provisional application No. 62/899,347, filed Sep. 12, 2019. The disclosures of all of the aforementioned applications are incorporated by reference in their entireties.

Throughout this application various publications, patents, and/or patent applications are referenced. The disclosures of the publications, patents and/or patent applications are hereby incorporated by reference in their entireties into this application in order to more fully describe the state of the art to which this disclosure pertains.

### SEQUENCE LISTING

The instant application contains a Sequence Listing which has been submitted electronically in ASCII format and is hereby incorporated by reference in its entirety. Said ASCII copy, created on Apr. 7, 2020, is named 01223-0018-00PCT_ST25.txt and is 143 KB in size.

### TECHNICAL FIELD

The present disclosure provides multi-specific antigen binding protein complexes having immunoglobulin-like antigen binding activity, methods for their production, pharmaceutical compositions, and uses thereof.

### INTRODUCTION

Tumors are known to contain heterogenous cell types including malignant, stromal and immune cells, which play various roles in tumor vascularization, growth and metastasis. Tumor metastasis is a complex process that involves tumor cells over-expressing proteases that degrade cellular structures that surround tumor cells which promotes cell invasion and metastasis. In particular, certain classes of human proteases that play a role in tumor progression include serine proteases, metalloproteases, cysteine proteases and aspartyl proteases. Tumor cells produce several different proteases which are secreted and accumulate in the extracellular vicinity of a tumor mass. The cells in and near a tumor, and the secreted proteases in close proximity to the tumor are part of the tumor microenvironment. Tumor extracts containing certain proteases that are associated with poor patient prognosis, including type II transmembrane serine proteases, urokinase plasminogen activator (uPA) systems, metalloproteases including MMPs and ADAMs, and cysteine proteases including cysteine cathepsins.

Bispecific antibodies that are designed to generate immune cell synapse can bind antigens on effector cells and tumor-associated antigens expressed by tumors which leads to tumor-selective cytotoxic cell killing. However, bispecific antibodies sometimes bind to healthy cells that express low levels of tumor-associated antigens, resulting in on-target off-tumor cell killing. To overcome this problem, activatable bispecific antibodies have been developed to utilize the proteases located in the tumor microenvironment. Protease-activatable antibodies (also known as activatable probodies) carry a cleavable peptide with a protease-recognizable sequence. The cleavable peptide in an intact state interferes with antibody binding capacity to effector cells but does not interfere with binding capacity to the tumor-associated antigen. The protease-activatable antibodies selectively bind in an inactive state to tumor-associated antigens expressed by tumor and healthy cells however cell killing is not initiated because the cleavable linker is intact. The cleavable linker is not cleaved until the bispecific antibody is in close proximity to a tumor microenvironment containing a protease that can cleave the linker which generates the activated form of the antibody which in turn binds effector T cells, leading to cytotoxic cell killing, thus sparing healthy cells and tissues. The protease-activatable bispecific antibodies need not physically contact the tumor cell, nor enter the tumor cell, but rather need to be located in the tumor microenvironment which contains the tumor-secreted protease.

Disclosed herein are multi-specific antigen binding protein complexes comprising (i) two different Fab regions, each capable of binding two different epitopes, and (ii) an Fc region. The multi-specific antigen binding protein complexes may comprise two or three polypeptide chains that associate with each other to form a protein complex. In some embodiments, such as when two polypeptide chains form the complex, the multi-specific antigen binding protein complex may comprise a cleavable linker that reduces, inhibits, or prevents binding of one of the Fab regions to its target antigen unless the cleavable linker is cleaved (which would convert a two-chain complex to a three-chain complex). In some embodiments, the multi-specific antigen binding protein complexes are relatively simple compared to existing multi-specific antibodies in that only two or three polypeptide chains are used per complex, which is fewer than a standard human IgG molecule, which consists of four chains. In some embodiments, the positioning of the half Fab heavy and half Fab light regions on the two or three chains of the complexes according to the disclosure will favor efficient formation of heterodimer protein complexes that bind two different epitopes, or at least provide the public with a useful choice.

In some embodiments, the multi-specific antigen binding protein complexes can activate an immune cell in the presence of a target cell. In some embodiments, the multi-specific antigen binding protein complexes may provide one or more benefits, such as high specificity for activation of immune cells in the presence of cancer cells or a target tumor environment, and/or high efficacy in promoting immune cell-mediated target cell killing, or at least provide the public with a useful choice.

### SUMMARY

The present disclosure provides various embodiments of a two-chain multi-specific antigen binding protein complex that can bind two different epitopes on the same target antigen or two different epitopes on different target antigens at the same time, where the protein complex comprises: two different Fab regions, an Fc region, and a first and a second linker.

In one embodiment, the two different Fab regions and the Fc region are formed from the association of a first polypeptide chain with a second polypeptide chain, wherein each of the first and second polypeptide chains carries two different half Fab regions and a half Fc region, wherein the first polypeptide chain carries the first linker, the second polypeptide chain carries the second linker, and the second linker is cleavable.

In one embodiment, the first and second polypeptide chains associate with each other to form the multi-specific antigen binding protein complex having a first Fab region that is capable of binding a first epitope, a second Fab region that is capable of binding a second epitope that differs from the first epitope, a full Fc region that is capable of binding an Fc receptor, and a first and second linker.

In one embodiment, the first Fab region exhibits binding to the first target epitope and the second Fab region exhibits reduced binding to the second target epitope when the second linker is un-cleaved compared to when the second linker is cleaved. In one embodiment, the second Fab region exhibits increased binding to the second target epitope when the second linker is cleaved compared to when the second linker is un-cleaved. In one embodiment, upon cleavage of the second linker, the first Fab region and the second Fab region are capable of binding to the first and second target epitopes, respectively, at the same time.

In one embodiment, the first linker is cleavable with a matrix metalloprotease, wherein the matrix metalloprotease is MMP1, MMP2, MMP3, MMP8, MMP9, MMP11, MMP13, MMP14, or MT1-MMP (membrane type 1 matrix metalloproteinase).

In one embodiment, the first linker comprises the amino acid sequence of TSGSGGSGGSV (SEQ ID NO: 156), (SG)ₙ(SEQ ID NO: 157), (SGG)ₙ(SEQ ID NO: 158), (SGGG)ₙ (SEQ ID NO: 159), (SSG)ₙ(SEQ ID NO: 160), (GS)ₙ (SEQ ID NO: 161), (GGG)ₙ (SEQ ID NO: 162), (GSGGS)ₙ(SEQ ID NO: 163), (GSG)ₙ(SEQ ID NO: 164), (GGGGS)ₙ(SEQ ID NO: 165), (GGGS)ₙ(SEQ ID NO: 166), (GGGGSGS)ₙ(SEQ ID NO: 167), (GGGGSGGS)ₙ(SEQ ID NO: 168) or (GGS)ₙ(SEQ ID NO: 169), wherein n is an integer of 1-6.

In one embodiment, the second linker comprises the amino acid sequence of GGSGSGSGGSSGGGSGGGGS (DP linker), TSGSGGSGGSV (EG or EH linker), TSGSGGSPLGMGGSGSV (EI or EU linker), TSGSGGSPLGVGGSGSV (EJ or EV linker), TSGSGGSPAALGGSGSV (EK or EW linker), TSGSGGSPAGLGGSGSV (EL or EX linker), TSGSGGSPLGMVGV (EM or EY linker), TSGSGGSPLGVVGV (EN or EZ linker), TSGSGGSPAALVGV (EO or FA linker), TSGSGGSPAGLVGV (EP or FB linker), TSGSGGSPLGMVLV (EQ or FC linker), TSGSGGSPLGVVLV (ER or FD linker), TSGSGGSPAALVLV (ES or FE linker) or TSGSGGSPAGLVLV (ST or FF linker) (SEQ ID NOS; 11-24, respectively, or SEQ ID NOS:43-56, respectively).

In one embodiment of the two-chain multi-specific antigen binding protein complex, the two different Fab regions are arranged in-tandem, and the protein complex comprises: (a) a first polypeptide chain comprising (i) a first half Fab heavy region, (ii) a first linker, (iii) a second half Fab heavy region, and (iv) a first half Fc region, wherein the first half Fab heavy region comprises a first-variable region and first-constant region from a first Fab heavy chain, and wherein the second half Fab heavy region comprises a second-variable region and second-constant region from a second Fab heavy chain; and (b) a second polypeptide chain comprising (i) a first half Fab light region, (ii) a second linker, (iii) a second half Fab light region, and (iv) a second half Fc region, wherein the first half Fab light region comprises a first-variable region and first-constant region from a first Fab light chain, and wherein the second half Fab light region comprises a second-variable region and second-constant region from a second Fab light chain, and wherein the second linker is cleavable. Non-limiting examples of in-tandem protein complexes are shown in FIGS. 1, 5, 6 and 7.

In one embodiment of the two-chain multi-specific antigen binding protein complex, the two different Fab regions are arranged in a non-tandem manner, and the protein complex comprises: (a) a first polypeptide chain comprising (i) a first half Fab heavy region, (ii) a first half Fc region, (iii) a first linker, and (iv) a second half Fab heavy region, wherein the first half Fab heavy region comprises a first-variable region and first-constant region from a first Fab heavy chain, and wherein the second half Fab heavy region comprises a second-variable region and second-constant region from a second Fab heavy chain; and (b) a second polypeptide chain comprising (i) a first half Fab light region, (ii) a second half Fc region, (iii) a second linker, and (iv) a second half Fab light region, wherein the first half Fab light region comprises a first-variable region and first-constant region from a first Fab light chain, and wherein the second half Fab light region comprises a second-variable region and second-constant region from a second Fab light chain, and wherein the second linker is cleavable. Non-limiting examples of non-tandem protein complexes are shown in FIGS. 3, 8, 9 and 10.

In one embodiment, any of the two-chain multi-specific antigen binding protein complexes described herein comprise (i) a first polypeptide chain having a hole mutation in the first half Fc region and a second polypeptide chain having a knob mutation in the second half Fc region, or (ii) a first polypeptide chain having a knob mutation in the first half Fc region and a second polypeptide chain having a hole mutation in the second half Fc region.

In one embodiment, any of the two-chain multi-specific antigen binding protein complexes described herein comprise first and second polypeptide chains having any one or any combination of two or more knob and hole mutations comprising T366Y, T366W, T366S, L368A, T394S, T394W, F405A, F405W, Y407A, Y407V and/or Y407T according to Kabat numbering.

In one embodiment, any of the two-chain multi-specific antigen binding protein complexes described herein comprise a first Fab region that binds the first target epitope with a dissociation constant K_{d} of 10⁻⁵M or less, or 10⁻⁶M or less, or 10⁻⁷ M or less, or 10⁻⁸M or less, or 10⁻⁹M or less, or 10⁻¹⁰ M or less.

In one embodiment, any of the two-chain multi-specific antigen binding protein complexes described herein comprise a second Fab region wherein upon cleavage of the second linker the second Fab region binds the second target epitope with a dissociation constant K_{d} of 10⁻⁵M or less, or 10⁻⁶ M or less, or 10⁻⁷ M or less, or 10⁻⁸ M or less, or 10⁻⁹M or less, or 10⁻¹⁰ M or less.

In one embodiment of the two-chain multi-specific antigen binding protein complex, the two different Fab regions are arranged in-tandem, and the protein complex comprises: (a) a first polypeptide chain which comprises (e.g., in N- to C-terminal order): (i) a first heavy chain variable domain comprising an amino acid sequence that is at least 95% identical to SEQ ID NO:1; (ii) a first heavy chain constant domain comprising an amino acid sequence that is at least 95% identical to SEQ ID NO:2; (iii) a first linker comprising an amino acid sequence that is at least 95% identical to SEQ ID NO:3; (iv) a second heavy chain variable domain comprising an amino acid sequence that is at least 95% identical to SEQ ID NO:4; (v) a second heavy chain constant domain comprising an amino acid sequence that is at least 95% identical to SEQ ID NO:5; (vi) a first hinge sequence comprising an amino acid sequence that is at least 95% identical to SEQ ID NO:6; (vii) a first CH2 domain comprising an amino acid sequence that is at least 95% identical to SEQ ID NO:7; and (viii) a first CH3 domain comprising an amino acid sequence that is at least 95% identical to SEQ ID NO:8; and (b) a second polypeptide chain which comprises (e.g., in N- to C-terminal order): (i) a first light chain variable domain comprising an amino acid sequence that is at least 95% identical to SEQ ID NO:9; (ii) a first light chain constant domain comprising an amino acid sequence that is at least 95% identical to SEQ ID NO:10; (iii) a second linker comprising an amino acid sequence that is at least 95% identical to any one of SEQ ID NOS:11-24; (iv) a second light chain variable domain comprising an amino acid sequence that is at least 95% identical to SEQ ID NO:25; (v) a second light chain constant domain comprising an amino acid sequence that is at least 95% identical to SEQ ID NO:26; (vi) a second hinge sequence comprising an amino acid sequence that is at least 95% identical to SEQ ID NO:27; (vii) a second CH2 domain comprising an amino acid sequence that is at least 95% identical to SEQ ID NO:28; and (viii) a second CH3 domain comprising an amino acid sequence that is at least 95% identical to SEQ ID NO:29 (e.g., Kv6.1, FIGS. 31A-C).

In one embodiment of the two-chain multi-specific antigen binding protein complex, the two different Fab regions are arranged in a non-tandem manner, and the protein complex comprises: (a) a first polypeptide chain which comprises (e.g., in N- to C-terminal order): (i) a first heavy chain variable domain comprising an amino acid sequence that is at least 95% identical to SEQ ID NO:30; (ii) a first heavy chain constant domain comprising an amino acid sequence that is at least 95% identical to SEQ ID NO:31; (iii) a first hinge comprising an amino acid sequence that is at least 95% identical to SEQ ID NO:32; (iv) a first CH2 domain comprising an amino acid sequence that is at least 95% identical to SEQ ID NO:33; (v) a first CH3 domain comprising an amino acid sequence that is at least 95% identical to SEQ ID NO:34; (vi) a first linker sequence comprising an amino acid sequence that is at least 95% identical to SEQ ID NO:35; (vii) a second heavy chain variable domain comprising an amino acid sequence that is at least 95% identical to SEQ ID NO:36; and (viii) a second heavy chain constant domain comprising an amino acid sequence that is at least 95% identical to SEQ ID NO:37; and (b) a second polypeptide chain which comprises (e.g., in N- to C-terminal order): (i) a first light chain variable domain comprising an amino acid sequence that is at least 95% identical to SEQ ID NO:38; (ii) a first light chain constant domain comprising an amino acid sequence that is at least 95% identical to SEQ ID NO:39; (iii) a second hinge comprising an amino acid sequence that is at least 95% identical to SEQ ID NO:40; (iv) a second CH2 domain comprising an amino acid sequence that is at least 95% identical to SEQ ID NO:41; (v) a second CH3 domain comprising an amino acid sequence that is at least 95% identical to SEQ ID NO:42; (vi) a second linker sequence comprising an amino acid sequence that is at least 95% identical to any one of SEQ ID NOS:43-56; (vii) a second light chain variable domain comprising an amino acid sequence that is at least 95% identical to SEQ ID NO:57; and (viii) a second light chain constant domain comprising an amino acid sequence that is at least 95% identical to SEQ ID NO:58 (e.g., Kv6.2, FIGS. 32A-C).

The present disclosure provides a pharmaceutical composition comprising any one of the two-chain multi-specific antigen binding protein complexes described herein and a pharmaceutically-acceptable excipient.

In one embodiment, any of the two-chain multi-specific antigen binding protein complexes described herein can be used as a medicament.

The present disclosure provides one or more nucleic acids encoding (i) the first polypeptide chain of any one of the two-chain multi-specific antigen binding protein complexes described herein, and encoding (ii) the second polypeptide chain of any of the two-chain multi-specific antigen binding protein complexes described herein.

The present disclosure provides one or more vectors comprising any of the one or more nucleic acids described herein operably linked to one or more promoters.

The present disclosure provides a host cell harboring any of the one or more vectors described herein.

The present disclosure provides a method for preparing any of the two-chain multi-specific antigen binding protein complexes described herein, comprising: culturing a population of the host cell described herein under conditions suitable for expressing the first and second polypeptide chains. In one embodiment, the method further comprises: isolating (e.g., recovering) the first and second polypeptide chains. In one embodiment, the method further comprises subjecting the first and second polypeptide chains to conditions suitable for associating the first and second polypeptide chains with each other to form the two-chain multi-specific antigen binding protein complex. In one embodiment, the formed two-chain multi-specific antigen binding protein complex comprises a hetero-dimeric molecule.

The present disclosure provides (i) a first nucleic acid encoding the first polypeptide chain of any one of the two-chain multi-specific antigen binding protein complexes described herein, and (ii) a second nucleic acid encoding the second polypeptide chain of any of the two-chain multi-specific antigen binding protein complexes described herein.

The present disclosure provides (i) a first vector operably linked to the first nucleic acid encoding the first polypeptide chain of any one of the two-chain multi-specific antigen binding protein complexes described herein, and (ii) a second vector operably linked to the second nucleic acid encoding the second polypeptide chain of any of the two-chain multi-specific antigen binding protein complexes described herein. In one embodiment, the first vector is a first expression vector comprising at least one promoter which is operably linked to the first nucleic acid encoding the first polypeptide chain. In one embodiment, the second vector is a second expression vector comprising at least one promoter which is operably linked to the second nucleic acid encoding the second polypeptide chain.

The present disclosure provides a host cell harboring any of the first and second vectors described herein.

The present disclosure provides method for preparing any of the two-chain multi-specific antigen binding protein complexes described herein, comprising: culturing a population of the host cell harboring the first and second vectors under conditions suitable for expressing the first and second polypeptide chains, wherein the first vector is operably linked to the first nucleic acid encoding the first polypeptide chain of any one of the two-chain multi-specific antigen binding protein complexes described herein, and wherein the second vector is operably linked to the second nucleic acid encoding the second polypeptide chain of any of the two-chain multi-specific antigen binding protein complexes described herein. In one embodiment, the method further comprises: isolating (recovering) the first and second polypeptide chains. In one embodiment, the method further comprises subjecting the first and second polypeptide chains to conditions suitable for associating the first and second polypeptide chains with each other to form the two-chain multi-specific antigen binding protein complex. In one embodiment, the formed two-chain multi-specific antigen binding protein complex comprises a hetero-dimeric molecule.

The present disclosure provides (i) a first host cell harboring the first vector comprising a promoter operably linked to the first nucleic acid encoding the first polypeptide chain of any one of the two-chain multi-specific antigen binding protein complexes described herein, and (ii) a second host cell harboring the second vector comprising a promoter operably linked to the second nucleic acid encoding the second polypeptide chain of any one of the two-chain multi-specific antigen binding protein complexes described herein.

The present disclosure provides a method for preparing any of the two-chain multi-specific antigen binding protein complexes described herein, comprising: (a) culturing a population of the first host cell harboring the first vector under conditions suitable for expressing the first polypeptide chain, and (b) culturing a population of the second host cell harboring the second vector under conditions suitable for expressing the second polypeptide chain. In one embodiment, the method further comprises: isolating (recovering) the first and second polypeptide chains. In one embodiment, the method further comprises subjecting the first and second polypeptide chains to conditions suitable for associating the first and second polypeptide chains with each other to form the two-chain multi-specific antigen binding protein complex. In one embodiment, the formed two-chain multi-specific antigen binding protein complex comprises a hetero-dimeric molecule.

The present disclosure provides a method for treating a disease in a subject, comprising: administering to the subject a therapeutically effective amount of any one of the two-chain multi-specific antigen binding protein complexes described herein.

In one embodiment the disease comprises a cancer of the: prostate, breast, ovary, head and neck, bladder, skin, colorectal, anus, rectum, pancreas, lung (including non-small cell lung and small cell lung cancers), leiomyoma, brain, glioma, glioblastoma, esophagus, liver, kidney, stomach, colon, cervix, uterus, endometrium, vulva, larynx, vagina, bone, nasal cavity, paranasal sinus, nasopharynx, oral cavity, oropharynx, larynx, hypolarynx, salivary glands, ureter, urethra, penis, or testis.

In one embodiment, the disease comprises a hematologic cancer, wherein the hematologic cancer is B chronic lymphocytic leukemia (B-CLL), B and T acute lymphocytic leukemia (ALL), acute myeloid leukemia (AML), chronic lymphocytic leukemia (CLL), chronic myelogenous leukemia (CIVIL), hairy cell leukemia (HCL), myeloproliferative disorder/neoplasm (MPDS), myelodysplasia syndrome, non-Hodgkin's lymphoma (NHL) including Burkitt's lymphoma (BL), Waldenstrom's Macroglobulinemia, mantle cell lymphoma, AIDS-related lymphoma, Hodgkin's Lymphoma (HL), T cell lymphoma (TCL), multiple myeloma (MM), plasma cell myeloma, plamocytoma, giant cell myeloma, heavy-chain myeloma, or light chain or Bence-Jones myeloma.

The present disclosure provides a method for binding a first and a second target epitope, comprising: (a) contacting the first target epitope with any of the two-chain multi-specific antigen binding protein complexes described herein, wherein the second linker is in an un-cleaved state and the protein complex is inactive; and (b) binding the first target epitope with the first Fab region, wherein the first Fab region binds the first target epitope and the second Fab region exhibits reduced binding to the second target epitope when the second linker is in the un-cleaved state. In one embodiment, the method further comprises: (c) cleaving the second linker to generate an activated two-chain multi-specific antigen binding protein complex wherein the second Fab region can bind to the second target epitope. In one embodiment, the method further comprises: (d) contacting the second target epitope with the activated two-chain multi-specific antigen binding protein complex; and (e) binding the second target epitope with the second Fab region.

In one embodiment, the first target epitope comprises a cell surface antigen expressed by a tumor or cancer cell.

In one embodiment, second epitope comprises a cell surface antigen expressed by an effector T cell. In one embodiment, the activated two-chain multi-specific antigen binding protein complex forms a cell synapse by binding the cell surface antigen expressed by the tumor or cancer cell and by binding the cell surface antigen expressed by the effector T cell.

In one embodiment, the effector T cell in the cell synapse kills the tumor or cancer cell by mediating cytotoxic cell killing.

The present disclosure provides various embodiments of a three-chain multi-specific antigen binding protein complex that can bind two different epitopes on the same target antigen or two different epitopes on different target antigens at the same time, where the protein complex comprises: two different Fab regions, an Fc region, and a first and a second linker.

In one embodiment, the two different Fab regions and the Fc region are formed from the association of a first, second and third polypeptide chains.

In one embodiment, the first, second and third polypeptide chains associate with each other to form the multi-specific antigen binding protein complex having a first Fab region that is capable of binding a first epitope, a second Fab region that is capable of binding a second epitope that differs from the first epitope, a full Fc region that is capable of binding an Fc receptor, and a first and second linker.

In one embodiment of the three-chain multi-specific antigen binding protein complex, the two different Fab regions are arranged in-tandem, and the protein complex comprises: (a) a first polypeptide chain comprising (i) a first half Fab heavy region, (ii) a first linker, (iii) a second half Fab heavy region, and (iv) a first half Fc region, wherein the first half Fab heavy region comprises a first-variable region and first-constant region from a first Fab heavy chain, and wherein the second half Fab heavy region comprises a second-variable region and second-constant region from a second Fab heavy chain; (b) a second polypeptide chain comprising (i) a first half Fab light region, wherein the first half Fab light region comprises a first-variable region and first-constant region from a first Fab light chain; and (c) a third polypeptide chain comprising (i) a second half Fab light region, and (ii) a second half Fc region, wherein the second half Fab light region comprises a second-variable region and second-constant region from a second Fab light chain. Non-limiting examples of in-tandem protein complexes are shown in FIGS. 2, 11, 12 and 13.

In one embodiment of the three-chain multi-specific antigen binding protein complex, the two different Fab regions are arranged in a non-tandem manner, and the protein complex comprises: (a) a first polypeptide chain comprising (i) a first half Fab heavy region, (ii) a first half Fc region, (iii) a first linker, and (iv) a second half Fab heavy region, wherein the first half Fab heavy region comprises a first-variable region and first-constant region from a first Fab heavy chain, and wherein the second half Fab heavy region comprises a second-variable region and second-constant region from a second Fab heavy chain; and (b) a second polypeptide chain comprising (i) a first half Fab light region, (ii) a second half Fc region, wherein the first half Fab light region comprises a first-variable region and first-constant region from a first Fab light chain; and (c) a third polypeptide chain comprising (i) a second half Fab light region, wherein the second half Fab light region comprises a second-variable region and second-constant region from a second Fab light chain. Non-limiting examples of non-tandem protein complexes are shown in FIGS. 4, 14, 15 and 16.

In one embodiment, any of the three-chain multi-specific antigen binding protein complexes having the two different Fab regions are arranged in-tandem as described herein comprise (i) a first polypeptide chain having a hole mutation in the first half Fc region and a third polypeptide chain having a knob mutation in the second half Fc region, or (ii) a first polypeptide chain having a knob mutation in the first half Fc region and a third polypeptide chain having a hole mutation in the second half Fc region.

In one embodiment, any of the three-chain multi-specific antigen binding protein complexes having the two different Fab regions are arranged in-tandem as described herein comprise first and third polypeptide chains having any one or any combination of two or more knob and hole mutations comprising T366Y, T366W, T366S, L368A, T394S, T394W, F405A, F405W, Y407A, Y407V and/or Y407T according to Kabat numbering.

In one embodiment, any of the three-chain multi-specific antigen binding protein complexes having the two different Fab regions are arranged in a non-tandem manner as described herein comprise (i) a first polypeptide chain having a hole mutation in the first half Fc region and a second polypeptide chain having a knob mutation in the second half Fc region, or (ii) a first polypeptide chain having a knob mutation in the first half Fc region and a second polypeptide chain having a hole mutation in the second half Fc region.

In one embodiment, any of the three-chain multi-specific antigen binding protein complexes having the two different Fab regions are arranged in a non-tandem manner as described herein comprise first and second polypeptide chains having any one or any combination of two or more knob and hole mutations comprising T366Y, T366W, T366S, L368A, T394S, T394W, F405A, F405W, Y407A, Y407V and/or Y407T according to Kabat numbering.

In one embodiment, any of the three-chain multi-specific antigen binding protein complexes described herein comprise a first Fab region that binds the first target epitope with a dissociation constant K_{d} of 10⁻⁵M or less, or 10⁻⁶ M or less, or 10⁻⁷M or less, or 10⁻⁸M or less, or 10⁻⁹M or less, or 10⁻¹⁰ M or less.

In one embodiment, any of the three-chain multi-specific antigen binding protein complexes described herein comprise a second Fab region that binds the second target epitope with a dissociation constant K_{d} of 10⁻⁵M or less, or 10⁻⁶ M or less, or 10⁻⁷M or less, or 10⁻⁸M or less, or 10⁻⁹ M or less, or 10⁻¹⁰ M or less.

In one embodiment of the three-chain multi-specific antigen binding protein complex, the two different Fab regions are arranged in-tandem, and the protein complex comprises: (a) a first polypeptide chain which comprises (e.g., in N- to C-terminal order): (i) a first heavy chain variable domain comprising an amino acid sequence that is at least 95% identical to SEQ ID NO:59; (ii) a first heavy chain constant domain comprising an amino acid sequence that is at least 95% identical to SEQ ID NO:60; (iii) a first linker comprising an amino acid sequence that is at least 95% identical to SEQ ID NO:61; (iv) a second heavy chain variable domain comprising an amino acid sequence that is at least 95% identical to SEQ ID NO:62; (v) a second heavy chain constant domain comprising an amino acid sequence that is at least 95% identical to SEQ ID NO:63; (vi) a first hinge sequence comprising an amino acid sequence that is at least 95% identical to SEQ ID NO:64; (vii) a first CH2 domain comprising an amino acid sequence that is at least 95% identical to SEQ ID NO:65; and (viii) a first CH3 domain comprising an amino acid sequence that is at least 95% identical to SEQ ID NO:66; and (b) a second polypeptide chain which comprises (e.g., in N- to C-terminal order): (i) a first light chain variable domain comprising an amino acid sequence that is at least 95% identical to SEQ ID NO:67; (ii) a first light chain constant domain comprising an amino acid sequence that is at least 95% identical to SEQ ID NO:68; and (c) a third polypeptide chain which comprises (e.g., in N- to C-terminal order): (i) a second light chain variable domain comprising an amino acid sequence that is at least 95% identical to SEQ ID NO:69; (ii) a second light chain constant domain comprising an amino acid sequence that is at least 95% identical to SEQ ID NO:70; (iii) a second hinge sequence comprising an amino acid sequence that is at least 95% identical to SEQ ID NO:71; (iv) a second CH2 domain comprising an amino acid sequence that is at least 95% identical to SEQ ID NO:72; and (v) a second CH3 domain comprising an amino acid sequence that is at least 95% identical to SEQ ID NO:73 (e.g., Kv5.1, FIGS. 33A-B).

In one embodiment of the three-chain multi-specific antigen binding protein complex, the two different Fab regions are arranged in-tandem, and the protein complex comprises: (a) a first polypeptide chain which comprises (e.g., in N- to C-terminal order): (i) a first heavy chain variable domain comprising an amino acid sequence that is at least 95% identical to SEQ ID NO:105; (ii) a first heavy chain constant domain comprising an amino acid sequence that is at least 95% identical to SEQ ID NO:106; (iii) a first linker comprising an amino acid sequence that is at least 95% identical to SEQ ID NO:107; (iv) a second heavy chain variable domain comprising an amino acid sequence that is at least 95% identical to SEQ ID NO:108; (v) a second heavy chain constant domain comprising an amino acid sequence that is at least 95% identical to SEQ ID NO:109; (vi) a first hinge sequence comprising an amino acid sequence that is at least 95% identical to SEQ ID NO:110; (vii) a first CH2 domain comprising an amino acid sequence that is at least 95% identical to SEQ ID NO:111; and (viii) a first CH3 domain comprising an amino acid sequence that is at least 95% identical to SEQ ID NO:112; and (b) a second polypeptide chain which comprises (e.g., in N- to C-terminal order): (i) a first light chain variable domain comprising an amino acid sequence that is at least 95% identical to SEQ ID NO:113; (ii) a first light chain constant domain comprising an amino acid sequence that is at least 95% identical to SEQ ID NO:114; and (c) a third polypeptide chain which comprises (e.g., in N- to C-terminal order): (i) a second light chain variable domain comprising an amino acid sequence that is at least 95% identical to SEQ ID NO:115; (ii) a second light chain constant domain comprising an amino acid sequence that is at least 95% identical to SEQ ID NO:116; (iii) a second hinge sequence comprising an amino acid sequence that is at least 95% identical to SEQ ID NO:117; (iv) a second CH2 domain comprising an amino acid sequence that is at least 95% identical to SEQ ID NO:118; and (v) a second CH3 domain comprising an amino acid sequence that is at least 95% identical to SEQ ID NO:119 (e.g., Kv5.1, FIGS. 36A-B).

In one embodiment of the three-chain multi-specific antigen binding protein complex, the two different Fab regions are arranged in-tandem, and the protein complex comprises: (a) a first polypeptide chain which comprises (e.g., in N- to C-terminal order): (i) a first heavy chain variable domain comprising an amino acid sequence that is at least 95% identical to SEQ ID NO:135; (ii) a first heavy chain constant domain comprising an amino acid sequence that is at least 95% identical to SEQ ID NO:136; (iii) a first linker comprising an amino acid sequence that is at least 95% identical to SEQ ID NO:137; (iv) a second heavy chain variable domain comprising an amino acid sequence that is at least 95% identical to SEQ ID NO:138; (v) a second heavy chain constant domain comprising an amino acid sequence that is at least 95% identical to SEQ ID NO:139; (vi) a first hinge sequence comprising an amino acid sequence that is at least 95% identical to SEQ ID NO:140; (vii) a first CH2 domain comprising an amino acid sequence that is at least 95% identical to SEQ ID NO:141; and (viii) a first CH3 domain comprising an amino acid sequence that is at least 95% identical to SEQ ID NO:142; and (b) a second polypeptide chain which comprises (e.g., in N- to C-terminal order): (i) a first light chain variable domain comprising an amino acid sequence that is at least 95% identical to SEQ ID NO:143; (ii) a first light chain constant domain comprising an amino acid sequence that is at least 95% identical to SEQ ID NO:144; and (c) a third polypeptide chain which comprises (e.g., in N- to C-terminal order): (i) a second light chain variable domain comprising an amino acid sequence that is at least 95% identical to SEQ ID NO:145; (ii) a second light chain constant domain comprising an amino acid sequence that is at least 95% identical to SEQ ID NO:146; (iii) a second hinge sequence comprising an amino acid sequence that is at least 95% identical to SEQ ID NO:147; (iv) a second CH2 domain comprising an amino acid sequence that is at least 95% identical to SEQ ID NO:148; and (v) a second CH3 domain comprising an amino acid sequence that is at least 95% identical to SEQ ID NO:149 (e.g., Kv5.1, FIGS. 38A-B).

In one embodiment of the three-chain multi-specific antigen binding protein complex, the two different Fab regions are arranged in a non-tandem manner, and the protein complex comprises: (a) a first polypeptide chain which comprises (e.g., in N- to C-terminal order): (i) a first heavy chain variable domain comprising an amino acid sequence that is at least 95% identical to SEQ ID NO:74; (ii) a first heavy chain constant domain comprising an amino acid sequence that is at least 95% identical to SEQ ID NO:75; (iii) a first hinge comprising an amino acid sequence that is at least 95% identical to SEQ ID NO:76; (iv) a first CH2 domain comprising an amino acid sequence that is at least 95% identical to SEQ ID NO:77; (v) a first CH3 domain comprising an amino acid sequence that is at least 95% identical to SEQ ID NO:78; (vi) a first linker sequence comprising an amino acid sequence that is at least 95% identical to SEQ ID NO:79 or 80; (vii) a second heavy chain variable domain comprising an amino acid sequence that is at least 95% identical to SEQ ID NO:81; and (viii) a second heavy chain constant domain comprising an amino acid sequence that is at least 95% identical to SEQ ID NO:82; and (b) a second polypeptide chain which comprises (e.g., in N- to C-terminal order): (i) a first light chain variable domain comprising an amino acid sequence that is at least 95% identical to SEQ ID NO:83; (ii) a first light chain constant domain comprising an amino acid sequence that is at least 95% identical to SEQ ID NO:84; (iii) a second hinge comprising an amino acid sequence that is at least 95% identical to SEQ ID NO:85; (iv) a second CH2 domain comprising an amino acid sequence that is at least 95% identical to SEQ ID NO:86; (v) a second CH3 domain comprising an amino acid sequence that is at least 95% identical to SEQ ID NO:87; and (c) (i) a second light chain variable domain comprising an amino acid sequence that is at least 95% identical to SEQ ID NO:88; and (ii) a second light chain constant domain comprising an amino acid sequence that is at least 95% identical to SEQ ID NO:89 (e.g., Kv4.33, FIGS. 34A-B).

In one embodiment of the three-chain multi-specific antigen binding protein complex, the two different Fab regions are arranged in a non-tandem manner, and the protein complex comprises: (a) a first polypeptide chain which comprises (e.g., in N- to C-terminal order): (i) a first heavy chain variable domain comprising an amino acid sequence that is at least 95% identical to SEQ ID NO:90; (ii) a first heavy chain constant domain comprising an amino acid sequence that is at least 95% identical to SEQ ID NO:91; (iii) a first hinge comprising an amino acid sequence that is at least 95% identical to SEQ ID NO:92; (iv) a first CH2 domain comprising an amino acid sequence that is at least 95% identical to SEQ ID NO:93; (v) a first CH3 domain comprising an amino acid sequence that is at least 95% identical to SEQ ID NO:94; (vi) a first linker sequence comprising an amino acid sequence that is at least 95% identical to SEQ ID NO:95; (vii) a second heavy chain variable domain comprising an amino acid sequence that is at least 95% identical to SEQ ID NO:96; and (viii) a second heavy chain constant domain comprising an amino acid sequence that is at least 95% identical to SEQ ID NO:97; and (b) a second polypeptide chain which comprises (e.g., in N- to C-terminal order): (i) a first light chain variable domain comprising an amino acid sequence that is at least 95% identical to SEQ ID NO:98 (ii) a first light chain constant domain comprising an amino acid sequence that is at least 95% identical to SEQ ID NO:99; (iii) a second hinge comprising an amino acid sequence that is at least 95% identical to SEQ ID NO:100; (iv) a second CH2 domain comprising an amino acid sequence that is at least 95% identical to SEQ ID NO:101; (v) a second CH3 domain comprising an amino acid sequence that is at least 95% identical to SEQ ID NO:102; and (c) (i) a second light chain variable domain comprising an amino acid sequence that is at least 95% identical to SEQ ID NO:103; and (ii) a second light chain constant domain comprising an amino acid sequence that is at least 95% identical to SEQ ID NO:104 (e.g., Kv4.33, FIGS. 35A-B).

In one embodiment of the three-chain multi-specific antigen binding protein complex, the two different Fab regions are arranged in a non-tandem manner, and the protein complex comprises: (a) a first polypeptide chain which comprises (e.g., in N- to C-terminal order): (i) a first heavy chain variable domain comprising an amino acid sequence that is at least 95% identical to SEQ ID NO:120; (ii) a first heavy chain constant domain comprising an amino acid sequence that is at least 95% identical to SEQ ID NO:121; (iii) a first hinge comprising an amino acid sequence that is at least 95% identical to SEQ ID NO:122; (iv) a first CH2 domain comprising an amino acid sequence that is at least 95% identical to SEQ ID NO:123; (v) a first CH3 domain comprising an amino acid sequence that is at least 95% identical to SEQ ID NO:124; (vi) a first linker sequence comprising an amino acid sequence that is at least 95% identical to SEQ ID NO:125 (vii) a second heavy chain variable domain comprising an amino acid sequence that is at least 95% identical to SEQ ID NO:126; and (viii) a second heavy chain constant domain comprising an amino acid sequence that is at least 95% identical to SEQ ID NO:127; and (b) a second polypeptide chain which comprises (e.g., in N- to C-terminal order): (i) a first light chain variable domain comprising an amino acid sequence that is at least 95% identical to SEQ ID NO:128 (ii) a first light chain constant domain comprising an amino acid sequence that is at least 95% identical to SEQ ID NO:129; (iii) a second hinge comprising an amino acid sequence that is at least 95% identical to SEQ ID NO:130; (iv) a second CH2 domain comprising an amino acid sequence that is at least 95% identical to SEQ ID NO:131; (v) a second CH3 domain comprising an amino acid sequence that is at least 95% identical to SEQ ID NO:132; and (c) (i) a second light chain variable domain comprising an amino acid sequence that is at least 95% identical to SEQ ID NO:133; and (ii) a second light chain constant domain comprising an amino acid sequence that is at least 95% identical to SEQ ID NO:134 (e.g., Kv4.33, FIGS. 37A-B).

The present disclosure provides a pharmaceutical composition comprising any one of the three-chain multi-specific antigen binding protein complexes described herein and a pharmaceutically-acceptable excipient.

In one embodiment, any of the three-chain multi-specific antigen binding protein complexes described herein can be used as a medicament.

The present disclosure provides one or more nucleic acids encoding (i) the first polypeptide chain of any one of the three-chain multi-specific antigen binding protein complexes described herein, and encoding (ii) the second polypeptide chain of any of the three-chain multi-specific antigen binding protein complexes described herein, and encoding (iii) the third polypeptide chain of any of the three-chain multi-specific antigen binding protein complexes described herein.

The present disclosure provides a vector operably linked to the one or more nucleic acids encoding the first polypeptide chain of any one of the three-chain multi-specific antigen binding protein complexes described herein, and encoding the second polypeptide chain of any of the three-chain multi-specific antigen binding protein complexes described herein, and encoding the third polypeptide chain of any of the three-chain multi-specific antigen binding protein complexes described herein.

The present disclosure provides a host cell harboring the vector which is operably linked to the one or more nucleic acids encoding the first polypeptide chain of any one of the three-chain multi-specific antigen binding protein complexes described herein, and encoding the second polypeptide chain of any of the three-chain multi-specific antigen binding protein complexes described herein, and encoding the third polypeptide chain of any of the three-chain multi-specific antigen binding protein complexes described herein.

The present disclosure provides a method for preparing any of the three-chain multi-specific antigen binding protein complexes described herein, comprising: culturing a population of the host cell harboring the vector under conditions suitable for expressing the first, second and third polypeptide chains, wherein the vector is operably linked to the one or more nucleic acids encoding the first polypeptide chain of any one of the three-chain multi-specific antigen binding protein complexes described herein, and encoding the second polypeptide chain of any of the three-chain multi-specific antigen binding protein complexes described herein, and encoding the third polypeptide chain of any of the three-chain multi-specific antigen binding protein complexes described herein. In one embodiment, the method further comprises: isolating (recovering) the first, second and third polypeptide chains. In one embodiment, the method further comprises subjecting the first, second and third polypeptide chains to conditions suitable for associating the first, second and third polypeptide chains with each other to form/assemble the three-chain multi-specific antigen binding protein complex. In one embodiment, the formed/assembled three-chain multi-specific antigen binding protein complex comprises a hetero-dimeric molecule.

The present disclosure provides (i) a first nucleic acid encoding the first polypeptide chain of any one of the three-chain multi-specific antigen binding protein complexes described herein, and (ii) a second nucleic acid encoding the second polypeptide chain of any of the three-chain multi-specific antigen binding protein complexes described herein, (ii) a third nucleic acid encoding the third polypeptide chain of any of the three-chain multi-specific antigen binding protein complexes described herein.

The present disclosure provides a vector system comprising a first vector operably linked to the first nucleic acid and optionally operably linked to any one or any combination of the second and/or third nucleic acid(s), and a second vector operably linked to any one or any combination of the second and/or third nucleic acid(s), wherein the first nucleic acid encodes the first polypeptide chain of any one of the three-chain multi-specific antigen binding protein complexes described herein, wherein the second nucleic acid encodes the second polypeptide chain of any of the three-chain multi-specific antigen binding protein complexes described herein, and wherein the third nucleic acid encodes the third polypeptide chain of any of the three-chain multi-specific antigen binding protein complexes described herein.

In one embodiment, (i) a first vector is operably linked to the first nucleic acid encoding the first polypeptide chain of any one of the three-chain multi-specific antigen binding protein complexes described herein, and (ii) a second vector operably linked to the second nucleic acid encoding the second polypeptide chain of any of the three-chain multi-specific antigen binding protein complexes described herein, and the second vector operably linked to the third nucleic acid encoding the third polypeptide chain of any of the three-chain multi-specific antigen binding protein complexes described herein. In one embodiment, the first vector is a first expression vector comprising at least one promoter which is operably linked to the first nucleic acid encoding the first polypeptide chain. In one embodiment, the second vector is a second expression vector comprising at least one promoter which is operably linked to the second nucleic acid encoding the second polypeptide chain and is operably linked to the third nucleic acid encoding the third polypeptide chain.

The present disclosure provides a host cell harboring the vector system, wherein the vector system comprises a first vector operably linked to the first nucleic acid and optionally operably linked to any one or any combination of the second and/or third nucleic acid(s), and a second vector operably linked to any one or any combination of the second and/or third nucleic acid(s), wherein the first nucleic acid encodes the first polypeptide chain of any one of the three-chain multi-specific antigen binding protein complexes described herein, wherein the second nucleic acid encodes the second polypeptide chain of any of the three-chain multi-specific antigen binding protein complexes described herein, and wherein the third nucleic acid encodes the third polypeptide chain of any of the three-chain multi-specific antigen binding protein complexes described herein.

In one embodiment, the host cell harbors the first and second vectors, wherein (i) the first vector is operably linked to the first nucleic acid encoding the first polypeptide chain of any one of the three-chain multi-specific antigen binding protein complexes described herein, and (ii) the second vector operably linked to the second nucleic acid encoding the second polypeptide chain of any of the three-chain multi-specific antigen binding protein complexes described herein, and the second vector is operably linked to the third nucleic acid encoding the third polypeptide chain of any of the three-chain multi-specific antigen binding protein complexes described herein.

The present disclosure provides method for preparing any of the three-chain multi-specific antigen binding protein complexes described herein, comprising: culturing a population of the host cell harboring the first and second vectors under conditions suitable for expressing the first, second and third polypeptide chains, wherein (i) the first vector is operably linked to the first nucleic acid encoding the first polypeptide chain of any one of the three-chain multi-specific antigen binding protein complexes described herein, and (ii) the second vector operably linked to the second nucleic acid encoding the second polypeptide chain of any of the three-chain multi-specific antigen binding protein complexes described herein, and the second vector is operably linked to the third nucleic acid encoding the third polypeptide chain of any of the three-chain multi-specific antigen binding protein complexes described herein. In one embodiment, the method further comprises: isolating (recovering) the first, second and third polypeptide chains. In one embodiment, the method further comprises subjecting the first, second and third polypeptide chains to conditions suitable for associating the first, second and third polypeptide chains with each other to form/assemble the three-chain multi-specific antigen binding protein complex. In one embodiment, the formed/assembled three-chain multi-specific antigen binding protein complex comprises a hetero-dimeric molecule.

The present disclosure provides a method for treating a disease in a subject, comprising: administering to the subject a therapeutically effective amount of any one of the three-chain multi-specific antigen binding protein complexes described herein.

In one embodiment the disease comprises a cancer of the: prostate, breast, ovary, head and neck, bladder, skin, colorectal, anus, rectum, pancreas, lung (including non-small cell lung and small cell lung cancers), leiomyoma, brain, glioma, glioblastoma, esophagus, liver, kidney, stomach, colon, cervix, uterus, endometrium, vulva, larynx, vagina, bone, nasal cavity, paranasal sinus, nasopharynx, oral cavity, oropharynx, larynx, hypolarynx, salivary glands, ureter, urethra, penis, or testis.

In one embodiment, the disease comprises a hematologic cancer, wherein the hematologic cancer is B chronic lymphocytic leukemia (B-CLL), B and T acute lymphocytic leukemia (ALL), acute myeloid leukemia (AML), chronic lymphocytic leukemia (CLL), chronic myelogenous leukemia (CIVIL), hairy cell leukemia (HCL), myeloproliferative disorder/neoplasm (MPDS), myelodysplasia syndrome, non-Hodgkin's lymphoma (NHL) including Burkitt's lymphoma (BL), Waldenstrom's Macroglobulinemia, mantle cell lymphoma, AIDS-related lymphoma, Hodgkin's Lymphoma (HL), T cell lymphoma (TCL), multiple myeloma (MM), plasma cell myeloma, plamocytoma, giant cell myeloma, heavy-chain myeloma, or light chain or Bence-Jones myeloma.

The present disclosure provides a method for binding a first and a second target epitope, comprising: (a) contacting the first target epitope and the second target epitope with any of the three-chain multi-specific antigen binding protein complexes described herein, and (b) binding the first target epitope with the first Fab region and binding the second target epitope with the second Fab region.

In one embodiment, the first target epitope comprises a cell surface antigen expressed by a tumor or cancer cell.

In one embodiment, second epitope comprises a cell surface antigen expressed by an effector T cell. In one embodiment, the three-chain multi-specific antigen binding protein complex forms a cell synapse by binding the cell surface antigen expressed by the tumor or cancer cell and by binding the cell surface antigen expressed by the effector T cell.

In one embodiment, the effector T cell in the cell synapse kills the tumor or cancer cell by mediating cytotoxic cell killing.

### BRIEF DESCRIPTION OF THE FIGURES

FIG. 1 is a schematic showing a non-limiting embodiment of a protein complex comprising 2 polypeptide chains with two different Fab regions arranged in an in-tandem manner (e.g., Kv6.1).

FIG. 2 is a schematic showing a non-limiting embodiment of a protein complex comprising 3 polypeptide chains with two different Fab regions arranged in an in-tandem manner (e.g., Kv5.1).

FIG. 3 is a schematic showing a non-limiting embodiment of a protein complex comprising 2 polypeptide chains with two different Fab regions arranged in a non-tandem manner (e.g., Kv6.2).

FIG. 4 is a schematic showing a non-limiting embodiment of a protein complex comprising 3 polypeptide chains with two different Fab regions arranged in a non-tandem manner (e.g., Kv4.33).

FIG. 5 is a schematic showing a non-limiting embodiment of a protein complex comprising 2 polypeptide chains with two different Fab regions arranged in an in-tandem manner (e.g., an alternative arrangement).

FIG. 6 is a schematic showing a non-limiting embodiment of a protein complex comprising 2 polypeptide chains with two different Fab regions arranged in an in-tandem manner (e.g., an alternative arrangement).

FIG. 7 is a schematic showing a non-limiting embodiment of a protein complex comprising 2 polypeptide chains with two different Fab regions arranged in an in-tandem manner (e.g., an alternative arrangement).

FIG. 8 is a schematic showing a non-limiting embodiment of a protein complex comprising 2 polypeptide chains with two different Fab regions arranged in a non-tandem manner (e.g., an alternative arrangement).

FIG. 9 is a schematic showing a non-limiting embodiment of a protein complex comprising 2 polypeptide chains with two different Fab regions arranged in a non-tandem manner (e.g., an alternative arrangement).

FIG. 10 is a schematic showing a non-limiting embodiment of a protein complex comprising 2 polypeptide chains with two different Fab regions arranged in a non-tandem manner (e.g., an alternative arrangement).

FIG. 11 is a schematic showing a non-limiting embodiment of a protein complex comprising 3 polypeptide chains with two different Fab regions arranged in an in-tandem manner (e.g., an alternative arrangement).

FIG. 12 is a schematic showing a non-limiting embodiment of a protein complex comprising 3 polypeptide chains with two different Fab regions arranged in an in-tandem manner (e.g., an alternative arrangement).

FIG. 13 is a schematic showing a non-limiting embodiment of a protein complex comprising 3 polypeptide chains with two different Fab regions arranged in an in-tandem manner (e.g., an alternative arrangement).

FIG. 14 is a schematic showing a non-limiting embodiment of a protein complex comprising 3 polypeptide chains with two different Fab regions arranged in a non-tandem manner (e.g., an alternative arrangement).

FIG. 15 is a schematic showing a non-limiting embodiment of a protein complex comprising 3 polypeptide chains with two different Fab regions arranged in a non-tandem manner (e.g., an alternative arrangement).

FIG. 16 is a schematic showing a non-limiting embodiment of a protein complex comprising 3 polypeptide chains with two different Fab regions arranged in a non-tandem manner (e.g., an alternative arrangement).

FIG. 17A is an SPR sensorgram showing binding of a bispecific antibody with EGFR. The bispecific antibody comprises a 3-chain non-tandem protein complex (Kv4.33) having an anti-EGFR first Fab region and an anti-PD-L1 second Fab region.

FIG. 17B is an SPR sensorgram showing binding of a bispecific antibody with EGFR. The bispecific antibody comprises a 3-chain in-tandem protein complex (Kv5.1) having an anti-EFGR first Fab region and an anti-PD-L1 second Fab region.

FIG. 17C is an SPR sensorgram showing binding of a control anti-EGFR antibody (a mouse-human chimeric MoAb; see FIG. 43) with EGFR.

FIG. 17D is an SPR sensorgram showing binding of an anti-EGFR mAb (parental antibody) with EGFR.

FIG. 18A is an SPR sensorgram showing binding of a bispecific antibody with PD-L1. The bispecific antibody comprises a 3-chain non-tandem protein complex (Kv4.33) having an anti-EGFR first Fab region and an anti-PD-L1 second Fab region.

FIG. 18B is an SPR sensorgram showing binding of a bispecific antibody with PD-L1. The bispecific antibody comprises a 3-chain in-tandem protein complex (Kv5.1) having an anti-EFGR first Fab region and an anti-PD-L1 second Fab region.

FIG. 18C is an SPR sensorgram showing binding of an anti-PD-L1 mAb (parental antibody) with PD-L1.

FIG. 19A is an SPR sensorgram showing binding of a bispecific antibody with CD38. The bispecific antibody comprises a 3-chain non-tandem protein complex (Kv4.33) having an anti-CD38 first Fab region and an anti-CD3 second Fab region.

FIG. 19B is an SPR sensorgram showing binding of a bispecific antibody with CD38. The bispecific antibody comprises a 3-chain in-tandem protein complex (Kv5.1) having an anti-CD38 first Fab region and an anti-CD3 second Fab region.

FIG. 19C is an SPR sensorgram showing binding of an anti-CD38 mAb (parental antibody) with CD38.

FIG. 19D is an SPR sensorgram showing binding of a bispecific antibody with CD38. The bispecific antibody comprises a 2-chain in-tandem protein complex (Kv6.1) having an anti-CD38 first Fab region and an anti-CD3 second Fab region.

FIG. 19E is an SPR sensorgram showing binding of a bispecific antibody with CD38. The bispecific antibody comprises a 2-chain non-tandem protein complex (Kv6.2) having an anti-CD38 first Fab region and an anti-CD3 second Fab region.

FIG. 20A is an SPR sensorgram showing binding of a bispecific antibody with BCMA. The bispecific antibody comprises a 3-chain non-tandem protein complex (Kv4.33) having an anti-BCMA first Fab region and an anti-CD3 second Fab region.

FIG. 20B is an SPR sensorgram showing binding of an anti-BCMA mAb (parental antibody) with BCMA.

FIG. 21A is an SPR sensorgram showing binding of a bispecific antibody with immobilized EGFR. The bispecific antibody comprises a 3-chain non-tandem protein complex (Kv4.33) having an anti-EGFR first Fab region and an anti-PD-L1 second Fab region.

FIG. 21B is an SPR sensorgram showing binding of a bispecific antibody with immobilized EGFR. The bispecific antibody comprises a 3-chain in-tandem protein complex (Kv5.1) having an anti-EFGR first Fab region and an anti-PD-L1 second Fab region.

FIG. 21C is an SPR sensorgram showing binding of a bispecific antibody with immobilized PD-L1. The bispecific antibody comprises a 3-chain non-tandem protein complex (Kv4.33) having an anti-EGFR first Fab region and an anti-PD-L1 second Fab region.

FIG. 21D is an SPR sensorgram showing binding of a bispecific antibody with immobilized PD-L1. The bispecific antibody comprises a 3-chain in-tandem protein complex (Kv5.1) having an anti-EFGR first Fab region and an anti-PD-L1 second Fab region.

FIG. 22A is an SPR sensorgram showing binding of a bispecific antibody with both EGFR and PD-L1 antigen binding domains. The EGFR antigen is immobilized, which binds a bispecific antibody comprising a 3-chain in-tandem protein complex (Kv5.1) having an anti-EFGR first Fab region and an anti-PD-L1 second Fab region, and the PD-L1 antigen is in the mobile phase.

FIG. 22B is reference-subtracted sensorgram of the rectangle area from FIG. 22A.

FIG. 23A is an SPR sensorgram showing binding of a bispecific antibody with both EGFR and PD-L1 antigen binding domains. The PD-L1 antigen is immobilized, which binds a bispecific antibody comprising a 3-chain in-tandem protein complex (Kv5.1) having an anti-EFGR first Fab region and an anti-PD-L1 second Fab region, and the EGFR antigen is in the mobile phase.

FIG. 23B is reference-subtracted sensorgram of the rectangle area from FIG. 23A.

FIG. 24A is an anti-human Fc Western blot (reducing condition) of various two chain in-tandem (Kv6.1) CD38/CD3 bispecific antibodies each carrying a different cleavable linker, and cleaved with MMP9 protease for 1 hour. The arrows designate the cleavage product. The cleavable linker sequences for EL EJ, EK, EL EM, EN, EO, EP, EQ, ER, ES, ET and EG are listed in Table 1 in Example 4.

FIG. 24B is an anti-human Fc Western blot (reducing condition) of various two chain non-tandem (Kv6.2) CD38/CD3 bispecific antibodies each carrying a different cleavable linker, and cleaved with MMP9 protease for 1 hour. The arrows designate the cleavage product. The cleavable linker sequences for EU, EV, EW, EX, EY, EZ, FA, FB, FC, FD, FE, FF and EH are listed in Table 1 in Example 4.

FIG. 24C is an anti-human Fc Western blot (reducing condition) of various two chain non-tandem (Kv6.2) CD38/CD3 bispecific antibodies each carrying a different cleavable linker, and cleaved with MMP9 protease for 2 hours. The arrows designate the cleavage product. The cleavable linker sequences for EU, EV, EW, EX, EY, EZ, FA, FB, FC, FD, FE, FF and EH are listed in Table 1 in Example 4.

FIG. 24D is an anti-human Fc Western blot (reducing condition) of various two chain non-tandem (Kv6.2) CD38/CD3 bispecific antibodies each carrying a different cleavable linker, and cleaved with MMP2 protease for 1 hour. The arrows designate the cleavage product. The cleavable linker sequences for EU, EV, EW, EX, EY, EZ, FA, FB, FC, FD, FE, FF and EH are listed in Table 1 in Example 4.

FIG. 24E is an anti-human Fc Western blot (reducing condition) of various two chain non-tandem (Kv6.2) CD38/CD3 bispecific antibodies each carrying a different cleavable linker, and cleaved with MMP2 protease for 2 hours. The arrows designate the cleavage product. The cleavable linker sequences for EU, EV, EW, EX, EY, EZ, FA, FB, FC, FD, FE, FF and EH are listed in Table 1 in Example 4.

FIG. 25A shows dose-dependent binding curves of various intact two-chain and three-chain, and in-tandem and non-tandem, CD38/CD3 bispecific antibodies binding to CD3-expressing T cells.

FIG. 25B shows dose-dependent binding curves of an intact BCMA/CD3 bispecific antibody (three-chain non-tandem; Kv4.33) binding to CD3-expressing T cells.

FIG. 25C shows dose-dependent binding curves of intact BCMA/CD3 bispecific antibodies (three-chain tandem Kv5.1 and non-tandem Kv4.33) binding to CD3-expressing T cells.

FIG. 26A shows dose-dependent binding curves of various two-chain in-tandem (Kv6.1) CD38/CD3 bispecific antibodies each carrying a different cleavable linker sequence, cleaved with MMP9 protease or intact, binding to CD3-expressing T cells. The binding capability of a three-chain (Kv5.1) in-tandem bispecific antibody is included for comparison.

FIG. 26B shows dose-dependent binding curves of various two-chain non-tandem (Kv6.2) CD38/CD3 bispecific antibodies each carrying a different cleavable linker sequence, cleaved with MMP9 protease or intact, binding to CD3-expressing T cells. The binding capability of a three-chain (Kv4.33) non-tandem bispecific antibody is included for comparison.

FIG. 27A shows an IFNγ release profile of three-chain in-tandem (Kv5.1) and non-tandem (Kv4.33) CD38/CD3 bispecific antibodies from an antibody-mediated tumor associated antigen-dependent T cell cytotoxicity assay.

FIG. 27B shows an IL2 release profile of three-chain in-tandem (Kv5.1) and non-tandem (Kv4.33) CD38/CD3 bispecific antibodies from an antibody-mediated tumor associated antigen-dependent T cell cytotoxicity assay.

FIG. 27C shows an TFNα release profile of three-chain in-tandem (Kv5.1) and non-tandem (Kv4.33) CD38/CD3 bispecific antibodies from an antibody-mediated tumor associated antigen-dependent T cell cytotoxicity assay.

FIG. 27D is a bar graph showing a side-by-side comparison of cytokine release profiles of the three-chain in-tandem (Kv5.1) and non-tandem (Kv4.33) CD38/CD3 bispecific antibodies from the antibody-mediated tumor associated antigen-dependent T cell cytotoxicity assays shown in FIGS. 27A-C.

FIG. 28A is a bar graph showing the results of an in vitro dose response T cell activation assay testing different concentrations of three chain in-tandem (Kv5.1) and non-tandem (Kv4.33), BCMA/CD3 and CD38/CD3, bispecific antibodies.

FIG. 28B is a bar graph showing the results of an in vitro dose response T cell activation assay, in the absence of tumor cells, and testing different concentrations of three chain in-tandem (Kv5.1) and non-tandem (Kv4.33), BCMA/CD3 and CD38/CD3, bispecific antibodies.

FIG. 28C is a bar graph showing the results of an in vitro dose response T cell activation assay that also shows target specificity of a three chain non-tandem (Kv4.33) BCMA/CD3 bispecific antibody.

FIG. 28D is a bar graph showing the results of an in vitro dose response T cell activation assay that also shows target specificity of a three chain non-tandem (Kv4.33) CD38/CD3 bispecific antibody.

FIG. 28E is a bar graph showing the results of an in vitro dose response T cell activation assay that also shows target specificity of a three chain in-tandem (Kv5.1) CD38/CD3 bispecific antibody.

FIG. 28F is a bar graph showing the results of an in vitro dose response T cell activation assay of a mixture of control parental anti-BCMA mAb and anti-CD3 mAb.

FIG. 28G is a bar graph showing the results of an in vitro dose response T cell activation assay of a mixture of control parental anti-CD38 mAb and anti-CD3 mAb.

FIG. 29A shows the results of an in vitro tumor associated antigen (TAA) dependent T cell cytotoxicity assay comparing the killing activity of RPMI8226 cells by two different three chain bispecific antibodies that bind CD38/CD3: in tandem (Kv4.33) and non-tandem (Kv5.1).

FIG. 29B shows the results of an in vitro tumor associated antigen (TAA) dependent T cell cytotoxicity assay comparing the killing activity of MM1.R cells by two different three chain bispecific antibodies that bind CD38/CD3 or BCMA/CD3: in tandem (Kv4.33) and non-tandem (Kv5.1). The killing activity of a combination of anti-BCMA mAb and anti-CD3 mAb is included for comparison.

FIG. 29C shows the results of an in vitro tumor associated antigen (TAA) dependent T cell cytotoxicity assay comparing the killing activity of MM1.R cells by three different three chain bispecific antibodies that bind CD38/CD3 or BCMA/CD3: in tandem (Kv4.33) and non-tandem (Kv5.1). The killing activity of a combination of anti-CD38 mAb and anti-CD3 mAb is included for comparison.

FIG. 30A is a bar graph showing the results of CD38 expression level characterization of six cell lines transduced to express GFP and firefly luciferase using flow cytometry.

FIG. 30B is a graph showing donor effector cell cytotoxicity T cell and NK cell quantification by flow cytometry.

FIG. 30C is a series of 6 graphs showing the results of in vitro dose-dependent cytotoxicity of CD38/CD3 three-chain bispecific antibody (in-tandem Kv5.1) compared to Darzalex against CD38(+) tumor cell lines by cytotoxic T cells or NK cells.

FIG. 31A is the amino acid sequences of various regions of the first polypeptide chain of a two chain in-tandem bispecific antibody (Kv6.1) that binds CD38 and CD3.

FIG. 31B is the amino acid sequences of various regions of the second polypeptide chain of the two chain in-tandem bispecific antibody (Kv6.2) shown in FIG. 31A including embodiments of different second linker sequences.

FIG. 31C is a continuation of FIG. 31B showing the amino acid sequences of various regions of the second polypeptide chain of the two chain in-tandem bispecific antibody (Kv6.2).

FIG. 32A is the amino acid sequences of various regions of the first polypeptide chain of a two chain non-tandem bispecific antibody (Kv6.2) that binds CD38 and CD3.

FIG. 32B is the amino acid sequences of various regions of the second polypeptide chain of the two chain non-tandem bispecific antibody (Kv6.2) shown in FIG. 32A including embodiments of different second linker sequences.

FIG. 32C is a continuation of FIG. 32B showing the amino acid sequences of various regions of the second polypeptide chain of the two chain non-tandem bispecific antibody (Kv6.2).

FIG. 33A is the amino acid sequences of various regions of the first polypeptide chain of a three chain in-tandem bispecific antibody (Kv5.1) that binds CD38 and CD3.

FIG. 33B is the amino acid sequences of various regions of the second and third polypeptides chain of the two chain in-tandem bispecific antibody (Kv5.1) shown in FIG. 33A.

FIG. 34A is the amino acid sequences of various regions of the first polypeptide chain of a three chain non-tandem bispecific antibody (Kv4.33) that binds CD38 and CD3.

FIG. 34B is the amino acid sequences of various regions of the second and third polypeptides chain of the two chain non-tandem bispecific antibody (Kv4.33) shown in FIG. 33A.

FIG. 35A is the amino acid sequences of various regions of the first polypeptide chain of a three chain non-tandem bispecific antibody (Kv4.33) that binds BCMA and CD3.

FIG. 35B is the amino acid sequences of various regions of the second and third polypeptides chain of the two chain non-tandem bispecific antibody (Kv4.33) shown in FIG. 35A.

FIG. 36A is the amino acid sequences of various regions of the first polypeptide chain of a three chain in-tandem bispecific antibody (Kv5.1) that binds EGFR and PD-L1.

FIG. 36B is the amino acid sequences of various regions of the second and third polypeptides chain of the two chain in-tandem bispecific antibody (Kv5.1) shown in FIG. 36A.

FIG. 37A is the amino acid sequences of various regions of the first polypeptide chain of a three chain non-tandem bispecific antibody (Kv4.33) that binds PD-L1 and EGFR.

FIG. 37B is the amino acid sequences of various regions of the second and third polypeptides chain of the two chain non-tandem bispecific antibody (Kv4.33) shown in FIG. 37A.

FIG. 38A is the amino acid sequences of various regions of the first polypeptide chain of a three chain in-tandem bispecific antibody (Kv5.1) that binds BCMA and CD3.

FIG. 38B is the amino acid sequences of various regions of the second and third polypeptides chain of the two chain in-tandem bispecific antibody (Kv5.1) shown in FIG. 38A.

### DETAILED DESCRIPTION

### Definitions

Unless defined otherwise, technical and scientific terms used herein have meanings that are commonly understood by those of ordinary skill in the art unless defined otherwise. Generally, terminologies pertaining to techniques of cell and tissue culture, molecular biology, immunology, microbiology, genetics, transgenic cell production, protein chemistry and nucleic acid chemistry and hybridization described herein are well known and commonly used in the art. The methods and techniques provided herein are generally performed according to conventional procedures well known in the art and as described in various general and more specific references that are cited and discussed herein unless otherwise indicated. See, e.g., Sambrook et al. Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992). A number of basic texts describe standard antibody production processes, including, Borrebaeck (ed) Antibody Engineering, 2nd Edition Freeman and Company, N Y, 1995; McCafferty et al. Antibody Engineering, A Practical Approach IRL at Oxford Press, Oxford, England, 1996; and Paul (1995) Antibody Engineering Protocols Humana Press, Towata, N.J., 1995; Paul (ed.), Fundamental Immunology, Raven Press, N.Y, 1993; Coligan (1991) Current Protocols in Immunology Wiley/Greene, NY; Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Coding Monoclonal Antibodies: Principles and Practice (2nd ed.) Academic Press, New York, N.Y., 1986, and Kohler and Milstein Nature 256: 495-497, 1975. All of the references cited herein are incorporated herein by reference in their entireties. Enzymatic reactions and enrichment/purification techniques are also well known and are performed according to manufacturer's specifications, as commonly accomplished in the art or as described herein. The terminology used in connection with, and the laboratory procedures and techniques of, analytical chemistry, synthetic organic chemistry, and medicinal and pharmaceutical chemistry described herein are well known and commonly used in the art. Standard techniques can be used for chemical syntheses, chemical analyses, pharmaceutical preparation, formulation, and delivery, and treatment of patients.

The headings provided herein are not limitations of the various aspects of the disclosure, which aspects can be understood by reference to the specification as a whole.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative (e.g., “or”) herein is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

As used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition.

The terms “peptide”, “polypeptide” and “protein” and other related terms used herein are used interchangeably and refer to a polymer of amino acids and are not limited to any particular length. Polypeptides may comprise natural and non-natural amino acids. Polypeptides include recombinant or chemically-synthesized forms. Polypeptides also include precursor molecules that have not yet been subjected to cleavage, for example cleavage by a secretory signal peptide or by non-enzymatic cleavage at certain amino acid residues. Polypeptides include mature molecules that have undergone cleavage. These terms encompass native and artificial proteins, protein fragments and polypeptide analogs (such as muteins, variants, chimeric proteins and fusion proteins) of a protein sequence as well as post-translationally, or otherwise covalently or non-covalently, modified proteins. Two or more polypeptides (e.g., 2 or 3 polypeptide chains) can associate with each other, via covalent and/or non-covalent association, to form a multimeric polypeptide complex (e.g., multi-specific antigen binding protein complex). Association of the polypeptide chains can also include peptide folding. Thus, a polypeptide complex can be dimeric, trimeric, tetrameric, or higher order complexes depending on the number of polypeptide chains that form the complex.

The terms “nucleic acid”, “polynucleotide” and “oligonucleotide” and other related terms used herein are used interchangeably and refer to polymers of nucleotides and are not limited to any particular length. Nucleic acids include recombinant and chemically-synthesized forms. Nucleic acids include DNA molecules (cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. Nucleic acid molecule can be single-stranded or double-stranded. In one embodiment, the nucleic acid molecules of the disclosure comprise a contiguous open reading frame encoding an antibody, or a fragment or scFv, derivative, mutein, or variant thereof.

The term “recover” or “recovery” or “recovering”, and other related terms, refers to obtaining a protein (e.g., an antibody or an antigen binding portion thereof), from host cell culture medium or from host cell lysate or from the host cell membrane. In one embodiment, the protein is expressed by the host cell as a recombinant protein fused to a secretion signal peptide sequence which mediates secretion of the expressed protein. The secreted protein can be recovered from the host cell medium. In one embodiment, the protein is expressed by the host cell as a recombinant protein that lacks a secretion signal peptide sequence which can be recovered from the host cell lysate. In one embodiment, the protein is expressed by the host cell as a membrane-bound protein which can be recovered using a detergent to release the expressed protein from the host cell membrane. In one embodiment, irrespective of the method used to recover the protein, the protein can be subjected to procedures that remove cellular debris from the recovered protein. For example, the recovered protein can be subjected to chromatography, gel electrophoresis and/or dialysis. In one embodiment, the chromatography comprises any one or any combination or two or more procedures including affinity chromatography, hydroxyapatite chromatography, ion-exchange chromatography, reverse phase chromatography and/or chromatography on silica. In one embodiment, affinity chromatography comprises protein A or G (cell wall components from Staphylococcus aureus).

The term “isolated” refers to a protein (e.g., an antibody or an antigen binding portion thereof) or polynucleotide that is substantially free of other cellular material. A protein may be rendered substantially free of naturally associated components (or components associated with a cellular expression system or chemical synthesis methods used to produce the antibody) by isolation, using protein purification techniques well known in the art. The term isolated also refers in some embodiments to protein or polynucleotides that are substantially free of other molecules of the same species, for example other protein or polynucleotides having different amino acid or nucleotide sequences, respectively. The purity of homogeneity of the desired molecule can be assayed using techniques well known in the art, including low resolution methods such as gel electrophoresis and high resolution methods such as HPLC or mass spectrophotometry. In one embodiment, the multi-specific antigen binding protein complexes, or antigen binding portions thereof, of the present disclosure are isolated.

An “antigen binding protein” and related terms used herein refers to a protein comprising a portion that binds to an antigen and, optionally, a scaffold or framework portion that allows the antigen binding portion to adopt a conformation that promotes binding of the antigen binding protein to the antigen. Examples of antigen binding proteins include antibodies, antibody fragments (e.g., an antigen binding portion of an antibody), antibody derivatives, and antibody analogs. The antigen binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen binding protein as well as wholly synthetic scaffolds comprising, for example, a biocompatible polymer. See, for example, Korndorfer et al., 2003, Proteins: Structure, Function, and Bioinformatics, Volume 53, Issue 1:121-129; Roque et al., 2004, Biotechnol. Prog. 20:639-654. In addition, peptide antibody mimetics (“PAMs”) can be used, as well as scaffolds based on antibody mimetics utilizing fibronection components as a scaffold.

An antigen binding protein can have, for example, the structure of an immunoglobulin. In one embodiment, an “immunoglobulin” refers to a tetrameric molecule composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa or lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site such that an intact immunoglobulin has two antigen binding sites. In one embodiment, an antigen binding protein can be a synthetic molecule having a structure that differs from a tetrameric immunoglobulin molecule but still binds a target antigen or binds two or more target antigens. For example, a synthetic antigen binding protein can comprise antibody fragments, 1-6 or more polypeptide chains, asymmetrical assemblies of polypeptides, or other synthetic molecules.

The variable regions of immunoglobulin chains exhibit the same general structure of relatively conserved framework regions (FR) joined by three hypervariable regions, also called complementarity determining regions or CDRs. From N-terminus to C-terminus, both light and heavy chains comprise the domains FR1, CDR1, FR2, CDR2, FR3, CDR3 and FR4.

One or more CDRs may be incorporated into a molecule either covalently or noncovalently to make it an antigen binding protein. An antigen binding protein may incorporate the CDR(s) as part of a larger polypeptide chain, may covalently link the CDR(s) to another polypeptide chain, or may incorporate the CDR(s) noncovalently. The CDRs permit the antigen binding protein to specifically bind to a particular antigen of interest.

The assignment of amino acids to each domain is in accordance with the definitions of Kabat et al. in Sequences of Proteins of Immunological Interest, 5′ Ed., US Dept. of Health and Human Services, PHS, NIH, NIH Publication no. 91-3242, 1991 (“Kabat numbering”). Other numbering systems for the amino acids in immunoglobulin chains include IMGT® (international ImMunoGeneTics information system; Lefranc et al, Dev. Comp. Immunol. 29:185-203; 2005) and AHo (Honegger and Pluckthun, J. Mol. Biol. 309(3):657-670; 2001); Chothia (Al-Lazikani et al., 1997 Journal of Molecular Biology 273:927-948; Contact (Maccallum et al., 1996 Journal of Molecular Biology 262:732-745, and Aho (Honegger and Pluckthun 2001 Journal of Molecular Biology 309:657-670.

An “antibody” and “antibodies” and related terms used herein refers to an intact immunoglobulin or to an antigen binding portion thereof that binds specifically to an antigen. Antigen binding portions may be produced by recombinant DNA techniques or by enzymatic or chemical cleavage of intact antibodies. Antigen binding portions include, inter alia, Fab, Fab′, F(ab′)2, Fv, domain antibodies (dAbs), and complementarity determining region (CDR) fragments, single-chain antibodies (scFv), chimeric antibodies, diabodies, triabodies, tetrabodies, and polypeptides that contain at least a portion of an immunoglobulin that is sufficient to confer specific antigen binding to the polypeptide.

Antibodies include recombinantly produced antibodies and antigen binding portions. Antibodies include non-human, chimeric, humanized and fully human antibodies. Antibodies include monospecific, multispecific (e.g., bispecific, trispecific and higher order specificities). Antibodies include tetrameric antibodies, light chain monomers, heavy chain monomers, light chain dimers, heavy chain dimers. Antibodies include F(ab′)2 fragments, Fab′ fragments and Fab fragments. Antibodies include single domain antibodies, monovalent antibodies, single chain antibodies, single chain variable fragment (scFv), camelized antibodies, affibodies, disulfide-linked Fvs (sdFv), anti-idiotypic antibodies (anti-Id), minibodies. Antibodies include monoclonal and polyclonal populations.

An “antigen binding domain,” “antigen binding region,” or “antigen binding site” and other related terms used herein refer to a portion of an antigen binding protein that contains amino acid residues (or other moieties) that interact with an antigen and contribute to the antigen binding protein's specificity and affinity for the antigen. For an antibody that specifically binds to its antigen, this will include at least part of at least one of its CDR domains.

The terms “specific binding”, “specifically binds” or “specifically binding” and other related terms, as used herein in the context of an antibody or antigen binding protein (e.g., multi-specific antigen binding protein complex) or antibody fragment, refer to non-covalent or covalent preferential binding to an antigen relative to other molecules or moieties (e.g., an antibody specifically binds to a particular antigen relative to other available antigens). In one embodiment, an antibody specifically binds to a target antigen if it binds to the antigen with a dissociation constant K_{D} of 10⁻⁵ M or less, or 10⁻⁷ M or less, or 10⁻⁷ M or less, or 10⁻⁸ M or less, or 10⁻⁹ M or less, or 10⁻¹⁰ M or less.

In one embodiment, a dissociation constant (K_{D}) can be measured using a BIACORE surface plasmon resonance (SPR) assay. Surface plasmon resonance refers to an optical phenomenon that allows for the analysis of real-time interactions by detection of alterations in protein concentrations within a biosensor matrix, for example using the BIACORE system (Biacore Life Sciences division of GE Healthcare, Piscataway, N.J.).

An “epitope” and related terms as used herein refers to a portion of an antigen that is bound by an antigen binding protein (e.g., by an antibody or an antigen binding portion thereof). An epitope can comprise portions of two or more antigens that are bound by an antigen binding protein. An epitope can comprise non-contiguous portions of an antigen or of two or more antigens (e.g., amino acid residues that are not contiguous in an antigen's primary sequence but that, in the context of the antigen's tertiary and quaternary structure, are near enough to each other to be bound by an antigen binding protein). Generally, the variable regions, particularly the CDRs, of an antibody interact with the epitope.

An “antibody fragment”, “antibody portion”, “antigen-binding fragment of an antibody”, or “antigen-binding portion of an antibody” and other related terms used herein refer to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)2; Fd; and Fv fragments, as well as dAb; diabodies; linear antibodies; single-chain antibody molecules (e.g. scFv); polypeptides that contain at least a portion of an antibody that is sufficient to confer specific antigen binding to the polypeptide. Antigen binding portions of an antibody may be produced by recombinant DNA techniques or by enzymatic or chemical cleavage of intact antibodies. Antigen binding portions include, inter alia, Fab, Fab′, F(ab′)2, Fv, domain antibodies (dAbs), and complementarity determining region (CDR) fragments, chimeric antibodies, diabodies, triabodies, tetrabodies, and polypeptides that contain at least a portion of an immunoglobulin that is sufficient to confer antigen binding properties to the antibody fragment.

The terms “Fab”, “Fab fragment” and other related terms refers to a monovalent fragment comprising a variable light chain region (V_{L}), constant light chain region (C_{L}), variable heavy chain region (V_{H}), and first constant region (C_{H1}). A half Fab heavy region comprises a variable heavy chain region (V_{H}) and first constant region (C_{H1}). A half Fab light region comprises a variable light chain region (V_{L}) and constant light chain region (C_{L}). A Fab is capable of binding an antigen. An F(ab′)2 fragment is a bivalent fragment comprising two Fab fragments linked by a disulfide bridge at the hinge region. A F(Ab′)₂ has antigen binding capability. An Fd fragment comprises V_{H} and C_{H1} regions. An Fv fragment comprises V_{L} and V_{H} regions. An Fv can bind an antigen. A dAb fragment has a V_{H} domain, a V_{L} domain, or an antigen-binding fragment of a V_{H} or V_{L} domain (U.S. Pat. Nos. 6,846,634 and 6,696,245; U.S. published Application Nos. 2002/02512, 2004/0202995, 2004/0038291, 2004/0009507, 2003/0039958; and Ward et al., Nature 341:544-546, 1989).

A single-chain antibody (scFv) is an antibody in which a V_{L} and a V_{H} region are joined via a linker (e.g., a synthetic sequence of amino acid residues) to form a continuous protein chain. Preferably the linker is long enough to allow the protein chain to fold back on itself and form a monovalent antigen binding site (see, e.g., Bird et al., 1988, Science 242:423-26 and Huston et al., 1988, Proc. Natl. Acad. Sci. USA 85:5879-83).

Diabodies are bivalent antibodies comprising two polypeptide chains, wherein each polypeptide chain comprises V_{H} and V_{L} domains joined by a linker that is too short to allow for pairing between two domains on the same chain, thus allowing each domain to pair with a complementary domain on another polypeptide chain (see, e.g., Holliger et al., 1993, Proc. Natl. Acad. Sci. USA 90:6444-48, and Poljak et al., 1994, Structure 2:1121-23). If the two polypeptide chains of a diabody are identical, then a diabody resulting from their pairing will have two identical antigen binding sites. Polypeptide chains having different sequences can be used to make a diabody with two different antigen binding sites. Similarly, tribodies and tetrabodies are antibodies comprising three and four polypeptide chains, respectively, and forming three and four antigen binding sites, respectively, which can be the same or different.

The term “human antibody” refers to antibodies that have one or more variable and constant regions derived from human immunoglobulin sequences. In one embodiment, all of the variable and constant domains are derived from human immunoglobulin sequences (e.g., a fully human antibody). These antibodies may be prepared in a variety of ways, examples of which are described below, including through recombinant methodologies or through immunization with an antigen of interest of a mouse that is genetically modified to express antibodies derived from human heavy and/or light chain-encoding genes.

A “humanized” antibody refers to an antibody having a sequence that differs from the sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In one embodiment, certain amino acids in the framework and constant domains of the heavy and/or light chains of the non-human species antibody are mutated to produce the humanized antibody. In another embodiment, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its antigen, or the changes to the amino acid sequence that are made are conservative changes, such that the binding of the humanized antibody to the antigen is not significantly worse than the binding of the non-human antibody to the antigen. Examples of how to make humanized antibodies may be found in U.S. Pat. Nos. 6,054,297, 5,886,152 and 5,877,293.

The term “chimeric antibody” and related terms used herein refers to an antibody that contains one or more regions from a first antibody and one or more regions from one or more other antibodies. In one embodiment, one or more of the CDRs are derived from a human antibody. In another embodiment, all of the CDRs are derived from a human antibody. In another embodiment, the CDRs from more than one human antibody are mixed and matched in a chimeric antibody. For instance, a chimeric antibody may comprise a CDR1 from the light chain of a first human antibody, a CDR2 and a CDR3 from the light chain of a second human antibody, and the CDRs from the heavy chain from a third antibody. In another example, the CDRs originate from different species such as human and mouse, or human and rabbit, or human and goat. One skilled in the art will appreciate that other combinations are possible.

Further, the framework regions may be derived from one of the same antibodies, from one or more different antibodies, such as a human antibody, or from a humanized antibody. In one example of a chimeric antibody, a portion of the heavy and/or light chain is identical with, homologous to, or derived from an antibody from a particular species or belonging to a particular antibody class or subclass, while the remainder of the chain(s) is/are identical with, homologous to, or derived from an antibody (-ies) from another species or belonging to another antibody class or subclass. Also included are fragments of such antibodies that exhibit the desired biological activity (i.e., the ability to specifically bind a target antigen).

As used herein, the term “variant” polypeptides and “variants” of polypeptides refers to a polypeptide comprising an amino acid sequence with one or more amino acid residues inserted into, deleted from and/or substituted into the amino acid sequence relative to a reference polypeptide sequence. Polypeptide variants include fusion proteins. In the same manner, a variant polynucleotide comprises a nucleotide sequence with one or more nucleotides inserted into, deleted from and/or substituted into the nucleotide sequence relative to another polynucleotide sequence. Polynucleotide variants include fusion polynucleotides.

As used herein, the term “derivative” of a polypeptide is a polypeptide (e.g., an antibody) that has been chemically modified, e.g., via conjugation to another chemical moiety such as, for example, polyethylene glycol, albumin (e.g., human serum albumin), phosphorylation, and glycosylation. Unless otherwise indicated, the term “antibody” includes, in addition to antibodies comprising two full-length heavy chains and two full-length light chains, derivatives, variants, fragments, and muteins thereof, examples of which are described below.

The term “Fc” or “Fc region” as used herein refers to the portion of an antibody heavy chain constant region beginning in or after the hinge region and ending at the C-terminus of the heavy chain. The Fc region comprises at least a portion of the CH2 and CH3 regions, and may or may not include a portion of the hinge region. Two polypeptide chains each carrying a half Fc region can dimerize to form an Fc region. An Fc region can bind Fc cell surface receptors and some proteins of the immune complement system. An Fc region exhibits effector function, including any one or any combination of two or more activities including complement-dependent cytotoxicity (CDC), antibody-dependent cell-mediated cytotoxicity (ADCC), antibody-dependent phagocytosis (ADP), opsonization and/or cell binding. An Fc region can bind an Fc receptor, including FcγRI (e.g., CD64), FcγRII (e.g., CD32) and/or FcγRIII (e.g., CD16a).

The term “labeled antibody” or related terms as used herein refers to antibodies and their antigen binding portions thereof that are unlabeled or joined to a detectable label or moiety for detection, wherein the detectable label or moiety is radioactive, colorimetric, antigenic, enzymatic, a detectable bead (such as a magnetic or electrodense (e.g., gold) bead), biotin, streptavidin or protein A. A variety of labels can be employed, including, but not limited to, radionuclides, fluorescers, enzymes, enzyme substrates, enzyme cofactors, enzyme inhibitors and ligands (e.g., biotin, haptens).

The “percent identity” or “percent homology” and related terms used herein refers to a quantitative measurement of the similarity between two polypeptide or between two polynucleotide sequences. The percent identity between two polypeptide sequences is a function of the number of identical amino acids at aligned positions that are shared between the two polypeptide sequences, taking into account the number of gaps, and the length of each gap, which may need to be introduced to optimize alignment of the two polypeptide sequences. In a similar manner, the percent identity between two polynucleotide sequences is a function of the number of identical nucleotides at aligned positions that are shared between the two polynucleotide sequences, taking into account the number of gaps, and the length of each gap, which may need to be introduced to optimize alignment of the two polynucleotide sequences. A comparison of the sequences and determination of the percent identity between two polypeptide sequences, or between two polynucleotide sequences, may be accomplished using a mathematical algorithm. For example, the “percent identity” or “percent homology” of two polypeptide or two polynucleotide sequences may be determined by comparing the sequences using the GAP computer program (a part of the GCG Wisconsin Package, version 10.3 (Accelrys, San Diego, Calif.)) using its default parameters. Expressions such as “comprises a sequence with at least X % identity to Y” with respect to a test sequence mean that, when aligned to sequence Y as described above, the test sequence comprises residues identical to at least X % of the residues of Y.

In one embodiment, the amino acid sequence of a test antibody may be similar but not identical to any of the amino acid sequences of the polypeptides that make up the multi-specific antigen binding protein complexes described herein. The similarities between the test antibody and the polypeptides can be at least 95%, or at or at least 96% identical, or at least 97% identical, or at least 98% identical, or at least 99% identical, to any of the polypeptides that make up the multi-specific antigen binding protein complexes described herein. In one embodiment, similar polypeptides can contain amino acid substitutions within a heavy and/or light chain. In one embodiment, the amino acid substitutions comprise one or more conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference in its entirety. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine.

Antibodies can be obtained from sources such as serum or plasma that contain immunoglobulins having varied antigenic specificity. If such antibodies are subjected to affinity purification, they can be enriched for a particular antigenic specificity. Such enriched preparations of antibodies usually are made of less than about 10% antibody having specific binding activity for the particular antigen. Subjecting these preparations to several rounds of affinity purification can increase the proportion of antibody having specific binding activity for the antigen. Antibodies prepared in this manner are often referred to as “monospecific.” Monospecific antibody preparations can be made up of about 10%, 20%, 30%, 40%, 50%, 60%, 70%, 75%, 80%, 85%, 90%, 95%, 97%, 99%, or 99.9% antibody having specific binding activity for the particular antigen. Antibodies can be produced using recombinant nucleic acid technology as described below.

A “vector” and related terms used herein refers to a nucleic acid molecule (e.g., DNA or RNA) which can be operably linked to foreign genetic material (e.g., nucleic acid transgene). Vectors can be used as a vehicle to introduce foreign genetic material into a cell (e.g., host cell). Vectors can include at least one restriction endonuclease recognition sequence for insertion of the transgene into the vector. Vectors can include at least one gene sequence that confers antibiotic resistance or a selectable characteristic to aid in selection of host cells that harbor a vector-transgene construct. Vectors can be single-stranded or double-stranded nucleic acid molecules. Vectors can be linear or circular nucleic acid molecules. A donor nucleic acid used for gene editing methods employing zinc finger nuclease, TALEN or CRISPR/Cas can be a type of a vector. One type of vector is a “plasmid,” which refers to a linear or circular double stranded extrachromosomal DNA molecule which can be linked to a transgene, and is capable of replicating in a host cell, and transcribing and/or translating the transgene. A viral vector typically contains viral RNA or DNA backbone sequences which can be linked to the transgene. The viral backbone sequences can be modified to disable infection but retain insertion of the viral backbone and the co-linked transgene into a host cell genome. Examples of viral vectors include retroviral, lentiviral, adenoviral, adeno-associated, baculoviral, papovaviral, vaccinia viral, herpes simplex viral and Epstein Barr viral vectors. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome.

An “expression vector” is a type of vector that can contain one or more regulatory sequences, such as inducible and/or constitutive promoters and enhancers. Expression vectors can include ribosomal binding sites and/or polyadenylation sites. Regulatory sequences direct transcription, or transcription and translation, of a transgene linked to the expression vector which is transduced into a host cell. The regulatory sequence(s) can control the level, timing and/or location of expression of the transgene. The regulatory sequence can, for example, exert its effects directly on the transgene, or through the action of one or more other molecules (e.g., polypeptides that bind to the regulatory sequence and/or the nucleic acid). Regulatory sequences can be part of a vector. Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-3606.

A transgene is “operably linked” to a vector when there is linkage between the transgene and the vector to permit functioning or expression of the transgene sequences contained in the vector. In one embodiment, a transgene is “operably linked” to at least one regulatory sequence when the regulatory sequence affects the expression (e.g., the level, timing, or location of expression) of the transgene.

The terms “transfected” or “transformed” or “transduced” or other related terms used herein refer to a process by which exogenous nucleic acid (e.g., transgene) is transferred or introduced into a host cell. A “transfected” or “transformed” or “transduced” host cell is one which has been transfected, transformed or transduced with exogenous nucleic acid (transgene). The host cell includes the primary subject cell and its progeny.

The terms “host cell” or “or a population of host cells” or related terms as used herein refer to a cell (or a population thereof) into which foreign (exogenous or transgene) nucleic acids have been introduced. The foreign nucleic acids can include an expression vector operably linked to a transgene, and the host cell can be used to express the nucleic acid and/or polypeptide encoded by the foreign nucleic acid (transgene). A host cell (or a population thereof) can be a cultured cell or can be extracted from a subject. The host cell (or a population thereof) includes the primary subject cell and its progeny without any regard for the number of passages. Progeny cells may or may not harbor identical genetic material compared to the parent cell. Host cells encompass progeny cells. In one embodiment, a host cell describes any cell (including its progeny) that has been modified, transfected, transduced, transformed, and/or manipulated in any way to express an antibody, as disclosed herein. In one example, the host cell (or population thereof) can be introduced with an expression vector operably linked to a nucleic acid encoding the desired antibody, or an antigen binding portion thereof, described herein. Host cells and populations thereof can harbor an expression vector that is stably integrated into the host's genome, or can harbor an extrachromosomal expression vector. In one embodiment, host cells and populations thereof can harbor an extrachromosomal vector that is present after several cell divisions or is present transiently and is lost after several cell divisions.

Transgenic host cells can be prepared using non-viral methods, including well-known designer nucleases including zinc finger nucleases, TALENS or CRISPR/Cas. A transgene can be introduced into a host cell's genome using genome editing technologies such as zinc finger nuclease. A zinc finger nuclease includes a pair of chimeric proteins each containing a non-specific endonuclease domain of a restriction endonuclease (e.g., FokI) fused to a DNA-binding domain from an engineered zinc finger motif. The DNA-binding domain can be engineered to bind a specific sequence in the host's genome and the endonuclease domain makes a double-stranded cut. The donor DNA carries the transgene, for example any of the nucleic acids encoding a CAR or DAR construct described herein, and flanking sequences that are homologous to the regions on either side of the intended insertion site in the host cell's genome. The host cell's DNA repair machinery enables precise insertion of the transgene by homologous DNA repair. Transgenic mammalian host cells have been prepared using zinc finger nucleases (U.S. Pat. Nos. 9,597,357, 9,616,090, 9,816,074 and 8,945,868). A transgenic host cell can be prepared using TALEN (Transcription Activator-Like Effector Nucleases) which are similar to zinc finger nucleases in that they include a non-specific endonuclease domain fused to a DNA-binding domain which can deliver precise transgene insertion. Like zinc finger nucleases, TALEN also introduce a double-strand cut into the host's DNA. Transgenic host cells can be prepared using CRISPR (Clustered Regularly Interspaced Short Palindromic Repeats). CRISPR employs a Cas endonuclease coupled to a guide RNA for target specific donor DNA integration. The guide RNA includes a conserved multi-nucleotide containing protospacer adjacent motif (PAM) sequence upstream of the gRNA-binding region in the target DNA and hybridizes to the host cell target site where the Cas endonuclease cleaves the double-stranded target DNA. The guide RNA can be designed to hybridize to a specific target site. Similar to zinc finger nuclease and TALEN, the CRISPR/Cas system can be used to introduce site specific insertion of donor DNA having flanking sequences that have homology to the insertion site. Examples of CRISPR/Cas systems used to modify genomes are described for example in U.S. Pat. Nos. 8,697,359, 10,000,772, 9,790,490, and U. S. Patent Application Publication No. US 2018/0346927. In one embodiment, transgenic host cells can be prepared using zinc finger nuclease, TALEN or CRISPR/Cas system, and the host target site can be a TRAC gene (T Cell Receptor Alpha Constant). The donor DNA can include for example any of the nucleic acids encoding a CAR or DAR construct described herein. Electroporation, nucleofection or lipofection can be used to co-deliver into the host cell the donor DNA with the zinc finger nuclease, TALEN or CRISPR/Cas system.

A host cell can be a prokaryote, for example, E. coli, or it can be a eukaryote, for example, a single-celled eukaryote (e.g., a yeast or other fungus), a plant cell (e.g., a tobacco or tomato plant cell), an mammalian cell (e.g., a human cell, a monkey cell, a hamster cell, a rat cell, a mouse cell, or an insect cell) or a hybridoma. In one embodiment, a host cell can be introduced with an expression vector operably linked to a nucleic acid encoding a desired antibody thereby generating a transfected/transformed host cell which is cultured under conditions suitable for expression of the antibody by the transfected/transformed host cell, and optionally recovering the antibody from the transfected/transformed host cells (e.g., recovery from host cell lysate) or recovery from the culture medium. In one embodiment, host cells comprise non-human cells including CHO, BHK, NS0, SP2/0, and YB2/0. In one embodiment, host cells comprise human cells including HEK293, HT-1080, Huh-7 and PER.C6. Examples of host cells include the COS-7 line of monkey kidney cells (ATCC CRL 1651) (see Gluzman et al., 1981, Cell 23: 175), L cells, C127 cells, 3T3 cells (ATCC CCL 163), Chinese hamster ovary (CHO) cells or their derivatives such as Veggie CHO and related cell lines which grow in serum-free media (see Rasmussen et al., 1998, Cytotechnology 28:31) or CHO strain DX-B 11, which is deficient in DHFR (see Urlaub et al., 1980, Proc. Natl. Acad. Sci. USA 77:4216-20), HeLa cells, BHK (ATCC CRL 10) cell lines, the CV1/EBNA cell line derived from the African green monkey kidney cell line CV1 (ATCC CCL 70) (see McMahan et al., 1991, EMBO J. 10:2821), human embryonic kidney cells such as 293, 293 EBNA or MSR 293, human epidermal A431 cells, human Colo 205 cells, other transformed primate cell lines, normal diploid cells, cell strains derived from in vitro culture of primary tissue, primary explants, HL-60, U937, HaK or Jurkat cells. In one embodiment, host cells include lymphoid cells such as Y0, NS0 or Sp20. In one embodiment, a host cell is a mammalian host cell, but is not a human host cell. Typically, a host cell is a cultured cell that can be transformed or transfected with a polypeptide-encoding nucleic acid, which can then be expressed in the host cell. The phrase “transgenic host cell” or “recombinant host cell” can be used to denote a host cell that has been transformed or transfected with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but also to the progeny or potential progeny of such a cell. Because certain modifications may occur in succeeding generations due to, e.g., mutation or environmental influence, such progeny may not, in fact, be identical to the parent cell, but are still included within the scope of the term as used herein.

Polypeptides of the present disclosure (e.g., antibodies and antigen binding proteins) can be produced using any method known in the art. In one example, the polypeptides are produced by recombinant nucleic acid methods by inserting a nucleic acid sequence (e.g., DNA) encoding the polypeptide into a recombinant expression vector which is introduced into a host cell and expressed by the host cell under conditions promoting expression.

General techniques for recombinant nucleic acid manipulations are described for example in Sambrook et al., in Molecular Cloning: A Laboratory Manual, Vols. 1-3, Cold Spring Harbor Laboratory Press, 2 ed., 1989, or F. Ausubel et al., in Current Protocols in Molecular Biology (Green Publishing and Wiley-Interscience: New York, 1987) and periodic updates, herein incorporated by reference in their entireties. The nucleic acid (e.g., DNA) encoding the polypeptide is operably linked to an expression vector carrying one or more suitable transcriptional or translational regulatory elements derived from mammalian, viral, or insect genes. Such regulatory elements include a transcriptional promoter, an optional operator sequence to control transcription, a sequence encoding suitable mRNA ribosomal binding sites, and sequences that control the termination of transcription and translation. The expression vector can include an origin or replication that confers replication capabilities in the host cell. The expression vector can include a gene that confers selection to facilitate recognition of transgenic host cells (e.g., transformants).

The recombinant DNA can also encode any type of protein tag sequence that may be useful for purifying the protein. Examples of protein tags include but are not limited to a histidine tag, a FLAG tag, a myc tag, an HA tag, or a GST tag. Appropriate cloning and expression vectors for use with bacterial, fungal, yeast, and mammalian cellular hosts can be found in Cloning Vectors: A Laboratory Manual, (Elsevier, N.Y., 1985).

The expression vector construct can be introduced into the host cell using a method appropriate for the host cell. A variety of methods for introducing nucleic acids into host cells are known in the art, including, but not limited to, electroporation; transfection employing calcium chloride, rubidium chloride, calcium phosphate, DEAE-dextran, or other substances; viral transfection; non-viral transfection; microprojectile bombardment; lipofection; and infection (e.g., where the vector is an infectious agent). Suitable host cells include prokaryotes, yeast, mammalian cells, or bacterial cells.

Suitable bacteria include gram negative or gram positive organisms, for example, E. coli or Bacillus spp. Yeast, preferably from the Saccharomyces species, such as S. cerevisiae, may also be used for production of polypeptides. Various mammalian or insect cell culture systems can also be employed to express recombinant proteins. Baculovirus systems for production of heterologous proteins in insect cells are reviewed by Luckow and Summers, (Bio/Technology, 6:47, 1988). Examples of suitable mammalian host cell lines include endothelial cells, COS-7 monkey kidney cells, CV-1, L cells, C127, 3T3, Chinese hamster ovary (CHO), human embryonic kidney cells, HeLa, 293, 293T, and BHK cell lines. Purified polypeptides are prepared by culturing suitable host/vector systems to express the recombinant proteins. For many applications, the small size of many of the polypeptides disclosed herein would make expression in E. coli as the preferred method for expression. The protein is then purified from culture media or cell extracts.

Antibodies and antigen binding proteins disclosed herein can also be produced using cell-translation systems. For such purposes the nucleic acids encoding the polypeptide must be modified to allow in vitro transcription to produce mRNA and to allow cell-free translation of the mRNA in the particular cell-free system being utilized (eukaryotic such as a mammalian or yeast cell-free translation system or prokaryotic such as a bacterial cell-free translation system.

Nucleic acids encoding any of the various polypeptides disclosed herein may be synthesized chemically. Codon usage may be selected so as to improve expression in a cell. Such codon usage will depend on the cell type selected. Specialized codon usage patterns have been developed for E. coli and other bacteria, as well as mammalian cells, plant cells, yeast cells and insect cells. See for example: Mayfield et al., Proc. Natl. Acad. Sci. USA. 2003 100(2):438-42; Sinclair et al. Protein Expr. Purif. 2002 (1):96-105; Connell N D. Curr. Opin. Biotechnol. 2001 12(5):446-9; Makrides et al. Microbiol. Rev. 1996 60(3):512-38; and Sharp et al. Yeast. 1991 7(7):657-78.

Antibodies and antigen binding proteins described herein can also be produced by chemical synthesis (e.g., by the methods described in Solid Phase Peptide Synthesis, 2nd ed., 1984, The Pierce Chemical Co., Rockford, Ill.). Modifications to the protein can also be produced by chemical synthesis.

Antibodies and antigen binding proteins described herein can be purified by isolation/purification methods for proteins generally known in the field of protein chemistry. Non-limiting examples include extraction, recrystallization, salting out (e.g., with ammonium sulfate or sodium sulfate), centrifugation, dialysis, ultrafiltration, adsorption chromatography, ion exchange chromatography, hydrophobic chromatography, normal phase chromatography, reversed-phase chromatography, gel filtration, gel permeation chromatography, affinity chromatography, electrophoresis, countercurrent distribution or any combinations of these. After purification, polypeptides may be exchanged into different buffers and/or concentrated by any of a variety of methods known to the art, including, but not limited to, filtration and dialysis.

The purified antibodies and antigen binding proteins described herein are preferably at least 65% pure, at least 75% pure, at least 85% pure, more preferably at least 95% pure, and most preferably at least 98% pure. Regardless of the exact numerical value of the purity, the polypeptide is sufficiently pure for use as a pharmaceutical product.

In certain embodiments, the antibodies and antigen binding proteins herein can further comprise post-translational modifications. Exemplary post-translational protein modifications include phosphorylation, acetylation, methylation, ADP-ribosylation, ubiquitination, glycosylation, carbonylation, sumoylation, biotinylation or addition of a polypeptide side chain or of a hydrophobic group. As a result, the modified polypeptides may contain non-amino acid elements, such as lipids, poly- or mono-saccharide, and phosphates. A preferred form of glycosylation is sialylation, which conjugates one or more sialic acid moieties to the polypeptide. Sialic acid moieties improve solubility and serum half-life while also reducing the possible immunogenicity of the protein. See Raju et al. Biochemistry. 2001 31; 40(30):8868-76.

In one embodiment, the antibodies and antigen binding proteins described herein can be modified to become soluble polypeptides which comprises linking the antibodies and antigen binding proteins to non-proteinaceous polymers. In one embodiment, the non-proteinaceous polymer comprises polyethylene glycol (“PEG”), polypropylene glycol, or polyoxyalkylenes, in the manner as set forth in U.S. Pat. Nos. 4,640,835; 4,496,689; 4,301,144; 4,670,417; 4,791,192 or 4,179,337.

PEG is a water soluble polymer that is commercially available or can be prepared by ring-opening polymerization of ethylene glycol according to methods well known in the art (Sandler and Karo, Polymer Synthesis, Academic Press, New York, Vol. 3, pages 138-161). The term “PEG” is used broadly to encompass any polyethylene glycol molecule, without regard to size or to modification at an end of the PEG, and can be represented by the formula: X—O(CH₂CH₂O)ₙ—CH₂CH₂OH (1), where n is 20 to 2300 and X is H or a terminal modification, e.g., a C₁₋₄ alkyl. In one embodiment, the PEG terminates on one end with hydroxy or methoxy, i.e., X is H or CH₃ (“methoxy PEG”). A PEG can contain further chemical groups which are necessary for binding reactions; which results from the chemical synthesis of the molecule; or which is a spacer for optimal distance of parts of the molecule. In addition, such a PEG can consist of one or more PEG side-chains which are linked together. PEGs with more than one PEG chain are called multiarmed or branched PEGs. Branched PEGs can be prepared, for example, by the addition of polyethylene oxide to various polyols, including glycerol, pentaerythriol, and sorbitol. For example, a four-armed branched PEG can be prepared from pentaerythriol and ethylene oxide. Branched PEG are described in, for example, EP-A 0 473 084 and U.S. Pat. No. 5,932,462. One form of PEGs includes two PEG side-chains (PEG2) linked via the primary amino groups of a lysine (Monfardini et al., Bioconjugate Chem. 6 (1995) 62-69).

The serum clearance rate of PEG-modified polypeptide may be modulated (e.g., increased or decreased) by about 10%, 20%, 30%, 40%, 50%, 60%, 70%, 80%, or even 90%, relative to the clearance rate of the unmodified antibodies and antigen binding proteins binding polypeptides. The PEG-modified antibodies and antigen binding proteins may have a half-life (tv2) which is enhanced relative to the half-life of the unmodified polypeptide. The half-life of PEG-modified polypeptide may be enhanced by at least 10%, 20%, 30%, 40%, 50%, 60%, 70%, 80%, 90%, 100%, 125%, 150%, 175%, 200%, 250%, 300%, 400% or 500%, or even by 1000% relative to the half-life of the unmodified antibodies and antigen binding proteins. In some embodiments, the protein half-life is determined in vitro, such as in a buffered saline solution or in serum. In other embodiments, the protein half-life is an in vivo half-life, such as the half-life of the protein in the serum or other bodily fluid of an animal.

The present disclosure provides therapeutic compositions comprising any of the multi-specific antigen binding protein complexes described herein in an admixture with a pharmaceutically-acceptable excipient. An excipient encompasses carriers, stabilizers and excipients. Excipients of pharmaceutically acceptable excipients includes for example inert diluents or fillers (e.g., sucrose and sorbitol), lubricating agents, glidants, and anti-adhesives (e.g., magnesium stearate, zinc stearate, stearic acid, silicas, hydrogenated vegetable oils, or talc). Additional examples include buffering agents, stabilizing agents, preservatives, non-ionic detergents, anti-oxidants and isotonifiers.

Therapeutic compositions and methods for preparing them are well known in the art and are found, for example, in “Remington: The Science and Practice of Pharmacy” (20th ed., ed. A. R. Gennaro A R., 2000, Lippincott Williams & Wilkins, Philadelphia, Pa.). Therapeutic compositions can be formulated for parenteral administration may, and can for example, contain excipients, sterile water, saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the antibody (or antigen binding protein thereof) described herein. Nanoparticulate formulations (e.g., biodegradable nanoparticles, solid lipid nanoparticles, liposomes) may be used to control the biodistribution of the antibody (or antigen binding protein thereof). Other potentially useful parenteral delivery systems include ethylene-vinyl acetate copolymer particles, osmotic pumps, implantable infusion systems, and liposomes. The concentration of the antibody (or antigen binding protein thereof) in the formulation varies depending upon a number of factors, including the dosage of the drug to be administered, and the route of administration.

The multi-specific antigen binding protein complexes (or antigen binding protein thereof) may be optionally administered as a pharmaceutically acceptable salt, such as non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, and the like. In one example, the antibody (or antigen binding protein thereof) is formulated in the presence of sodium acetate to increase thermal stability.

The multi-specific antigen binding protein complexes (or antigen binding protein thereof) may be formulated for oral use include tablets containing the active ingredient(s) in a mixture with non-toxic pharmaceutically acceptable excipients. Formulations for oral use may also be provided as chewable tablets, or as hard gelatin capsules wherein the active ingredient is mixed with an inert solid diluent, or as soft gelatin capsules wherein the active ingredient is mixed with water or an oil medium.

The term “subject” as used herein refers to human and non-human animals, including vertebrates, mammals and non-mammals. In one embodiment, the subject can be human, non-human primates, simian, ape, murine (e.g., mice and rats), bovine, porcine, equine, canine, feline, caprine, lupine, ranine or piscine.

The term “administering”, “administered” and grammatical variants refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The terms “effective amount”, “therapeutically effective amount” or “effective dose” or related terms may be used interchangeably and refer to an amount of antibody or an antigen binding protein (e.g., multi-specific antigen binding protein complexes) that when administered to a subject, is sufficient to effect a measurable improvement or prevention of a disease or disorder associated with tumor or cancer antigen expression. Therapeutically effective amounts of antibodies provided herein, when used alone or in combination, will vary depending upon the relative activity of the antibodies and combinations (e.g., in inhibiting cell growth) and depending upon the subject and disease condition being treated, the weight and age and sex of the subject, the severity of the disease condition in the subject, the manner of administration and the like, which can readily be determined by one of ordinary skill in the art.

In one embodiment, a therapeutically effective amount will depend on certain aspects of the subject to be treated and the disorder to be treated and may be ascertained by one skilled in the art using known techniques. In general, the polypeptide is administered at about 0.01 g/kg to about 50 mg/kg per day, preferably 0.01 mg/kg to about 30 mg/kg per day, most preferably 0.1 mg/kg to about 20 mg/kg per day. The polypeptide may be administered daily (e.g., once, twice, three times, or four times daily) or preferably less frequently (e.g., weekly, every two weeks, every three weeks, monthly, or quarterly). In addition, as is known in the art, adjustments for age as well as the body weight, general health, sex, diet, time of administration, drug interaction, and the severity of the disease may be necessary.

The present disclosure provides methods for treating a subject having a disease associated with expression (e.g., over-expression) of one or more tumor-associated antigens. The disease comprises cancer or tumor cells expressing the tumor-associated antigens. In one embodiment, the cancer or tumor includes cancer of the prostate, breast, ovary, head and neck, bladder, skin, colorectal, anus, rectum, pancreas, lung (including non-small cell lung and small cell lung cancers), leiomyoma, brain, glioma, glioblastoma, esophagus, liver, kidney, stomach, colon, cervix, uterus, endometrium, vulva, larynx, vagina, bone, nasal cavity, paranasal sinus, nasopharynx, oral cavity, oropharynx, larynx, hypolarynx, salivary glands, ureter, urethra, penis and testis.

In one embodiment, the cancer comprises hematological cancers, including leukemias, lymphomas, myelomas and B cell lymphomas. Hematologic cancers include multiple myeloma (MM), non-Hodgkin's lymphoma (NHL) including Burkitt's lymphoma (BL), B chronic lymphocytic leukemia (B-CLL), systemic lupus erythematosus (SLE), B and T acute lymphocytic leukemia (ALL), acute myeloid leukemia (AML), chronic lymphocytic leukemia (CLL), diffuse large B cell lymphoma, chronic myelogenous leukemia (CML), hairy cell leukemia (HCL), follicular lymphoma, Waldenstrom's Macroglobulinemia, mantle cell lymphoma, Hodgkin's Lymphoma (HL), plasma cell myeloma, precursor B cell lymphoblastic leukemia/lymphoma, plasmacytoma, giant cell myeloma, plasma cell myeloma, heavy-chain myeloma, light chain or Bence-Jones myeloma, lymphomatoid granulomatosis, post-transplant lymphoproliferative disorder, an immunoregulatory disorder, rheumatoid arthritis, myasthenia gravis, idiopathic thrombocytopenia purpura, anti-phospholipid syndrome, Chagas' disease, Grave's disease, Wegener's granulomatosis, poly-arteritis nodosa, Sjogren's syndrome, pemphigus vulgaris, scleroderma, multiple sclerosis, anti-phospholipid syndrome, ANCA associated vasculitis, Goodpasture's disease, Kawasaki disease, autoimmune hemolytic anemia, and rapidly progressive glomerulonephritis, heavy-chain disease, military or immunocyte-associated amyloidosis, and monoclonal gammopathy of undetermined significance.

As used herein, a “linker” in the context of a polypeptide refers to a segment of amino acids that connects a first region (e.g., variable or constant heavy or light domain, half Fab region, or Fc region) to a second region (e.g., variable or constant heavy or light domain, half Fab region, or Fc region). Variable and constant heavy and light chain domains of immunoglobulins are not considered linkers. In some embodiments, a linker can be distinguished from the first and second regions because it does not comprise sequence that naturally occurs adjacent to the first and second regions. In some embodiments, a linker is recognized by a protease, or one or more proteases. In some embodiments, a linker substantially lacks known binding activities other than being recognized by a protease, or one or more proteases. Exemplary linker sequences are provided elsewhere herein.

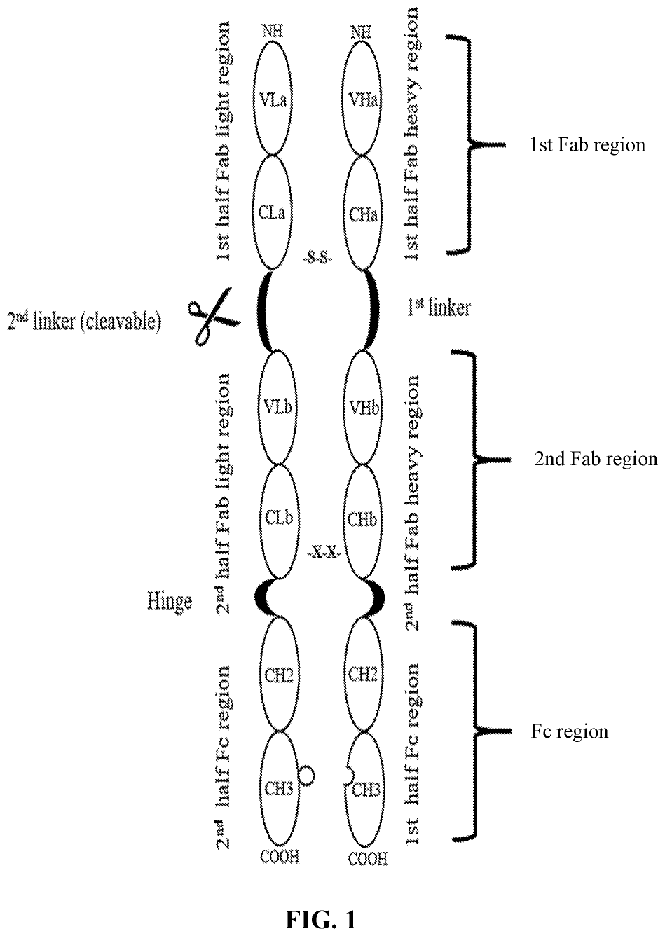

The term “in-tandem” as used herein with respect to regions in a protein complex, such as Fab regions, means that the regions are arranged head-to-tail without an intervening different region (e.g., Fe region) between the Fab regions. The in-tandem Fab regions may be separated by a linker. The exemplary structure illustrated in FIG. 1 comprises in-tandem Fab regions.

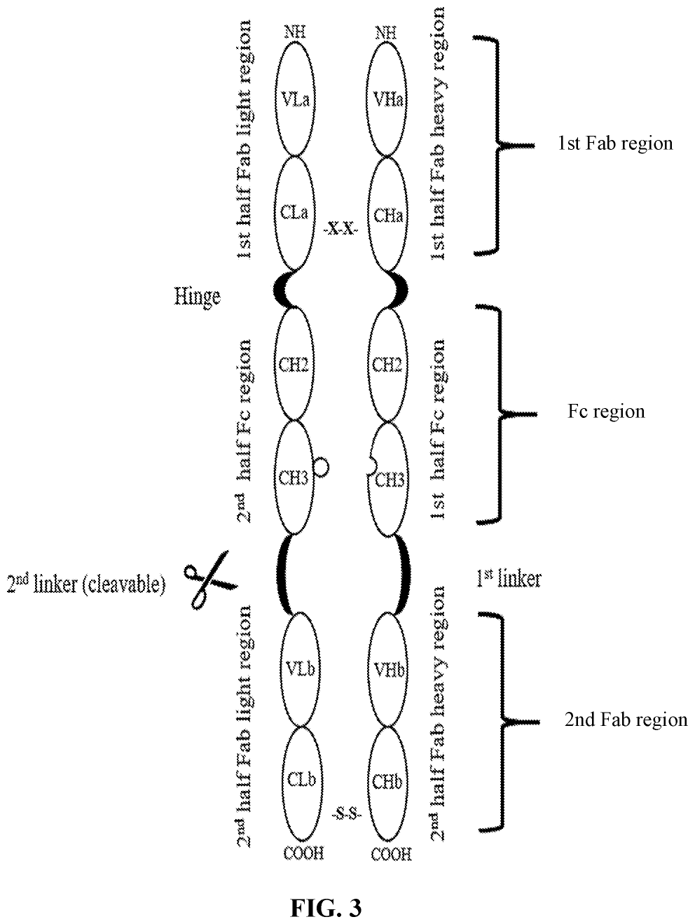

The term“non-tandem” as used herein with respect to regions in a protein complex, such as Fab regions, means that the regions are not in an “in-tandem” arrangement. The exemplary structure illustrated in FIG. 3 comprises non-tandem Fab regions. In some embodiments, an Fc region, or a CH2 or CH3 domain, is located between Fab regions that are in a non-tandem arrangement.

The present disclosure provides various embodiments of multi-specific antigen binding protein complexes, compositions comprising the multi-specific antigen binding protein complexes including pharmaceutical compositions and kits, and methods of making them and using the same. The terms “multi-specific antigen binding protein complex” and “protein complex” may be used interchangeably herein and refer to protein complexes formed from association of multiple polypeptide chains wherein the protein complexes comprise two different Fab regions each capable of binding two different epitopes and wherein the protein complexes comprise an Fc region. In one embodiment, the multi-specific antigen binding protein complexes each comprise two or three polypeptide chains that associate with each other to form a protein complex. In one embodiment, the two different Fab regions are arranged in an in-tandem or non-tandem manner. In one embodiment, the protein complex comprises an Fc region that exhibits Fc effector function or the Fc region contains a mutation that reduces/eliminates Fc effector function. In one embodiment, the multi-specific antigen binding protein complex comprises a bispecific antibody that binds two different epitopes.

The present disclosure provides several embodiments of the multi-specific antigen binding protein complexes that are simple, being composed of either two or three polypeptide chains that assemble with each other to form heterodimeric or heterotrimeric molecules that can bind two different epitopes. In the two-chain embodiment, the two polypeptide chains can be modified to include mutations, e.g., in the Fc region, that favor interchain association between the first and second polypeptide chains to increase the yield of hetero-dimeric molecules, rather than formation of homo-dimeric molecules. In one embodiment, the modifications include introduction of additional interchain disulfide bonds, optimization of interchain disulfide bonds and/or interchain steric complementarity, e.g., comprising knob-in-hole structures. In the three chain embodiment, a first polypeptide chain associates with a second and third chain to form a heterodimeric molecule, wherein the first, second and third polypeptide chains are modified in a manner similar to the 2-chain embodiments to favor formation of hetero-dimeric complexes.

Another feature of the multi-specific antigen binding protein complexes is that, for example in the two chain embodiment, the half Fab heavy regions (e.g., VH and CH domains) are carried on the first polypeptide chain, and the half Fab light regions (e.g., VL and CL domains) are carried on the second polypeptide chain, so that association between the two chains more closely resemble association between heavy and light chains in a naturally-occurring immunoglobulin molecule. In the same manner, in the three chain embodiment, the half Fab heavy regions (e.g., VH and CH domains) are carried on the first polypeptide chain, and the half Fab light regions (e.g., VL and CL domains) are carried on the second and third polypeptide chains. Thus, the simplicity of the two and three chain embodiments, and the positioning of the half Fab heavy and half Fab light regions on the two chains can favor efficient formation of heterodimer protein complexes that bind two different epitopes.

Exemplary immunoglobulin constant sequences suitable for use in multi-specific antigen binding protein complexes according to this disclosure include the following. In some embodiments, a heavy chain constant domain (e.g., CHa or CHb) comprises SEQ ID NO:2, 5, 31, 37, 60, 63, 75, 82, 91, 97, 91, 97, 106, 109, 121, 127, 136, or 139, or a sequence at least 95%, 96%, 97%, 98%, or 99% identical thereto. In some embodiments, a hinge sequence comprises SEQ ID NO: 6, 32, 40, 64, 71, 76, 92, 100, 110, 117, 122, 130, 140, or 147, or a sequence at least 95%, 96%, 97%, 98%, or 99% identical thereto. In some embodiments, a CH2 domain comprises SEQ ID NO: 7, 33, 65, 72, 77, 93, 101, 111, 118, 123, 131, 141, or 148, or a sequence at least 95%, 96%, 97%, 98%, or 99% identical thereto. In some embodiments, a CH3 domain comprises SEQ ID NO: 8, 34, 66, 73, 78, 94, 102, 112, 119, 124, 132, 142, or 149, a sequence at least 95%, 96%, 97%, 98%, or 99% identical thereto. In some embodiments, a light chain constant domain (e.g., CLa or CLb) comprises SEQ ID NO: 10, 39, 58, 68, 70, 84, 89, 99, 104, 114, 116, 134, 144, or 146, or a sequence at least 95%, 96%, 97%, 98%, or 99% identical thereto. Additional embodiments of such constant sequences and combinations thereof are described elsewhere herein and may be used generally in the disclosed multi-specific antigen binding protein complexes, regardless of whether they are described in the context of complexes with particular variable domains or other elements. In some embodiments, two or more, or each, of a CH1 domain (e.g., CHa or CHb), hinge domain, CH2 domain, and CH3 domain are present in a multi-specific antigen binding protein complex in a combination shown in any of the figures.

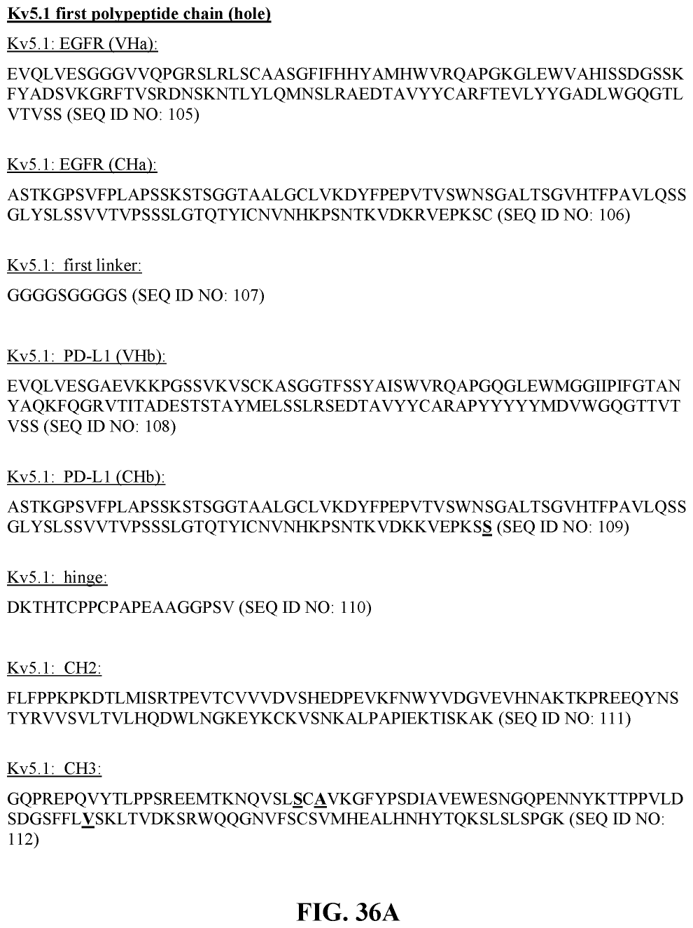

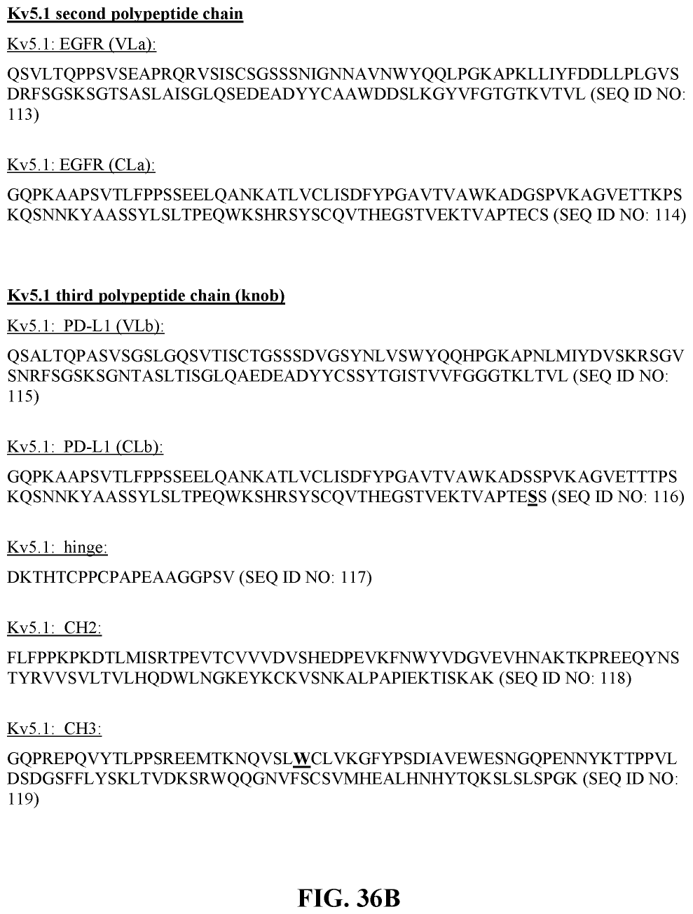

Exemplary immunoglobulin variable sequences suitable for use in multi-specific antigen binding protein complexes according to this disclosure include the following. In some embodiments, a heavy chain variable domain (e.g., VHa or VHb) comprises SEQ ID NO: 1, 4, 30, 36, 59, 62, 74, 81, 105, 108, 120, 126, 135, or 138, or a sequence at least 95%, 96%, 97%, 98%, or 99% identical thereto. In some embodiments, a light chain variable domain (e.g., VLa or VLb) comprises SEQ ID NO: 9, 25, 38, 57, 67, 69, 83, 88, 98, 103, 113, 115, 128, 133, 143, or 145, or a sequence at least 95%, 96%, 97%, 98%, or 99% identical thereto. In some embodiments, the multi-specific antigen binding protein complex comprises a pair of a VH and a VL that form an antigen binding site, wherein the VH and VL respectively comprise the sequences of SEQ ID NOs: 1 and 9; 4 and 25; 30 and 38; 36 and 57; 59 and 67; 62 and 69; 74 and 83; 81 and 88; 90 and 98; 96 and 103; 105 and 113; 108 and 115; 120 and 128; 126 and 133; 135 and 143; or 138 and 145. In some embodiments, the multi-specific antigen binding protein complex comprises a pair of a VH and a VL that form an antigen binding site, wherein the VH and VL respectively comprise sequences having at least 95%, 96%, 97%, 98%, or 99% identity to the sequences of SEQ ID NOs: 1 and 9; 4 and 25; 30 and 38; 36 and 57; 59 and 67; 62 and 69; 74 and 83; 81 and 88; 90 and 98; 96 and 103; 105 and 113; 108 and 115; 120 and 128; 126 and 133; 135 and 143; or 138 and 145. In some embodiments, the multi-specific antigen binding protein complex comprises a pair of a VH and a VL that form an antigen binding site selected from a VHa and a VLa sequence shown in a figure or in related panels of a figure (e.g., FIG. 36A shows a VHa sequence and FIG. 36B shows a VLa sequence that pairs with the VHa of FIG. 36A). In some embodiments, the multi-specific antigen binding protein complex comprises a pair of a VH and a VL that form an antigen binding site selected from a VHb and a VLb sequence shown in a figure or in related panels of a figure. In some embodiments, the multi-specific antigen binding protein complex comprises a first pair of a VH and a VL that form an antigen binding site selected from a VHa and a VLa sequence shown in a figure or in related panels of a figure and a second pair of a VH and a VL that form an antigen binding site selected from a VHb and a VLb sequence shown in a figure or in related panels of a figure.

In some embodiments, a heavy chain variable domain (e.g., VHa or VHb) comprises the complementarity-determining regions (CDRs) of SEQ ID NO: 1, 4, 30, 36, 59, 62, 74, 81, 105, 108, 120, 126, 135, or 138. In some embodiments, a light chain variable domain (e.g., VLa or VLb) comprises the complementarity-determining regions (CDRs) of SEQ ID NO: 9, 25, 38, 57, 67, 69, 83, 88, 98, 103, 113, 115, 128, 133, 143, or 145. In some embodiments, the multi-specific antigen binding protein complex comprises a pair of a VH and a VL that form an antigen binding site, wherein the VH and VL respectively comprise the CDRs of SEQ ID NOs: 1 and 9; 4 and 25; 30 and 38; 36 and 57; 59 and 67; 62 and 69; 74 and 83; 81 and 88; 90 and 98; 96 and 103; 105 and 113; 108 and 115; 120 and 128; 126 and 133; 135 and 143; or 138 and 145. In some embodiments, the multi-specific antigen binding protein complex comprises a pair of a VH and a VL that form an antigen binding site, wherein the CDRs of the VH and VL are the CDRs of a VHa and a VLa sequence shown in a figure or in related panels of a figure (e.g., FIG. 36A shows a VHa sequence and FIG. 36B shows a VLa sequence that pairs with the VHa of FIG. 36A). In some embodiments, the multi-specific antigen binding protein complex comprises a pair of a VH and a VL that form an antigen binding site, wherein the CDRs of the VH and VL are the CDRs of a VHb and a VLb sequence shown in a figure or in related panels of a figure. In some embodiments, the multi-specific antigen binding protein complex comprises a first pair of a VH and a VL that form an antigen binding site, wherein the CDRs of the first pair are the CDRs of a VHa and a VLa sequence shown in a figure or in related panels of a figure, and a second pair of a VH and a VL that form an antigen binding site, wherein the CDRs of the second pair are the CDRs of a VHb and a VLb sequence shown in a figure or in related panels of a figure. In any of the foregoing embodiments, the VH and/or VL sequences may further comprise framework sequences having at least 95%, 96%, 97%, 98%, or 99% identity to the framework sequences of the VH and/or VL sequence disclosed herein that contains the CDRs.

The present disclosure provides various embodiments of an “activatable” multi-specific antigen binding protein complex, or “activatable” protein complex, which refers to multiple polypeptide chains associated with each other to form a protein complex in which at least one of the polypeptide chains carries a cleavable linker and the protein complex comprises two different antigen binding domains in which one or both are activatable with cleavage of the cleavable linker. In its intact (un-cleaved) state, the first antigen binding domain can bind its target epitope however the intact cleavable linker inhibits the second antigen binding domain from binding its target antigen, thus rendering the protein complex in an inactive state. Upon cleavage of the cleavable linker, the second antigen binding domain can bind its target antigen. Thus, cleavage of the cleavable linker results in an activated multi-specific antigen binding protein complex or activated protein complex. The cleavable linker can be a peptide linker that is cleavable with a protease, esterase, reductive condition, or oxidative condition. In its in-active state, the multi-specific antigen binding protein complexes can be administered to a subject in need of tumor treatment, as the first antigen binding domain binds to its target epitope (e.g., an antigen expressed by tumor cells) but cell killing is not initiated until the cleavable linker is cleaved. In one embodiment, the cleavable linker is cleaved by a cleaving condition (e.g., a protease) that is present in the tumor microenvironment which generates the activated form of the protein complex which in turn permits the second antigen binding domain to bind its target epitope (e.g., a surface antigen expressed by effector T cells). The activated protein complex, when bound to first and second epitopes at the same time, can form an immune cell synapse which can lead to cytotoxic cell killing. Additionally, when the activated protein complex includes a functional Fc region, then the Fc region can bind an Fc receptor, thereby forming a three-way immune cell synapse comprising the protein complex binding at the same time to an effector T cell, a tumor cell expressing a target tumor antigen, and an Fc receptor-expressing cell (e.g., macrophage, natural killer cell or dendritic cell). The three-way immune cell synapse can mediate cytotoxic cell killing. It is not necessary for the intact protease-activatable protein complexes to physically contact the tumor cell, nor enter the tumor cell, but rather the intact protease-activatable protein complexes are converted to activated protein complexes when located in the tumor microenvironment which contains the cleaving condition (e.g., tumor-secreted protease).

The multi-specific antigen binding protein complexes having a three polypeptide chain configuration do not carry a cleavable linker and are already in an “activated state. The three polypeptide chain protein complexes can bind first and second epitopes at the same time, and the Fc region has effector function, and in manner similar to the two chain protein complex the three-chain protein complex can form an immune cell synapse which can lead to cytotoxic cell killing.

The present disclosure also provides various embodiments of multi-specific antigen binding protein complexes or protein complexes comprising any three polypeptide chain configuration that can be used to bind a first and second target epitope at the same time, thereby blocking binding to the first and second epitope by competing antigen binding molecules (e.g., antibodies).

The multi-specific antigen binding protein complexes are bispecific antibodies that can be used treat a disease or disorder associated with tumor or cancer antigen expression. The protein complexes described herein comprise two different Fab regions and a mutated Fc region (although embodiments having functional Fc regions are described herein). The protein complexes mediate formation of an immune cell synapse by binding two different antigens at the same time, for example antigens on effector cells and tumor-associated antigens expressed by tumors, which brings the effector cell (e.g., T cell) in close proximity to the tumor cell which leads to tumor cell-selective cell killing.

The multi-specific antigen binding protein complexes carry two Fab regions where, in the activated form or in the three-chain configuration, at least one or both of the Fab regions binds its cognate target antigen with an affinity level similar to that exhibited by the parent antibody from which the Fab region(s) are derived. The multi-specific antigen binding protein complexes, in the activated form or in the three-chain configuration, induce antigen-specific cytokine release, induce cytotoxicity, and induce T-cell activation.

The present disclosure provides a multi-specific antigen binding protein complex that can bind two different epitopes on the same target antigen or two different epitopes on different target antigens, comprising: two different Fab regions, an Fc region and a cleavable linker. In one embodiment, the two different Fab regions, the Fc region and the cleavable linker are formed from two polypeptide chains and wherein each polypeptide chain carries two different half Fab regions and a half Fc region (FIGS. 1 and 3). One of the polypeptide chains carries a cleavable peptide linker. In one embodiment, the one polypeptide chain carries a single cleavable peptide linker. The first and second polypeptide chains can associate with each other to form the multi-specific antigen binding protein complex having a first Fab region that is capable of binding a first epitope and having a second Fab region that is capable of binding a second epitope that differs from the first epitope. The first and second polypeptide chains can associate with each other to form the multi-specific antigen binding protein complex having an Fc region that is capable of binding an Fc receptor, or exhibits reduced or null binding to an Fc receptor. The first and second polypeptide chains can associate with each other via covalent and/or non-covalent bonds to form the multi-specific antigen binding protein complex. In one embodiment, the covalent bond comprises a disulfide bond. In one embodiment, the non-covalent bond comprises steric or electrostatic complementarity. In one embodiment, the first Fab region exhibits binding to its target epitope and the second Fab region exhibits reduced binding to its target epitope when the cleavable linker is in the un-cleaved state (e.g., intact cleavable linker). In one embodiment, the second Fab region exhibits reduced binding to its target epitope due to steric hinderance when the cleavable linker is in the un-cleaved state. In one embodiment, the second Fab regions exhibits increased binding to its target epitope (e.g., activated) when the cleavable linker is cleaved. Upon cleavage of the cleavable linker, the first Fab region and the second Fab region are capable of binding to the first and second target epitopes, respectively, at the same time. In one embodiment, the first and second polypeptide chains carry a first half Fab region and a second half Fab region arranged in-tandem, and a half Fc region. In one embodiment, the first and second polypeptide chains carry a first half Fab region and a second half Fab region and an intervening a half Fc region (e.g., first and second half Fab regions arranged in a non-tandem manner). In one embodiment, the Fc region is mutated which reduces or eliminates binding to an Fc receptor.

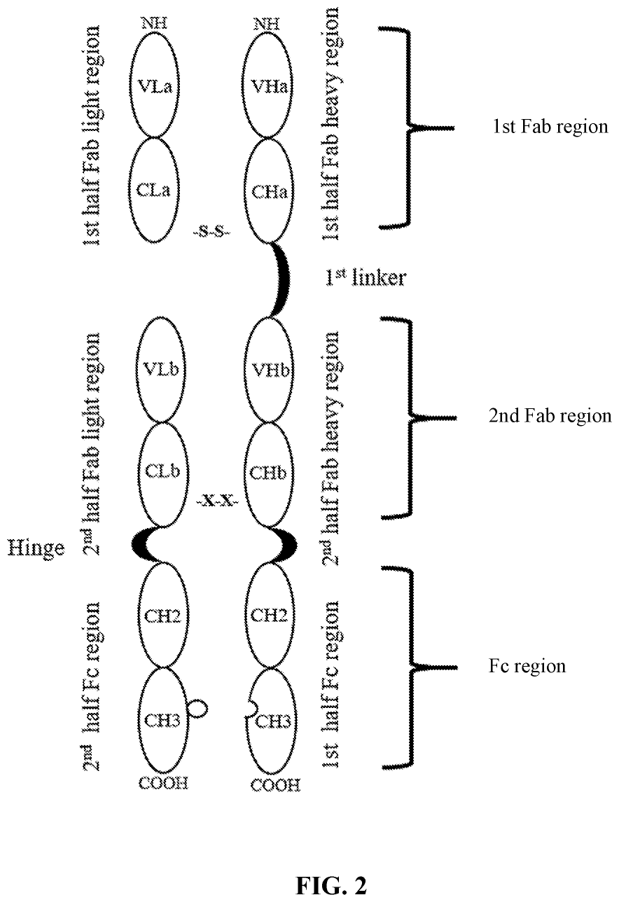

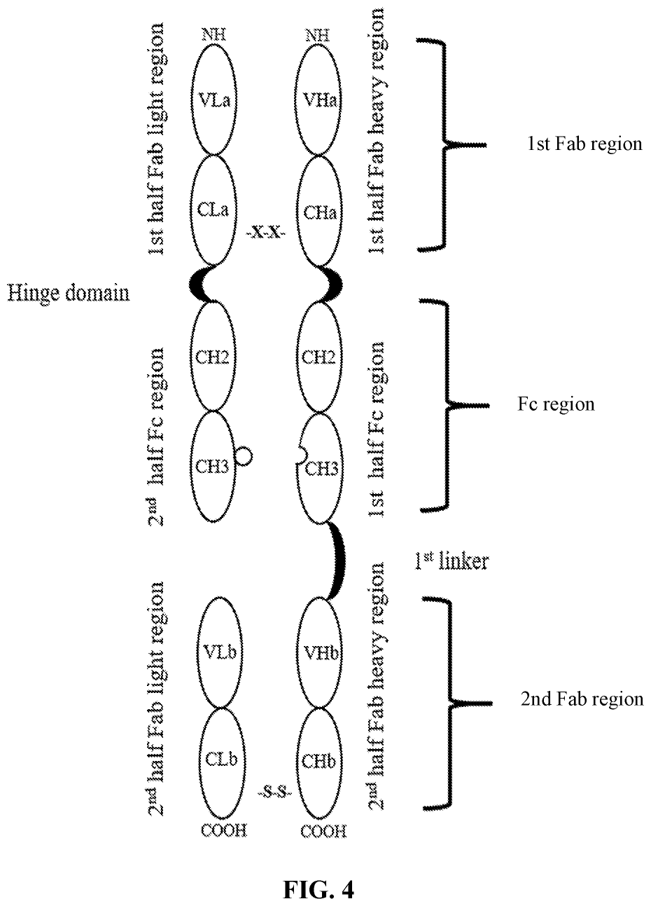

The present disclosure provides a multi-specific antigen binding protein complex that can bind two different epitopes on the same target antigen or two different epitopes on different target antigens, comprising: two different Fab regions and an Fc region. In one embodiment, the two different Fab regions and the Fc region are formed from three polypeptide chains (FIGS. 2 and 4). The first polypeptide chain can carry a half Fab heavy region of the first and second half Fabs and a first half Fc region. The first polypeptide chain can carry a first peptide linker. The second and third polypeptide chains together can carry a half Fab light region of the first and second half Fabs and a second half Fc region. The first, second and third polypeptide chains can associate with each other to form the multi-specific antigen binding protein complex having a first Fab region that is capable of binding a first epitope and having a second Fab region that is capable of binding a second epitope that differs from the first epitope. The first, second and third polypeptide chains can associate with each other to form the multi-specific antigen binding protein complex having an Fc region that is capable of binding an Fc receptor, or exhibits reduced or null binding to an Fc receptor. The first, second and third polypeptide chains can associate with each other via covalent and/or non-covalent bonds to form the multi-specific antigen binding protein complex. In one embodiment, the covalent bond comprises a disulfide bond. In one embodiment, the non-covalent bond comprises steric or electrostatic complementarity. In one embodiment, the first Fab region exhibits binding to its target epitope and the second Fab region exhibits binding to its target epitope. In one embodiment, the first Fab region and the second Fab region are capable of binding to the first and second target epitopes, respectively, at the same time. In one embodiment, the first, second and third polypeptide chains carry a first half Fab region and a second half Fab region arranged in-tandem, and a half Fc region. In one embodiment, the first, second and third polypeptide chains carry a first half Fab region and a second half Fab region and an intervening a half Fc region (e.g., first and second half Fab regions arranged in a non-tandem manner). In one embodiment, the Fc region is mutated which reduces or eliminates binding to an Fc receptor.

The present disclosure provides a two chain multi-specific antigen binding protein complex that can bind two different epitopes on the same target antigen or two different epitopes on different target antigens, comprising: (a) a first polypeptide chain comprising (i) a first half Fab heavy region, (ii) a first linker, (iii) a second half Fab heavy region, and (iv) a first half Fc region, wherein the first half Fab heavy region comprises a first-variable region and first-constant region from a first Fab heavy chain, and wherein the second half Fab heavy region comprises a second-variable region and second-constant region from a second Fab heavy chain; and (b) a second polypeptide chain comprising (i) a first half Fab light region, (ii) a second linker, (iii) a second half Fab light region, and (iv) a second half Fc region, wherein the first half Fab light region comprises a first-variable region and first-constant region from a first Fab light chain, and wherein the second half Fab light region comprises a second-variable region and second-constant region from a second Fab light chain, and wherein the second linker is cleavable or is not cleavable. In one embodiment, the first and second polypeptide chains each comprise first and second half Fab regions arranged in an in-tandem manner (FIG. 1).

In one embodiment, the two chain multi-specific antigen binding protein complex comprises: (a) a first polypeptide chain comprising 5 regions ordered from the amino terminus to the carboxyl terminus: (i) a first heavy chain variable region (VHa) and a first heavy chain constant region (CHa), (ii) a first linker (L1), (iii) a second heavy chain variable region (VHb) and a second heavy chain constant region (CHb), (iv) a first hinge region, and (v) a first Fc region comprising a first CH2 region and a first CH3 region; and (b) a second polypeptide chain comprising 5 regions ordered from the amino terminus to the carboxyl terminus: (i) a first light chain variable region (VLa) and a first light chain constant region (CLa), (ii) a second linker (L2), (iii) a second light chain variable region (VLb) and a second light chain constant region (CLb), (iv) a second hinge region, and (v) a second Fc region comprising a second CH2 region and a second CH3 region, wherein the first and second polypeptide chains associate with each other to form the multi-specific antigen binding protein complex having a first Fab region that is capable of binding a first epitope and having a second Fab region that is capable of binding a second epitope that differs from the first epitope, and wherein the first linker is not cleavable and the second linker is cleavable. Optionally, the second linker is not cleavable. In one embodiment, the first and second polypeptide chains each comprise first and second half Fab regions arranged in an in-tandem manner (FIG. 1).

The first and second polypeptide chains can associate with each other to form the multi-specific antigen binding protein complex having an Fc region that is capable of binding an Fc receptor. In one embodiment, the Fc region is mutated which reduces or eliminates binding to an Fc receptor. In one embodiment, the Fc region comprises an equivalent to a LALA or LALA-PG mutation (e.g., aligns with L234A, L235A, P329G) which reduces effector function. The first and second polypeptide chains can associate with each other via covalent and/or non-covalent bonds to form the multi-specific antigen binding protein complex. In one embodiment, the covalent bond comprises a disulfide bond. In one embodiment, the non-covalent bond comprises steric complementarity (e.g., knob-in-hole) or electrostatic complementarity. In one embodiment, a knob-in-hole structure is located in the Fc region as shown in FIG. 1.

The present disclosure provides a multi-specific antigen binding protein complex comprising a first polypeptide chain which comprises (e.g., in N- to C-terminal order) a heavy chain variable domain comprising an amino acid sequence that is at least 95% identical to, at least 96% identical to, at least 97% identical to, at least 98% identical to, at least 99% identical to, or 100% identical to SEQ ID NO:1; a heavy chain constant domain (e.g., SEQ ID NO:2 or a sequence at least 95%, 96%, 97%, 98%, or 99% identical thereto); a first linker (e.g., SEQ ID NO:3 or a sequence at least 95%, 96%, 97%, 98%, or 99% identical thereto); an amino acid sequence that is at least 95% identical to, at least 96% identical to, at least 97% identical to, at least 98% identical to, at least 99% identical to, or 100% identical to SEQ ID NO:4; a heavy chain constant domain (e.g., SEQ ID NO:5 or a sequence at least 95%, 96%, 97%, 98%, or 99% identical thereto); a hinge sequence (e.g., SEQ ID NO:6 or a sequence at least 95%, 96%, 97%, 98%, or 99% identical thereto); a CH2 domain (e.g., SEQ ID NO:7 or a sequence at least 95%, 96%, 97%, 98%, or 99% identical thereto); and a CH3 domain (e.g., SEQ ID NO:8 or a sequence at least 95%, 96%, 97%, 98%, or 99% identical thereto); and a second polypeptide chain which comprises (e.g., in N- to C-terminal order) an amino acid sequence that is at least 95% identical to, at least 96% identical to, at least 97% identical to, at least 98% identical to, at least 99% identical to, or 100% identical to SEQ ID NO:9; a light chain constant domain (e.g., SEQ ID NO:10 or a sequence at least 95%, 96%, 97%, 98%, or 99% identical thereto); a second linker (e.g., any one of SEQ ID NOs:11-24 or a sequence at least 95%, 96%, 97%, 98%, or 99% identical thereto); an amino acid sequence that is at least 95% identical to, at least 96% identical to, at least 97% identical to, at least 98% identical to, at least 99% identical to, or 100% identical to SEQ ID NO:25; a light chain constant domain (e.g., SEQ ID NO:26 or a sequence at least 95%, 96%, 97%, 98%, or 99% identical thereto); a hinge sequence (e.g., SEQ ID NO:27 or a sequence at least 95%, 96%, 97%, 98%, or 99% identical thereto); a CH2 domain (e.g., SEQ ID NO:28 or a sequence at least 95%, 96%, 97%, 98%, or 99% identical thereto); and a CH3 domain (e.g., SEQ ID NO:29 or a sequence at least 95%, 96%, 97%, 98%, or 99% identical thereto).

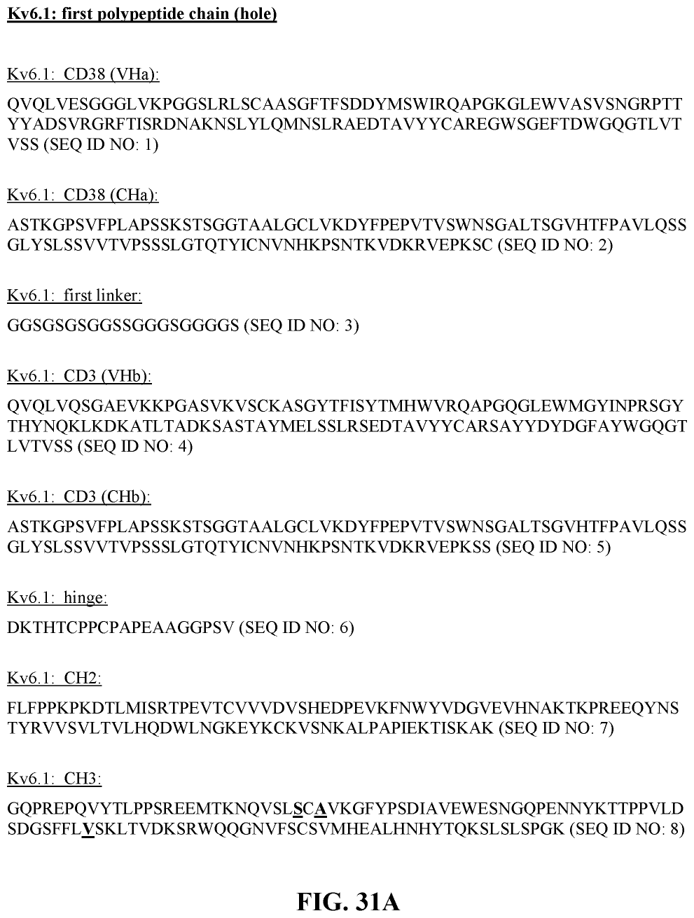

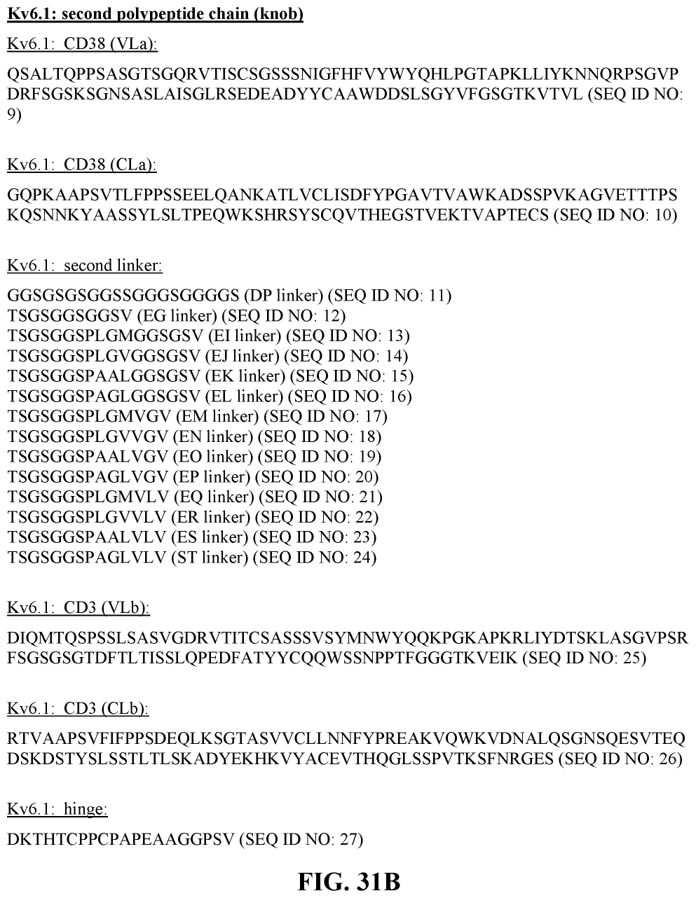

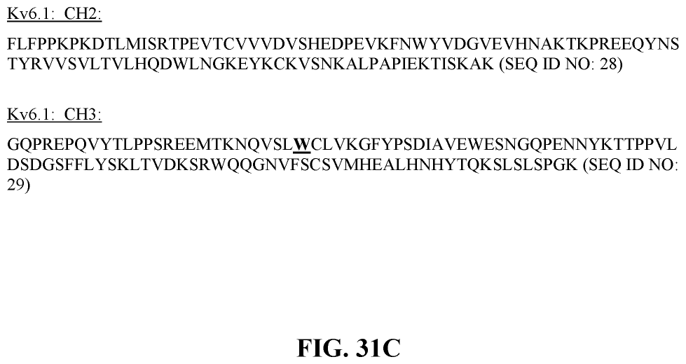

The present disclosure provides a multi-specific antigen binding protein complex comprising a first polypeptide chain (or a portion thereof) which comprises an amino acid sequence that is at least 95% identical to, at least 96% identical to, at least 97% identical to, at least 98% identical to, or at least 99% identical to the amino acid sequences shown in FIGS. 31A-C (e.g., Kv6.1, CD38/CD3).

The present disclosure provides a multi-specific antigen binding protein complex comprising a second polypeptide chain (or a portion thereof) which comprises an amino acid sequence that is at least 95% identical to, at least 96% identical to, at least 97% identical to, at least 98% identical to, or at least 99% identical to the amino acid sequences shown in FIGS. 31A-C (e.g., Kv6.1, CD38/CD3).

The first Fab region can bind to its target epitope and the second Fab region exhibits reduced binding to its target epitope when the cleavable linker is in the un-cleaved state. The second Fab regions exhibits increased binding to its target epitope (e.g., activated) when the cleavable linker is cleaved. In one embodiment, upon cleavage of the cleavable linker, the first Fab region and the second Fab region are capable of binding to the first and second target epitopes, respectively, at the same time.

In one embodiment, the first and second linkers comprise peptide linkers. In one embodiment, the multi-specific antigen binding protein complex comprises a single cleavable linker. In one embodiment, the linker is cleavable with a protease selected from a group consisting of a matrix metalloprotease (MMP), MMP1, MMP2, MMP3, MMP8, MMP9, MMP11, MMP13, MMP14, MT1-MMP (membrane type 1 matrix metalloproteinase), ADAM protease, urokinase plasminogen activator (uPA), serine proteases, cysteine proteases, aspartate proteases, threonine proteases, cathepsins, cathepsin B, cathepsin C, cathepsin D, cathepsin E, cathepsin K and cathepsin L. In one embodiment, a cleavable linker comprises an amino acid sequence that is cleavable with at least one matrix metalloprotease (U.S. published application No. 2015/0087810). In one embodiment, the linker can be cleavable with other types of proteases which are listed in FIGS. 31A-C or 32A-C. In one embodiment, the cleavable linker comprises the amino acid sequence selected from a group consisting of GGSGSGSGGSSGGGSGGGGS (DP linker), TSGSGGSGGSV (EG or EH linker), TSGSGGSPLGMGGSGSV (EI or EU linker), TSGSGGSPLGVGGSGSV (EJ or EV linker), TSGSGGSPAALGGSGSV (EK or EW linker), TSGSGGSPAGLGGSGSV (EL or EX linker), TSGSGGSPLGMVGV (EM or EY linker), TSGSGGSPLGVVGV (EN or EZ linker), TSGSGGSPAALVGV (EO or FA linker), TSGSGGSPAGLVGV (EP or FB linker), TSGSGGSPLGMVLV (EQ or FC linker), TSGSGGSPLGVVLV (ER or FD linker), TSGSGGSPAALVLV (ES or FE linker) or TSGSGGSPAGLVLV (ST or FF linker) (SEQ ID NOS; 11-24, respectively, or SEQ ID NOS:43-56, respectively).

In one embodiment, first linker comprises an amino acid sequence selected from a group consisting of: (SG)ₙ (SEQ ID NO: 157), (SGG)ₙ (SEQ ID NO: 158), (SGGG)ₙ (SEQ ID NO: 159), (SSG)ₙ (SEQ ID NO: 160), (GS)ₙ (SEQ ID NO: 161), (GGG)ₙ (SEQ ID NO: 162), (GSGGS)ₙ (SEQ ID NO: 163), (GSG)ₙ (SEQ ID NO: 164), (GGGGS)ₙ (SEQ ID NO: 165), (GGGS)ₙ (SEQ ID NO: 166), (GGGGSGS)ₙ (SEQ ID NO: 167), (GGGGSGGS)ₙ (SEQ ID NO: 168), and (GGS)ₙ (SEQ ID NO: 169), where n is an integer of 1-6. In one embodiment, the first linker comprises an amino acid sequence of TSGSGGSGGSV (SEQ ID NO: 156).

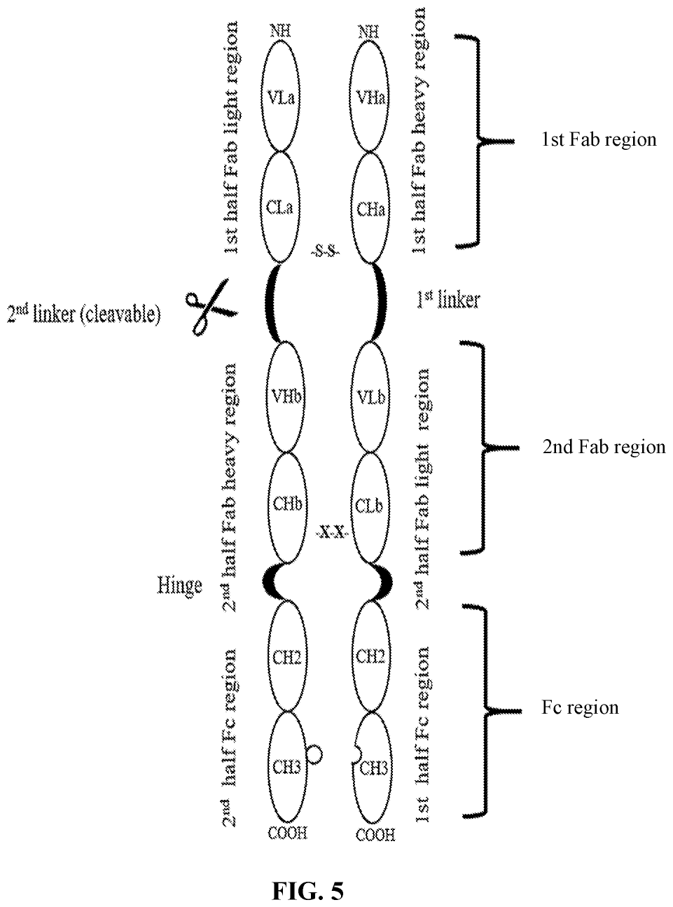

One skilled in the art will appreciate that two chain multi-specific antigen binding protein complexes can comprise polypeptide chains having alternative arrangements are possible. In one example, the first polypeptide chain comprises: a first-variable region (VHa) and a first-constant region (CHa) from a first Fab heavy chain, a first or second linker (L1 or L2), a second-variable region (VLb) and a second-constant region (CLb) from a second Fab light chain, and a first Fc region (CH2 and CH3). The second polypeptide chain comprises: a first-variable region (VLa) and a first-constant region (CLa) from a first Fab light chain, a second or first linker (L2 or L1), a second-variable region (VHb) and a second-constant region (CHb) from a second Fab heavy chain, and a second Fc region (CH2 and CH3). In one embodiment, the first or second linker is cleavable (FIG. 5). Optionally, the first and second linkers are not cleavable.

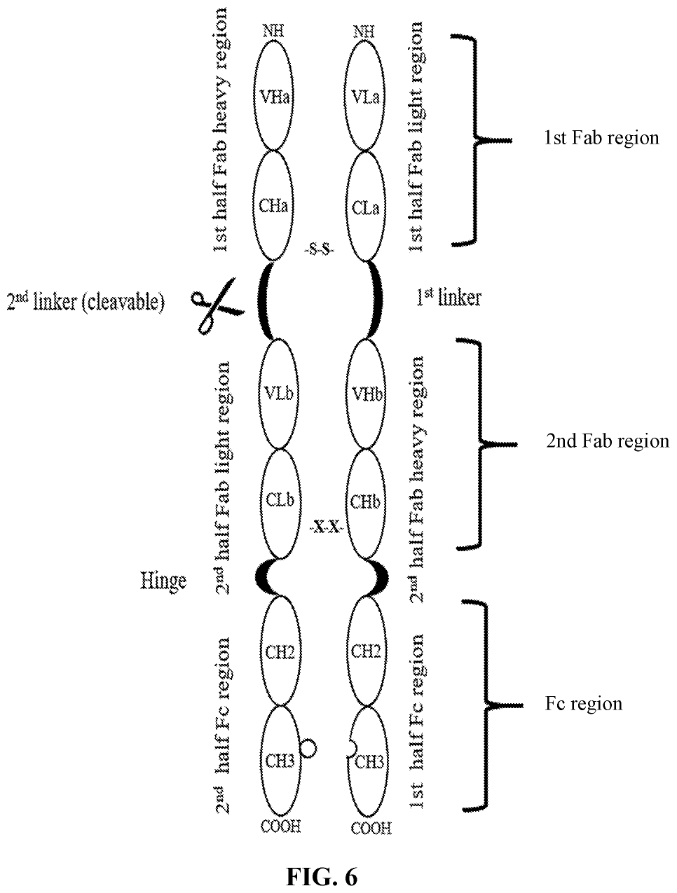

In another example, the first polypeptide chain comprises: a first-variable region (VLa) and a first-constant region (CLa) from a first Fab light chain, a first or second linker (L1 or L2), a second-variable region (VHb) and a second-constant region (CHb) from a second Fab heavy chain, and a first Fc region (CH2 and CH3). The second polypeptide chain comprises: a first-variable region (VHa) and a first-constant region (CHa) from a first Fab heavy chain, a second or first linker (L2 or L1), a second-variable region (VLb) and a second-constant region (CLb) from a second Fab light chain, and a second Fc region (CH2 and CH3). In one embodiment, the first or second linker is cleavable (FIG. 6). Optionally, the first and second linkers are not cleavable.

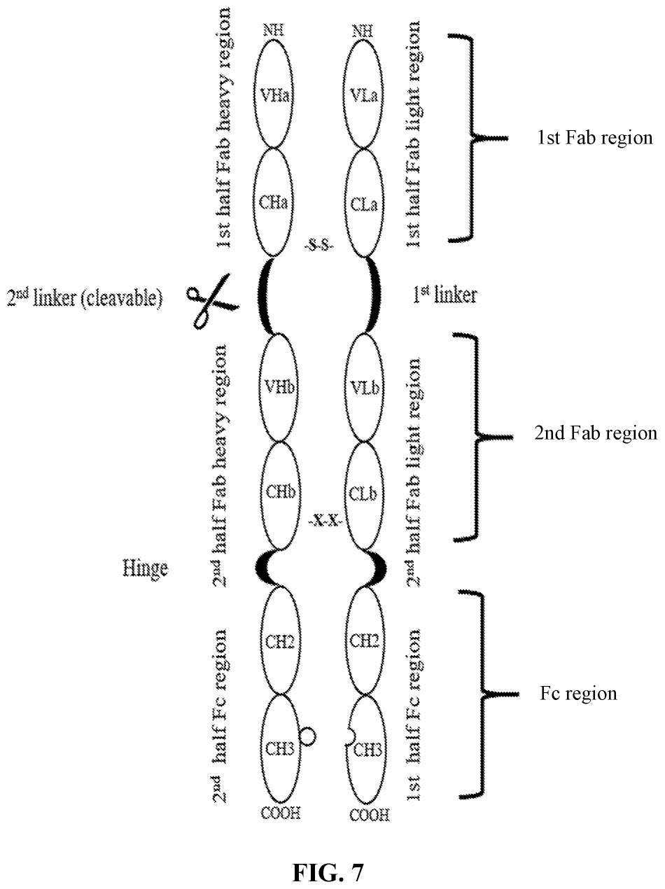

In yet another example, the first polypeptide chain comprises: a first-variable region (VLa) and a first-constant region (CLa) from a first Fab light chain, a first or second linker (L1 or L2), a second-variable region (VLb) and a second-constant region (CLb) from a second Fab light chain, and a first Fc region (CH2 and CH3). The second polypeptide chain comprises: a first-variable region (VHa) and a first-constant region (CHa) from a first Fab heavy chain, a second or first linker (L2 or L1), a second-variable region (VHb) and a second-constant region (CHb) from a second Fab heavy chain, and a second Fc region (CH2 and CH3). In one embodiment, the first or second linker is cleavable (FIG. 7). Optionally, the first and second linkers are not cleavable.

In one embodiment, the alternative protein complexes shown in FIGS. 5, 6 and 7, comprise a wild type Fc region or a mutant Fc region, wherein the mutant Fc region comprises an equivalent to a LALA or LALA-PG mutation (e.g., aligned at L234A, L235A, P329G) which reduces effector function. In one embodiment, the first or second linker is cleavable with a protease, such as a matrix metalloprotease. In one embodiment, the first or second linker comprises the amino acid sequence of one of the cleavable linkers according to SEQ ID NOS:11-24 or 43-56. In one embodiment, the first or second linker is not cleavable with a protease and comprises the amino acid sequence of one of the non-cleavable linkers according to SEQ ID NOS:3, 35, 61, 79, 80, 95, 107 or 125.

The present disclosure provides a two-chain multi-specific antigen binding protein complex that can bind two different epitopes on the same target antigen or two different epitopes on different target antigens, comprising: (a) a first polypeptide chain comprising (i) a first half Fab heavy region, (ii) a first half Fc region, (iii) a first linker, and (iv) a second half Fab heavy region, wherein the first half Fab heavy region comprises a first-variable region and first-constant region from a first Fab heavy chain, and wherein the second half Fab heavy region comprises a second-variable region and second-constant region from a second Fab heavy chain; and (b) a second polypeptide chain comprising (i) a first half Fab light region, (ii) a second half Fc region, (iii) a second linker, and (iv) a second half Fab light region, wherein the first half Fab light region comprises a first-variable region and first-constant region from a first Fab light chain, and wherein the second half Fab light region comprises a second-variable region and second-constant region from a second Fab light chain, and wherein the second linker is cleavable or is not cleavable. In one embodiment, the first and second polypeptide chains each comprise first and second half Fab regions arranged in a non-tandem manner (FIG. 3).

In one embodiment, the two chain multi-specific antigen binding protein complex that can bind two different epitopes on the same target antigen or two different epitopes on different target antigens, comprising: (a) a first polypeptide chain comprising 5 regions ordered from the amino terminus to the carboxyl terminus: (i) a first heavy chain variable region (VHa) and a first heavy chain constant region (CHa), (ii) a first hinge region, (iii) a first Fc region comprising a first CH2 region and a first CH3 region, (iv) a first linker (L1), and (v) a second heavy chain variable region (VHb) and a second heavy chain constant region (CHb); and (b) a second polypeptide chain comprising 5 regions ordered from the amino terminus to the carboxyl terminus: (i) a first light chain variable region (VLa) and a first light chain constant region (CLa), (ii) a second hinge region, (iii) a second Fc region comprising a second CH2 region and a second CH3 region, (iv) a second linker (L2), and (v) a second light chain variable region (VLb) and a second light chain constant region (CLb), wherein the first and second polypeptide chains associate with each other to form the multi-specific antigen binding protein complex having a first Fab region that is capable of binding a first epitope and having a second Fab region that is capable of binding a second epitope that differs from the first epitope, and wherein the second linker is cleavable. Optionally, the second linker is not cleavable. In one embodiment, the first and second polypeptide chains each comprise first and second half Fab regions arranged in a non-tandem manner (FIG. 3).

The first and second polypeptide chains can associate with each other to form the multi-specific antigen binding protein complex having an Fc region that is capable of binding an Fc receptor. In one embodiment, the Fc region comprises an equivalent to a LALA or LALA-PG mutation (e.g., aligned at L234A, L235A, P329G) which reduces effector function. The first and second polypeptide chains can associate with each other via covalent and/or non-covalent bonds to form the multi-specific antigen binding protein complex. In one embodiment, the covalent bond comprises a disulfide bond. In one embodiment, the non-covalent bond comprises steric complementarity (e.g., knob-in-hole) or electrostatic complementarity. In one embodiment, a knob-in-hole structure is located in the full Fc domain as shown in FIG. 3.

The present disclosure provides a multi-specific antigen binding protein complex comprising a first polypeptide chain which comprises (e.g., in N- to C-terminal order) a heavy chain variable domain comprising an amino acid sequence that is at least 95% identical to, at least 96% identical to, at least 97% identical to, at least 98% identical to, at least 99% identical to, or 100% identical to SEQ ID NO:30; a heavy chain constant domain (e.g., SEQ ID NO:31 or a sequence at least 95%, 96%, 97%, 98%, or 99% identical thereto); a hinge sequence (e.g., SEQ ID NO:32 or a sequence at least 95%, 96%, 97%, 98%, or 99% identical thereto); a CH2 domain (e.g., SEQ ID NO:33 or a sequence at least 95%, 96%, 97%, 98%, or 99% identical thereto); a CH3 domain (e.g., SEQ ID NO:34 or a sequence at least 95%, 96%, 97%, 98%, or 99% identical thereto); a first linker (e.g., SEQ ID NO:35 or a sequence at least 95%, 96%, 97%, 98%, or 99% identical thereto); an amino acid sequence that is at least 95% identical to, at least 96% identical to, at least 97% identical to, at least 98% identical to, at least 99% identical to, or 100% identical to SEQ ID NO:36; and a heavy chain constant domain (e.g., SEQ ID NO:37 or a sequence at least 95%, 96%, 97%, 98%, or 99% identical thereto); and a second polypeptide chain which comprises (e.g., in N- to C-terminal order) an amino acid sequence that is at least 95% identical to, at least 96% identical to, at least 97% identical to, at least 98% identical to, at least 99% identical to, or 100% identical to SEQ ID NO:38; a light chain constant domain (e.g., SEQ ID NO:39 or a sequence at least 95%, 96%, 97%, 98%, or 99% identical thereto); a hinge sequence (e.g., SEQ ID NO:40 or a sequence at least 95%, 96%, 97%, 98%, or 99% identical thereto); a CH2 domain (e.g., SEQ ID NO:41 or a sequence at least 95%, 96%, 97%, 98%, or 99% identical thereto); a CH3 domain (e.g., SEQ ID NO:42 or a sequence at least 95%, 96%, 97%, 98%, or 99% identical thereto); a second linker (e.g., any one of SEQ ID NOs:43-56 or a sequence at least 95%, 96%, 97%, 98%, or 99% identical thereto); an amino acid sequence that is at least 95% identical to, at least 96% identical to, at least 97% identical to, at least 98% identical to, at least 99% identical to, or 100% identical to SEQ ID NO:57; and a light chain constant domain (e.g., SEQ ID NO:58 or a sequence at least 95%, 96%, 97%, 98%, or 99% identical thereto).

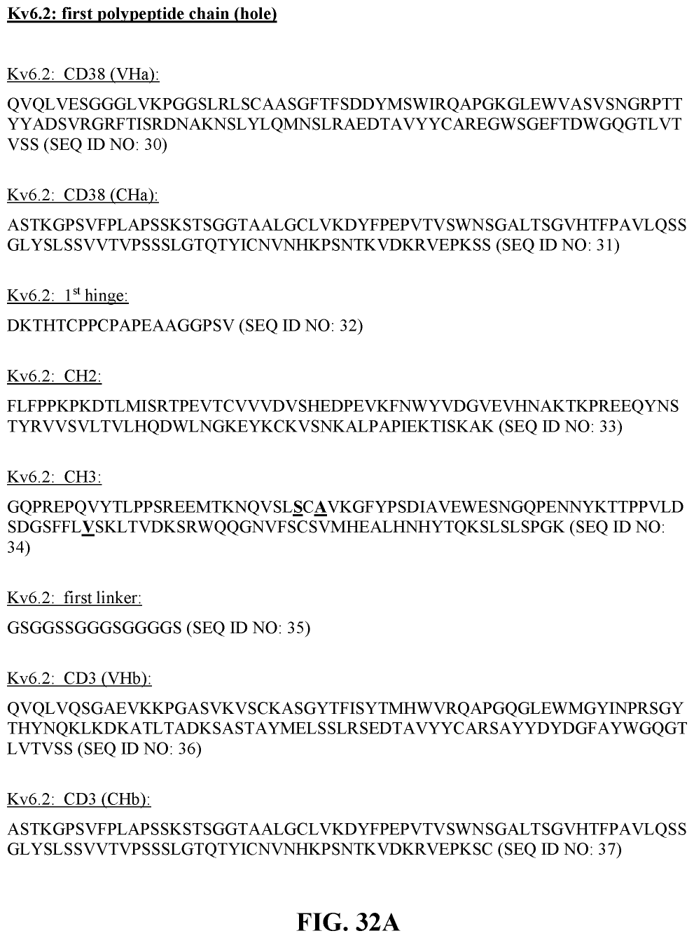

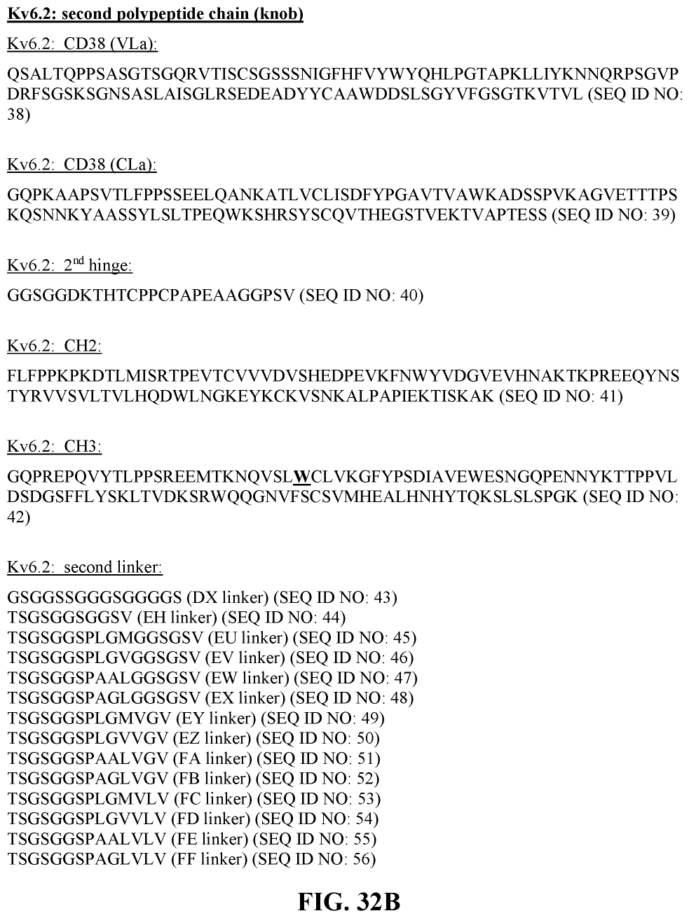

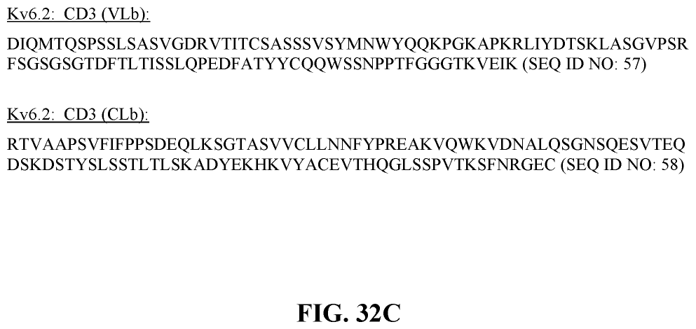

The present disclosure provides a multi-specific antigen binding protein complex comprising a first polypeptide chain (or a portion thereof) which comprises an amino acid sequence that is at least 95% identical to, at least 96% identical to, at least 97% identical to, at least 98% identical to, or at least 99% identical to the amino acid sequences shown in FIGS. 32A-C (e.g., Kv6.2, CD38/CD3).

The present disclosure provides a multi-specific antigen binding protein complex comprising a second polypeptide chain (or a portion thereof) which comprises an amino acid sequence that is at least 95% identical to, at least 96% identical to, at least 97% identical to, at least 98% identical to, or at least 99% identical to the amino acid sequences FIGS. 32A-C (e.g., Kv6.2, CD38/CD3).

The first Fab region can bind to its target epitope and the second Fab region exhibits reduced binding to its target epitope when the cleavable linker is in the un-cleaved state. The second Fab regions exhibits increased binding to its target epitope (e.g., activated) when the cleavable linker is cleaved. In one embodiment, upon cleavage of the cleavable linker, the first Fab region and the second Fab region are capable of binding to the first and second target epitopes, respectively, at the same time.

In one embodiment, the first and second linkers comprise peptide linkers. In one embodiment, the multi-specific antigen binding protein complex comprises a single cleavable linker. In one embodiment, the linker is cleavable with a protease selected from a group consisting of a matrix metalloprotease (MMP), MMP1, MMP2, MMP3, MMP8, MMP9, MMP11, MMP13, MMP14, MT1-MMP (membrane type 1 matrix metalloproteinase), ADAM protease, urokinase plasminogen activator (uPA), serine proteases, cysteine proteases, aspartate proteases, threonine proteases, cathepsins, cathepsin B, cathepsin C, cathepsin D, cathepsin E, cathepsin K and cathepsin L. In one embodiment, a cleavable linker comprises an amino acid sequence that is cleavable with at least one matrix metalloprotease (U.S. published application No. 2015/0087810). In one embodiment, the linker can be cleavable with other types of proteases which are listed in FIGS. 31A-C and 32A-C. In one embodiment, the cleavable linker comprises the amino acid sequence selected from a group consisting of GGSGSGSGGSSGGGSGGGGS (DP linker), TSGSGGSGGSV (EG or EH linker), TSGSGGSPLGMGGSGSV (EI or EU linker), TSGSGGSPLGVGGSGSV (EJ or EV linker), TSGSGGSPAALGGSGSV (EK or EW linker), TSGSGGSPAGLGGSGSV (EL or EX linker), TSGSGGSPLGMVGV (EM or EY linker), TSGSGGSPLGVVGV (EN or EZ linker), TSGSGGSPAALVGV (EO or FA linker), TSGSGGSPAGLVGV (EP or FB linker), TSGSGGSPLGMVLV (EQ or FC linker), TSGSGGSPLGVVLV (ER or FD linker), TSGSGGSPAALVLV (ES or FE linker) or TSGSGGSPAGLVLV (ST or FF linker) (SEQ ID NOS; 11-24, respectively, or SEQ ID NOS:43-56, respectively).

In one embodiment, first linker comprises an amino acid sequence selected from a group consisting of: (SG)ₙ (SEQ ID NO: 157), (SGG)ₙ (SEQ ID NO: 158), (SGGG)ₙ (SEQ ID NO: 159), (SSG)ₙ (SEQ ID NO: 160), (GS)ₙ (SEQ ID NO: 161), (GGG)ₙ (SEQ ID NO: 162), (GSGGS)ₙ (SEQ ID NO: 163), (GSG)ₙ (SEQ ID NO: 164), (GGGGS)ₙ (SEQ ID NO: 165), (GGGS)ₙ (SEQ ID NO: 166), (GGGGSGS)ₙ (SEQ ID NO: 167), (GGGGSGGS)ₙ (SEQ ID NO: 168), and (GGS)ₙ (SEQ ID NO: 169), where n is an integer of 1-6. In one embodiment, the first linker comprises an amino acid sequence of TSGSGGSGGSV (SEQ ID NO: 156).

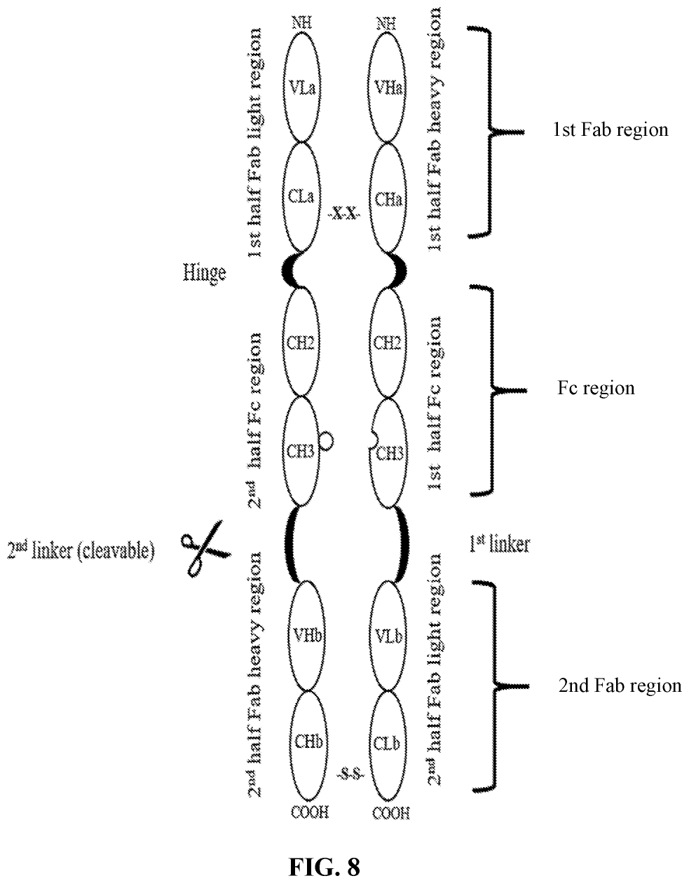

One skilled in the art will appreciate that two chain multi-specific antigen binding protein complexes can comprise polypeptide chains having alternative arrangements are possible. In one example, the first polypeptide chain comprises a first-variable region (VHa) and a first-constant region (CHa) from a first Fab heavy chain, a first Fc region (CH2 and CH3), a first or second linker (L1 or L2), a second-variable region (VLb) and a second-constant region (CLb) from a second Fab light chain. The second polypeptide chain comprises a first-variable region (VLa) and a first-constant region (CLa) from a first Fab light chain, a second Fc region (CH2 and CH3), a second or first linker (L2 or L1), and a second-variable region (VHb) and a second-constant region (CHb) from a second Fab heavy chain. In one embodiment, the first or second linker is cleavable (FIG. 8). Optionally, the first and second linkers are not cleavable.

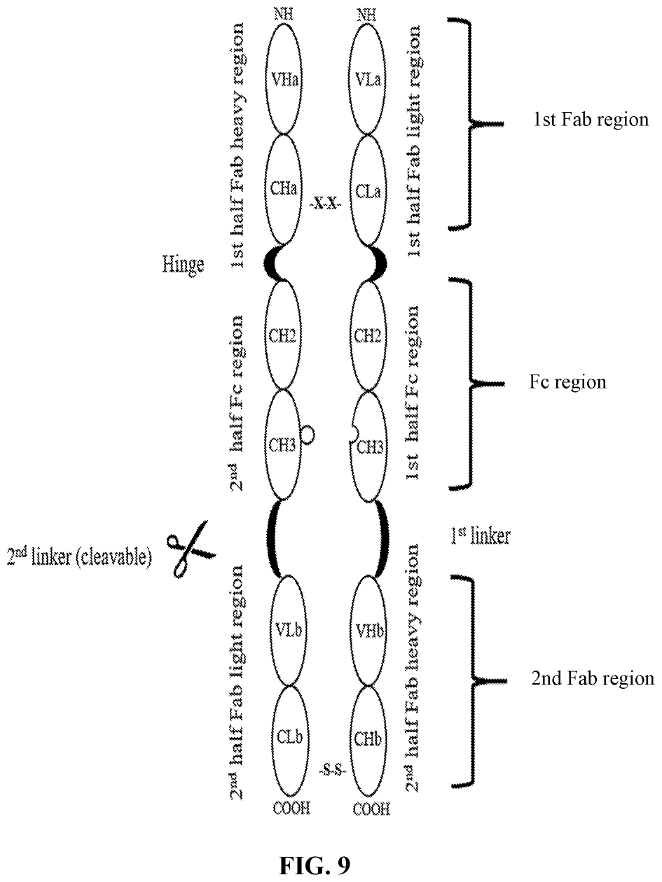

In another example, the first polypeptide chain comprises a first-variable region (VLa) and a first-constant region (CLa) from a first Fab light chain, a first Fc region (CH2 and CH3), a first or second linker (L1 or L2), a second-variable region (VHb) and a second-constant region (CHb) from a second Fab heavy chain. The second polypeptide chain comprises a first-variable region (VHa) and a first-constant region (CHa) from a first Fab heavy chain, a second Fc region (CH2 and CH3), a second or first linker (L2 or L1), and a second-variable region (VLb) and a second-constant region (CLb) from a second Fab light chain. In one embodiment, the first or second linker is cleavable (FIG. 9). Optionally, the first and second linkers are not cleavable.

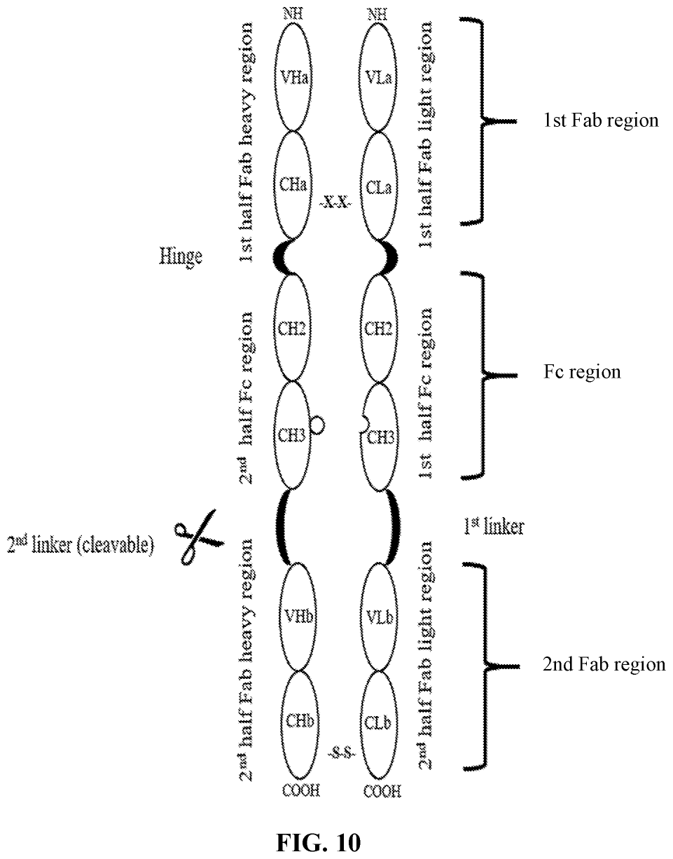

In yet another example, the first polypeptide chain comprises a first-variable region VLa) and a first-constant region (CLa) from a first Fab light chain, a first Fc region (CH2 and CH3), a first or second linker (L1 or L2), a second-variable region (VLb) and a second-constant region (CLb) from a second Fab light chain. The second polypeptide chain comprises a first-variable region (VHa) and a first-constant region (CHa) from a first Fab heavy chain, a second Fc region (CH2 and CH3), a second or first linker (L2 or L1), and a second-variable region (VHb) and a second-constant region (CHb) from a second Fab heavy chain. In one embodiment, the first or second linker is cleavable (FIG. 10). Optionally, the first and second linkers are not cleavable.

In one embodiment, the alternative protein complexes shown in FIGS. 8, 9 and 10, comprise a wild type Fc region or a mutant Fc region, wherein the mutant Fc region comprises an equivalent to a LALA or LALA-PG mutation (e.g., aligned at L234A, L235A, P329G) which reduces effector function. In one embodiment, the first or second linker is cleavable with a protease, such as a matrix metalloprotease. In one embodiment, the first or second linker comprises the amino acid sequence of one of the cleavable linkers according to SEQ ID NOS:11-24 or 43-56. In one embodiment, the first or second linker is not cleavable with a protease and comprises the amino acid sequence of one of the non-cleavable linkers according to SEQ ID NOS:3, 35, 61, 79, 80, 95, 107 or 125.

The present disclosure provides a three chain multi-specific antigen binding protein complex that can bind two different epitopes on the same target antigen or two different epitopes on different target antigens, comprising: (a) a first polypeptide chain comprising (i) a first half Fab heavy region, (ii) a first linker, (iii) a second half Fab heavy region, and (iv) a first half Fc region, wherein the first half Fab heavy region comprises a first-variable region and first-constant region from a first Fab heavy chain, and wherein the second half Fab heavy region comprises a second-variable region and second-constant region from a second Fab heavy chain; (b) a second polypeptide chain comprising a first half Fab light region which comprises a first-variable region and first-constant region from a first Fab light chain; and (c) a third polypeptide chain comprising (i) a second half Fab light region, and (ii) a second half Fc region, wherein the second half Fab light region comprises a second-variable region and second-constant region from a second Fab light chain. In one embodiment, the first polypeptide chain comprises first and second half Fab regions arranged in an in-tandem manner, and the second and third polypeptide chains are assembled with the first polypeptide chain and comprise first and second half Fab regions arranged in an in-tandem manner (FIG. 2).

In one embodiment, the three chain multi-specific antigen binding protein complex comprises: (a) a first polypeptide chain comprising 5 regions ordered from the amino terminus to the carboxyl terminus: (i) a first heavy chain variable region (VHa) and a first heavy chain constant region (CHa), (ii) a first linker (L1), (iii) a second heavy chain variable region (VHb) and a second heavy chain constant region (CHb), (iv) a first hinge region, and (v) a first Fc region comprising a first CH2 region and a first CH3 region; (b) a second polypeptide chain comprising a first light chain variable region (VLa) and a first light chain constant region (CLa); and (c) a third polypeptide chain comprising 3 regions ordered from the amino terminus to the carboxyl terminus: (i) a second light chain variable region (VLb) and a second light chain constant region (CLb), (ii) a second hinge region, and (iii) a second Fc region comprising a second CH2 region and a second CH3 region, wherein the first, second and third polypeptide chains associate with each other to form the multi-specific antigen binding protein complex having a first Fab region that is capable of binding a first epitope and having a second Fab region that is capable of binding a second epitope that differs from the first epitope. In one embodiment, the first polypeptide chain comprises first and second half Fab regions arranged in an in-tandem manner, and the second and third polypeptide chains are assembled with the first polypeptide chain and comprise first and second half Fab regions arranged in an in-tandem manner (FIG. 2).

The first, second and third polypeptide chains can associate with each other to form the multi-specific antigen binding protein complex having an Fc region that is capable of binding an Fc receptor. In one embodiment, the Fc region comprises an equivalent to a LALA or LALA-PG mutation (e.g., aligns with L234A, L235A, P329G) which reduces effector function. The first polypeptide chain can associate with the second and third polypeptide chains via covalent and/or non-covalent bonds to form the multi-specific antigen binding protein complex. In one embodiment, the covalent bond comprises a disulfide bond. In one embodiment, the non-covalent bond comprises steric complementarity (e.g., knob-in-hole) or electrostatic complementarity. In one embodiment, a knob-in-hole structure is located in the full Fc domain as shown in FIG. 2.

The present disclosure provides a multi-specific antigen binding protein complex comprising a first polypeptide chain (or a portion thereof) which comprises an amino acid sequence that is at least 95% identical to, at least 96% identical to, at least 97% identical to, at least 98% identical to, or at least 99% identical to the amino acid sequences shown in FIGS. 32A-B (e.g., CD38/CD3) or 35A-B (e.g., EGFR/PD-L1).

The present disclosure provides a multi-specific antigen binding protein complex comprising a second polypeptide chain (or a portion thereof) which comprises an amino acid sequence that is at least 95% identical to, at least 96% identical to, at least 97% identical to, at least 98% identical to, or at least 99% identical to the amino acid sequences shown in FIGS. 32A-B (e.g., CD38/CD3) or 35A-B (e.g., EGFR/PD-L1).

The present disclosure provides a multi-specific antigen binding protein complex comprising a third polypeptide chain (or a portion thereof) which comprises an amino acid sequence that is at least 95% identical to, at least 96% identical to, at least 97% identical to, at least 98% identical to, or at least 99% identical to the amino acid sequences shown in FIGS. 32A-B (e.g., CD38/CD3) or 35A-B (e.g., EGFR/PD-L1).

The present disclosure provides a multi-specific antigen binding protein complex comprising a first polypeptide chain which comprises (e.g., in N- to C-terminal order) a heavy chain variable domain comprising an amino acid sequence that is at least 95% identical to, at least 96% identical to, at least 97% identical to, at least 98% identical to, at least 99% identical to, or 100% identical to SEQ ID NO:59; a heavy chain constant domain (e.g., SEQ ID NO:60 or a sequence at least 95%, 96%, 97%, 98%, or 99% identical thereto); a first linker (e.g., SEQ ID NO:61 or a sequence at least 95%, 96%, 97%, 98%, or 99% identical thereto); an amino acid sequence that is at least 95% identical to, at least 96% identical to, at least 97% identical to, at least 98% identical to, at least 99% identical to, or 100% identical to SEQ ID NO:62; and a heavy chain constant domain (e.g., SEQ ID NO:63 or a sequence at least 95%, 96%, 97%, 98%, or 99% identical thereto); a hinge sequence (e.g., SEQ ID NO:64 or a sequence at least 95%, 96%, 97%, 98%, or 99% identical thereto); a CH2 domain (e.g., SEQ ID NO:65 or a sequence at least 95%, 96%, 97%, 98%, or 99% identical thereto); a CH3 domain (e.g., SEQ ID NO:66 or a sequence at least 95%, 96%, 97%, 98%, or 99% identical thereto); a second polypeptide chain which comprises (e.g., in N- to C-terminal order) an amino acid sequence that is at least 95% identical to, at least 96% identical to, at least 97% identical to, at least 98% identical to, at least 99% identical to, or 100% identical to SEQ ID NO:67; a light chain constant domain (e.g., SEQ ID NO:68 or a sequence at least 95%, 96%, 97%, 98%, or 99% identical thereto); and a third polypeptide chain which comprises (e.g., in N- to C-terminal order) an amino acid sequence that is at least 95% identical to, at least 96% identical to, at least 97% identical to, at least 98% identical to, at least 99% identical to, or 100% identical to SEQ ID NO:69; and a light chain constant domain (e.g., SEQ ID NO:70 or a sequence at least 95%, 96%, 97%, 98%, or 99% identical thereto), a hinge sequence (e.g., SEQ ID NO:71 or a sequence at least 95%, 96%, 97%, 98%, or 99% identical thereto); a CH2 domain (e.g., SEQ ID NO:72 or a sequence at least 95%, 96%, 97%, 98%, or 99% identical thereto); and a CH3 domain (e.g., SEQ ID NO:73 or a sequence at least 95%, 96%, 97%, 98%, or 99% identical thereto).

In one embodiment, the first Fab region and the second Fab region are capable of binding to the first and second target epitopes, respectively, at the same time.

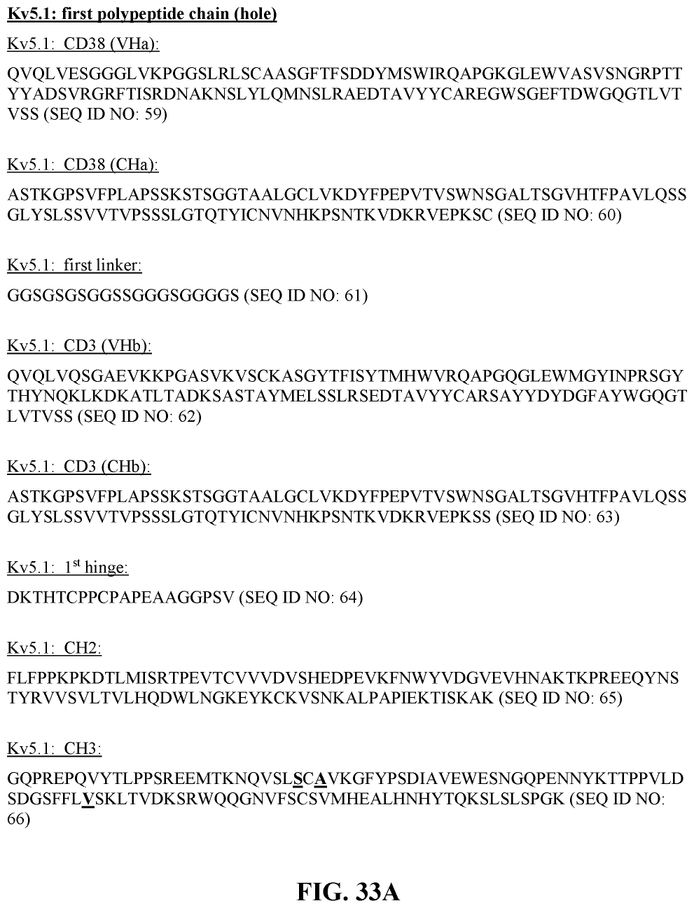

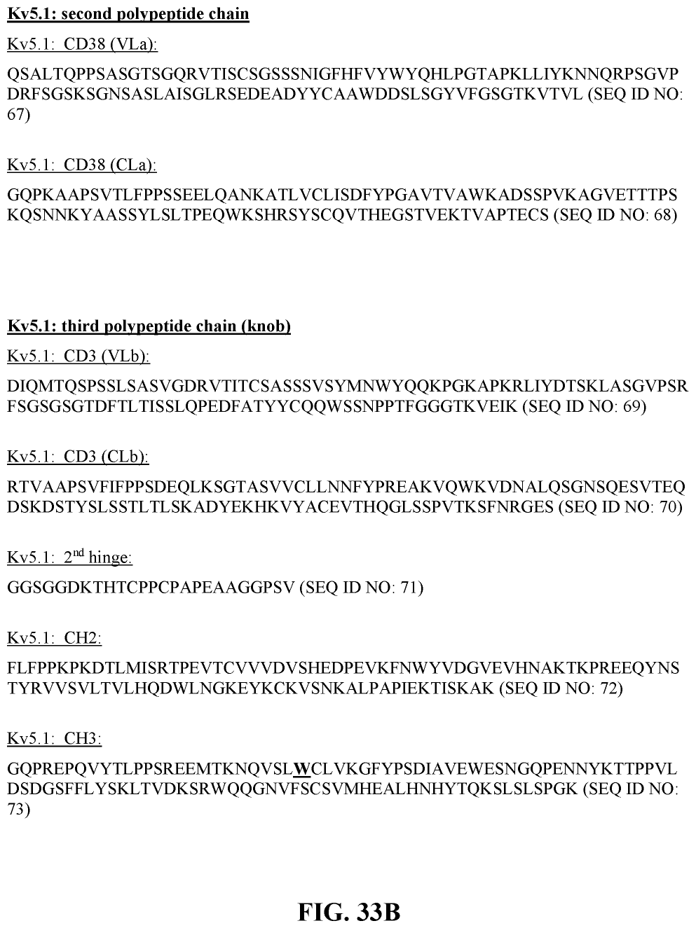

The present disclosure provides a multi-specific antigen binding protein complex comprising a first polypeptide chain (or a portion thereof) which comprises an amino acid sequence that is at least 95% identical to, at least 96% identical to, at least 97% identical to, at least 98% identical to, or at least 99% identical to the amino acid sequences shown in FIGS. 33A-B (e.g., Kv5.1, CD38/CD3).

The present disclosure provides a multi-specific antigen binding protein complex comprising a second polypeptide chain (or a portion thereof) which comprises an amino acid sequence that is at least 95% identical to, at least 96% identical to, at least 97% identical to, at least 98% identical to, or at least 99% identical to the amino acid sequences FIGS. 33A-B (e.g., Kv5.1, CD38/CD3).

The present disclosure provides a multi-specific antigen binding protein complex comprising a third polypeptide chain (or a portion thereof) which comprises an amino acid sequence that is at least 95% identical to, at least 96% identical to, at least 97% identical to, at least 98% identical to, or at least 99% identical to the amino acid sequences FIGS. 33A-B (e.g., Kv5.1, CD38/CD3).

The present disclosure provides a multi-specific antigen binding protein complex comprising a first polypeptide chain which comprises (e.g., in N- to C-terminal order) a heavy chain variable domain comprising an amino acid sequence that is at least 95% identical to, at least 96% identical to, at least 97% identical to, at least 98% identical to, at least 99% identical to, or 100% identical to SEQ ID NO:105; a heavy chain constant domain (e.g., SEQ ID NO:106 or a sequence at least 95%, 96%, 97%, 98%, or 99% identical thereto); a first linker (e.g., SEQ ID NO:107 or a sequence at least 95%, 96%, 97%, 98%, or 99% identical thereto); an amino acid sequence that is at least 95% identical to, at least 96% identical to, at least 97% identical to, at least 98% identical to, at least 99% identical to, or 100% identical to SEQ ID NO:108; a heavy chain constant domain (e.g., SEQ ID NO:109 or a sequence at least 95%, 96%, 97%, 98%, or 99% identical thereto); a hinge sequence (e.g., SEQ ID NO:110 or a sequence at least 95%, 96%, 97%, 98%, or 99% identical thereto); a CH2 domain (e.g., SEQ ID NO:111 or a sequence at least 95%, 96%, 97%, 98%, or 99% identical thereto); and a CH3 domain (e.g., SEQ ID NO:112 or a sequence at least 95%, 96%, 97%, 98%, or 99% identical thereto); a second polypeptide chain which comprises (e.g., in N- to C-terminal order) an amino acid sequence that is at least 95% identical to, at least 96% identical to, at least 97% identical to, at least 98% identical to, at least 99% identical to, or 100% identical to SEQ ID NO:113; and a light chain constant domain (e.g., SEQ ID NO:114 or a sequence at least 95%, 96%, 97%, 98%, or 99% identical thereto); and a third polypeptide chain which comprises (e.g., in N- to C-terminal order) an amino acid sequence that is at least 95% identical to, at least 96% identical to, at least 97% identical to, at least 98% identical to, at least 99% identical to, or 100% identical to SEQ ID NO:115; a light chain constant domain (e.g., SEQ ID NO:116 or a sequence at least 95%, 96%, 97%, 98%, or 99% identical thereto); a hinge sequence (e.g., SEQ ID NO:117 or a sequence at least 95%, 96%, 97%, 98%, or 99% identical thereto); a CH2 domain (e.g., SEQ ID NO:118 or a sequence at least 95%, 96%, 97%, 98%, or 99% identical thereto); and a CH3 domain (e.g., SEQ ID NO:119 or a sequence at least 95%, 96%, 97%, 98%, or 99% identical thereto).

In one embodiment, the first Fab region and the second Fab region are capable of binding to the first and second target epitopes, respectively, at the same time.

The present disclosure provides a multi-specific antigen binding protein complex comprising a first polypeptide chain (or a portion thereof) which comprises an amino acid sequence that is at least 95% identical to, at least 96% identical to, at least 97% identical to, at least 98% identical to, or at least 99% identical to the amino acid sequences shown in FIGS. 36A-B (e.g., Kv5.1, EGFR/PD-L1).

The present disclosure provides a multi-specific antigen binding protein complex comprising a second polypeptide chain (or a portion thereof) which comprises an amino acid sequence that is at least 95% identical to, at least 96% identical to, at least 97% identical to, at least 98% identical to, or at least 99% identical to the amino acid sequences FIGS. 36A-B (e.g., Kv5.1, EGFR/PD-L1).

The present disclosure provides a multi-specific antigen binding protein complex comprising a third polypeptide chain (or a portion thereof) which comprises an amino acid sequence that is at least 95% identical to, at least 96% identical to, at least 97% identical to, at least 98% identical to, or at least 99% identical to the amino acid sequences FIGS. 36A-B (e.g., Kv5.1, EGFR/PD-L1).

The present disclosure provides a multi-specific antigen binding protein complex comprising a first polypeptide chain which comprises (e.g., in N- to C-terminal order) a heavy chain variable domain comprising an amino acid sequence that is at least 95% identical to, at least 96% identical to, at least 97% identical to, at least 98% identical to, at least 99% identical to, or 100% identical to SEQ ID NO:135; a heavy chain constant domain (e.g., SEQ ID NO:136 or a sequence at least 95%, 96%, 97%, 98%, or 99% identical thereto); a first linker (e.g., SEQ ID NO:137 or a sequence at least 95%, 96%, 97%, 98%, or 99% identical thereto); an amino acid sequence that is at least 95% identical to, at least 96% identical to, at least 97% identical to, at least 98% identical to, at least 99% identical to, or 100% identical to SEQ ID NO:138; and a heavy chain constant domain (e.g., SEQ ID NO:139 or a sequence at least 95%, 96%, 97%, 98%, or 99% identical thereto); a hinge sequence (e.g., SEQ ID NO:140 or a sequence at least 95%, 96%, 97%, 98%, or 99% identical thereto); a CH2 domain (e.g., SEQ ID NO:141 or a sequence at least 95%, 96%, 97%, 98%, or 99% identical thereto); a CH3 domain (e.g., SEQ ID NO:142 or a sequence at least 95%, 96%, 97%, 98%, or 99% identical thereto); a second polypeptide chain which comprises (e.g., in N- to C-terminal order) an amino acid sequence that is at least 95% identical to, at least 96% identical to, at least 97% identical to, at least 98% identical to, at least 99% identical to, or 100% identical to SEQ ID NO:143; a light chain constant domain (e.g., SEQ ID NO:144 or a sequence at least 95%, 96%, 97%, 98%, or 99% identical thereto); and a third polypeptide chain which comprises (e.g., in N- to C-terminal order) an amino acid sequence that is at least 95% identical to, at least 96% identical to, at least 97% identical to, at least 98% identical to, at least 99% identical to, or 100% identical to SEQ ID NO:145; and a light chain constant domain (e.g., SEQ ID NO:146 or a sequence at least 95%, 96%, 97%, 98%, or 99% identical thereto), a hinge sequence (e.g., SEQ ID NO:147 or a sequence at least 95%, 96%, 97%, 98%, or 99% identical thereto); a CH2 domain (e.g., SEQ ID NO:148 or a sequence at least 95%, 96%, 97%, 98%, or 99% identical thereto); and a CH3 domain (e.g., SEQ ID NO:149 or a sequence at least 95%, 96%, 97%, 98%, or 99% identical thereto).

In one embodiment, the first Fab region and the second Fab region are capable of binding to the first and second target epitopes, respectively, at the same time.

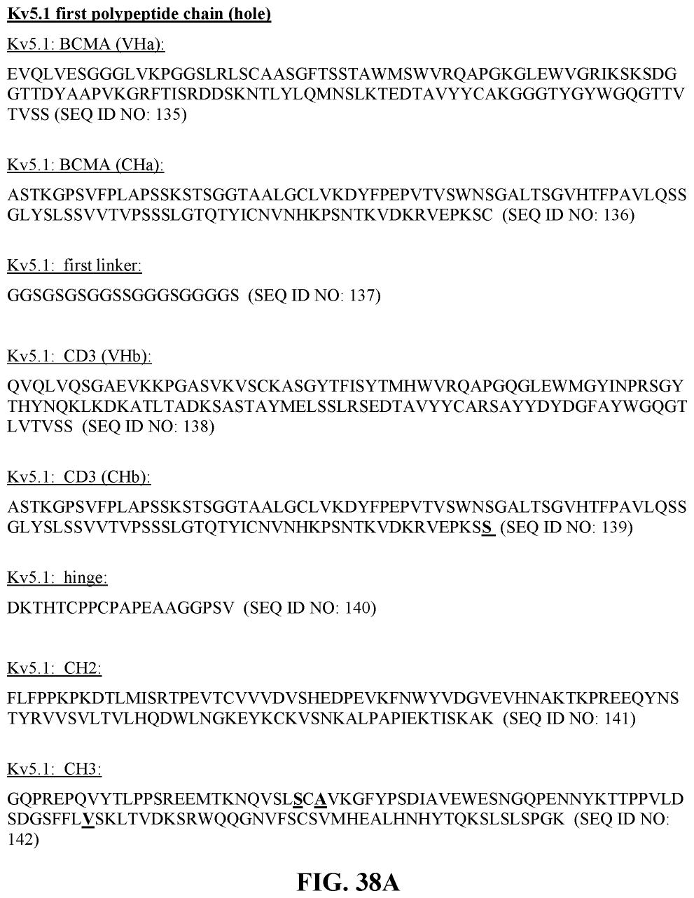

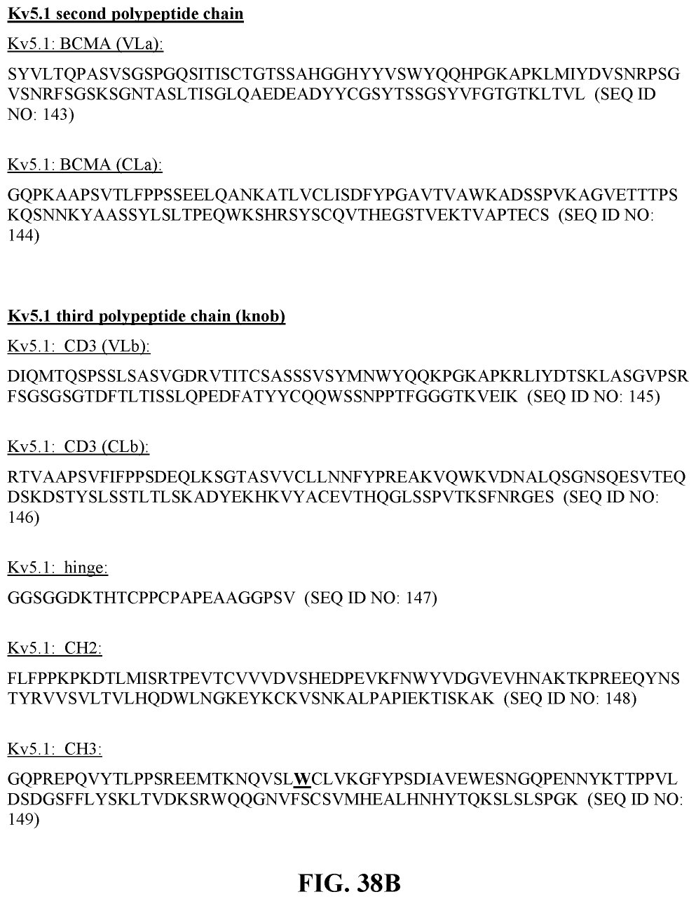

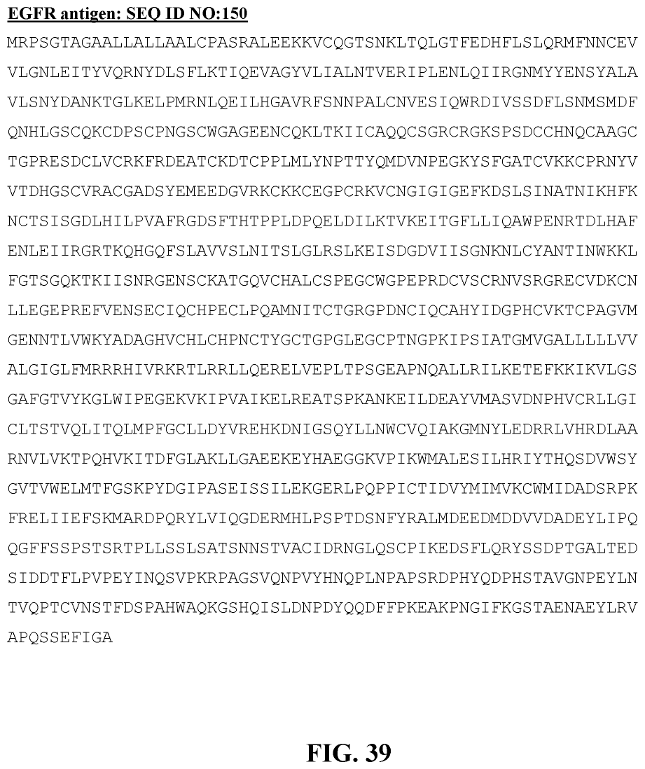

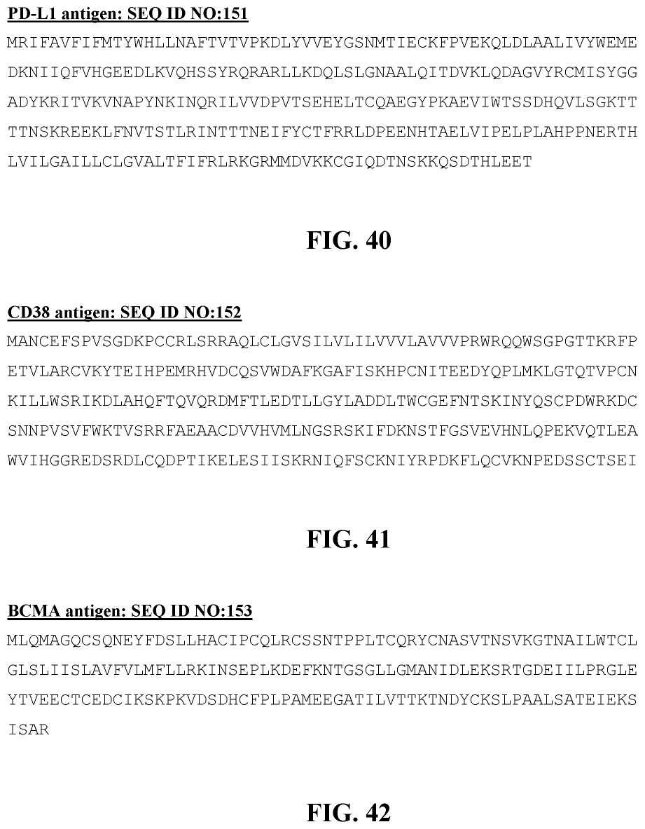

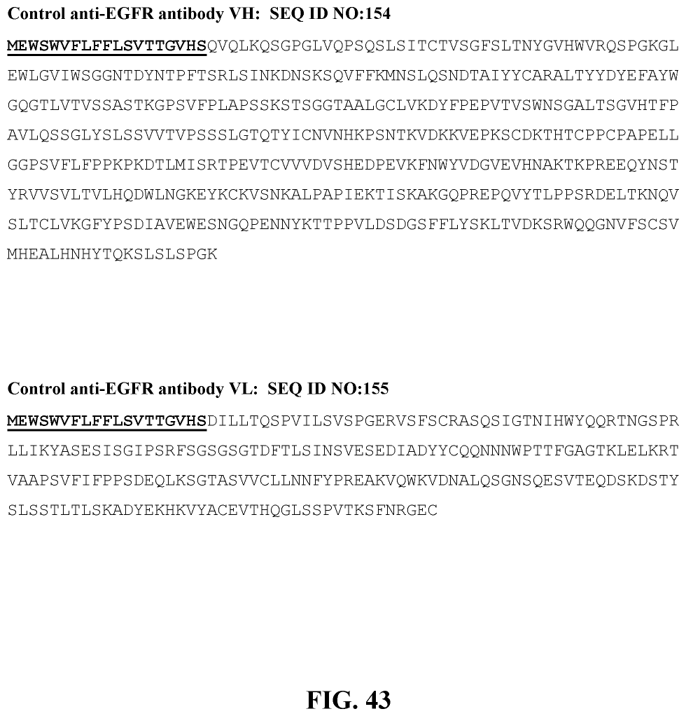

The present disclosure provides a multi-specific antigen binding protein complex comprising a first polypeptide chain (or a portion thereof) which comprises an amino acid sequence that is at least 95% identical to, at least 96% identical to, at least 97% identical to, at least 98% identical to, or at least 99% identical to the amino acid sequences shown in FIGS. 38A-B (e.g., Kv5.1, BCMA/CD3).

The present disclosure provides a multi-specific antigen binding protein complex comprising a second polypeptide chain (or a portion thereof) which comprises an amino acid sequence that is at least 95% identical to, at least 96% identical to, at least 97% identical to, at least 98% identical to, or at least 99% identical to the amino acid sequences FIGS. 38A-B (e.g., Kv5.1, BCMA/CD3).

The present disclosure provides a multi-specific antigen binding protein complex comprising a third polypeptide chain (or a portion thereof) which comprises an amino acid sequence that is at least 95% identical to, at least 96% identical to, at least 97% identical to, at least 98% identical to, or at least 99% identical to the amino acid sequences FIGS. 38A-B (e.g., Kv5.1, BCMA/CD3).

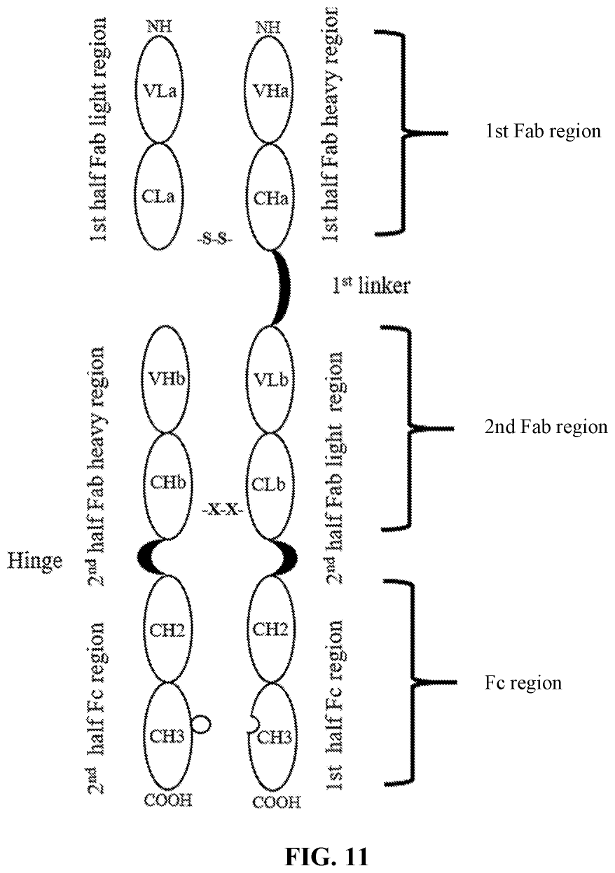

One skilled in the art will appreciate that three chain multi-specific antigen binding protein complexes can comprise polypeptide chains having alternative arrangements are possible. In one example, the first polypeptide chain comprises: a first-variable region (VHa) and a first-constant region (CHa) from a first Fab heavy chain, a first linker (L1), a second-variable region (VLb) and a second-constant region (CLb) from a second Fab light chain, and a first Fc region (CH2 and CH3). The second polypeptide chain comprises: a first-variable region (VLa) and a first-constant region (CLa) from a first Fab light chain. The third polypeptide chain comprises: a second-variable region (VHb) and a second-constant region (CHb) from a second Fab heavy chain, and a second Fc region (CH2 and CH3). In one embodiment, the first linker is not cleavable (FIG. 11).

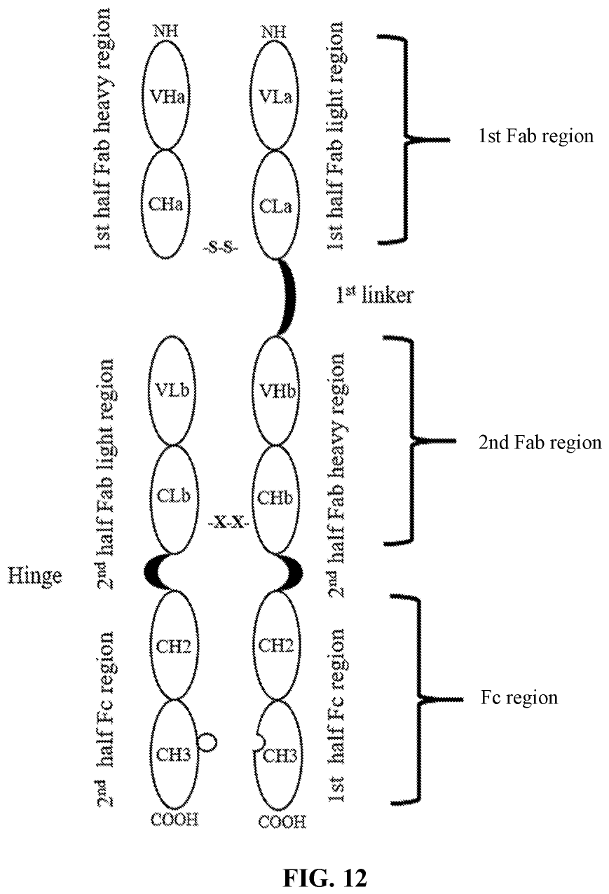

In another example, the first polypeptide chain comprises: a first-variable region (VLa) and a first-constant region (CLa) from a first Fab light chain, a first linker (L1), a second-variable region (VHb) and a second-constant region (CHb) from a second Fab heavy chain, and a first Fc region (CH2 and CH3). The second polypeptide chain comprises: a first-variable region (VHa) and a first-constant region (CHa) from a first Fab heavy chain. The third polypeptide chains comprises: a second-variable region (VLb) and a second-constant region (CLb) from a second Fab light chain, and a second Fc region (CH2 and CH3). In one embodiment, the first linker is not cleavable (FIG. 12).

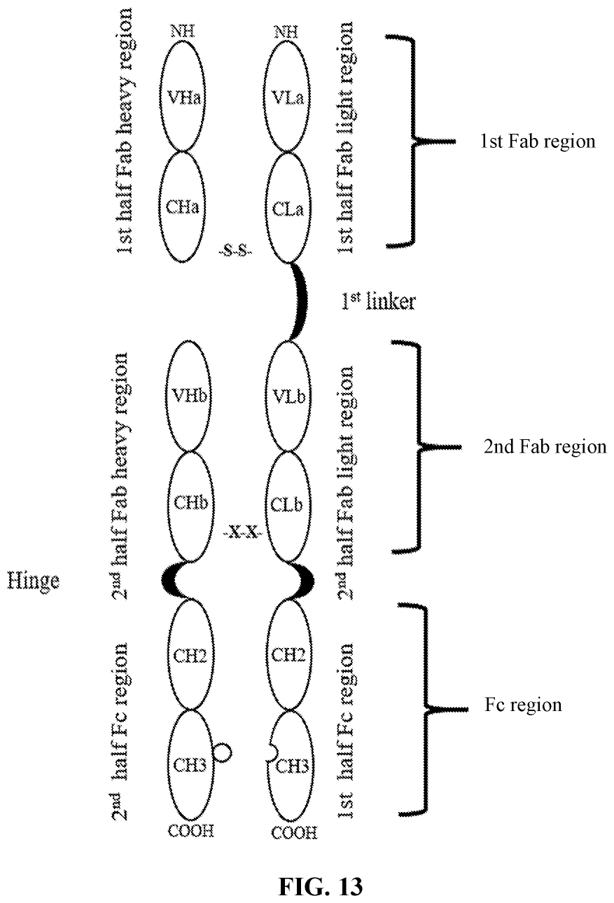

In yet another example, the first polypeptide chain comprises: a first-variable region (VLa) and a first-constant region (CLa) from a first Fab light chain, a first linker (L1), a second-variable region (VLb) and a second-constant region (CLb) from a second Fab light chain, and a first Fc region (CH2 and CH3). The second polypeptide chain comprises: a first-variable region (VHa) and a first-constant region (CHa) from a first Fab heavy chain. The third polypeptide chain comprises: a second-variable region (VHb) and a second-constant region (CHb) from a second Fab heavy chain, and a second Fc region (CH2 and CH3). In one embodiment, the first linker is not cleavable (FIG. 13).

In one embodiment, the alternative protein complexes shown in FIGS. 11, 12 and 13, comprise a wild type Fc region or a mutant Fc region, wherein the mutant Fc region comprises an equivalent to a LALA or LALA-PG mutation (e.g., aligned at L234A, L235A, P329G) which reduces effector function. In one embodiment, the first or second linker is cleavable with a protease, such as a matrix metalloprotease. In one embodiment, the first or second linker comprises the amino acid sequence of one of the cleavable linkers according to SEQ ID NOS:11-24 or 43-56. In one embodiment, the first or second linker is not cleavable with a protease and comprises the amino acid sequence of one of the non-cleavable linkers according to SEQ ID NOS:3, 35, 61, 79, 80, 95, 107 or 125.

The present disclosure provides a three chain multi-specific antigen binding protein complex that can bind two different epitopes on the same target antigen or two different epitopes on different target antigens, comprising: (a) a first polypeptide chain comprising (i) a first half Fab heavy region, (ii) a first half Fc region, (iii) a first linker, and (iv) a second half Fab heavy region, wherein the first half Fab heavy region comprises a first-variable region and first-constant region from a first Fab heavy chain, and wherein the second half Fab heavy region comprises a second-variable region and second-constant region from a second Fab heavy chain; (b) a second polypeptide chain comprising (i) a first half Fab light region, and (ii) a second half Fc region; and (c) a third polypeptide chain comprising a second half Fab light region, wherein the first half Fab light region comprises a first-variable region and first-constant region from a first Fab light chain, and wherein the second half Fab light region comprises a second-variable region and second-constant region from a second Fab light chain. In one embodiment, the first polypeptide chain comprises first and second half Fab regions arranged in a non-tandem manner, and the second and third polypeptide chains are assembled with the first polypeptide chain and comprise first and second half Fab regions arranged in a non-tandem manner (FIG. 4).

In one embodiment, the three chain multi-specific antigen binding protein complex comprises: (a) a first polypeptide chain comprising 5 regions ordered from the amino terminus to the carboxyl terminus: (i) a first heavy chain variable region (VHa) and a first heavy chain constant region (CHa), (ii) a first hinge region, (iii) a first Fc region comprising a first CH2 region and a first CH3 region, (iv) a first linker (L1), and (v) a second heavy chain variable region (VHb) and a second heavy chain constant region (CHb); and (b) a second polypeptide chain comprising three regions ordered from the amino terminus to the carboxyl terminus: (i) a first light chain variable region (VLa) and a first light chain constant region (CLa), (ii) a second hinge region, (iii) a second Fc region comprising a second CH2 region and a second CH3 region; and (c) a third polypeptide chain comprising a region ordered from the amino terminus to the carboxyl terminus: a second light chain variable region (VLb) and a second light chain constant region (CLb), wherein the first, second and third polypeptide chains associate with each other to form the multi-specific antigen binding protein complex having a first Fab region that is capable of binding a first epitope and having a second Fab region that is capable of binding a second epitope that differs from the first epitope. In one embodiment, the first polypeptide chain comprises first and second half Fab regions arranged in a non-tandem manner, and the second and third polypeptide chains are assembled with the first polypeptide chain and comprise first and second half Fab regions arranged in a non-tandem manner (FIG. 4).

The first, second and third polypeptide chains can associate with each other to form the multi-specific antigen binding protein complex having an Fc region that is capable of binding an Fc receptor. In one embodiment, the Fc region comprises an equivalent to a LALA or LALA-PG mutation (e.g., aligns with L234A, L235A, P329G) which reduces effector function. The first polypeptide chain can associate with the second and third polypeptide chains via covalent and/or non-covalent bonds to form the multi-specific antigen binding protein complex. In one embodiment, the covalent bond comprises a disulfide bond. In one embodiment, the non-covalent bond comprises steric complementarity (e.g., knob-in-hole) or electrostatic complementarity. In one embodiment, a knob-in-hole structure is located in the full Fc domain as shown in FIG. 4.

The present disclosure provides a multi-specific antigen binding protein complex comprising a first polypeptide chain (or a portion thereof) which comprises an amino acid sequence that is at least 95% identical to, at least 96% identical to, at least 97% identical to, at least 98% identical to, or at least 99% identical to the amino acid sequences shown in FIGS. 33A-B (e.g., Kv4.33, CD38/CD3), 34A-B (e.g., Kv4.33, BCMA/CD3) and 36A-B (e.g., Kv4.33, PD-L1/EGFR).

The present disclosure provides a multi-specific antigen binding protein complex comprising a second polypeptide chain (or a portion thereof) which comprises an amino acid sequence that is at least 95% identical to, at least 96% identical to, at least 97% identical to, at least 98% identical to, or at least 99% identical to the amino acid sequences shown in FIGS. 33A-B (e.g., Kv4.33, CD38/CD3), 34A-B (e.g., Kv4.33, BCMA/CD3) and 36A-B (e.g., Kv4.33, PD-L1/EGFR).

The present disclosure provides a multi-specific antigen binding protein complex comprising a third polypeptide chain (or a portion thereof) which comprises an amino acid sequence that is at least 95% identical to, at least 96% identical to, at least 97% identical to, at least 98% identical to, or at least 99% identical to the amino acid sequences shown in FIGS. 33A-B (e.g., Kv4.33, CD38/CD3), 34A-B (e.g., Kv4.33, BCMA/CD3) and 36A-B (e.g., Kv4.33, PD-L1/EGFR).

The present disclosure provides a multi-specific antigen binding protein complex comprising a first polypeptide chain which comprises (e.g., in N- to C-terminal order) a heavy chain variable domain comprising an amino acid sequence that is at least 95% identical to, at least 96% identical to, at least 97% identical to, at least 98% identical to, at least 99% identical to, or 100% identical to SEQ ID NO:74; a heavy chain constant domain (e.g., SEQ ID NO:75 or a sequence at least 95%, 96%, 97%, 98%, or 99% identical thereto); a hinge sequence (e.g., SEQ ID NO:76 or a sequence at least 95%, 96%, 97%, 98%, or 99% identical thereto); a CH2 domain (e.g., SEQ ID NO:77 or a sequence at least 95%, 96%, 97%, 98%, or 99% identical thereto); a CH3 domain (e.g., SEQ ID NO:78 or a sequence at least 95%, 96%, 97%, 98%, or 99% identical thereto); a first linker (e.g., SEQ ID NO:79 or 80 or a sequence at least 95%, 96%, 97%, 98%, or 99% identical thereto); an amino acid sequence that is at least 95% identical to, at least 96% identical to, at least 97% identical to, at least 98% identical to, at least 99% identical to, or 100% identical to SEQ ID NO:81; and a heavy chain constant domain (e.g., SEQ ID NO:82 or a sequence at least 95%, 96%, 97%, 98%, or 99% identical thereto); a second polypeptide chain which comprises (e.g., in N- to C-terminal order) an amino acid sequence that is at least 95% identical to, at least 96% identical to, at least 97% identical to, at least 98% identical to, at least 99% identical to, or 100% identical to SEQ ID NO:83; a light chain constant domain (e.g., SEQ ID NO:84 or a sequence at least 95%, 96%, 97%, 98%, or 99% identical thereto); a hinge sequence (e.g., SEQ ID NO:85 or a sequence at least 95%, 96%, 97%, 98%, or 99% identical thereto); a CH2 domain (e.g., SEQ ID NO:86 or a sequence at least 95%, 96%, 97%, 98%, or 99% identical thereto); and a CH3 domain (e.g., SEQ ID NO:87 or a sequence at least 95%, 96%, 97%, 98%, or 99% identical thereto); and a third polypeptide chain which comprises (e.g., in N- to C-terminal order) an amino acid sequence that is at least 95% identical to, at least 96% identical to, at least 97% identical to, at least 98% identical to, at least 99% identical to, or 100% identical to SEQ ID NO:88; and a light chain constant domain (e.g., SEQ ID NO:89 or a sequence at least 95%, 96%, 97%, 98%, or 99% identical thereto).

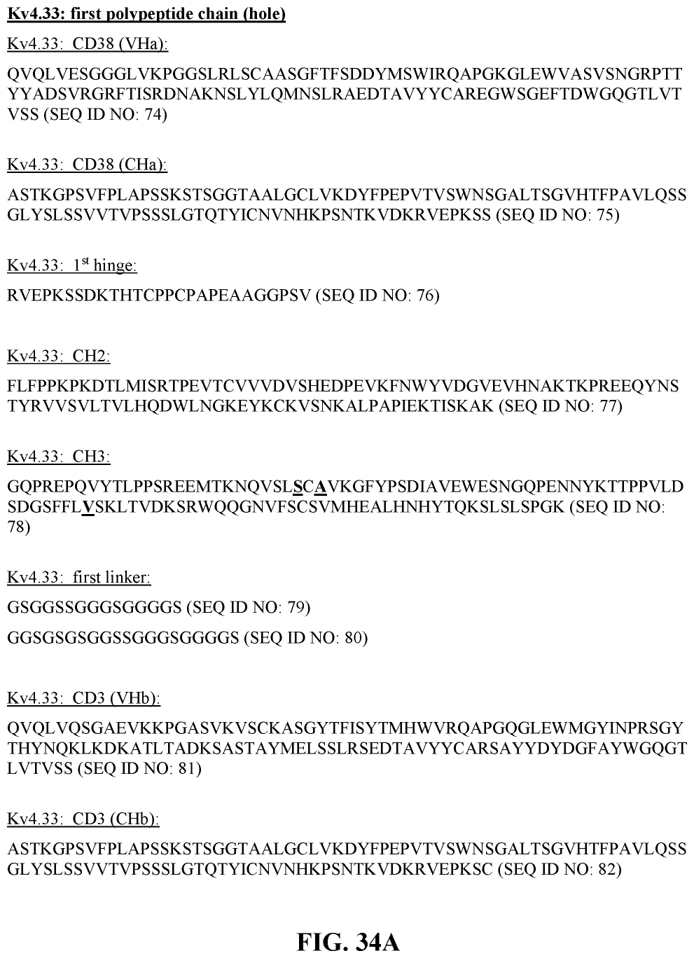

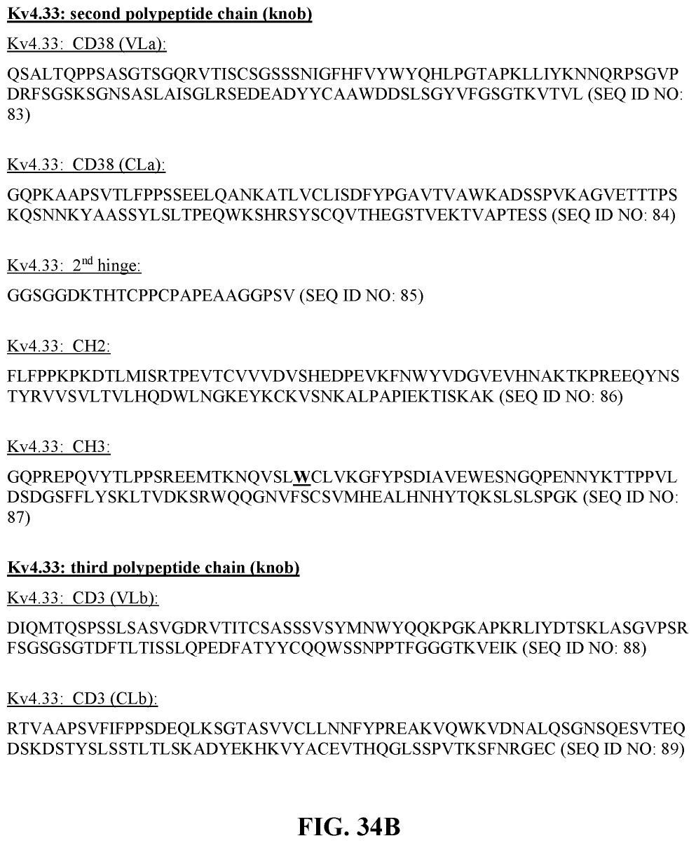

The present disclosure provides a multi-specific antigen binding protein complex comprising a first polypeptide chain (or a portion thereof) which comprises an amino acid sequence that is at least 95% identical to, at least 96% identical to, at least 97% identical to, at least 98% identical to, or at least 99% identical to the amino acid sequences shown in FIGS. 34A-B (e.g., Kv4.33, CD38/CD3).

The present disclosure provides a multi-specific antigen binding protein complex comprising a second polypeptide chain (or a portion thereof) which comprises an amino acid sequence that is at least 95% identical to, at least 96% identical to, at least 97% identical to, at least 98% identical to, or at least 99% identical to the amino acid sequences FIGS. 34A-B (e.g., Kv4.33, CD38/CD3).

The present disclosure provides a multi-specific antigen binding protein complex comprising a third polypeptide chain (or a portion thereof) which comprises an amino acid sequence that is at least 95% identical to, at least 96% identical to, at least 97% identical to, at least 98% identical to, or at least 99% identical to the amino acid sequences FIGS. 34A-B (e.g., Kv4.33, CD38/CD3).

The present disclosure provides a multi-specific antigen binding protein complex comprising a first polypeptide chain which comprises (e.g., in N- to C-terminal order) a heavy chain variable domain comprising an amino acid sequence that is at least 95% identical to, at least 96% identical to, at least 97% identical to, at least 98% identical to, at least 99% identical to, or 100% identical to SEQ ID NO:90; a heavy chain constant domain (e.g., SEQ ID NO:91 or a sequence at least 95%, 96%, 97%, 98%, or 99% identical thereto); a hinge sequence (e.g., SEQ ID NO:92 or a sequence at least 95%, 96%, 97%, 98%, or 99% identical thereto); a CH2 domain (e.g., SEQ ID NO:93 or a sequence at least 95%, 96%, 97%, 98%, or 99% identical thereto); a CH3 domain (e.g., SEQ ID NO:94 or a sequence at least 95%, 96%, 97%, 98%, or 99% identical thereto); a first linker (e.g., SEQ ID NO:95 or a sequence at least 95%, 96%, 97%, 98%, or 99% identical thereto); an amino acid sequence that is at least 95% identical to, at least 96% identical to, at least 97% identical to, at least 98% identical to, at least 99% identical to, or 100% identical to SEQ ID NO:96; and a heavy chain constant domain (e.g., SEQ ID NO:97 or a sequence at least 95%, 96%, 97%, 98%, or 99% identical thereto); a second polypeptide chain which comprises (e.g., in N- to C-terminal order) an amino acid sequence that is at least 95% identical to, at least 96% identical to, at least 97% identical to, at least 98% identical to, at least 99% identical to, or 100% identical to SEQ ID NO:98; a light chain constant domain (e.g., SEQ ID NO:99 or a sequence at least 95%, 96%, 97%, 98%, or 99% identical thereto); a hinge sequence (e.g., SEQ ID NO:100 or a sequence at least 95%, 96%, 97%, 98%, or 99% identical thereto); a CH2 domain (e.g., SEQ ID NO:101 or a sequence at least 95%, 96%, 97%, 98%, or 99% identical thereto); and a CH3 domain (e.g., SEQ ID NO:102 or a sequence at least 95%, 96%, 97%, 98%, or 99% identical thereto); and a third polypeptide chain which comprises (e.g., in N- to C-terminal order) an amino acid sequence that is at least 95% identical to, at least 96% identical to, at least 97% identical to, at least 98% identical to, at least 99% identical to, or 100% identical to SEQ ID NO:103; and a light chain constant domain (e.g., SEQ ID NO:104 or a sequence at least 95%, 96%, 97%, 98%, or 99% identical thereto).

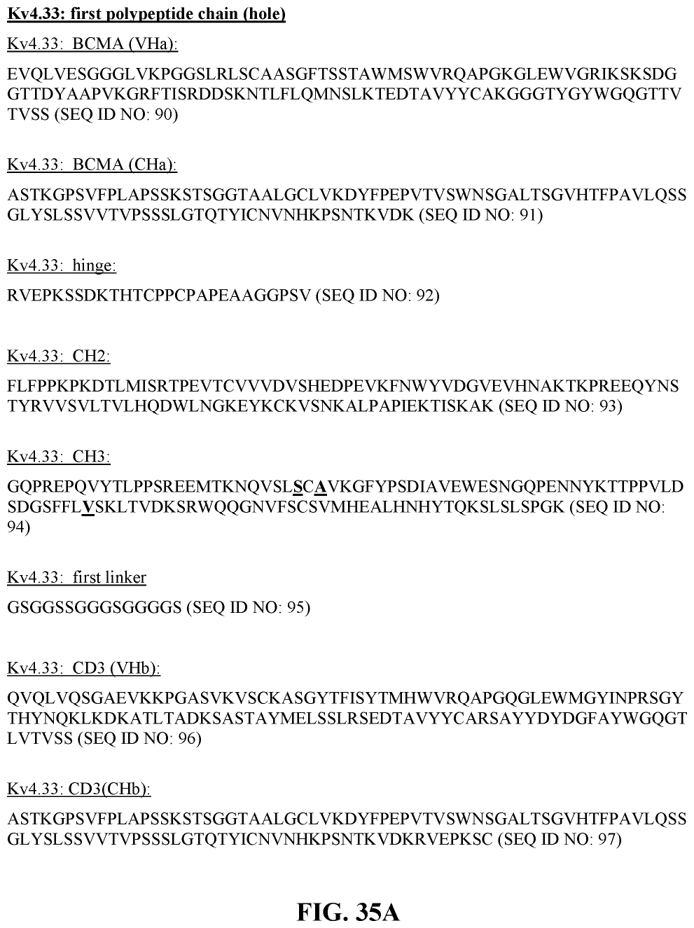

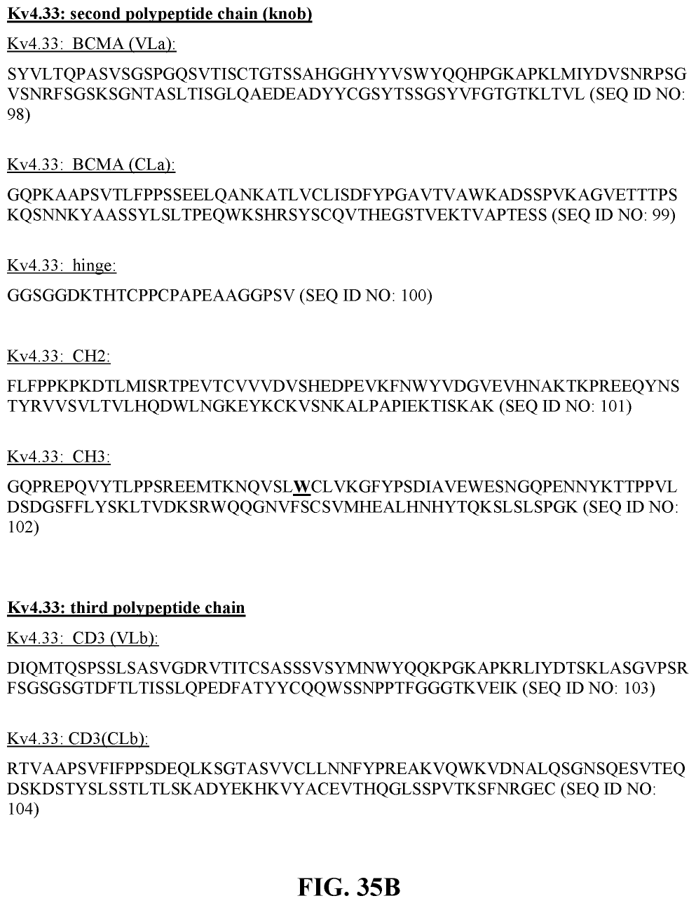

The present disclosure provides a multi-specific antigen binding protein complex comprising a first polypeptide chain (or a portion thereof) which comprises an amino acid sequence that is at least 95% identical to, at least 96% identical to, at least 97% identical to, at least 98% identical to, or at least 99% identical to the amino acid sequences shown in FIGS. 35A-B (e.g., Kv4.33, BCMA/CD3).

The present disclosure provides a multi-specific antigen binding protein complex comprising a second polypeptide chain (or a portion thereof) which comprises an amino acid sequence that is at least 95% identical to, at least 96% identical to, at least 97% identical to, at least 98% identical to, or at least 99% identical to the amino acid sequences FIGS. 35A-B (e.g., Kv4.33, BCMA/CD3).

The present disclosure provides a multi-specific antigen binding protein complex comprising a third polypeptide chain (or a portion thereof) which comprises an amino acid sequence that is at least 95% identical to, at least 96% identical to, at least 97% identical to, at least 98% identical to, or at least 99% identical to the amino acid sequences FIGS. 35A-B (e.g., Kv4.33, BCMA/CD3).

The present disclosure provides a multi-specific antigen binding protein complex comprising a first polypeptide chain which comprises (e.g., in N- to C-terminal order) a heavy chain variable domain comprising an amino acid sequence that is at least 95% identical to, at least 96% identical to, at least 97% identical to, at least 98% identical to, at least 99% identical to, or 100% identical to SEQ ID NO:120; a heavy chain constant domain (e.g., SEQ ID NO:121 or a sequence at least 95%, 96%, 97%, 98%, or 99% identical thereto); a hinge sequence (e.g., SEQ ID NO:122 or a sequence at least 95%, 96%, 97%, 98%, or 99% identical thereto); a CH2 domain (e.g., SEQ ID NO:123 or a sequence at least 95%, 96%, 97%, 98%, or 99% identical thereto); a CH3 domain (e.g., SEQ ID NO:124 or a sequence at least 95%, 96%, 97%, 98%, or 99% identical thereto); a first linker (e.g., SEQ ID NO:125 or a sequence at least 95%, 96%, 97%, 98%, or 99% identical thereto); an amino acid sequence that is at least 95% identical to, at least 96% identical to, at least 97% identical to, at least 98% identical to, at least 99% identical to, or 100% identical to SEQ ID NO:126; and a heavy chain constant domain (e.g., SEQ ID NO:127 or a sequence at least 95%, 96%, 97%, 98%, or 99% identical thereto); a second polypeptide chain which comprises (e.g., in N- to C-terminal order) an amino acid sequence that is at least 95% identical to, at least 96% identical to, at least 97% identical to, at least 98% identical to, at least 99% identical to, or 100% identical to SEQ ID NO:128; a light chain constant domain (e.g., SEQ ID NO:129 or a sequence at least 95%, 96%, 97%, 98%, or 99% identical thereto); a hinge sequence (e.g., SEQ ID NO:130 or a sequence at least 95%, 96%, 97%, 98%, or 99% identical thereto); a CH2 domain (e.g., SEQ ID NO:131 or a sequence at least 95%, 96%, 97%, 98%, or 99% identical thereto); and a CH3 domain (e.g., SEQ ID NO:132 or a sequence at least 95%, 96%, 97%, 98%, or 99% identical thereto); and a third polypeptide chain which comprises (e.g., in N- to C-terminal order) an amino acid sequence that is at least 95% identical to, at least 96% identical to, at least 97% identical to, at least 98% identical to, at least 99% identical to, or 100% identical to SEQ ID NO:133; and a light chain constant domain (e.g., SEQ ID NO:134 or a sequence at least 95%, 96%, 97%, 98%, or 99% identical thereto).

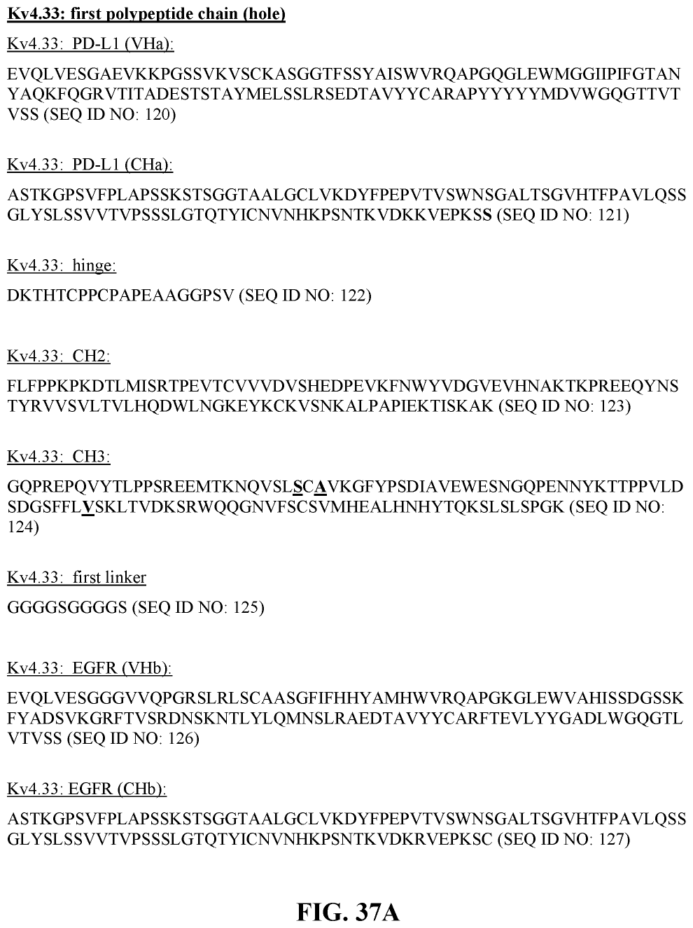

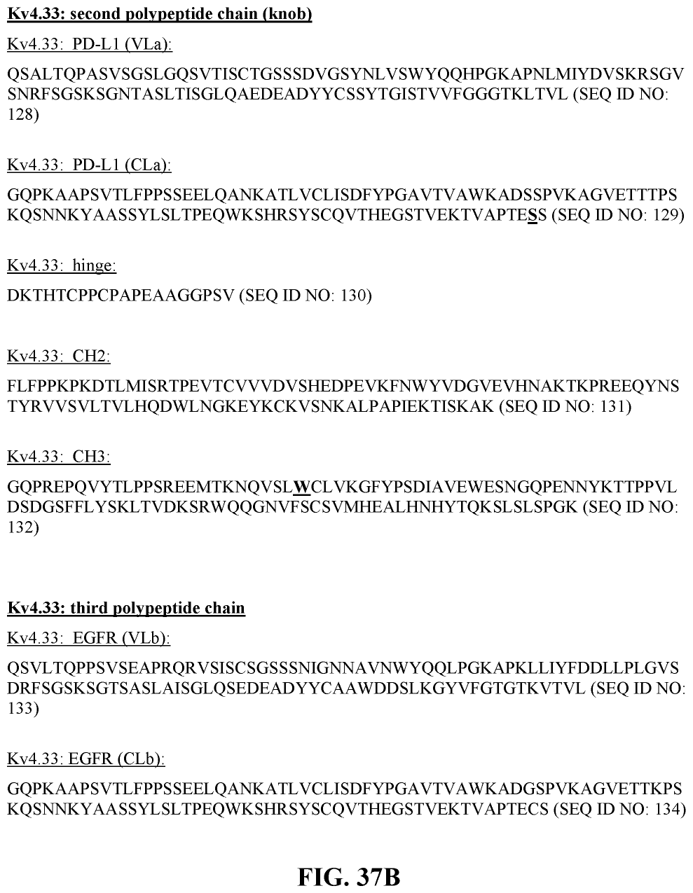

The present disclosure provides a multi-specific antigen binding protein complex comprising a first polypeptide chain (or a portion thereof) which comprises an amino acid sequence that is at least 95% identical to, at least 96% identical to, at least 97% identical to, at least 98% identical to, or at least 99% identical to the amino acid sequences shown in FIGS. 37A-B (e.g., Kv4.33, PDL1/EGFR).

The present disclosure provides a multi-specific antigen binding protein complex comprising a second polypeptide chain (or a portion thereof) which comprises an amino acid sequence that is at least 95% identical to, at least 96% identical to, at least 97% identical to, at least 98% identical to, or at least 99% identical to the amino acid sequences FIGS. 37A-B (e.g., Kv4.33, PDL1/EGFR).

The present disclosure provides a multi-specific antigen binding protein complex comprising a third polypeptide chain (or a portion thereof) which comprises an amino acid sequence that is at least 95% identical to, at least 96% identical to, at least 97% identical to, at least 98% identical to, or at least 99% identical to the amino acid sequences FIGS. 37A-B (e.g., Kv4.33, PDL1/EGFR).

In one embodiment, the first Fab region and the second Fab region are capable of binding to the first and second target epitopes, respectively, at the same time.

In one embodiment, the first polypeptide chain carries a first peptide linker. In one embodiment, the first peptide linker is not cleavable.

In one embodiment, first linker comprises an amino acid sequence selected from a group consisting of: (SG)ₙ (SEQ ID NO: 157), (SGG)ₙ (SEQ ID NO: 158), (SGGG)ₙ (SEQ ID NO: 159), (SSG)ₙ (SEQ ID NO: 160), (GS)ₙ (SEQ ID NO: 161), (GGG)ₙ (SEQ ID NO: 162), (GSGGS)ₙ (SEQ ID NO: 163), (GSG)ₙ (SEQ ID NO: 164), (GGGGS)ₙ (SEQ ID NO: 165), (GGGS)ₙ (SEQ ID NO: 166), (GGGGSGS)ₙ (SEQ ID NO: 167), (GGGGSGGS)ₙ (SEQ ID NO: 168), and (GGS)ₙ (SEQ ID NO: 169), where n is an integer of 1-6. In one embodiment, the first linker comprises an amino acid sequence of TSGSGGSGGSV (SEQ ID NO: 156).

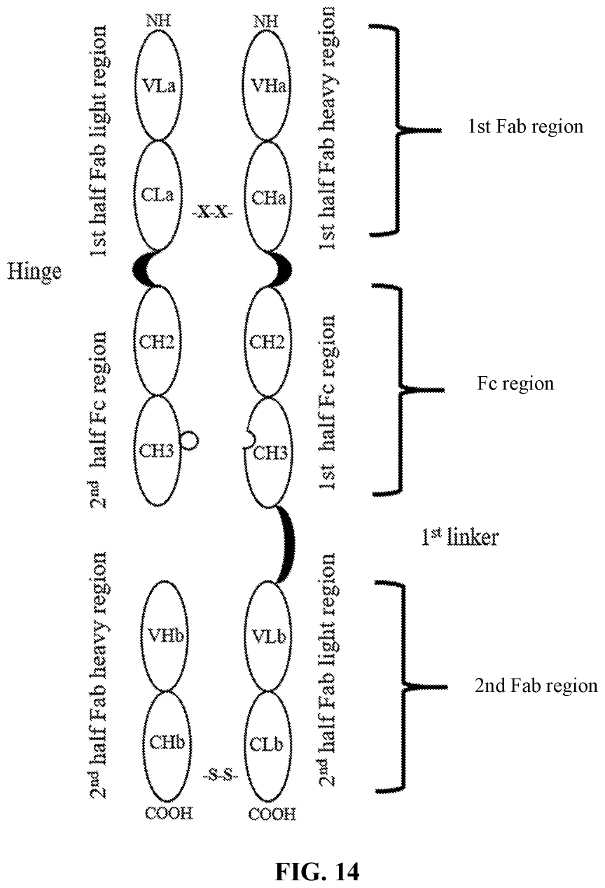

One skilled in the art will appreciate that three chain multi-specific antigen binding protein complexes can comprise polypeptide chains having alternative arrangements are possible. In one example, the first polypeptide chain comprises a first-variable region (VHa) and a first-constant region (CHa) from a first Fab heavy chain, a first Fc region (CH2 and CH3), a first linker (L1), a second-variable region (VLb) and a second-constant region (CLb) from a second Fab light chain. The second polypeptide chain comprises a first-variable region (VLa) and a first-constant region (CLa) from a first Fab light chain, and a second Fc region (CH2 and CH3). The third polypeptide chain comprises a second-variable region (VHb) and a second-constant region (CHb) from a second Fab heavy chain. In one embodiment, the first is not cleavable (FIG. 14).

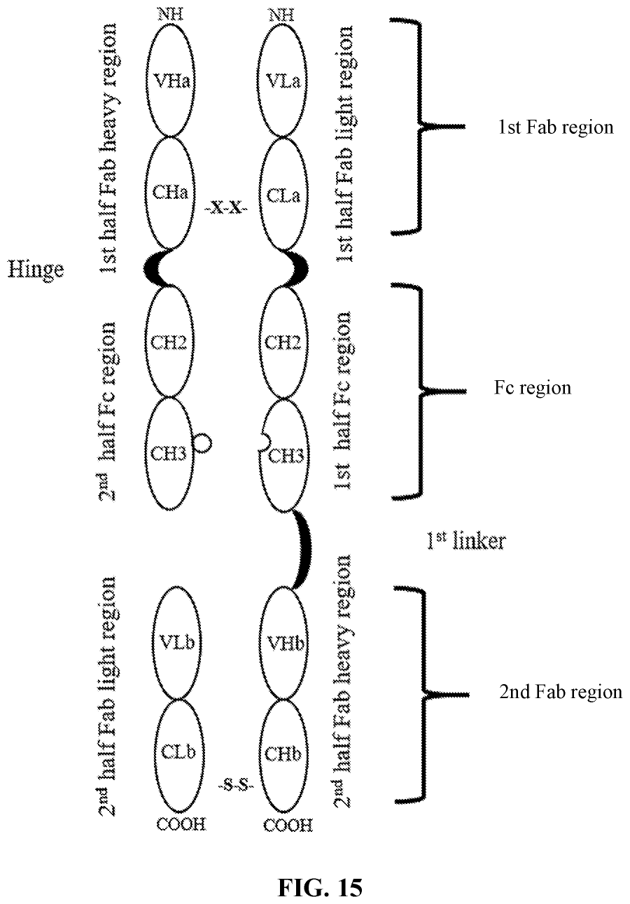

In another example, the first polypeptide chain comprises a first-variable region (VLa) and a first-constant region (CLa) from a first Fab light chain, a first Fc region (CH2 and CH3), a first linker (L1), a second-variable region (VHb) and a second-constant region (CHb) from a second Fab heavy chain. The second polypeptide chain comprises a first-variable region (VHa) and a first-constant region (CHa) from a first Fab heavy chain, and a second Fc region (CH2 and CH3). The third polypeptide chain comprises a second-variable region (VLb) and a second-constant region (CLb) from a second Fab light chain. In one embodiment, the first linker is not cleavable (FIG. 15).

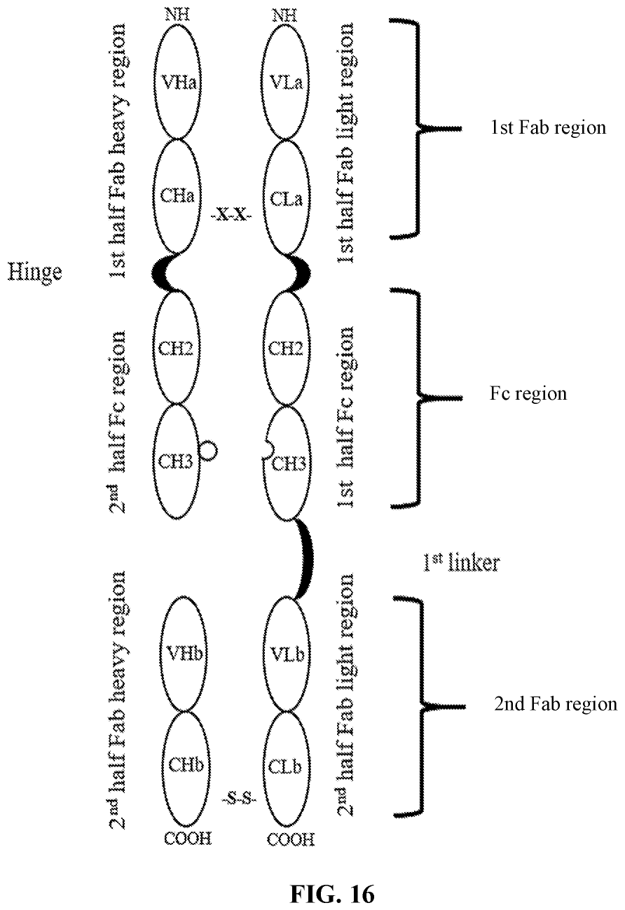

In yet another example, the first polypeptide chain comprises a first-variable region (VLa) and a first-constant region (CLa) from a first Fab light chain, a first Fc region (CH2 and CH3), a first linker (L1), a second-variable region (VLb) and a second-constant region (CLb) from a second Fab light chain. The second polypeptide chain comprises a first-variable region (VHa) and a first-constant region (CHa) from a first Fab heavy chain, and a second Fc region (CH2 and CH3). The third polypeptide chain comprises a second-variable region (VHb) and a second-constant region (CHb) from a second Fab heavy chain. In one embodiment, the first linker is not cleavable (FIG. 16).

In one embodiment, the alternative protein complexes shown in FIGS. 14, 15 and 16, comprise a wild type Fc region or a mutant Fc region, wherein the mutant Fc region comprises an equivalent to a LALA or LALA-PG mutation (e.g., aligned at L234A, L235A, P329G) which reduces effector function. In one embodiment, the first or second linker is cleavable with a protease, such as a matrix metalloprotease. In one embodiment, the first or second linker comprises the amino acid sequence of one of the cleavable linkers according to SEQ ID NOS:11-24 or 43-56. In one embodiment, the first or second linker is not cleavable with a protease and comprises the amino acid sequence of one of the non-cleavable linkers according to SEQ ID NOS:3, 35, 61, 79, 80, 95, 107 or 125.

The present disclosure provides a kit comprising: at least one of the multi-specific antigen binding protein complexes described herein, including a two polypeptide chain protein complex and/or a three polypeptide protein complex, that binds a first and second target epitope. In one embodiment, the kit comprises one or more adjunct compounds selected from a group consisting of Tris, phosphate, carbonate, stabilizers, excipients, biocides and bovine serum albumin. In one embodiment, the kit comprises one or more adjunct compounds selected from a group consisting of iris, phosphate, carbonate, stabilizers, excipients, biocides and bovine serum albumin. In one embodiment, the kit comprises one container which contains at least one multi-specific antigen binding protein complex and optionally one or more adjunct compound. In one embodiment, the kit comprises two or more containers, wherein one container contains at least one multi-specific antigen binding protein complex and a separate container contains one or more adjunct compounds.

The present disclosure provides nucleic acids that encode a first, second and/or third polypeptide chain that make up any of the multi-specific antigen binding protein complexes described herein.

The present disclosure provides a first and a second nucleic acid that encode a first and a second polypeptide chain, respectively, wherein the first and second polypeptide chains can assemble to form a two chain protein complex (e.g., shown in FIG. 1). The present disclosure provides a first nucleic acid that encodes a first polypeptide chain, comprising a first half Fab heavy region, a linker, a second half Fab heavy region, and a first half Fc region, wherein the first and second half heavy Fab regions are arranged in-tandem. The present disclosure provide a second nucleic acid that encodes a second polypeptide comprising a first half Fab light region, a cleavable linker, a second half Fab light region, and a second half Fc region, wherein the first and second half light Fab regions are arranged in-tandem. In one embodiment, the first nucleic acid encodes the first polypeptide chain comprising (i) a first half Fab heavy region, (ii) a first linker, (iii) a second half Fab heavy region, and (iv) a first half Fc region, wherein the first half Fab heavy region comprises a first-variable region and first-constant region from a first Fab heavy chain, and wherein the second half Fab heavy region comprises a second-variable region and second-constant region from a second Fab heavy chain. In one embodiment, the second nucleic acid encodes the second polypeptide chain comprising (i) a first half Fab light region, (ii) a second linker, (iii) a second half Fab light region, and (iv) a second half Fc region, wherein the first half Fab light region comprises a first-variable region and first-constant region from a first Fab light chain, and wherein the second half Fab light region comprises a second-variable region and second-constant region from a second Fab light chain, and wherein the second linker is cleavable. In one embodiment, the Fc region comprises an equivalent to a LALA or LALA-PG mutation (e.g., aligns with L234A, L235A, P329G) which reduces effector function. In one embodiment, the first and/or second nucleic acid further encodes a signal peptide for polypeptide secretion.

In one embodiment, the first nucleic acid encodes the first polypeptide chain comprising the amino acid sequences shown in FIGS. 31A-C (e.g., Kv6.1, CD38/CD3). In one embodiment, the second nucleic acid encodes the second polypeptide chain comprising the amino acid sequences shown in FIGS. 31A-C (e.g., Kv6.1, CD38/CD3).

The present disclosure provides a first and a second nucleic acid that encode a first and a second polypeptide chain, respectively, wherein the first and second polypeptide chains can assemble to form a two chain protein complex (e.g., shown in FIG. 3). The present disclosure provides a first nucleic acid that encodes a first polypeptide comprising a first half Fab heavy region, a first half Fc region, a linker, and a second half Fab heavy region, wherein the first and second half heavy Fab regions are arranged in a non-tandem manner. The present disclosure provides a second nucleic acid that encodes a second polypeptide comprising a first half Fab light region, a second half Fc region, a cleavable linker, and a second half Fab light region, wherein the first and second half heavy Fab regions are arranged in a non-tandem manner. In one embodiment, the first nucleic acid encodes the first polypeptide chain comprising (i) a first half Fab heavy region, (ii) a first half Fc region, (iii) a first linker, and (iv) a second half Fab heavy region, wherein the first half Fab heavy region comprises a first-variable region and first-constant region from a first Fab heavy chain, and wherein the second half Fab heavy region comprises a second-variable region and second-constant region from a second Fab heavy chain. In one embodiment, the second nucleic acid encodes the second polypeptide chain comprising (i) a first half Fab light region, (ii) a second half Fc region, (iii) a second linker, and (iv) a second half Fab light region, wherein the first half Fab light region comprises a first-variable region and first-constant region from a first Fab light chain, and wherein the second half Fab light region comprises a second-variable region and second-constant region from a second Fab light chain, and wherein the second linker is cleavable. In one embodiment, the Fc region comprises an equivalent to a LALA or LALA-PG mutation (e.g., aligns with L234A, L235A, P329G) which reduces effector function. In one embodiment, the first and/or second nucleic acid further encodes a signal peptide for polypeptide secretion.

In one embodiment, the first nucleic acid encodes the first polypeptide chain comprising the amino acid sequences shown in FIGS. 32A-C (e.g., Kv6.1, CD38/CD3). In one embodiment, the second nucleic acid encodes the second polypeptide chain comprising the amino acid sequences shown in FIGS. 32A-C (e.g., Kv6.1, CD38/CD3).

The present disclosure provides a first, second and third nucleic acid that encode a first, second, and third polypeptide chain, respectively, wherein the first, second and third polypeptide chains can assemble to form a three chain protein complex (e.g., shown in FIG. 2). The present disclosure provides a nucleic acid that encodes a first polypeptide comprising a first half Fab heavy region, a linker, a second half Fab heavy region, and a first half Fc region, wherein the first and second half heavy Fab regions are arranged in-tandem. The present disclosure provide a nucleic acid that encodes a second polypeptide comprising a first half Fab light region. The present disclosure provides a nucleic acid that encodes a third polypeptide comprising a second half Fab light region, and a second half Fc region. In one embodiment, the first nucleic acid encodes the first polypeptide chain comprising (i) a first half Fab heavy region, (ii) a first linker, (iii) a second half Fab heavy region, and (iv) a first half Fc region, wherein the first half Fab heavy region comprises a first-variable region and first-constant region from a first Fab heavy chain, and wherein the second half Fab heavy region comprises a second-variable region and second-constant region from a second Fab heavy chain. In one embodiment, the second nucleic acid encodes the second polypeptide chain comprising a first half Fab light region which comprises a first-variable region and first-constant region from a first Fab light chain. In one embodiment, the third nucleic acid encodes the third polypeptide chain comprising (i) a second half Fab light region, and (ii) a second half Fc region, wherein the second half Fab light region comprises a second-variable region and second-constant region from a second Fab light chain. In one embodiment, the Fc region comprises an equivalent to a LALA or LALA-PG mutation (e.g., aligns with L234A, L235A, P329G) which reduces effector function. In one embodiment, the first, second and/or third nucleic acid further encodes a signal peptide for polypeptide secretion.

In one embodiment, the first nucleic acid encodes the first polypeptide chain comprising the amino acid sequences shown in FIGS. 33A-B (e.g., Kv5.1, CD38/CD3) or 36A-B (e.g., Kv5.1, EGFR/PD-L1) or 38A-B (e.g., Kv5.1, BCMA/CD3). In one embodiment, the second nucleic acid encodes the second polypeptide chain comprising the amino acid sequences shown in FIGS. 33A-B (e.g., Kv5.1, CD38/CD3) or 36A-B (e.g., Kv5.1, EGFR/PD-L1) or 38A-B (e.g., Kv5.1, BCMA/CD3). In one embodiment, the third nucleic acid encodes the third polypeptide chain comprising the amino acid sequences shown in FIGS. 33A-B (e.g., Kv5.1, CD38/CD3) or 36A-B (e.g., Kv5.1, EGFR/PD-L1) or 38A-B (e.g., Kv5.1, BCMA/CD3).

The present disclosure provides a first, second and third nucleic acid that encode a first, second, and third polypeptide chain, respectively, wherein the first, second and third polypeptide chains can assemble to form a three chain protein complex (e.g., shown in FIG. 4). The present disclosure provides a first nucleic acid that encodes a first polypeptide comprising a first half Fab heavy region, a first half Fc region, a linker, and a second half Fab heavy region, wherein the first and second half Fab heavy regions are arranged in a non-tandem manner. The present disclosure provides a second nucleic acid that encodes a second polypeptide comprising a first half Fab light region, and a second half Fc region. The present disclosure provides a third nucleic acid that encodes a third polypeptide comprising a second half Fab light region. In one embodiment, the first nucleic acid encodes the first polypeptide comprising (i) a first half Fab heavy region, (ii) a first half Fc region, (iii) a first linker, and (iv) a second half Fab heavy region, wherein the first half Fab heavy region comprises a first-variable region and first-constant region from a first Fab heavy chain, and wherein the second half Fab heavy region comprises a second-variable region and second-constant region from a second Fab heavy chain. In one embodiment, the second nucleic acid encodes the second polypeptide comprising (i) a first half Fab light region, and (ii) a second half Fc region, wherein the first half Fab light region comprises a first-variable region and first-constant region from a first Fab light chain. In one embodiment, the third nucleic acid encodes the third polypeptide comprising a second half Fab light region, wherein the second half Fab light region comprises a second-variable region and second-constant region from a second Fab light chain. In one embodiment, the Fc region comprises an equivalent to a LALA or LALA-PG mutation (e.g., aligns with L234A, L235A, P329G) which reduces effector function. In one embodiment, the first, second and/or third nucleic acid further encodes a signal peptide for polypeptide secretion.

In one embodiment, the first nucleic acid encodes the first polypeptide chain comprising the amino acid sequences shown in FIGS. 34A-B (e.g., Kv4.33, CD38/CD3) or 35A-B (e.g., Kv4.33, BCMA/CD3) or 37A-B (e.g., Kv4.33, PD-L1/EGFR). In one embodiment, the second nucleic acid encodes the second polypeptide chain comprising the amino acid sequences shown in FIGS. 34A-B (e.g., Kv4.33, CD38/CD3) or 35A-B (e.g., Kv4.33, BCMA/CD3) or 37A-B (e.g., Kv4.33, PD-L1/EGFR). In one embodiment, the third nucleic acid encodes the third polypeptide chain comprising the amino acid sequences shown in FIGS. 34A-B (e.g., Kv4.33, CD38/CD3) or 35A-B (e.g., Kv4.33, BCMA/CD3) or 37A-B (e.g., Kv4.33, PD-L1/EGFR).

The present disclosure provides individual vectors, including expression vectors, that are operably joined to one or more nucleic acids (e.g., nucleic acid transgene(s)) that encode a first, second and/or third polypeptide chain that make up any of the multi-specific antigen binding protein complexes described herein. In one embodiment, the expression vector comprises one or more promoters which control transcription of the nucleic acid encoding the first, second and/or third polypeptide chain.

In one embodiment, the vector comprises at least one regulatory sequence, for example a promoter and optionally an enhancer, that is operably joined to a nucleic acid that encodes a first, second or third polypeptide chain, wherein the promoter controls transcription of the nucleic acid encoding the first, second or third polypeptide chain in a mono-cistronic manner.

In one embodiment, the vector comprises a promoter (and optionally an enhancer) that is operably joined to any two or any combination of multiple nucleic acids that encode a first, second and/or third polypeptide chain, where the promoter controls transcription of a polycistronic transcript encoding the first, second and/or third polypeptide chains.

In one embodiment, the vector comprises multiple promoters (and optionally at least one enhancer sequence) to permit operably joining individual promoters to individual nucleic acids each encoding a first, second or third polypeptide chain, wherein multiple promoters within a single vector control transcription of different transcript encoding the first, second and/or third polypeptide chains.

In one embodiment, one vector is introduced into a host cell, wherein the vector within the host cell carries a promoter (and optionally an enhancer sequence) which is operably joined to one nucleic acid that encodes a polypeptide chain (e.g., first, second or third polypeptide chain). Thus, the host cell can express the first, second or third polypeptide chain that make up any of the multi-specific antigen binding protein complexes.

In one embodiment, one vector is introduced into a host cell, wherein the vector within the host cell carries a promoter (and optionally an enhancer sequence) which is operably joined to two or more nucleic acids that encode a first, second and/or third polypeptide chain. Thus, the host cell can express the first, second and/or third polypeptide chain that make up any of the multi-specific antigen binding protein complexes.

In one embodiment, multiple vectors are introduced into a host cell, wherein individual vectors within a host cell carry at least one promoter (and optionally an enhancer sequence), and one nucleic acid that encodes a polypeptide chain is joined to one promoter in one vector. Thus, individual host cells can express any two or any combination of the first, second and/or third polypeptide chains that make up any of the multi-specific antigen binding protein complexes.

The vectors comprise promoters that are inducible or constitutive promoters. The vectors and host cells can be selected to generate transgenic host cells that transiently or stably express any of the polypeptide chains described herein.

The present disclosure provides host cells that harbor a single vector that is operably joined to one or more nucleic acids that encode a first, second and/or third polypeptide chain that make up any of the multi-specific antigen binding protein complexes.

The present disclosure provides host cells that harbor two or more vectors each vector being operably joined to one or more nucleic acids that encode a first, second and/or third polypeptide chain that make up any of the multi-specific antigen binding protein complexes.

The host cell can be a bacterial or mammalian cell. In one embodiment, the host cell comprises a Chinese hamster ovary (CHO) cell.

In one embodiment, at least one vector is introduced into the host cell via lipofection (e.g., using a lipid surfactant); electroporation; transfection employing calcium chloride, rubidium chloride, calcium phosphate, DEAE-dextran, or other substances; viral transfection; non-viral transfection; microprojectile bombardment; and infection (e.g., where the vector is an infectious agent).

In one embodiment, host cells harbor two vectors, wherein the first vector is operably joined to nucleic acids encoding the first polypeptide and the second vector is operably joined to nucleic acid encoding the second polypeptide. The two vectors harbored by the host cells can be present at a molar ratio for first polypeptide:second polypeptide of 1:1, 1:1.5, 1.5:1, 1:2, 2:1, 1:3, or 3:1. Other molar ratios are possible as well known in the art.

In one embodiment, host cells harbor three vectors, wherein the first vector is operably joined to nucleic acids encoding the first polypeptide, the second vector is operably joined to nucleic acid encoding the second polypeptide, and the third vector is operably joined to nucleic acids encoding the third polypeptide. The three vectors harbored by the host cells can be present at a molar ratio for first polypeptide:second polypeptide:third polypeptide of 1:1:1, 1:1.5:1, 1:1:1.5, 1.5:1:1, 1:2:1, 1:1:2, 2:1:1, 1:3:1, 1:1:3, or 3:1:1. Other molar ratios are possible as well known in the art.

The present disclosure provide methods for preparing any of the multi-specific antigen binding protein complexes described herein, the method comprising: culturing a population of host cells, wherein individual host cells in the population harbor at least one expression vector that is operably linked to any one or any combination of two or more first, second and/or third nucleic acids encoding any one or any combination of two or more of the first, second and/or third polypeptide chains described herein, wherein the culturing is conducted under conditions suitable for expressing the polypeptide chains by the population of host cells.

In one embodiment, the nucleic acids encoding any one or any combination of two or more of the first, second and/or third polypeptide chains further encodes a signal peptide for secretion of the expressed polypeptide chains. In one embodiment, the culturing is conducted under conditions suitable for secretion of the first, second and/or third polypeptide chains by the population of host cells.

In one embodiment, the nucleic acids encoding any one or any combination of two or more of the first, second and/or third polypeptide chains further encodes an affinity tag sequence for enriching the polypeptides. Exemplary affinity tag sequences include histidine tag, FLAG tag, myc tag, HA tag, and GST tag.

In one embodiment, the method further comprises isolating the expressed first, second and/or third polypeptide chains.

In one embodiment, the culturing is conducted under conditions that are suitable for assembly or association of the first, second and/or third polypeptide chains to form the multi-specific antigen binding protein complexes. In one embodiment, the first and second polypeptide chains associate with each other to form heterodimeric protein complexes comprising first and second Fab regions and an Fc region. In one embodiment, the first, second and third polypeptide chains associate with each other to form heterodimeric protein complexes comprising first and second Fab regions and an Fc region.

In one embodiment, the method further comprises isolating or recovering the assembled multi-specific antigen binding protein complexes. In one embodiment, the isolating is conducted using affinity chromatography. In one embodiment, the isolating is conducted using affinity chromatography with protein A or G from Staphylococcus aureus, glutathione S-transferase (GST), or immuno-affinity. In one embodiment, one or more additional isolating steps are conducted which are selected from a group consisting of cation and/or anion exchange chromatography, hydrophobic interaction chromatography, mixed mode chromatography and hydroxyapatite chromatography.

In one embodiment, the assembled multi-specific antigen binding protein complexes comprise a first Fab region that is capable of binding a first epitope, and a second Fab region that is capable of binding a second epitope that differs from the first epitope. In one embodiment, the assembled multi-specific antigen binding protein complexes comprise an Fc region that is capable of binding an Fc receptor, comprising an Fc-gamma receptor.

The multi-specific antigen binding protein complexes can be prepared using transgenic host cell expression, phage display, yeast display and human antibody gene transgenic mice using methods that are well known in the art. In one embodiment, the yield of heterodimeric protein complexes using transgenic host cell expression can be about 20-80%, or about 30-90%, or about 40-95%, or about 50-99% of the total protein complexes formed.

In one embodiment, in the methods for preparing the multi-specific antigen binding protein complexes, the first nucleic acid encodes a first polypeptide chain comprising (i) a first half Fab heavy region, (ii) a first linker, (iii) a second half Fab heavy region, and (iv) a first half Fc region, wherein the first half Fab heavy region comprises a first-variable region and first-constant region from a first Fab heavy chain, wherein the second half Fab heavy region comprises a second-variable region and second-constant region from a second Fab heavy chain, and wherein the first half Fc region comprises CH2 and CH3. In one embodiment, the second nucleic acid encodes a second polypeptide chain comprising a second polypeptide chain comprising (i) a first half Fab light region, (ii) a second linker, (iii) a second half Fab light region, and (iv) a second half Fc region, wherein the first half Fab light region comprises a first-variable region and first-constant region from a first Fab light chain, and wherein the second half Fab light region comprises a second-variable region and second-constant region from a second Fab light chain, wherein the second half Fc region comprises CH2 and CH3, and wherein the second linker is cleavable. In one embodiment, the first and second polypeptide chains associate with each other to form a multi-specific antigen binding protein complex comprising a first full Fab domain, a second full Fab domain, and a full Fc domain.

In one embodiment, in the methods for preparing the multi-specific antigen binding protein complexes, the first nucleic acid encodes a first polypeptide chain comprising (i) a first half Fab heavy region, (ii) a first half Fc region, (iii) a first linker, and (iv) a second half Fab heavy region, wherein the first half Fab heavy region comprises a first-variable region and first-constant region from a first Fab heavy chain, wherein the first half Fc region comprises CH2 and CH3, and wherein the second half Fab heavy region comprises a second-variable region and second-constant region from a second Fab heavy chain. In one embodiment, the second nucleic acid encodes a second polypeptide chain comprising (i) a first half Fab light region, (ii) a second half Fc region, (iii) a second linker, and (iv) a second half Fab light region, wherein the first half Fab light region comprises a first-variable region and first-constant region from a first Fab light chain, wherein the second half Fc region comprises CH2 and CH3, wherein the second half Fab light region comprises a second-variable region and second-constant region from a second Fab light chain, and wherein the second linker is cleavable. In one embodiment, the first and second polypeptide chains associate with each other to form a multi-specific antigen binding protein complex comprising a first full Fab domain, a full Fc domain, and a second full Fab domain.

In one embodiment, in the methods for preparing the multi-specific antigen binding protein complexes, the first nucleic acid encodes a first polypeptide chain comprising (i) a first half Fab heavy region, (ii) a first linker, (iii) a second half Fab heavy region, and (iv) a first half Fc region, wherein the first half Fab heavy region comprises a first-variable region and first-constant region from a first Fab heavy chain, wherein the second half Fab heavy region comprises a second-variable region and second-constant region from a second Fab heavy chain, and wherein the first half Fc region comprises CH2 and CH3. In one embodiment, the second nucleic acid encodes a second polypeptide chain comprising a first half Fab light region which comprises a first-variable region and first-constant region from a first Fab light chain. In one embodiment, the third nucleic acid encodes a third polypeptide chain comprising (i) a second half Fab light region, and (ii) a second half Fc region, wherein the second half Fab light region comprises a second-variable region and second-constant region from a second Fab light chain, and wherein the second half Fc region comprises CH2 and CH3. In one embodiment, the first, second and third polypeptide chains associate with each other to form a multi-specific antigen binding protein complex comprising a first full Fab domain, a second full Fab domain, and a full Fc domain.

In one embodiment, in the methods for preparing the multi-specific antigen binding protein complexes, the first nucleic acid encodes a first polypeptide chain comprising (i) a first half Fab heavy region, (ii) a first half Fc region, (iii) a first linker, and (iv) a second half Fab heavy region, wherein the first half Fab heavy region comprises a first-variable region and first-constant region from a first Fab heavy chain, wherein the first half Fc region comprises CH2 and CH3, and wherein the second half Fab heavy region comprises a second-variable region and second-constant region from a second Fab heavy chain. In one embodiment, the second nucleic acid encodes a second polypeptide chain comprising (i) a first half Fab light region, and (ii) a second half Fc region, wherein the first half Fab light region comprises a first-variable region and first-constant region from a first Fab light chain, and wherein the second half Fc region comprises CH2 and CH3. In one embodiment, the third nucleic acid encodes a third polypeptide chain comprising a second half Fab light region which comprises a second-variable region and second-constant region from a second Fab light chain. In one embodiment, the first, second and third polypeptide chains associate with each other to form a multi-specific antigen binding protein complex comprising a first full Fab domain, a full Fc domain, and a second full Fab domain.

The present disclosure provide in vitro and in vivo methods for binding any of the multi-specific antigen binding protein complexes comprising two polypeptide chains which are described herein to a first target epitope, the method comprising: (a) contacting the first target epitope with a multi-specific antigen binding protein complex (a two-chain protein complex) in an inactive form which comprises a first Fab region, a second Fab region, an Fc region, and a cleavable linker in an un-cleaved state, wherein the first Fab region binds the first target epitope and the second Fab region exhibits reduced binding to a second target epitope when the cleavable linker is in the un-cleaved state; and (b) binding the first epitope to the first full Fab of the protein complex. In one embodiment, the multi-specific antigen binding protein complex used in step (a) comprises any of the two chain protein complexes described herein which includes a first cleavable linker. In one embodiment, the cleavable linker is cleavable with a matrix metalloprotease (MMP), MMP1, MMP2, MMP3, MMP8, MMP9, MMP11, MMP13, MMP14, MT1-MMP (membrane type 1 matrix metalloproteinase), a disintegrin and metalloproteinase (ADAM) protease, ADAM10, ADAM12, ADAM17, urokinase plasminogen activator (uPA), serine proteases, cysteine proteases, aspartate proteases, threonine proteases, cathepsins, cathepsin B, cathepsin C, cathepsin D, cathepsin E, cathepsin K or cathepsin L. In one embodiment, the first target epitope comprises a first soluble cell surface antigen or a first membrane-bound cell surface antigen.

In one embodiment, the method further comprises: cleaving the cleavable linker to generate an activated multi-specific antigen binding protein complex wherein the second Fab region can bind to the second target epitope. In one embodiment, the second target epitope comprises a second soluble cell surface antigen or a second membrane-bound cell surface antigen.

In one embodiment, the method further comprises: (a) contacting the second target epitope with the activated multi-specific antigen binding protein complex; and (b) binding the second target epitope with the second Fab region of the activated multi-specific antigen binding protein complex to form an activated multi-specific antigen binding protein complex bound to the first and second target epitopes. In one embodiment, after cleavage of the cleavable linker, the first Fab region and the second Fab region are capable of binding to the first and second target epitopes, respectively, at the same time.

In one embodiment, the method further comprises: detecting the activated multi-specific antigen binding protein complex bound to the first and second target epitopes.

In one embodiment, the first target epitope comprises a first antigen (e.g., surface antigen) expressed by a tumor or cancer cell. In one embodiment, the second epitope comprises second antigen (e.g., surface antigen) expressed by an effector T cell.

In one embodiment, the tumor or cancer cell that expresses the first target epitope also expresses one or more enzyme that cleaves the cleavable linker. In one embodiment, the tumor or cancer cell expresses one or a combination of two or more enzymes comprising a matrix metalloprotease (MMP), MMP1, MMP2, MMP3, MMP8, MMP9, MMP11, MMP13, MMP14, MT1-MMP (membrane type 1 matrix metalloproteinase), a disintegrin and metalloproteinase (ADAM) protease, ADAM10, ADAM12, ADAM17, urokinase plasminogen activator (uPA), serine proteases, cysteine proteases, aspartate proteases, threonine proteases, cathepsins, cathepsin B, cathepsin C, cathepsin D, cathepsin E, cathepsin K and/or cathepsin L.

In one embodiment, the method further comprises: forming a cell synapse by binding the first epitope (e.g., first antigen) expressed by the tumor or cancer cell with the first full Fab of the activated multi-specific antigen binding protein complex, and binding the second epitope (e.g., second antigen) expressed by the effector T cell with the second full Fab of the activated multi-specific antigen binding protein complex.

In one embodiment, the method further comprises: killing the tumor or cancer cell with the effector T cell in the cell synapse which mediates cytotoxic cell killing.

In one embodiment, the multi-specific antigen binding protein complex in the in-active form comprises two polypeptide chains associated with each other to form a first Fab region, a second Fab region, an Fc region and a cleavable linker. In one embodiment, each polypeptide chain comprises a first half Fab region, a second half Fab region and a half Fc region. In one embodiment, one of the polypeptide chains comprises a cleavable linker. In one embodiment, the first and second Fab regions are arranged in-tandem. In one embodiment, the cleavable linker is located between the first Fab region and the second Fab region (e.g., the first and second Fab regions are arranged in a non-tandem manner).

In one embodiment, the cleavable linker is cleavable with a peptide cleaving condition which is selected from a group selected from a protease, esterase, reductive condition and oxidative condition.

In one embodiment, the cleavable linker is cleavable with a protease selected from a group consisting of a matrix metalloprotease (MMP), MMP1, MMP2, MMP3, MMP8, MMP9, MMP11, MMP13, MMP14, MT1-MMP (membrane type 1 matrix metalloproteinase), a disintegrin and metalloproteinase (ADAM) protease, ADAM10, ADAM12, ADAM17, urokinase plasminogen activator (uPA), serine proteases, cysteine proteases, aspartate proteases, threonine proteases, cathepsins, cathepsin B, cathepsin C, cathepsin D, cathepsin E, cathepsin K and cathepsin L.

In one embodiment, the cleavable linker is cleavable with a peptide cleaving condition that is present in a tumor microenvironment.

In one embodiment, the peptide cleaving condition that is present in a tumor microenvironment comprise a protease selected from a group consisting of a matrix metalloprotease (MMP), MMP1, MMP2, MMP3, MMP8, MMP9, MMP11, MMP13, MMP14, MT1-MMP (membrane type 1 matrix metalloproteinase), a disintegrin and metalloproteinase (ADAM) protease, ADAM10, ADAM12, ADAM17, urokinase plasminogen activator (uPA), serine proteases, cysteine proteases, aspartate proteases, threonine proteases, cathepsins, cathepsin B, cathepsin C, cathepsin D, cathepsin E, cathepsin K and cathepsin L.

The present disclosure provide in vitro and in vivo methods for binding any of the multi-specific antigen binding protein complexes comprising three polypeptide chains which are described herein to a first target epitope and a second target epitope, the method comprising: contacting the first and second target epitopes with a multi-specific antigen binding protein complex (a three-chain protein complex) which comprises a first Fab region, a second Fab region, and an Fc region, wherein the first Fab region binds the first target epitope and the second Fab region bind the second target epitope. In one embodiment, the first Fab region and the second Fab region are capable of binding to the first and second target epitopes, respectively, at the same time. In one embodiment, the first target epitope comprises a first soluble cell surface antigen or a first membrane-bound cell surface antigen. In one embodiment, the second target epitope comprises a second soluble cell surface antigen or a second membrane-bound cell surface antigen.

In one embodiment, the first target epitope comprises a first antigen (e.g., surface antigen) expressed by a tumor or cancer cell. In one embodiment, the second epitope comprises second antigen (e.g., surface antigen) expressed by a tumor or cancer cell, or expressed by an effector T cell.

In one embodiment, the method further comprises: forming a cell synapse by binding the first epitope (e.g., first antigen) expressed by the tumor or cancer cell with the first full Fab of the activated multi-specific antigen binding protein complex, and binding the second epitope (e.g., second antigen) expressed by the tumor or cancer cell or expressed by the effector T cell with the second full Fab of the activated multi-specific antigen binding protein complex.

In one embodiment, method further comprises: killing the tumor or cancer cell with the effector T cell in the cell synapse which mediates cytotoxic cell killing.

In one embodiment, the multi-specific antigen binding protein complex comprises three polypeptide chains associated with each other to form a first Fab region, a second Fab region, and an Fc region. In one embodiment, the first and second Fab regions are arranged in-tandem or are arranged in a non-tandem manner.

The present disclosure provide methods treating a subject having a disease associated with expression or over-expression of a tumor-associated antigen, the method comprising: administering to the subject a therapeutic composition which comprises a multi-specific antigen binding protein complex that can bind two different epitopes on the same target antigen or two different epitopes on different target antigens. In one embodiment, the protein complex comprises: two different Fab regions, an Fc region and a cleavable linker. In one embodiment, the two different Fab regions, the Fc region and the cleavable linker are formed from two polypeptide chains and each polypeptide chain carries two different half Fab regions and a half Fc region. In one embodiment, the one polypeptide chain carries a single cleavable peptide linker. In one embodiment, the two different Fab regions, the Fc region and the cleavable linker are formed from three polypeptide chains, wherein the three polypeptide chains associate with each other to form a multi-specific antigen binding protein complex comprising a first full Fab domain, a full Fc domain, and a second full Fab domain, or wherein the three polypeptide chains associate with each other to form a multi-specific antigen binding protein complex comprising a first full Fab domain, a second full Fab domain, and a full Fc domain.

In one embodiment, the subject is administered a multi-specific antigen binding protein complex comprising: (a) a first polypeptide chain comprising (i) a first half Fab heavy region, (ii) a first linker, (iii) a second half Fab heavy region, and (iv) a first half Fc region, wherein the first half Fab heavy region comprises a first-variable region and first-constant region from a first Fab heavy chain, and wherein the second half Fab heavy region comprises a second-variable region and second-constant region from a second Fab heavy chain; and (b) a second polypeptide chain comprising (i) a first half Fab light region, (ii) a second linker, (iii) a second half Fab light region, and (iv) a second half Fc region, wherein the first half Fab light region comprises a first-variable region and first-constant region from a first Fab light chain, and wherein the second half Fab light region comprises a second-variable region and second-constant region from a second Fab light chain, and wherein the second linker is cleavable.

In one embodiment, the subject is administered a multi-specific antigen binding protein complex comprising: (a) a first polypeptide chain comprising (i) a first half Fab heavy region, (ii) a first half Fc region, (iii) a first linker, and (iv) a second half Fab heavy region, wherein the first half Fab heavy region comprises a first-variable region and first-constant region from a first Fab heavy chain, and wherein the second half Fab heavy region comprises a second-variable region and second-constant region from a second Fab heavy chain; and (b) a second polypeptide chain comprising (i) a first half Fab light region, (ii) a second half Fc region, (iii) a second linker, and (iv) a second half Fab light region, wherein the first half Fab light region comprises a first-variable region and first-constant region from a first Fab light chain, and wherein the second half Fab light region comprises a second-variable region and second-constant region from a second Fab light chain, and wherein the second linker is cleavable.

In one embodiment, the subject is administered a multi-specific antigen binding protein complex comprising: (a) a first polypeptide chain comprising (i) a first half Fab heavy region, (ii) a first linker, (iii) a second half Fab heavy region, and (iv) a first half Fc region, wherein the first half Fab heavy region comprises a first-variable region and first-constant region from a first Fab heavy chain, and wherein the second half Fab heavy region comprises a second-variable region and second-constant region from a second Fab heavy chain; (b) a second polypeptide chain comprising a first half Fab light region which comprises a first-variable region and first-constant region from a first Fab light chain; and (c) a third polypeptide chain comprising (i) a second half Fab light region, and (ii) a second half Fc region, wherein the second half Fab light region comprises a second-variable region and second-constant region from a second Fab light chain.

In one embodiment, the subject is administered a multi-specific antigen binding protein complex comprising: (a) a first polypeptide chain comprising (i) a first half Fab heavy region, (ii) a first half Fc region, (iii) a first linker, and (iv) a second half Fab heavy region, wherein the first half Fab heavy region comprises a first-variable region and first-constant region from a first Fab heavy chain, and wherein the second half Fab heavy region comprises a second-variable region and second-constant region from a second Fab heavy chain; (b) a second polypeptide chain comprising (i) a first half Fab light region, and (ii) a second half Fc region; and (c) a third polypeptide chain comprising a second half Fab light region, wherein the first half Fab light region comprises a first-variable region and first-constant region from a first Fab light chain, and wherein the second half Fab light region comprises a second-variable region and second-constant region from a second Fab light chain.

The present disclosure provides an in vitro cleavage-based method to detect protease activity and specificity for detecting, diagnosing, monitoring and/or staging a cancer or tumor. A tumor or cancer mass can be extracted from a subject and contacted with one or more different two-chain multi-specific antigen binding protein complexes described herein, each having a different cleavable linker (e.g., the second linker) with a known protease cleavage profile. The tumor or cancer mass produces one or more protease and is contacted with different two-chain multi-specific antigen binding protein complexes under conditions suitable for a protease(s) to cleave the cleavable linker on the two chain protein complex(es). A product resulting from cleavage of the linker can be detected using any suitable method. Thus, one or more type(s) of protease produced by the tumor or cancer mass can be identified. In one embodiment, the cleavable linker is cleavable with any one or any combination of two or more proteases selected from a matrix metalloprotease (MMP), MMP1, MMP2, MMP3, MMP8, MMP9, MMP11, MMP13, MMP14, MT1-MMP (membrane type 1 matrix metalloproteinase), ADAM protease, urokinase plasminogen activator (uPA), serine proteases, cysteine proteases, aspartate proteases, threonine proteases, cathepsins, cathepsin B, cathepsin C, cathepsin D, cathepsin E, cathepsin K and cathepsin L.

The present disclosure provides a method for detecting the presence of a protease produced by a tumor from a subject, the method comprising: (a) contacting (i) a tumor obtained from the subject with (ii) a multi-specific antigen binding protein complex that comprises two different Fab regions, an Fc region and a cleavable linker (e.g., any of the two-chain protein complexes described herein), wherein the tumor sample produces a protease, wherein the amino acid sequence of the cleavable linker may or may not be a substrate for cleavage by the protease produced by the tumor sample, and wherein the contacting is performed under conditions suitable for the protease to cleave the cleavable linker to generate a cleavage product when the protease cleaves the cleavable linker; and (b) detecting the cleavage product, thereby diagnosing the cancer in the subject. In one embodiment, the method further comprises: (c) identifying the type of protease produced by the tumor from the subject by detecting the cleavage product and correlating the cleavage product with the amino acid sequence of the cleavable linker. In one embodiment, by identifying the type of protease produced by the tumor in the subject, the cancer in the subject can be diagnosed. In one embodiment, the cleavage product can be detected by gel electrophoresis, Western blot analysis, immunology, immunohistochemistry, colorimetrically, spectrophotometrically, mass spectrophotometry, liquid chromatography, or by any combination thereof. In one embodiment, the tumor or cancer mass can be obtained from a prostate, breast, ovary, head and neck, bladder, skin, colorectal, anus, rectum, pancreas, lung (including non-small cell lung and small cell lung cancers), leiomyoma, brain, glioma, glioblastoma, esophagus, liver, kidney, stomach, colon, cervix, uterus, endometrium, vulva, larynx, vagina, bone, nasal cavity, paranasal sinus, nasopharynx, oral cavity, oropharynx, larynx, hypolarynx, salivary glands, ureter, urethra, penis, and testis. In one embodiment, the subject is a human, non-human primate, simian, ape, murine (e.g., mice and rats), bovine, porcine, equine, canine, feline, caprine, lupine, ranine or piscine. In one embodiment, the in vitro cleavage-based method can be used for detecting, diagnosing, monitoring and/or staging a cancer in the subject.

The present disclosure provides any multi-specific antigen binding protein complex described herein, wherein the first or second light chain variable regions comprise amino acid sequence from κ (kappa) or λ (lambda) chains.

The present disclosure provides any multi-specific antigen binding protein complex described herein, wherein the first or second heavy chain variable regions comprise amino acid sequences from μ (mu), γ (gamma), α (alpha), δ (delta) or ε (epsilon) chains.

The present disclosure provides any multi-specific antigen binding protein complex described herein, wherein the variable heavy region and constant heavy region are directly joined together without any intervening linker sequences which avoids introducing immunogenic sites.

The present disclosure provides any multi-specific antigen binding protein complex described herein, wherein the variable light region and constant light region are directly joined together without any intervening linker sequences which avoids introducing immunogenic sites.

The present disclosure provides any multi-specific antigen binding protein complex described herein, wherein the first Fab region can bind a first target epitope selected from a group consisting of a cell surface protein, cytokine, cytokine receptor, chemokine, and an enzyme. In one embodiment, the first target epitope comprises a disease-associated antigen on a cancer or tumor cell.

In one embodiment, the first Fab region can bind the first target epitope and exhibit a dissociation constant K_{d} of 10⁻⁵M or less, or 10⁻⁶ M or less, or 10⁻⁷M or less, or 10⁻⁸M or less, or 10⁻⁹M or less, or 10⁻¹⁰ M or less.

In one embodiment, the multi-specific antigen binding protein complex comprises a first Fab region, or a first and second Fab region, that can bind a first target epitope which is selected from a group consisting of B7-H3 (CD276), B7-H4, BCMA, BTLA, CCR2, CD19, CD20, CD27, CD30, CD32B, CD33, CD38, CD40L, CD47, CD123, CD137, CEA, cKIT, c-Met, CXCR3, CXCR5, CTLA4, DLL4, EGFR, EpCAM, ErbB3, ErbB2, gpA33, Her1, Her2, Her3, Her4, ICOS, IGF1R, IL1α, IL4, IL6R, IL13, IL17A/F, JAG1, KIR, KRAS, LAG3, MUC1, MUC2, MUC3, MUC4, MUC5AC, MUC5B, MUC7, OPRF, OPRI, OX40, P-cadherin, PD-1, PD-L1, PSMA, STAT3, TIM3, TNFa, VEGFR2 and WISP1.

The present disclosure provides any multi-specific antigen binding protein complex described herein, wherein the second Fab region is capable of binding a second target epitope selected from a group consisting of a cell surface antigen on a T cell (e.g., effector T cell), a NK cell, a monocyte, a neutrophil or a macrophage. In one embodiment, the second target epitope comprises a disease-associated antigen on a cancer or tumor cell.

In one embodiment, the second Fab region is capable of binding the second target epitope and exhibit a dissociation constant K_{d} of 10⁻⁵ M or less, or 10⁻⁶ M or less, or 10⁻⁷ M or less, or 10⁻⁸M or less, or 10⁻⁹M or less, or 10⁻¹⁰ M or less.

In one embodiment, the second Fab region binds the second epitope upon cleavage of the second linker.

In one embodiment, the second target epitope comprises: CD3, TCRα, TCRβ, CD28, or CTLA4 (e.g., expressed on T cells); or PD-L1 (a.k.a. B7-H1; e.g., expressed on macrophage and dendritic cells); or CD16 (a.k.a. FcγRIII; e.g., expressed on NK cells); or CD64 (a.k.a. FCγRI; e.g., expressed on macrophage, neutrophil or monocyte cells).

In one embodiment, the multi-specific antigen binding protein complex comprises a second Fab region that can bind a second target epitope which is selected from a group consisting of CD3, CD8, CD10, CD16a, CD19, CD20, CD21, CD22, CD33, CD79B, HAS (human serum albumin), IL13, IL17, IL17A and VEGF.

In one embodiment, the second full Fab comprises an antigen binding domain that is capable of binding CD3 and the antigen binding domain is derived from a known anti-CD3 antibody selected from a group consisting of muromonab-CD3 (OKT3), otelixizumab (TRX4), teplizumab (MGA031), visilizumab (Nuvion), SP34 or I2C, TR-66 or X35-3, VIT3, BMA030 (BW264/56), CLB-T3/3, CRIS7, YTH12.5, Fl 11-409, CLB-T3.4.2, TR-66, WT32, SPv-T3b, 11D8, XIII-141, XIII-46, XIII-87,12F6, T3/RW2-8C8, T3/RW2-4B6, OKT3D, M-T301, SMC2, F101.01, UCHT-1 and WT-31.e.

The present disclosure provides any multi-specific antigen binding protein complex described herein, comprising a first Fab region and a second Fab region that are capable of binding a pair of target epitopes selected from a group consisting of CD38 and CD3, BCMA and CD3, EFGR and PD-L1, and CD20 and CD47. The skilled artisan will appreciate that many other combinations are possible.

The present disclosure provides any multi-specific antigen binding protein complex described herein, wherein the Fc region comprises CH2 and CH3 sequences that are directly joined together without any intervening amino acids or peptide linker sequence.

In one embodiment, the Fc region exhibits effector function, including complement-dependent cytotoxicity (CDC), antibody-dependent cell-mediated cytotoxicity (ADCC) and/or antibody-dependent phagocytosis (ADP), and a mutation in the Fc region can increase or decrease any one or any combination of these functions. In one embodiment, the Fc region comprises a LALA-PG mutation (L234A, L235A, P329G) which reduces effector function.

In one embodiment, the Fc region mediates serum half-life of the protein complex, and a mutation in the Fc region can increase or decrease the serum half-life of the protein complex.

In one embodiment, the Fc region affects thermal stability of the protein complex, and mutation in the Fc region can increase or decrease the thermal stability of the protein complex.

The present disclosure provides any multi-specific antigen binding protein complex described herein, comprising one or more amino acid mutations in any half Fab region and/or any half Fc region that promotes formation of heterodimers in an assembled protein complex, where the mutations lead to introducing knob-in-hole structures (Ridgeway 1996 Protein Engineering 9(7):617-621), introducing additional interchain disulfide bonding (Carter 2011 Journal of Immunological Methods 248:7-15), and/or introducing new salt bridges.

The present disclosure provides any multi-specific antigen binding protein complex described herein, wherein the Fc region, or the first or second Fab region, includes mutations in any of the polypeptide chains that create a protrusion (e.g., knob) on one chain and a socket (e.g., hole) on the other chain so that the protrusion and socket associate with each other. In one embodiment, the protrusion and socket promote association between the polypeptide chains, for example to promote heterodimerization. In one embodiment, one of the polypeptide chains is mutated by substituting a small amino acid with a larger one to create a protrusion (e.g., in the first or second half Fc region). In one embodiment, another polypeptide chain is mutated by substituting a larger amino acid with a smaller one to create a socket (e.g., in the second or first half Fc region). In one embodiment, Fc region knob-in-hole mutations comprise a substitute mutation at any one Fc location or any combination of two or more Fc locations selected from a group consisting of T366, L368, T394, F405, Y407 and K409 (numbering is based on Kabat system). In one embodiment, Fc region knob-in-hole mutations comprise any one or any combination of two or more of the following mutations: T366Y, T366W, T366S, L368A, T394S, T394W, F405A, F405W, Y407A, Y407V, Y407T (numbering based on Kabat system).

The present disclosure provides any multi-specific antigen binding protein complex described herein, wherein any first half Fab heavy region and the first half Fab light region are capable of associating with each other to form at least one covalent bond (e.g., interchain disulfide bonding). Any of the half Fab heavy regions and/or half Fab light regions can be mutated to add the capability to form at least one disulfide bonds (e.g., see “S-S” in FIGS. 1-16), or to remove existing disulfide bonds (e.g., see “X-X” in FIGS. 1-16).

In one embodiment, a first half Fab heavy region and a first half Fab light region includes an amino acid modification (e.g., amino acid substitution; e.g., serine substituted with cysteine, or tyrosine substituted with cysteine) for forming an additional disulfide bond when the first half Fab heavy region and the first Fab light region associate with each other.

In one embodiment, a second half Fab heavy region and a second half Fab light region includes an amino acid modification (e.g., amino acid substitution; e.g., serine substituted with cysteine, or tyrosine substituted with cysteine) for forming an additional disulfide bond when the first half Fab heavy region and the first Fab light region associate with each other.

In one embodiment, a first half Fab heavy region and a first half Fab light region includes an amino acid modification (e.g., amino acid substitution) which removes the capability to form a covalent bond when the second half Fab heavy region and the second Fab light region associate with each other.

In one embodiment, a second half Fab heavy region and a second half Fab light region includes an amino acid modification (e.g., amino acid substitution) which removes the capability to form a disulfide bond when the second half Fab heavy region and the second Fab light region associate with each other.

The present disclosure provides any multi-specific antigen binding protein complex described herein, wherein any first half Fc region and the second half Fc region are capable of associating with each other to form at least one covalent bond (e.g., disulfide bond). Any of the half Fc regions can be mutated to add the capability to form additional disulfide bonds, or to remove existing disulfide bonds.

In one embodiment, a first half Fc region and a second half Fc region includes an amino acid modification (e.g., amino acid substitution; e.g., serine substituted with cysteine, or tyrosine substituted with cysteine) for forming an additional covalent bond when the first half Fc region and the second half Fc region associate with each other.

In one embodiment, a first half Fc region and a second half Fc region includes an amino acid modification (e.g., amino acid substitution) which removes the capability to form a covalent bond when the first half Fc region and the second half Fc region associate with each other.

The present disclosure provides any multi-specific antigen binding protein complex described herein, wherein the Fc region, or the first or second Fab region, includes mutations in any of the polypeptide chains that create a new interchain salt bridge. In one embodiment, a threonine residue at any position in the first, second or third polypeptide chain is replaced with glutamic acid, and an asparagine at a corresponding position in the paired polypeptide chain is replaced with lysine, thereby creating an interchain salt bridge.

The present disclosure provides any multi-specific antigen binding protein complex described herein, wherein the hinge region comprises any one or any combination of two or more regions comprising an upper, core or lower hinge sequences from an IgG1, IgG2, IgG3 or IgG4 immunoglobulin molecule. In one embodiment, the hinge region comprises an IgG1 upper hinge sequence EPKSCDKTHT (SEQ ID NO: 170). In one embodiment, the hinge region comprises an IgG1 core hinge sequence CPXC, wherein X is P, R or S (SEQ ID NO: 171). In one embodiment, the hinge region comprises a lower hinge/CH2 sequence PAPELLGGP (SEQ ID NO: 172). In one embodiment, the hinge is joined to an Fc region (CH2) having the amino acid sequence SVFLFPPKPKDT (SEQ ID NO: 173). In one embodiment, the hinge region includes the amino acid sequence of an upper, core and lower hinge and comprises EPKSCDKTHTCPPCPAP ELLGGP (SEQ ID NO: 174). In one embodiment, the hinge region comprises one, two, three or more cysteines that can form at least one, two, three or more interchain disulfide bonds.

The present disclosure provides any multi-specific antigen binding protein complex described herein, wherein the first linker comprises a peptide linker that permits association/assembly of the first and second polypeptide chains to form a multi-specific antigen binding protein complex that is capable of binding two or more different epitopes on the same target antigen or on different target antigens. The first linker length can be 1-50 amino acids which optionally can include at least one amino acid analog. In one embodiment, the first linker comprises predominantly any combination of two or more amino acids glycine, serine, alanine and/or threonine. In one embodiment, the first linker comprises at least one and up to four polymers of glycine-alanine, or alanine-serine, or other flexible linkers sequences. In one embodiment, first linker comprises an amino acid sequence selected from a group consisting of: (SG)n (SEQ ID NO: 157), (SGG)n (SEQ ID NO: 158), (SGGG)n (SEQ ID NO: 159), (SSG)n (SEQ ID NO: 160), (GS)n (SEQ ID NO: 161), (GGG)n (SEQ ID NO: 162), (GSGGS)n (SEQ ID NO: 163), (GSG)n (SEQ ID NO: 164), (GGGGS)n (SEQ ID NO: 165), (GGGS)n (SEQ ID NO: 166), (GGGGSGS)n (SEQ ID NO: 167), (GGGGSGGS)n (SEQ ID NO: 168), and (GGS)n (SEQ ID NO: 169), where n is an integer of 1-6. In one embodiment, the first linker comprises an amino acid sequence selected from a group consisting of: AKTTPKLEEGEFSEAR, AKTTPKLEEGEFSEARV, AKTTPKLGG, SAKTTPKLGG, AKTTPKLEEGEFSEARV, SAKTTP, SAKTTPKLGG, RADAAP, RADAAPTVS, RADAAAAGGPGS, RADAAAA(G₄S)₄, SAKTTP, SAKTTPKLGG, SAKTTPKLEEGEFSEARV, ADAAP, ADAAPTVSIFPP, TVAAP, TVAAPSVFIFPP, QPKAAP, QPKAAPSVTLFPP, AKTTPP, AKTTPPSVTPLAP, AKTTAP, AKTTAPSVYPLAP, ASTKGP, ASTKGPSVFPLAP, GENKVEYAPALMALS, GPAKELTPLKEAKVS and GHEAAAVMQVQYPAS (SEQ ID NOS:156-184, respectively).

In one embodiment, the second linker comprises an amino acid sequence that is at least 95%, or at least 96%, or at least 97%, or at least 98%, or at least 99% identical to any of the first linkers described supra.

In one embodiment, the first linker sequence can be derived from a heavy chain of any isotype, including for example Cγ1, Cγ2, Cγ3, Cγ4, Cα1, Cα2, Cδ, Cε, and Cμ. In one embodiment, the first linker sequence can be derived from an immunoglobulin-like polypeptide including TCR, FcR and KIR. In one embodiment, the first linker sequence can be derived from an immunoglobulin hinge region.

The present disclosure provides any multi-specific antigen binding protein complex described herein, wherein the second linker comprises a peptide linker that permits association/assembly of the first and second polypeptide chains to form a multi-specific antigen binding protein complex that is capable of binding two or more different epitopes on the same target antigen or on different target antigens. The second linker length can be 1-50 amino acids which optionally can include at least one amino acid analog.

In one embodiment, the second linker is intact (e.g, un-cleaved). In one embodiment, the intact second linker does not significantly increase or decrease the capability of the first Fab region to bind its target epitope. In one embodiment, the intact second linker reduces/restricts the capability of the second Fab region to bind its target epitope. In one embodiment, the intact second linker does not significantly increase or decrease the capability of the first Fab region to bind its target epitope and reduces/restricts the capability of the second Fab region to bind its target epitope. In one embodiment, the second Fab region exhibits reduced binding to the second epitope when the second linker is intact compared to a multi-specific antigen binding protein complex in which the second linker is cleaved. In one embodiment, the intact second linker renders the multi-specific antigen binding protein complex to be in an inactive state. In one embodiment, upon cleavage of the second linker the multi-specific antigen binding protein complex becomes an activated protein complex and the second Fab region can bind its target epitope. It is postulated that when the second linker in the protein complex is cleaved, the multi-specific protein complex undergoes a conformational change so that the second Fab region assumes a conformation that can bind its target epitope. Thus, the multi-specific antigen binding protein complex comprising a cleaved second linker becomes is an activated protein prodrug.

In one embodiment, the second linker includes an amino acid sequence that is cleavable with a cleaving condition which includes a protease, esterase, reductive condition, or oxidative condition. In one embodiment, the second linker is cleavable with a protease that is present in a tumor microenvironment or is cleavable with a reductive or oxidative condition that is present in a tumor microenvironment (Rakashanda et al., 2012 Biotechnology and Molecular Biology Review 7(4):90-101). In one embodiment, the cleaving condition comprises one protease or any combination of two or more proteases, including serine proteases, cysteine proteases, aspartate proteases, threonine proteases, glutamic acid proteases, metalloproteases, asparagine peptide lyases, serum proteases, matrix metalloproteinase (MMP), MMP1, MMP2, MMP3, MMP7, MMP8, MMP9, MMP11, MMP13, MMP14, MT1-MMP (membrane type 1 matrix metalloproteinase), urokinase plasminogen activator (uPA), enterokinase, prostate-specific antigen (PSA, hK3), interleukin-ιβ converting enzyme, thrombin, FAP (FAP-a), dipeptidyl peptidase, meprins, granzymes (e.g., granzyme B), dipeptidyl peptidase IV (DPPI V/CD26), a disintegrin and metalloproteinase (e.g., ADAM proteases), ADAM10, ADAM12, ADAM17, hepsin, cathepsins, cathepsin B, cathepsin C, cathepsin D, cathepsin E, cathepsin K, cathepsin L, kallikreins, hK1, hK10, hK15, plasmin, collagenase, Type IV collagenase, stromelysin, lysosomal enzyme, Factor Xa, chymotrypsin-like protease, trypsin-like protease, elastase-like protease, subtili sin-like protease, actinidain, bromelain, calpain, caspases, caspase-1, caspase-2, caspase-3, caspase-8, caspase-9, caspase-10, caspase-11, caspase-12, caspase-13, caspase-14, herpes simplex virus protease, HIV protease, CMV protease, Mirl-CP, papain, HIV-1 protease, HSV protease, CMV protease, chymosin, renin, pepsin, matriptase (e.g., matriptase 2, human ST14, or TMPRSS6), legumain, plasmepsin, nepenthesin, metalloexopeptidases, and/or metalloendopeptidases.

In one embodiment, the second linker comprises an amino acid sequence that is at least 95%, or at least 96%, or at least 97%, or at least 98%, or at least 99% identical to a peptide that is cleavable with a matrix metalloprotease protease, for example the amino acid sequence of GGSGSGSGGSSGGGSGGGGS (DP linker), TSGSGGSGGSV (EG or EH linker), TSGSGGSPLGMGGSGSV (EI or EU linker), TSGSGGSPLGVGGSGSV (EJ or EV linker), TSGSGGSPAALGGSGSV (EK or EW linker), TSGSGGSPAGLGGSGSV (EL or EX linker), TSGSGGSPLGMVGV (EM or EY linker), TSGSGGSPLGVVGV (EN or EZ linker), TSGSGGSPAALVGV (EO or FA linker), TSGSGGSPAGLVGV (EP or FB linker), TSGSGGSPLGMVLV (EQ or FC linker), TSGSGGSPLGVVLV (ER or FD linker), TSGSGGSPAALVLV (ES or FE linker) or TSGSGGSPAGLVLV (ST or FF linker) (SEQ ID NOS; 11-24, respectively, or SEQ ID NOS:43-56, respectively).

In one embodiment, the second linker comprises the amino acid sequence LEATA which is recognized and cleaved by MMP9. In one embodiment, the second linker comprises the amino acid sequence PR(S/T)(L/I)(S/T) which is recognized and cleaved by MMP9. In one embodiment, the second linker comprises the amino acid sequence SGSGGSPLGMGGSGSVD, SGSGGSPAGLGGSCSVD, or SGSGGSPAGLVGVD (SEQ ID NOS:185-187, respectively). In one embodiment, the second linker comprises the amino acid sequence GGAANLVRGG (SEQ ID NO:188) which is recognized and cleaved by MMP11.

The present disclosure provides any multi-specific antigen binding protein complex described herein, which binds an epitope or an antigen from a human. The present disclosure provides any multi-specific antigen binding protein complex described herein, which binds an epitope or an antigen from a human and can bind (e.g., cross-react) with an epitope or antigen (e.g., homologous antigen) from any one or any combination of non-human animals such as mouse, rat, goat, rabbit, hamster and/or monkey (e.g., cynomolgus, rhesus or macaque).

### EXAMPLES

The following examples are meant to be illustrative and can be used to further understand embodiments of the present disclosure and should not be construed as limiting the scope of the present teachings in any way.

### Example 1: BiaCore Binding Analysis

Binding Between Mobile Phase Target Antigens to Surface-Bound Bispecific Antibodies.

Kinetic interaction between three-chain or two-chain bispecific antibodies for individual antigens was measured at approximately 25° C. using surface plasmon resonance (SPR) analysis using a Biacore T200 surface plasmon resonance (GE Healthcare).

The binding kinetics of three-chain and two-chain bispecific antibodies to their respective antigen as a sole binding event was compared binding kinetics of their parental monoclonal antibodies. The bispecific antibodies were immobilized to a biosensor surface via amine coupled anti-Fc antibodies on a CM5 biosensor chip (GE Healthcare) according to manufacturer's recommendation. A concentration series, ranging from 0 to approximately 10× of the KD, of the individual antigen were applied as the analytes to the biosensor surface for 2 minutes for the association phase, followed by a buffer flow of 5 minutes for the dissociation phase.

The binding kinetics of immobilized bispecific antibodies, designed to bind PD-L1 and EGFR, to bind mobile phase EGFR or PD-L1 antigen as a sole binding event was compared binding kinetics of the parental anti-EGFR or anti-PD-L1 monoclonal antibody.

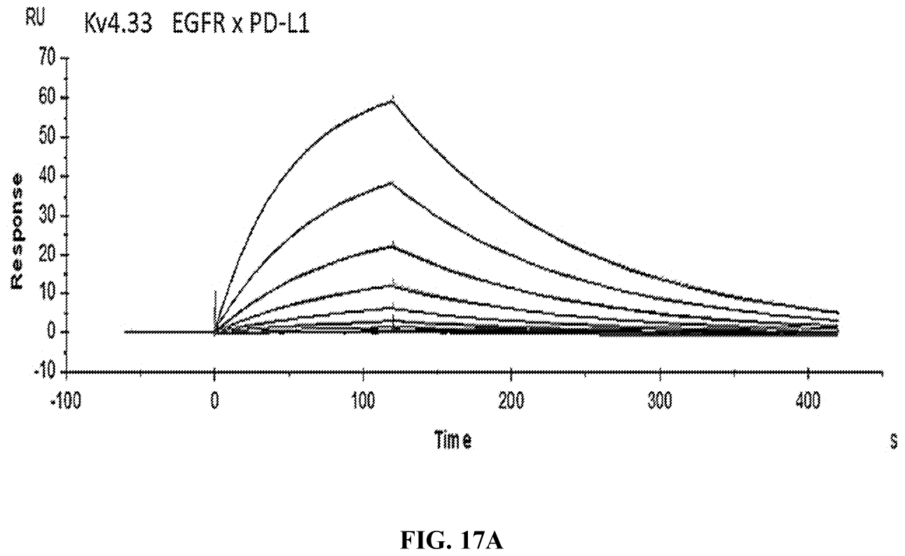

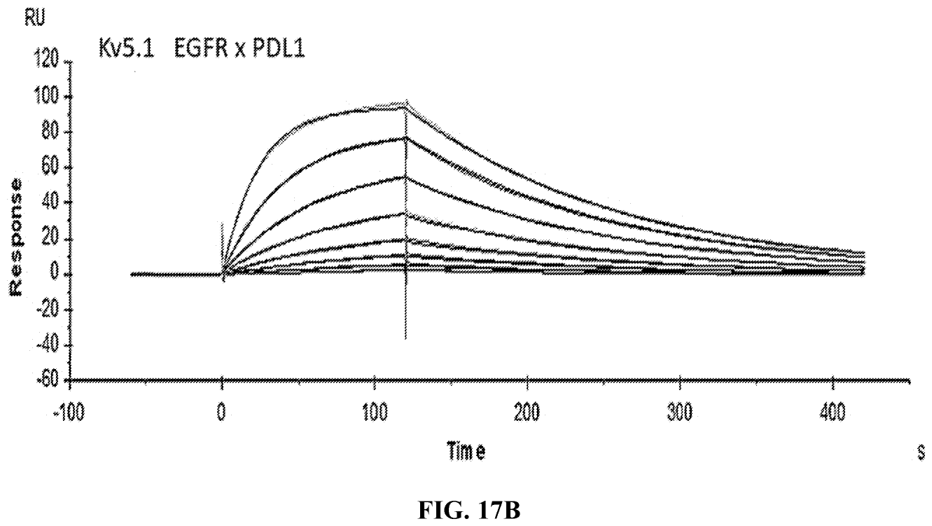

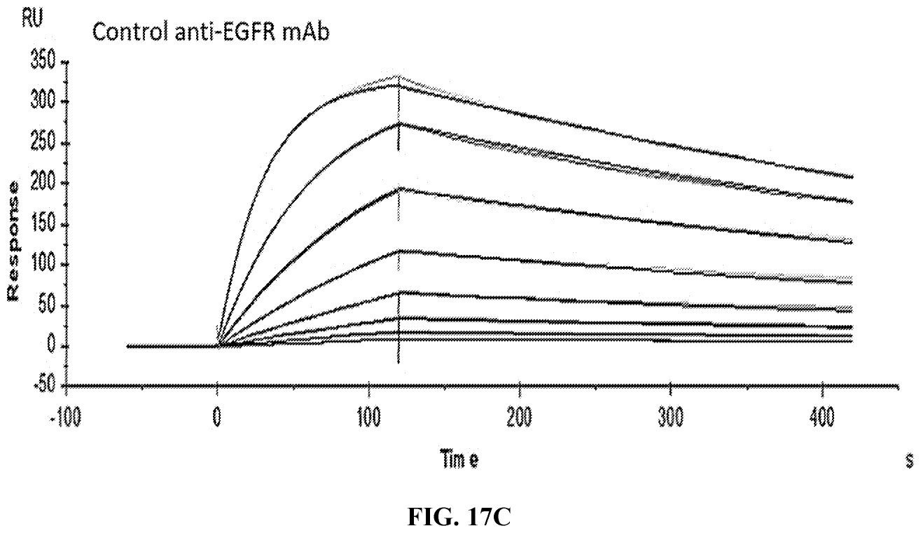

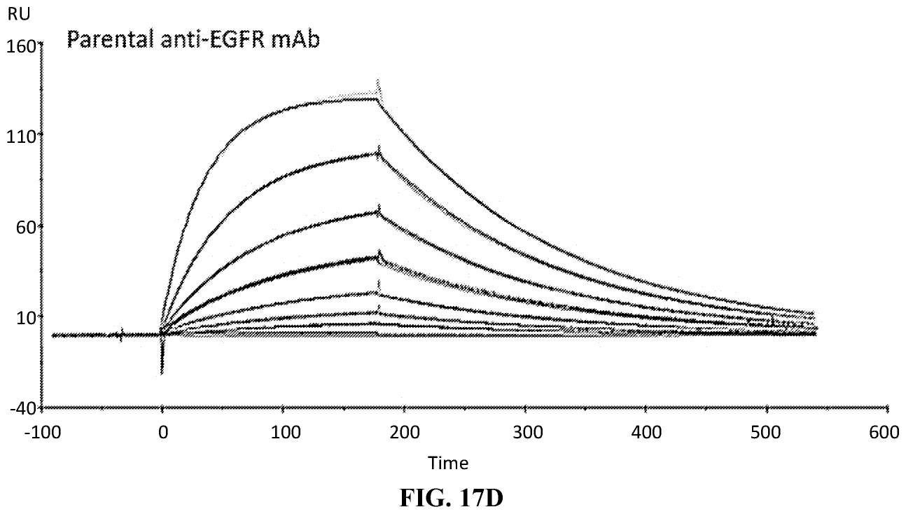

FIG. 17A shows a sensorgram of surface-bound three-chain non-tandem bispecific antibody (structure shown in FIG. 4, Kv4.33) binding to mobile phase EGFR antigen. FIG. 17B shows a sensorgram of surface-bound three-chain in-tandem bispecific antibody (structure shown in FIG. 2, Kv5.1) binding to mobile phase EGFR antigen. FIG. 17C shows a sensorgram of surface-bound control anti-EGFR antibody (see FIG. 43) binding to mobile phase EGFR antigen. FIG. 17D shows a sensorgram of surface-bound parental anti-EGFR monoclonal antibody (2DGA1) binding to mobile phase EGFR antigen. The parental monoclonal anti-EGFR antibody is described in U.S. Pat. No. 9,944,707 as antibody clone 2DGA1.

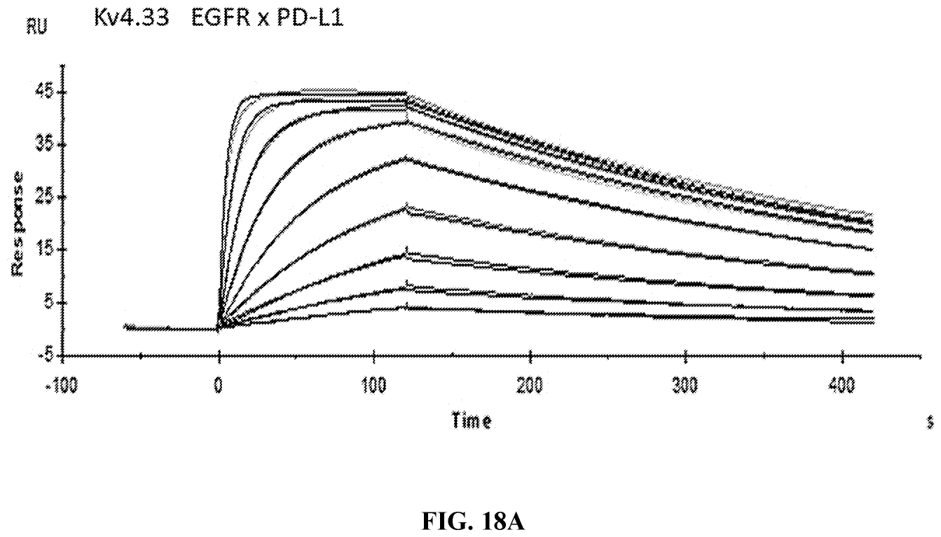

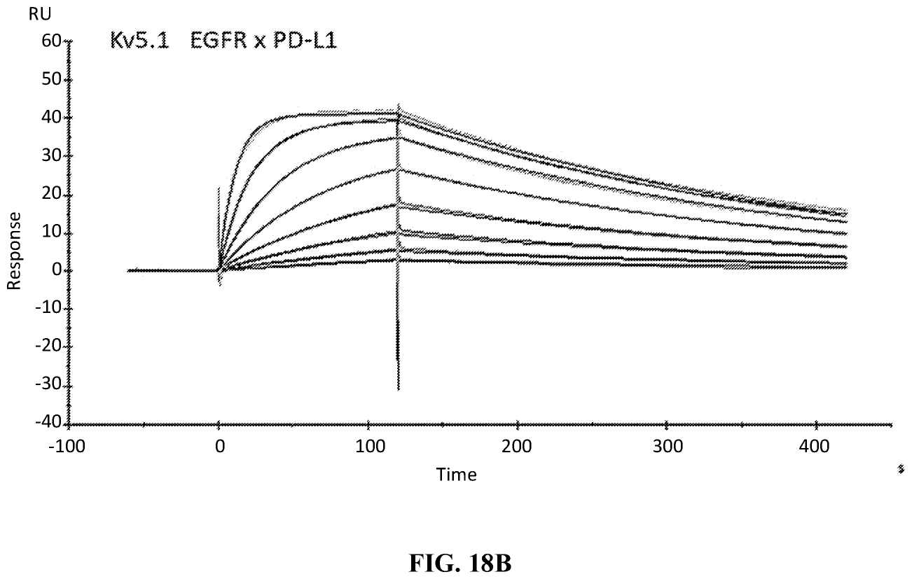

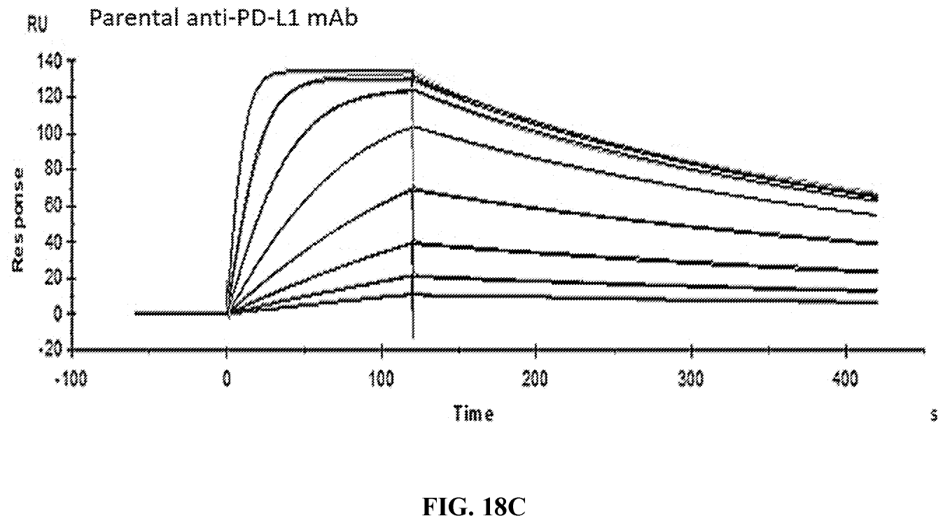

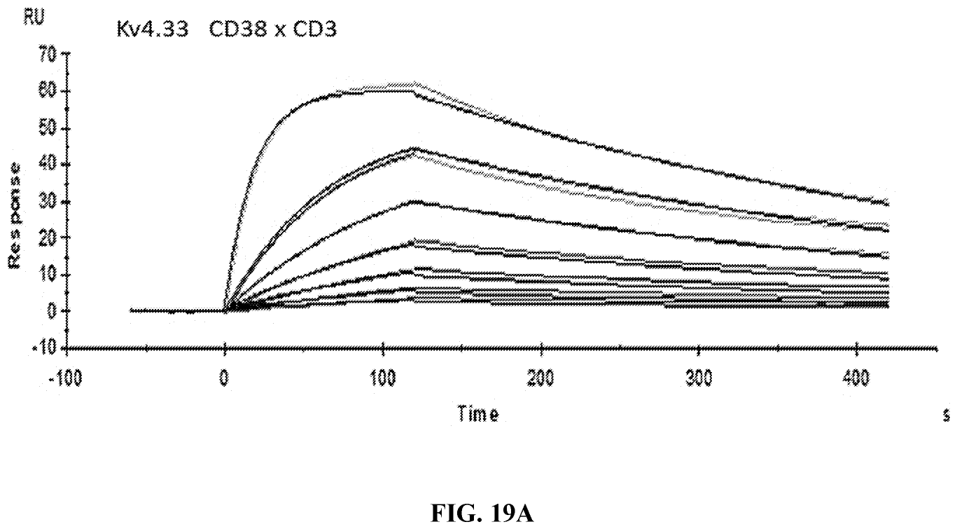

FIG. 18A shows a sensorgram of surface-bound three-chain non-tandem bispecific antibody (structure shown in FIG. 4, Kv4.33) binding to mobile phase PD-L1 antigen. FIG. 18B shows a sensorgram of surface-bound three-chain in-tandem bispecific antibody (structure shown in FIG. 2, Kv5.1) binding to mobile phase PD-L1 antigen. FIG. 18C shows a sensorgram of surface-bound parental anti-PD-L1 monoclonal antibody (SH1E2) binding to mobile phase PD-L1 antigen. The parental monoclonal anti-PD-L1 antibody is described in U.S. Pat. No. 9,175,082 as antibody clone SH1E2, and antibody clone H6b1L is also described in U.S. Pat. No. 9,175,082.

The binding kinetics of immobilized bispecific antibodies, designed to bind CD38 and CD3, to bind mobile phase CD38 antigen as a sole binding event was compared binding kinetics of the parental anti-CD38 monoclonal antibody.

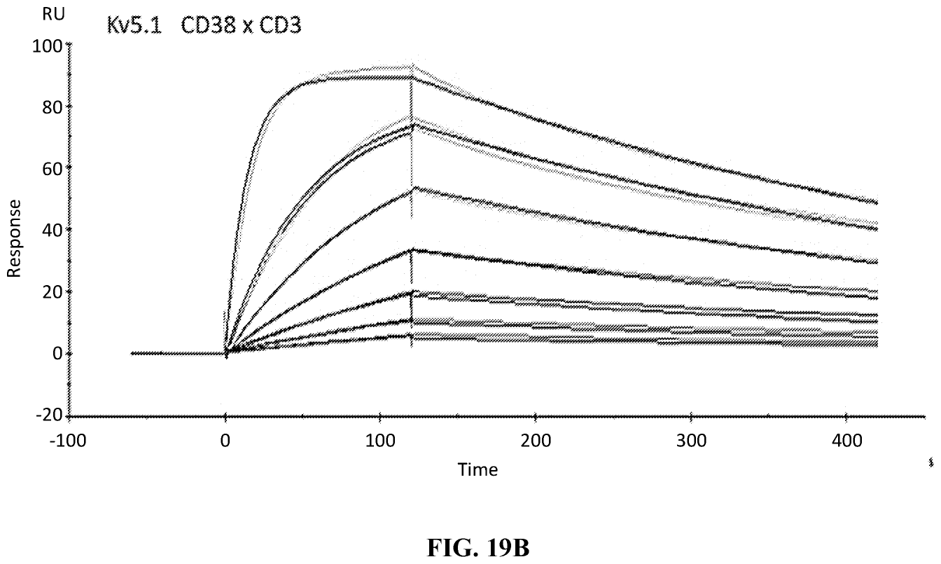

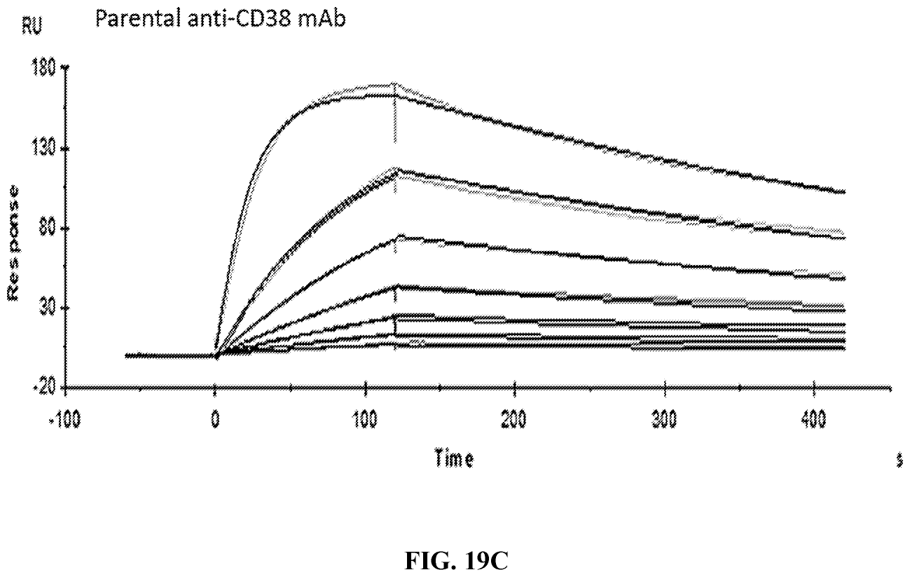

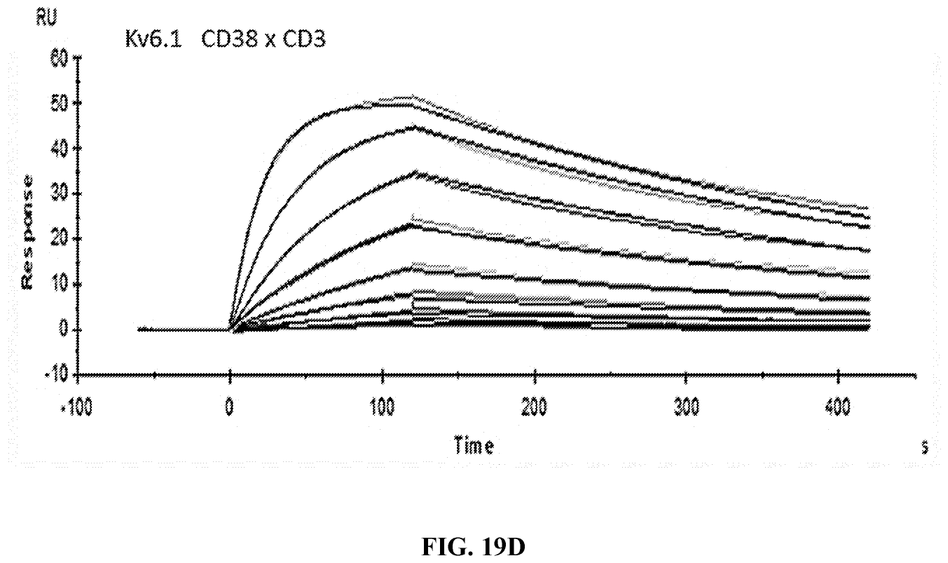

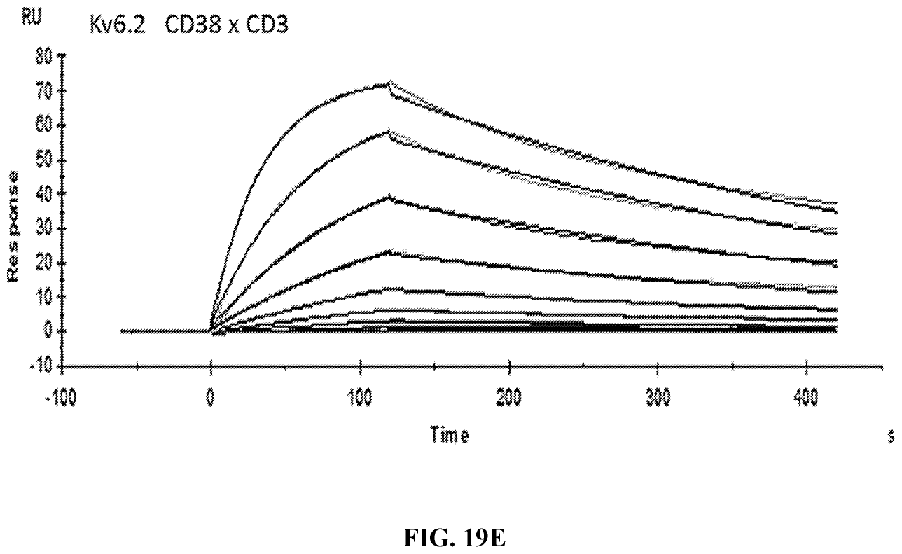

FIG. 19A shows a sensorgram of surface-bound three-chain non-tandem bispecific antibody (structure shown in FIG. 4, Kv4.33) binding to mobile phase CD38 antigen. FIG. 19B shows a sensorgram of surface-bound three-chain in-tandem bispecific antibody (structure shown in FIG. 2, Kv5.1) binding to mobile phase CD38 antigen. FIG. 19C shows a sensorgram of surface-bound parental anti-CD38 monoclonal antibody (3H10m1) binding to mobile phase CD38 antigen. FIG. 19D shows a sensorgram of surface-bound two-chain in-tandem bispecific antibody (intact, non-cleaved) (structure shown in FIG. 2, Kv6.1) binding to mobile phase CD38 antigen. FIG. 19E shows a sensorgram of surface-bound two-chain non-tandem bispecific antibody (intact, non-cleaved) (structure shown in FIG. 3, Kv6.2) binding to mobile phase CD38 antigen. The parental monoclonal anti-CD38 antibody is described in U.S. provisional application No. 62/825,983, filed Mar. 29, 2019, and PCT application No. PCT/US2020/025181, filed Mar. 27, 2020 (e.g., antibody clone 3H10m1).

The binding kinetics of an immobilized bispecific antibody, designed to bind BCMA and CD3, to bind mobile phase BCMA antigen as a sole binding event was compared binding kinetics of the parental anti-BCMA monoclonal antibody.

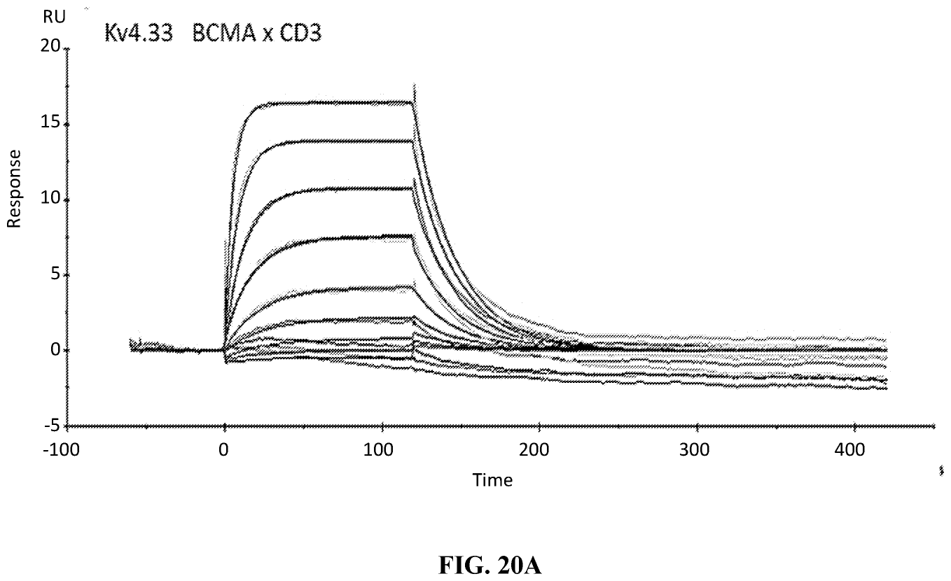

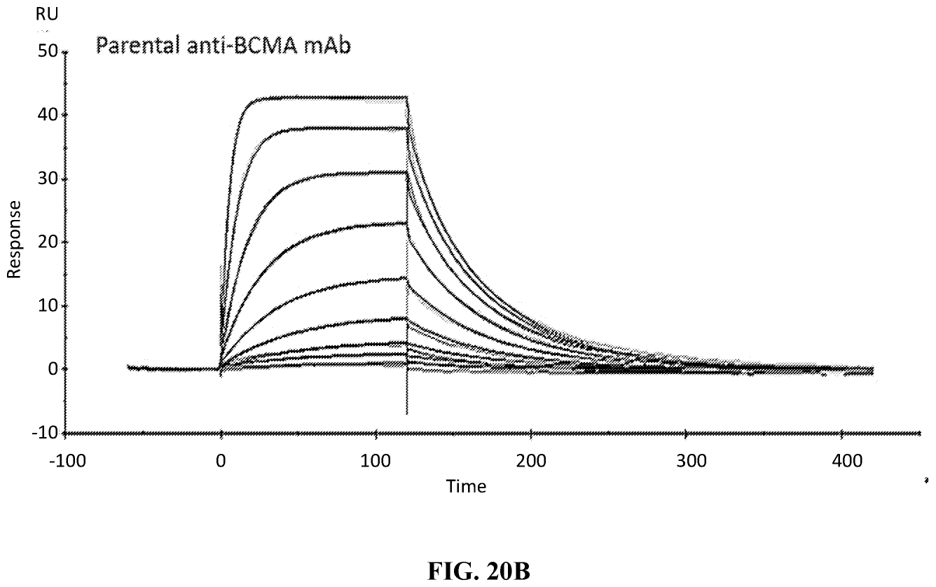

FIG. 20A shows a sensorgram of surface-bound three-chain non-tandem bispecific antibody (structure shown in FIG. 4, Kv4.33) binding to mobile phase BCMA antigen. FIG. 20B shows a sensorgram of surface-bound parental anti-BCMA monoclonal antibody (2C5) binding to mobile phase BCMA antigen. The parental monoclonal anti-BCMA antibody is described in U.S. provisional application No. 62/811,431, filed Feb. 27, 2019, and International Application No. PCT/US2020/19763, filed Feb. 25, 2020 (e.g., antibody clone 2C5).

For all BiaCore assays described in Examples 1, 2 and 3, the EGFR target antigen was obtained from Sino Biological (catalog #10001-H08H, NCBI accession NP 005219, Met 1-Ser 645, see FIG. 39), the PD-L1 target antigen was obtained from Sino Biological (catalog #10081-H08H, NCBI accession NP054862.1, Met 1-Thr 239, see FIG. 40), the CD38 target antigen was obtained from Sino Biological (catalog #10818-H08H NCBI accession NP 001766.2, Val 43-Ile 300, see FIG. 41), and the BMCA target antigen was obtained from Acro Biosystems (catalog # BCA-H522g, NCBI accession Q02223-1, Met 1-Ala 54, see FIG. 42).

A standard evaluation with the 1:1 binding model was applied to all kinetics series described above. The association and dissociation constants (k-on and k-off), the affinity (KD), maximal response (Rmax), as well as the fitting statistics (Chi\hat{ }2) are presented in Table 8, which appears at the end of the Examples section.

### Example 2: BiaCore Binding Analysis

Binding Between Mobile Phase Bispecific Antibodies to Surface-Bound Target Antigens.

Target antigens (EGFR or PD-L1) were immobilized to a biosensor surface via amine coupling onto the reference and test flow cell surface on a CM5 senor chip (GE Healthcare). A concentration series of the three-chain bispecific antibodies were applied to the test flow cells only, with a 2-minute association phase and a 5-minute dissociation phase.

The binding kinetics of immobilized EGFR or PD-L1 antigen to mobile phase bispecific antibodies designed to bind PD-L1 and EGFR as a sole binding event was compared to binding kinetics of the parental anti-EGFR or anti-PD-L1 monoclonal antibody.

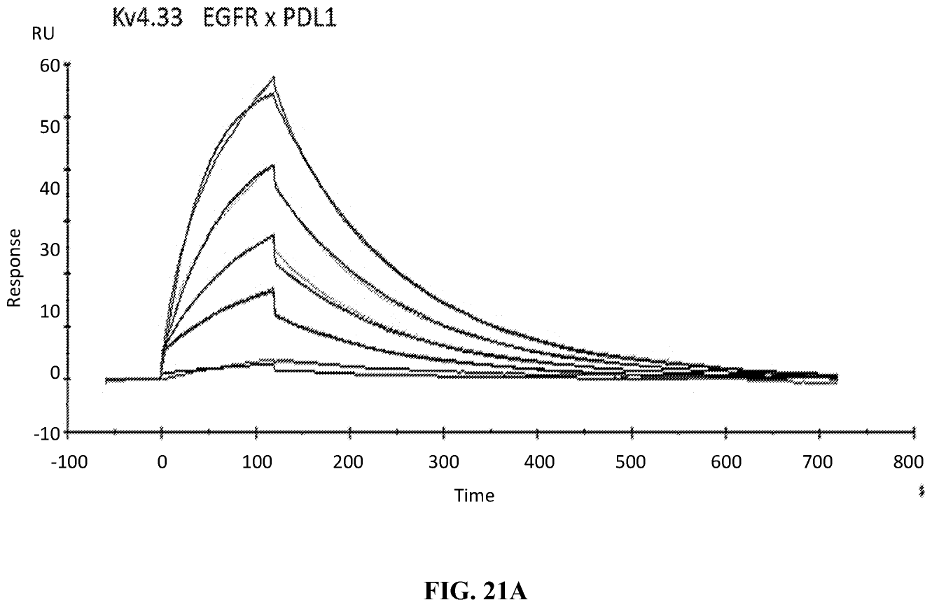

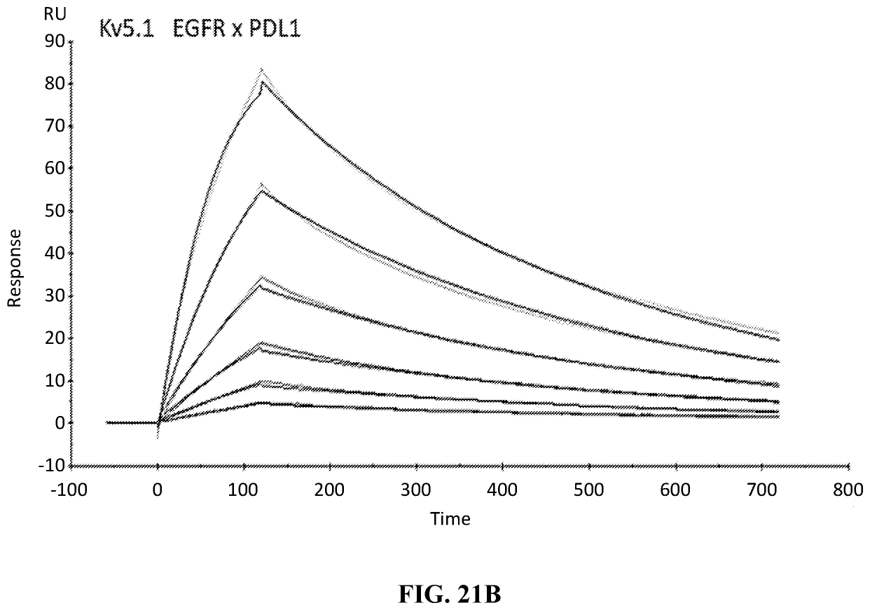

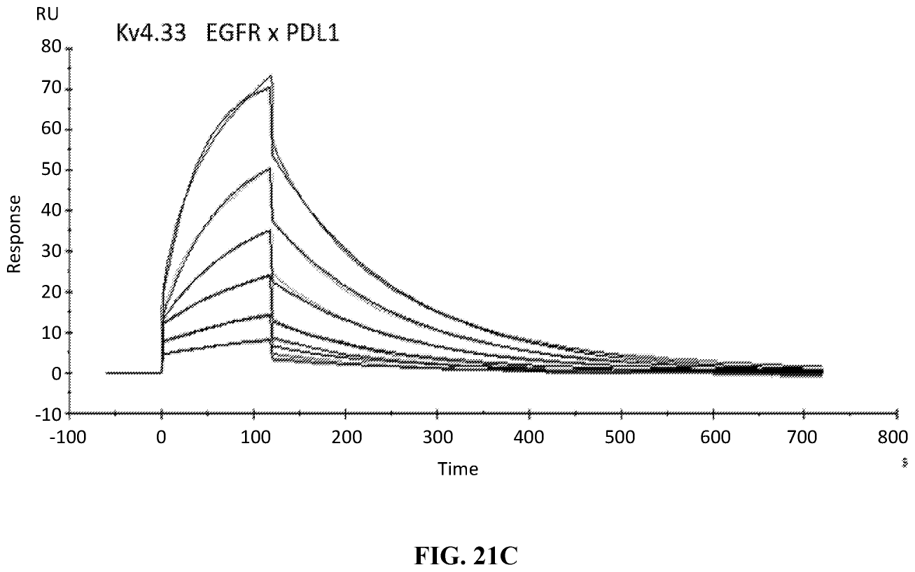

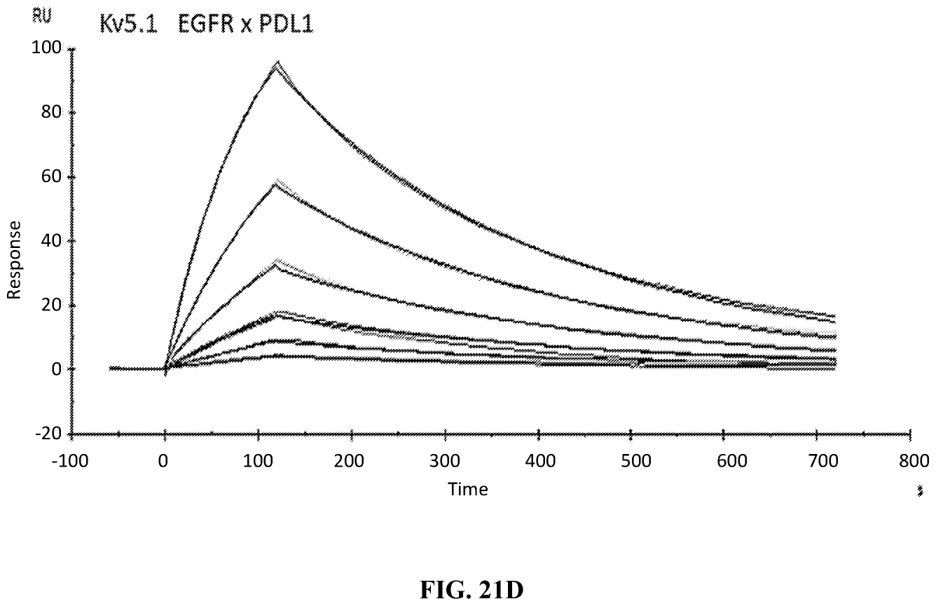

FIG. 21A shows a sensorgram of surface-bound EGFR antigen binding to the three-chain non-tandem bispecific antibody (structure shown in FIG. 4, Kv4.33). FIG. 21B shows a sensorgram of surface-bound EGFR antigen binding to the three-chain in-tandem bispecific antibody (structure shown in FIG. 2, Kv5.1). FIG. 21C shows a sensorgram of surface-bound PD-L1 antigen binding to the three-chain non-tandem bispecific antibody (structure shown in FIG. 4, Kv4.33). FIG. 21D shows a sensorgram of surface-bound PD-L1 antigen binding to the three-chain in-tandem bispecific antibody (structure shown in FIG. 2, Kv5.1).

A standard evaluation with the 1:1 binding model was applied to all kinetics series described above. The association and dissociation constants (k-on and k-off), the affinity (KD), maximal response (Rmax), as well as the fitting statistics (Chi\hat{ }2) are presented in Table 8, which appears at the end of the Examples section.

### Example 3: BiaCore Binding Analysis

Binding of Bispecific Antibodies to Two Target Antigens.

A sandwich method using the BiaCore system was used to determine binding kinetics of the three-chain bispecific antibodies to two different target antigens at the same time. Target antigen-1 (either EGFR or PD-L1) was immobilized by amine coupling onto a CM5 sensor chip surface. Then a three-chain bispecific antibody was immobilized to the reference and test flows by applying the same amount of the bispecific antibody for each cycle, followed by a concentration series of target antigen-2 (either PD-L1 or EGFR) as the analyte.

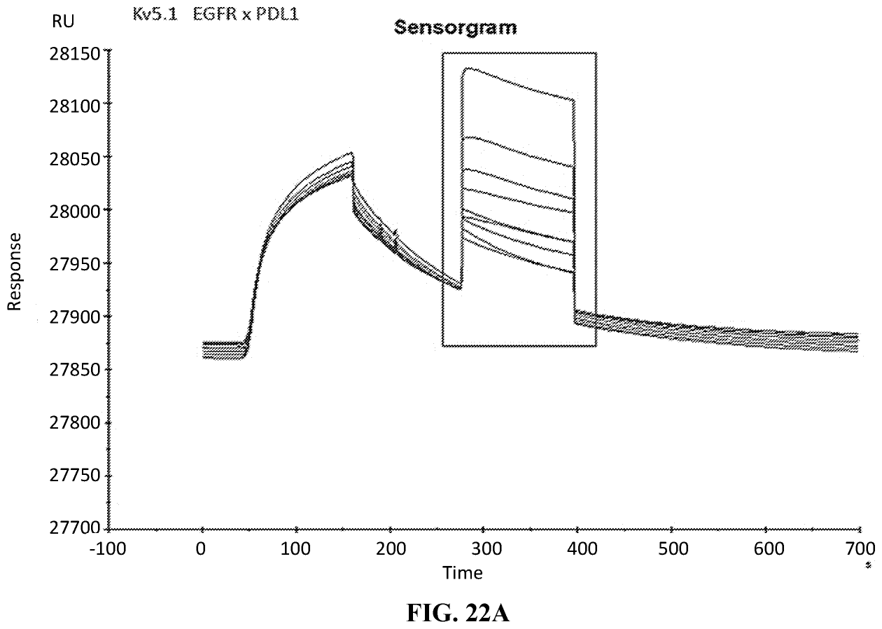

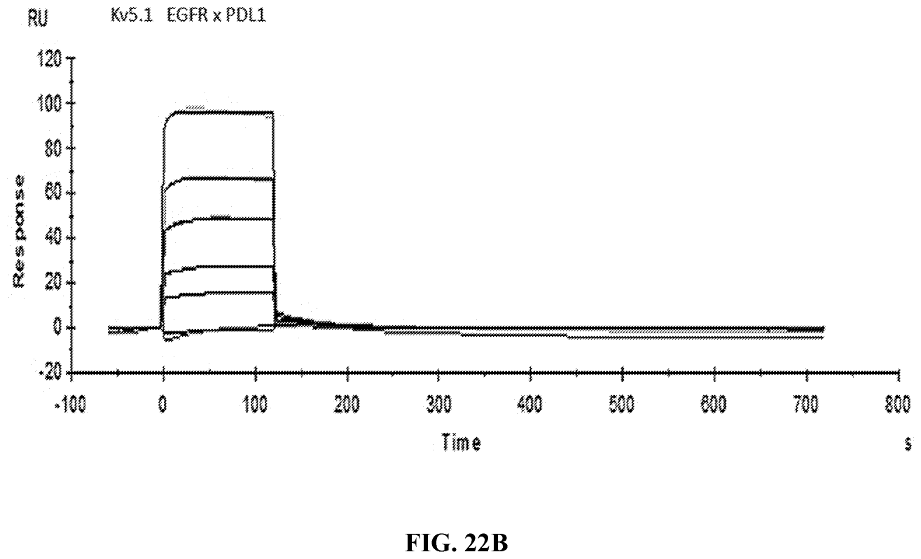

FIG. 22A shows a sensorgram of surface-bound EGFR antigen-1 binding to three-chain in-tandem bispecific antibody (structure shown in FIG. 2, Kv5.1) and binding PD-L1 antigen-2 at different concentrations in the mobile phase. The rectangle highlights the sensorgram time period that was subjected to kinetic analysis after subtraction of the reference flowcell responses. FIG. 22B shows the reference-subtracted sensorgram of the rectangle area from FIG. 22A.

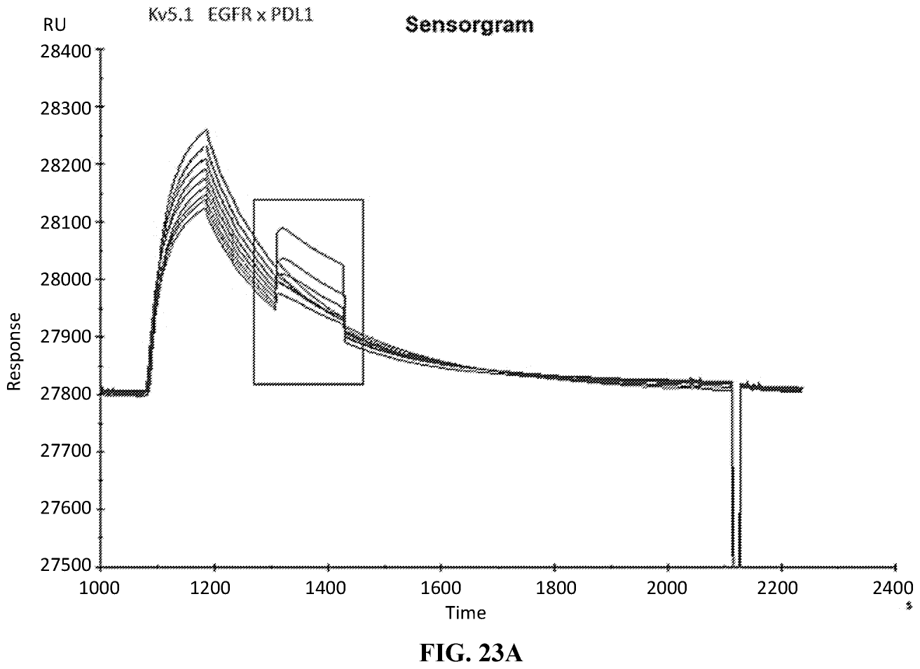

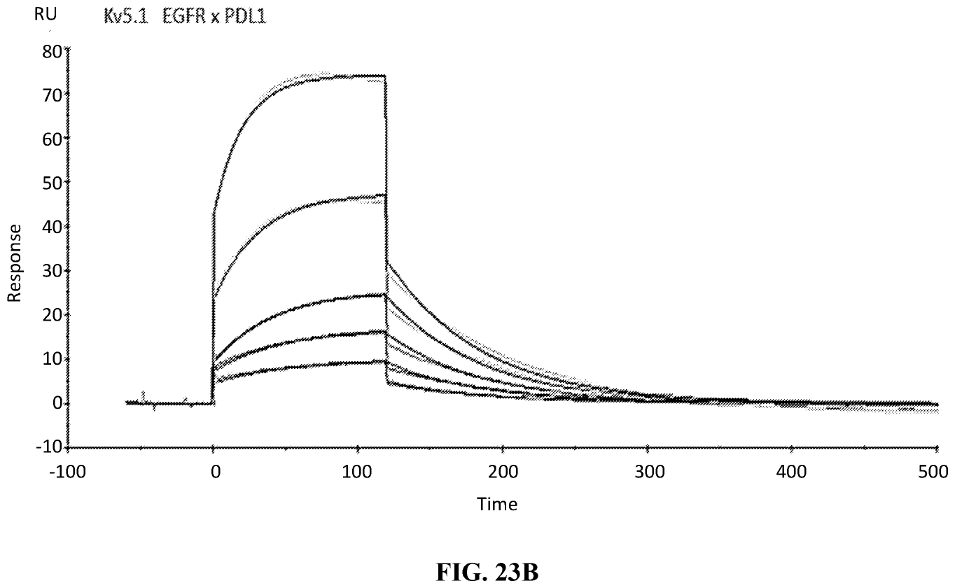

FIG. 23A shows a sensorgram of surface-bound PD-L1 antigen-1 binding to three-chain in-tandem bispecific antibody (structure shown in FIG. 2, Kv5.1) and binding EGFR antigen-2 at different concentrations in the mobile phase. The rectangle highlights the sensorgram time period that was subjected to kinetic analysis after subtraction of the reference flowcell responses. FIG. 23B shows the reference-subtracted sensorgram of the rectangle area from FIG. 22A.

A standard evaluation with the 1:1 binding model was applied to all kinetics series described above. The association and dissociation constants (k-on and k-off), the affinity (KD), maximal response (Rmax), as well as the fitting statistics (Chi\hat{ }2) were presented in Table 8, which appears at the end of the Examples section.

### Example 4: Cleavable Linkers

Design and Analysis of Cleavable Linkers for Bispecific Antibodies.

The cleavage efficiency of various linker sequences were designed and tested by including them on the two chain in-tandem and non-tandem bispecific antibodies (e.g., CD38/CD3). The linker sequences and code annotations are listed in Table 1 below.

TABLE 1

| | 2-chain | SEQ | 2-chain | SEQ |
|---|---|---|---|---|
| | in-tandem | ID | non-tandem | ID |
| Linker sequences: | Kv6.1 code | NO: | Kv6.2 code | NO: |
| TSGSGGSGGSV (non-cleavable) | EG | 12 | EH | 44 |
| TSGSGGSPLGMGGSGSV | EI | 13 | EU | 45 |
| TSGSGGSPLGVGGSGSV | EJ | 14 | EV | 46 |
| TSGSGGSPAALGGSGSV | EK | 15 | EW | 47 |
| TSGSGGSPAGLGGSGSV | EL | 16 | EX | 48 |
| TSGSGGSPLGMVGV | EM | 17 | EY | 49 |
| TSGSGGSPLGVVGV | EN | 18 | EZ | 50 |
| TSGSGGSPAALVGV | EO | 19 | FA | 51 |
| TSGSGGSPAGLVGV | EP | 20 | FB | 52 |
| TSGSGGSPLGMVLV | EQ | 21 | FC | 53 |
| TSGSGGSPLGVVLV | ER | 22 | FD | 54 |
| TSGSGGSPAALVLV | ES | 23 | FE | 55 |
| TSGSGGSPAGLVLV | ST | 24 | FF | 56 |

A series of MMP2/9 cleavable linkers were designed to include a combination of 1 or 2 units of flexible 5-amino acid linker of Glycine/Serine blend. A non-cleavable linker was also designed. Various two chain in-tandem (Kv6.1) and non-tandem (Kv6.2) bispecific antibodies were constructed to include one of these cleavable or non-cleavable linkers for transient CHO cell expression. The supernatant from the CHO cell culture was harvested, and the bispecific antibodies in the supernatant was processed by employing a one-step gravity protein A purification.

The linker cleavage efficiency was assessed by subjecting each of the protein A purified bispecific antibodies to a commercially-available MMP2 or MMP9 enzyme at conditions similar to the manufacturer recommended with some modifications. The Kv6.1 bispecific antibodies were digested in 50 mM Na Acetate, 50 mM Tris-HCl, and 10 mM CaCl₂) at pH6.8 with 2.7 nM MMP9 (CalbioChem) at 37° C. for 1 hr. The Kv6.2 bispecific antibodies were digested in 50 mM Na Acetate, 50 mM Tris-HCl, 10 mM CaCl₂), 0.05% BSA, and 0.05% Polysorbate 20 pH6.8, with 22 nM MMP9 (CalbioChem) or 3 U of MMP2 (Creative BioLab), at 37° C. for up to 2 hr.

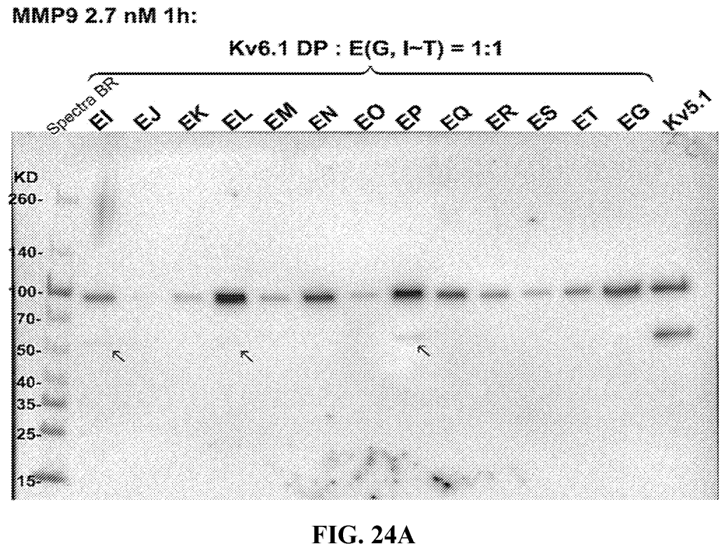

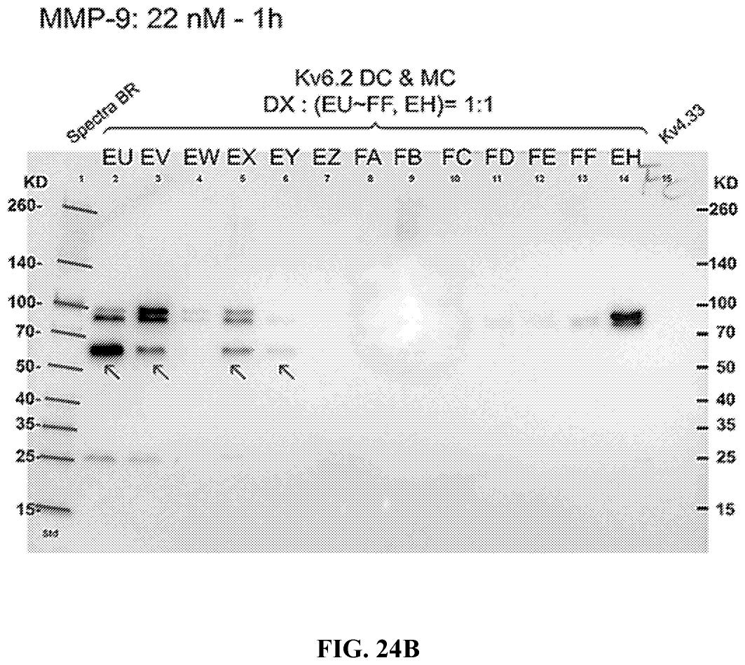

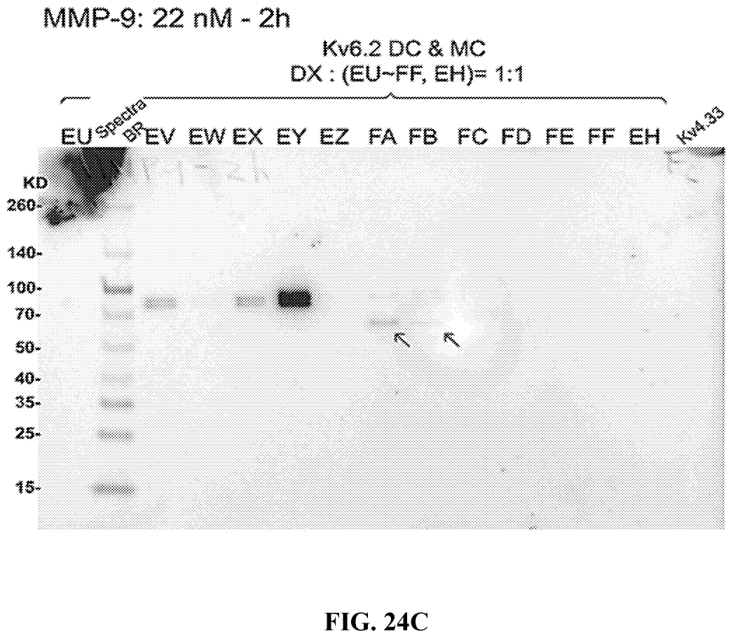

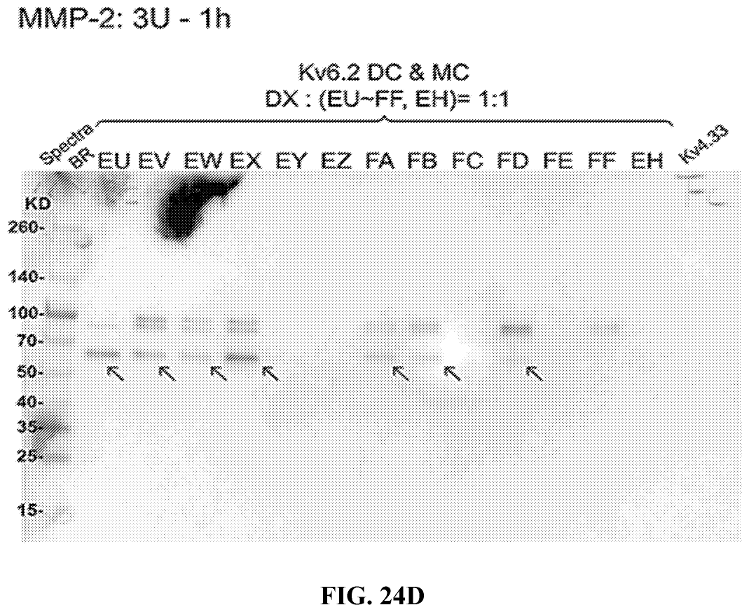

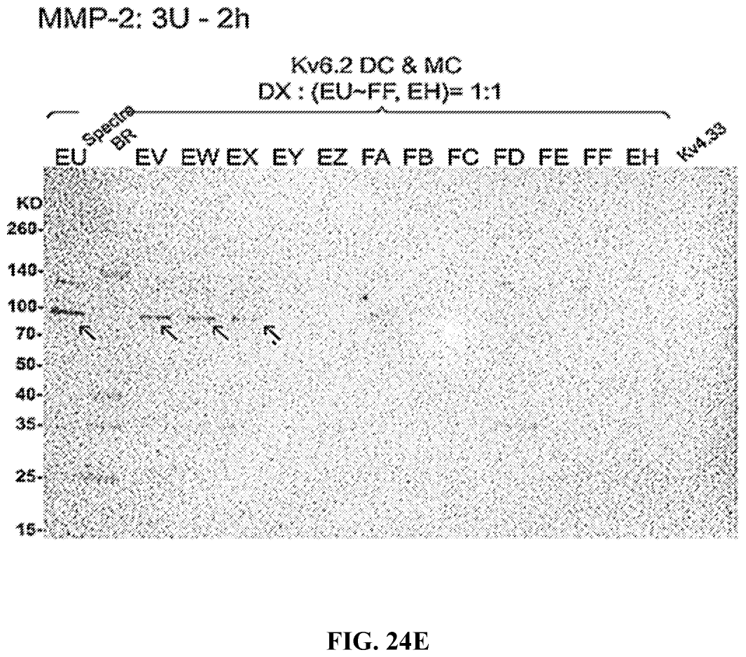

The protease-digested Kv6.1 and Kv6.2 bispecific antibodies were subjected to SDS-PAGE under reducing condition and subsequent anti-human Fc Western blot analysis to identify the presence of an Fc Fusion chain having an observed smaller molecular weight compared to the intact HC-Fc Fusion and LC-Fc Fusion chains. The marker-merged anti-human Fc Western blots of the protease-digested Kv6.1 and Kv6.2 bispecific antibodies are presented in FIGS. 24A-E.

The anti-human Fc Western blot (reducing condition) of the in-tandem (Kv6.1) bispecific antibodies digested with MMP9 (FIG. 24A) reveals detectable cleavage of the LC-Fc fusion chain in the bispecific antibodies containing linkers EI, EL or EP, after 1 hour of protease treatment. The arrows designate the cleavage product.

The anti-human Fc Western blot (reducing condition) of the non-tandem (Kv6.2) bispecific antibodies digested with MMP9 (FIGS. 24B and 24C) or MMP2 (FIGS. 24D and 24E) reveals detectable cleavage of the LC-Fc Fusion chain in the bispecific antibodies containing linkers EU, EV, EX or EY (FIG. 24B) after 1 hour digestion with MMP9, detectable cleavage in bispecific antibodies containing linkers FA or FB (FIG. 24C) after 2 hours digestion with MMP9. Cleavage of the non-tandem bispecific antibodies (Kv6.2) containing EU, EV, EW, EX, FA, FB or FD linkers is detectable with MMP2 treatment at 1 hour (FIG. 24D). Cleavage of the non-tandem bispecific antibodies (Kv6.2) containing EU, EV, EW, EX, FA, FB or FD linkers is detectable with MMP2 treatment at 1 hour (FIG. 24D) and up to 2 hours (FIG. 24E). The arrows designate the cleavage product.

The results indicate there is a difference in cleavage efficiency by the two related MMP protease enzymes. The results also indicate that the bispecific antibody format of the in-tandem (Kv6.1) and non-tandem (Kv6.2) design influence linker cleavage efficiency, because the same linker exhibits different cleavage efficiency in the in-tandem (Kv6.1) and non-tandem (Kv6.2) formats.

### Example 5: Cell Binding Analysis

Binding of Bispecific Antibodies to CD3-Expressing T Cells.

Binding of CD3 antigen on primary human T cells with various three-chain or two-chain bispecific antibodies was measured using an Intellicyt iQue screener flow cytometer. The bispecific antibodies tested included: three chain non-tandem bispecific antibodies (Kv4.33) that bind CD38 and CD3, or bind BCMA and CD3; three chain in-tandem bispecific antibody (Kv5.1) that binds CD38 and CD3; two chain non-tandem bispecific antibody (Kv6.2) that binds CD38 and CD3; and two chain in-tandem bispecific antibody (Kv6.1) that binds CD38 and CD3. For analysis of bispecific antibodies containing anti-CD38 antigen binding domain, freshly isolated human T cells were negatively selected for CD38-minus subpopulation prior to the analysis. For analysis of the three chain non-tandem bispecific antibody (Kv4.33) that binds BCMA/CD3, freshly isolated human T cells that were kept in ATC (Active T cell) media for less than one week were used. For both types of analyses, T cells with greater than 90% viability were washed once with FACS buffer, a DPBS based buffer containing 2% fetal bovine serum (FBS), and then adjusted to a cell density of 1E+6/mL in the same buffer. Approximately 25,000 cells were used to generate each data point.

For each dose-dependent curve of bispecific antibody or control mAb binding to CD3 on T cells, a series of 1:3 dilution, up to twelve times, of each bispecific antibody or control mAb were prepared in the FACS buffer. Equal volume of each bispecific antibody dilution and the T cells were mixed in a well of a 96-well plate. The concentration range for a particular bispecific antibody or control mAb were selected based on their estimated EC50 (concentration that gives half-maximal binding) towards CD3. After one hour of incubation of each concentration series of bispecific antibody or control mAb with their corresponding T cells, the mixtures were washed with FACS buffer and supernatant removed after centrifugation. An anti-human Fc-APC conjugate as secondary antibody were added to each bispecific antibody or control mAb/cell mixture at the manufacturer recommended titer and incubated for approximately 15-20 minutes. The bispecific antibody or control mAb/cell mixtures were washed and the supernatant removed as described above. The final bispecific/cell mixtures were resuspended in 25 uL of FACS buffer and subjected to FACS analysis on the flow cytometer with 10 uL of each suspension analyzed.

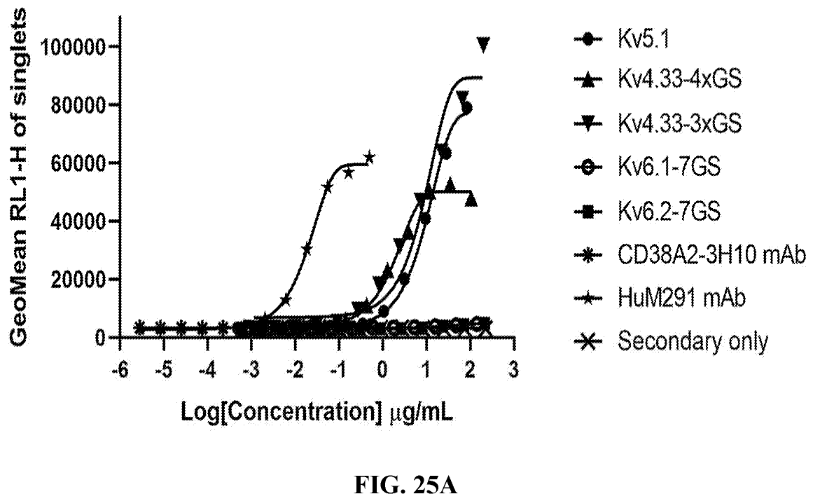

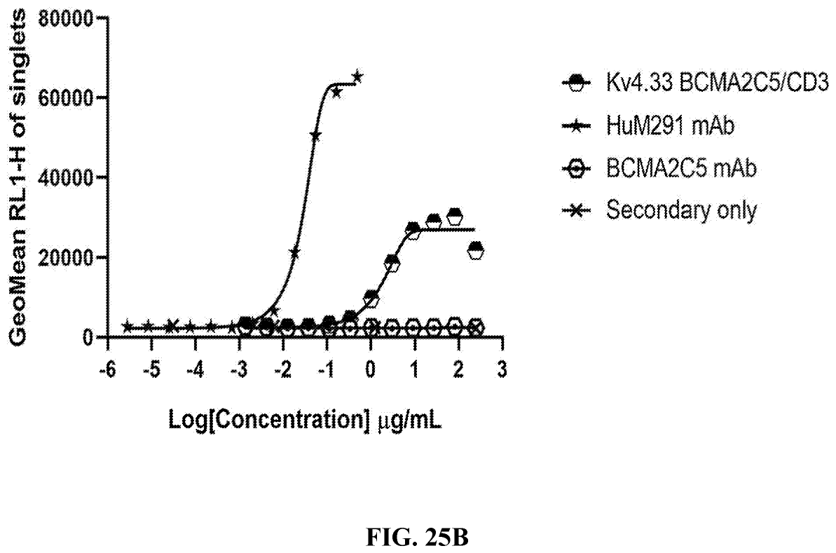

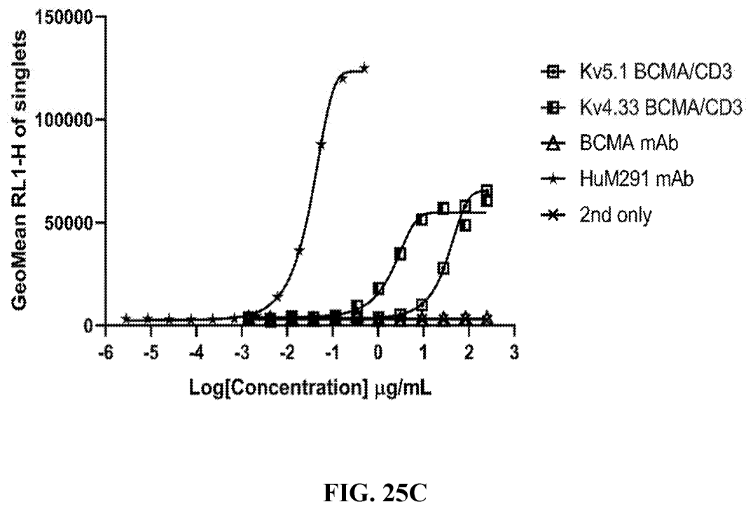

The dose-dependent binding curve of each bispecific antibody or control mAb binding to CD3 T cells were generated by plotting the Geomean of the fluorescence height of the detected singlet cells in the corresponding bispecific antibody or control mAb/cell mixture in each concentration series. EC50 of each bispecific or control mAb towards CD3 on T cells were determined after fitting the curve to the dose-dependent curve with a four-parameter linear regression (4PL) model. The binding curves of intact anti-CD3 containing bispecific antibodies toward CD3 on T cells are presented in FIGS. 25A, B and C. The EC50s of the control antibodies and the anti CD3-containing bispecific antibodies towards CD3 on T cells are listed in Tables 2 and 3A and B, respectively (see below).

TABLE 2

| | Control antibodies | | |
|---|---|---|---|
| Anti-CD3 | HuM291 | CD38 mAb | BCMA mAb |
| EC50 | 0.020 | N/A | N/A |
| R\hat{ }2 | 0.999 | N/A | N/A |

TABLE 3A

| | CD38/CD3 | | | | |
|---|---|---|---|---|---|
| | | Kv6.1 | Kv4.33 | Kv4.33 | Kv6.2 |
| Anti-CD3 | Kv5.1 | (7GS) | (4XGS) | (3XGS) | (7GS) |
| EC50 | 11.937 | 48.429 | 1.762 | 22.026 | 37.961 |
| R\hat{ }2 | 1.000 | 0.958 | 0.994 | 0.999 | 0.997 |

TABLE 3B

BCMA/CD3

| | Kv4.33 | |
|---|---|---|
| Anti-CD3 | (3XGS) | Kv5.1 |
| EC50 | 2.400 | 34.900 |
| R\hat{ }2 | 1.000 | 1.000 |

### Example 6: Cell Binding Analysis

Binding of CD3-Expressing T Cells to Bispecific Antibodies with Cleaved Linkers.

The binding of CD3-expressing T cells to bispecific antibodies with MMP2/9 cleavable linkers were analyzed. Bispecific antibodies that bind CD38 and CD3 and having two chain in-tandem (Kv6.1) and two chain non-tandem (Kv6.2) structures were analyzed as non-cleaved or cleaved forms.

Commercially-available MMP9 enzyme was used to digest the bispecific antibody at 1:200 Ab:Enzyme mass ratio in 50 mM Na Acetate, 50 mM Tris-HCl, 10 mM CaCl₂), 0.05% BSA, and 0.05% Polysorbate 20 pH6.8 at 16° C. overnight. Each digestion mixture was purified by protein-A affinity batch binding and gravity elution with 50 mM acetic acid, followed by a one-step titration with equal volume of 0.2 M Na Acetate pH5.5 to reach final buffer condition at 125 mM Na Acetate pH5.0. The pH-titrated protein A elution fractions of the MMP9-diggested CD38/CD3 bispecific antibodies were subjected to SDS-PAGE under non-reducing as well as reducing conditions for linker cleavage validation. Upon confirmation of complete cleavage of the cleavable linker by comparative reducing SDS-PAGE of the intact and digested bispecific antibodies, the titrated protein A elution fractions were quantified for protein concentration by UV280 absorption and prepared for T cell binding assay. The preparation of the concentration series and CD3-expressing CD38-minus human T cells, as well as the assay setup and analyses are described in Example 5 above.

Control non-cleavable antibodies that bind CD38/CD3 included two chain in-tandem (Kv6.1) and non-tandem (Kv6.2) molecules that carry a non-cleavable GS linker replacing the cleavable linker on the second polypeptide.

Control three chain antibodies that bind CD38/CD3 included in-tandem (Kv5.1) and non-tandem (Kv4.33) molecules.

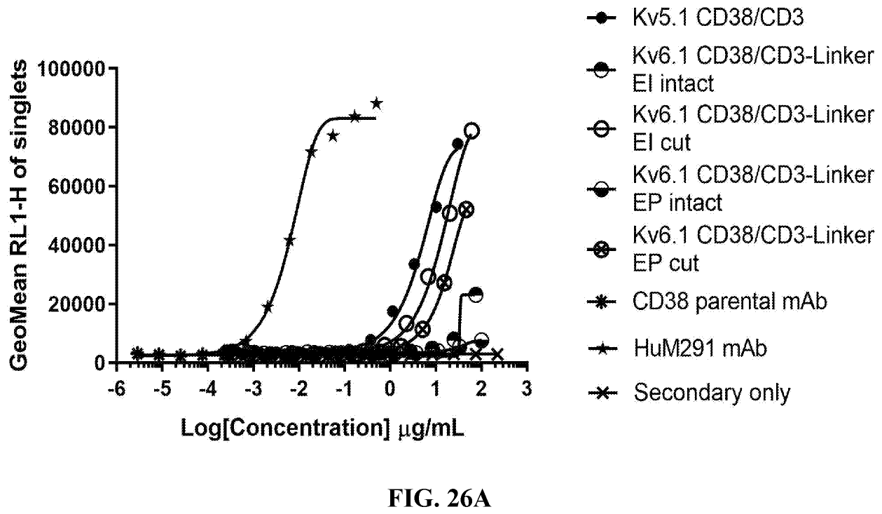

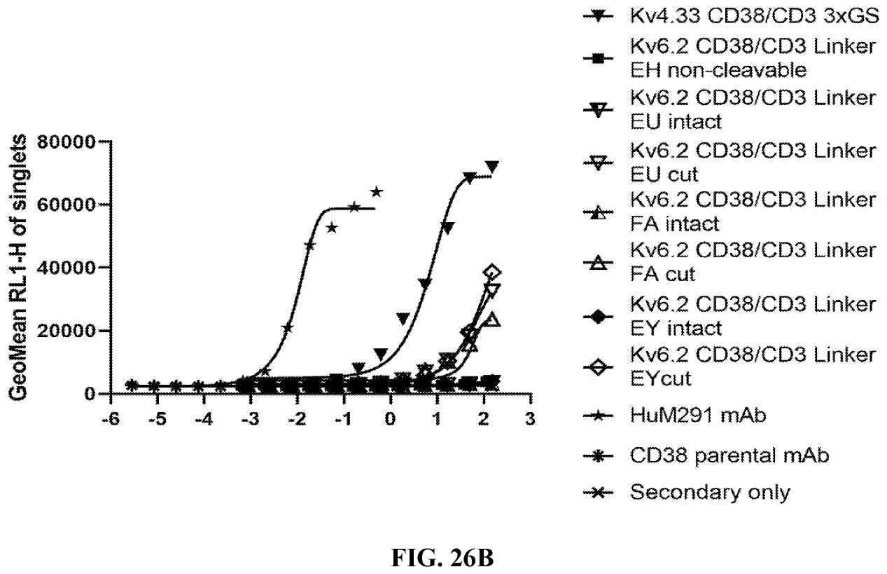

The CD3 binding curves of the bispecific antibodies with cleavable linkers before and after linker cleavage are presented in FIGS. 26A and B.

The results in FIG. 26A show that MMP9 protease cleavage of the two chain in-tandem bispecific antibodies (Kv6.1) carrying MMP2/9-cleavable linkers restores binding to CD3-expressing CD38-minus T cells, indicating that linker cleavage increased accessibility of the paratope of the second Fab region in these bispecific antibodies. Cleavage of the two chain in-tandem bispecific antibody (Kv6.1) carrying MMP2/9-cleavable linkers have a comparable affinity for CD3 antigen (e.g., within 10-fold difference) compared to the three chain in-tandem bispecific antibody (Kv5.1). The control non-cleavable bispecific antibody having an in-tandem structure (Kv6.1) exhibit greatly reduced binding to CD3 by more than 100-fold difference. Solid lines are dose-dependent 4 parameter logistic regression (4PL) fitted curves of corresponding data sets.

The results in FIG. 26B show that MMP9 protease cleavage of the two chain non-tandem bispecific antibodies (Kv6.2) carrying MMP2/9 cleavable linkers increased binding to CD3-expressing CD38-minus T cells. Cleavage of the two chain non-tandem bispecific antibody (Kv6.2) carrying MMP2/9-cleavable linkers have an affinity for CD3 antigen that is within 100-fold difference compared to the three chain non-tandem bispecific antibody (Kv4.33). The control non-cleavable bispecific antibody having a non-tandem structure (Kv6.2) exhibit greatly reduced binding to CD3 by more than 1000-fold difference. Solid lines are dose-dependent 4 parameter logistic regression (4PL) fitted curves of corresponding data sets.

The corresponding EC50 values of binding CD3 on T cells before and after linker cleavage of the various bispecific antibodies are listed in Tables 4 and 5 below.

TABLE 4

| | | 2-chain and 3-chain | | | | |
|---|---|---|---|---|---|---|
| Anti-CD3 | | In-tandem CD38/CD3 bispecific antibodies | | | | |
| Bispecific | | | Kv6.1 | Kv6.1 | Kv6.1 | CD3 mAb |
| antibody | | Kv5.1 | (7GS) | (EI) | (EP) | HuM291 |
| Intact | EC50 | 8.449 | >100 | >100 | >100 | 0.010 |
| | R\hat{ }2 | 0.999 | N/A | N/A | N/A | 0.995 |
| Cleaved | EC50 | N/A | N/A | 27.001 | 30.518 | N/A |
| | R\hat{ }2 | N/A | N/A | 0.999 | 1.000 | N/A |

TABLE 5

| | | 2-chain and 3-chain | | | | |
|---|---|---|---|---|---|---|
| Anti-CD3 | | Non-tandem CD38/CD3 bispecific antibodies | | | | |
| Bispecific | | Kv4.33 | Kv6.2 | Kv6.2 | Kv6.2 | Kv6.2 |
| antibody | | (3XGS) | (7GS) | (EU) | (EY) | (FA) |
| Intact | EC50 | 1.154 | >1000 | >100 | >100 | >100 |
| | R\hat{ }2 | 0.983 | N/A | N/A | N/A | N/A |
| Cleaved | EC50 | N/A | N/A | >1000 | >1000 | >1000 |
| | R\hat{ }2 | N/A | N/A | N/A | N/A | N/A |

### Example 7: Cytokine Release Analysis

Cytokine Release Induced by CD38/CD3 Bispecific Antibodies.

A multiplex MSD (Meso Scale Diagnostics) method was used to analyze cytokine release capability of CD38/CD3 bispecific antibodies. Unstimulated human T cells and RPMI8226 cells were mixed with various concentrations of CD38/CD3 bispecific antibodies or control antibodies in the redirected T cell cytotoxicity assays. Three cytokines were tested, including IFNγ, IL-2, and TNFα, in a T cell cytotoxicity which were assayed simultaneously for each redirected T cell cytotoxicity condition. The manufacturer recommended assay procedure was used in carrying out the assays with the following sample handling practice.

Previously frozen supernatants were thawed at room temperature and homogenized by inversion and brief centrifugation. For the series treated with bispecific antibody or control monoclonal antibody concentration gradients, where tumor target cells RPMI killings were observed, the homogenized supernatants were diluted 50 times prior to MSD plate loading. For the series as cell-only controls, i.e. tumor target cells only, T cells only, or the two-cell mixtures, where less cell death was observed and less cytokines were expected, the supernatants were diluted 10 times prior to MSD plate loading.

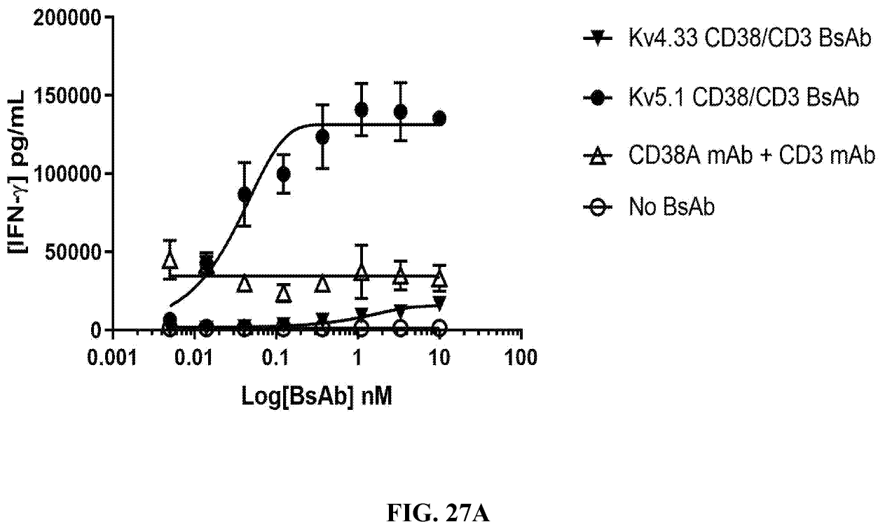

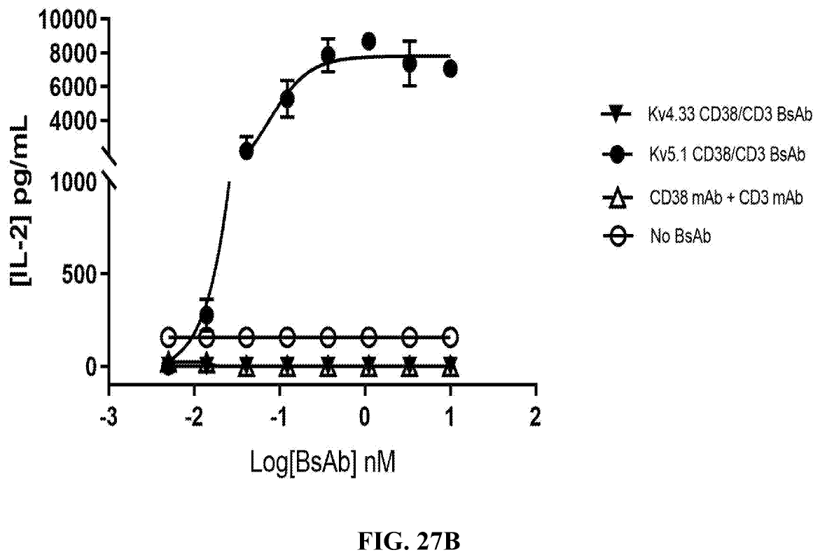

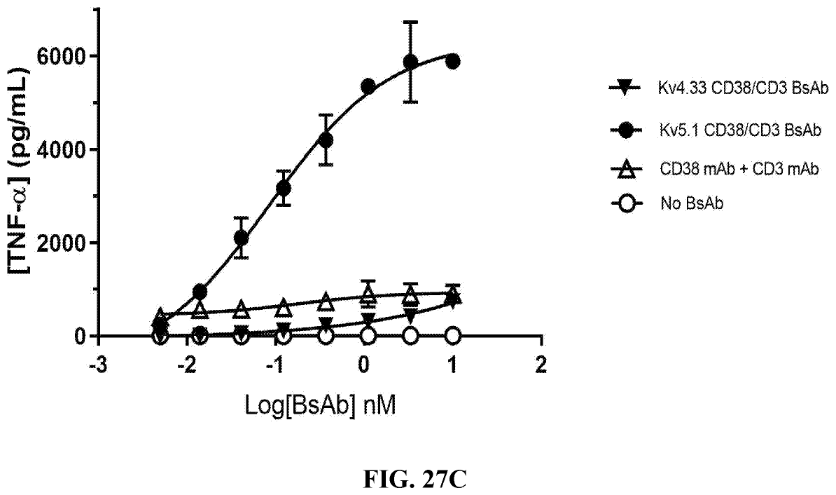

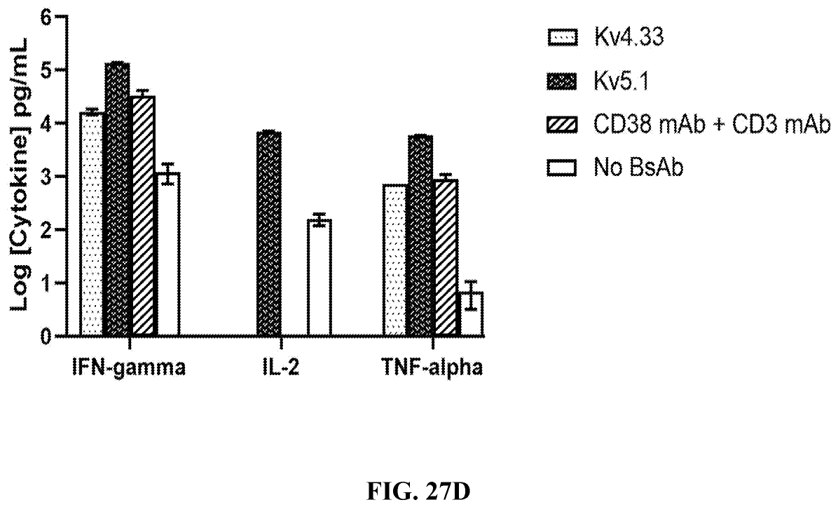

The cytokine release profiles of each CD38/CD3 bispecific antibody as well as the mAb and cell only controls against RPMI8226 cells are presented in FIGS. 27A-C. Solid lines in FIGS. 27A-C are dose-dependent 4 parameter non-linear regression (4PL) fitted curves of the corresponding data sets. A side-by-side comparison of each cytokine released in the presence of 10 nM CD38/CD3 bispecific antibody, or 5 nM of each of the parental monoclonal antibodies, are presented in FIG. 27D.

The results shown in FIGS. 27A-D indicate that the three chain in-tandem bispecific antibody (Kv5.1; structure illustrated in FIG. 2) can induce cytokine release of IFNγ, IL-2, and TNFα at a higher level compared to the three chain non-tandem bispecific antibody (Kv4.33; structure illustrated in FIG. 4).

### Example 8: In Vitro T Cell Activation Assay

Tumor-Associated Antigen-Dependent In Vitro T Cell Activation.

Freshly isolated unstimulated human T cells were mixed with CD38(+)/BCMA(+) myeloma cell line M1.R GFR/luc, and CD38/CD3 or BCMA/CD3 bispecific antibodies or parent monoclonal antibody. The T cells and tumor cells were mixed at a fixed E/T (effector cell/target cells) ratio which were reacted with each bispecific antibody at concentrations above or below the previously-determined EC50 of the given bispecific antibody.

Approximately 15,000 MM1.R cells and 150,000 primary human T cells per assay condition were incubated at 37° C. in a static incubator overnight in a 96-well plate in the presence of 1 pM, 10 pM, or 1 nM of each bispecific antibody or control parental mAb. Antigen-free control assays included T cell only with each of the corresponding bispecific antibody or control parental mAbs. Cells from the overnight incubation were pelleted by centrifugation and washed once with the RPMI media+10% FBS prior to FACS staining and analysis. Cell pellets were resuspended in FACS buffer and double-stained with anti-CD25-APC and anti-CD69-APC-Cy7 antibodies (BioLegend, Inc.) according to manufacturer recommended assay titer and procedure. Proper compensation control for double-color FACS assay as well as no-Ab controls were also included in the assay design.

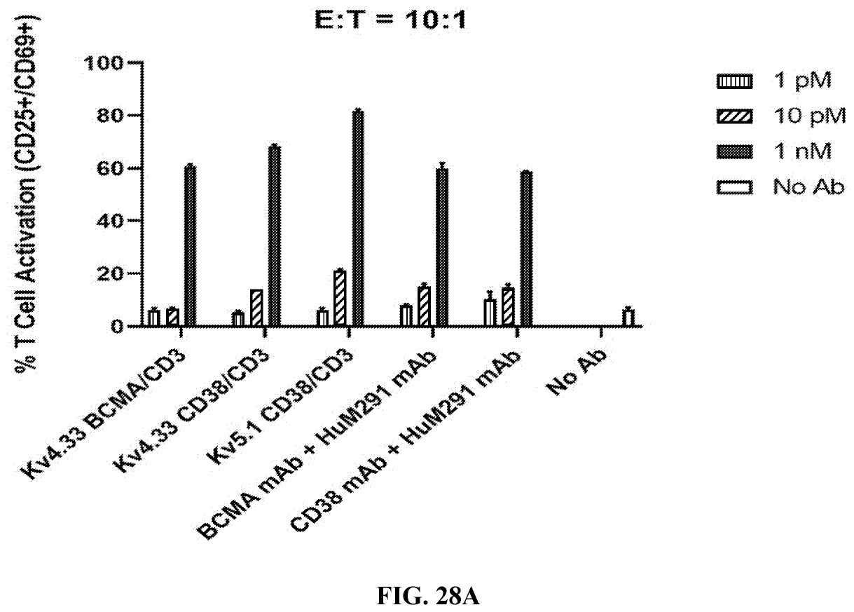

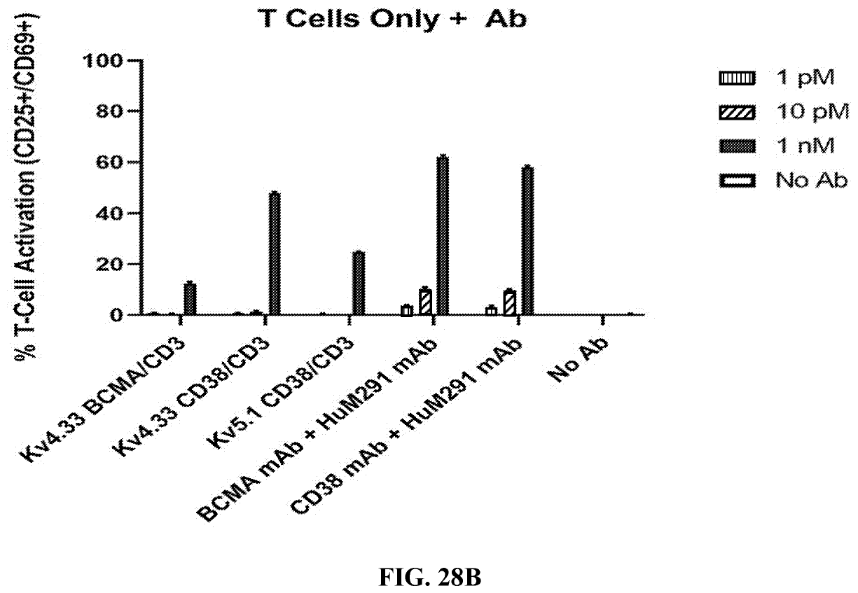

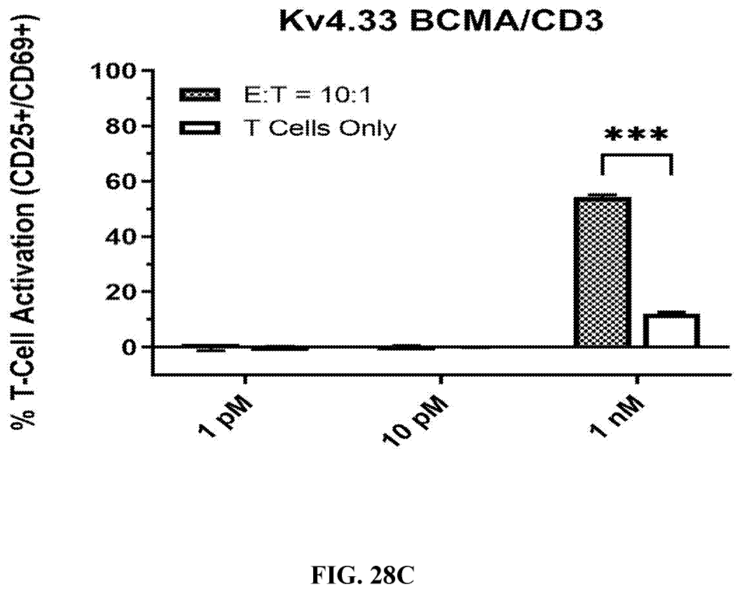

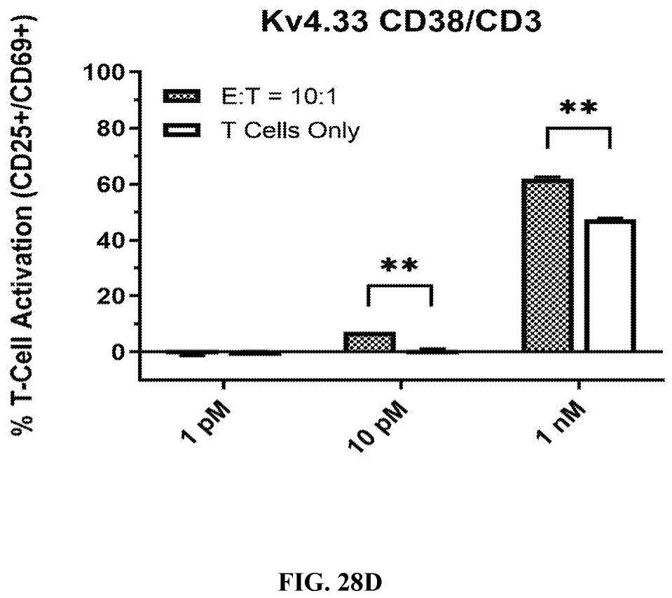

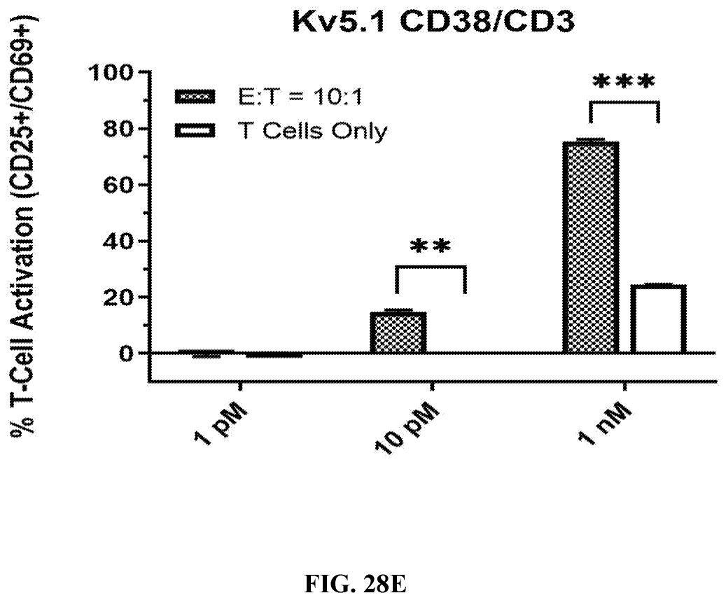

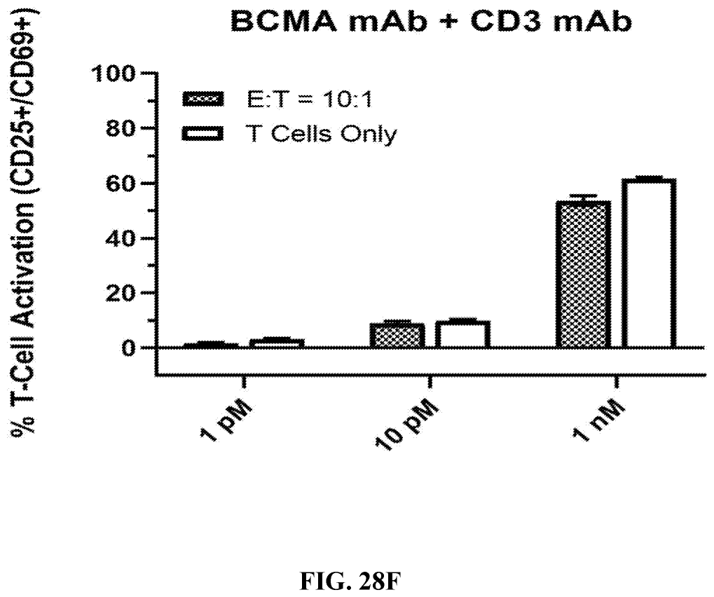

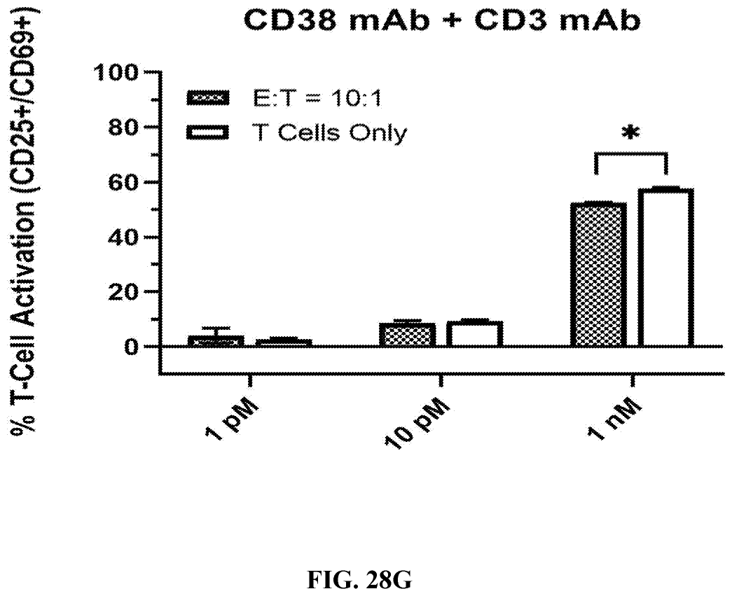

Prior to FACS analysis, the washed and double-stained cell pellets were resuspended in FACS buffer. Approximately ⅓ of (or 50,000-60,000) cells per assay condition were analyzed by the FACS instrument on BL-1 (GFP), RL-1 (CD25-APC), and RL-2 (CD69-APC-Cy7) channels. T cells were gated from the MM1.R target cells on the FSC-A (forward scattering—area) vs. BL1-H (height) scatter plot, with the BL1-H low population as the effector cells. The T cell population was then plotted on the RL-1 (CD25-APC) vs RL-2 (CD69-APC-Cy7) scatter plot after proper compensation was applied. The upper right quadrant, where both CD25(+) and CD69(+) are highly expressed, was defined as the activated T cell population. The percentage of the CD25(+)/CD69(+) T cells in all detected T cell populations was calculated as the percent activation for the corresponding assay condition and presented in FIGS. 28A and B. The results in FIG. 28A shows that the CD38/CD3 and BCMA/CD3 bispecific antibodies mediate TAA-dependent activation of unstimulated T cells in a dose dependent manner. The results in FIG. 28B show that, in the absence of tumor target, the CD38/CD3 and BCMA/CD3 bispecific antibodies elicit less T cell activation across the same concentration range tested in data shown in FIG. 28A. In the parental mAb conditions, the level of T cell activation was not dependent upon the presence of tumor antigen (shown in FIGS. 28A and B). The percent activation per assay condition was normalized by subtracting the level of activation observed in target cells and T cells only conditions. The normalized percent activation was plotted against the corresponding bispecific antibody or control parent mAb concentration and is presented in FIG. 28C-G.

The results show that bispecific antibody-mediated T cell activation is dose-dependent and target-specific for the three chain non-tandem BCMA/CD3 bispecific antibody (Kv4.33) (FIG. 28C), three chain non-tandem CD38/CD3 bispecific antibody (Kv4.33) (FIG. 28D), and three chain in-tandem CD38/CD3 bispecific antibody (Kv5.1) (FIG. 28E) at bispecific antibody concentrations greater than 25% of the IC50 of the previously characterized TAA-dependent redirected T cell cytotoxicity. Under the same tested concentration range, such tumor target-dependent T cell activation was not observed with control parental anti-BCMA mAb and anti-CD3 mAb (FIG. 28F) or control parental anti-CD38 mAb and anti-CD3 mAb (FIG. 28G). * p value <0.05; ** p value <0.01; *** p value <0.001.

### Example 9: In Vitro T Cell Cytotoxicity Assay

In Vitro Tumor Associated Antigen Dependent T Cell Cytotoxicity Assay

An in vitro tumor associated antigen (TAA) dependent T cell cytotoxicity assay was conducted to determine target cell killing capability of the bispecific antibodies using an equal amount of human T cells and CD38-expressing tumor cells at a fixed effector cells/target cells ratio and different concentrations of the CD38/CD3 bispecific antibodies and quantifying tumor cells expressing apoptosis markers.

Freshly isolated (unstimulated) human T cells were kept in RPMI media, supplemented with 10% FBS and 10 ng/mL IL-7 at approximately 2-5×10⁶ cells/mL at 37° C. static incubator overnight. Prior to the cytotoxicity assay setup, an assay of cytotoxic versus helper T cell ratio was conducted by quantifying the percentage of CD8(+) and CD4(+) subpopulations, respectively, in total CD3(+) T cell counts using a compensated three-color flow cytometry method. An approximate CD4(+)/CD8(+) ratio of 2:1 is a typical criterion for qualifying the isolated human T cells for the cytotoxicity assay.

Cells of the RPMI8226 cell line, a CD38 high-expressing human multiple myeloma cell line (ATCC, CCL-155), were labeled by transduction with pMYs-IRES, a retrovirus-derived vector with GFP and firefly luciferase genes and subsequent selection for a stable cell line of endogenous GFP and luciferase expression for fluorescence as well as luminescence detection. RPMI GFP-Luc cell and freshly isolated T cells of viability >90% on the day of the assay setup were each washed with RPMI media once and the density was adjusted to provide an E/T ratio of 20:1 in each assay condition, with approximately 15,000 target cells used per data point. A serial 1:3 dilution of the CD38/CD3 three chain in-tandem (Kv4.33) and non-tandem (Kv5.1) bispecific antibodies, as well as a mixture of the two parental mAb (anti-CD38 and antiCD3 mAbs), was prepared in RPMI media and added to the RPMI/T cell mixtures in 96-well plates. A series of target cell and T cell only wells were also setup as the secondary antigen only and no-bispecific antibody controls. RPMI only and T cell only wells were also included in the setup to assess the intrinsic apoptosis baseline of each cell type. To address the edge effect of plate-based assays, only the inner rows and columns of the 96-well plates were used for setting up the assays. The border rows and columns were filled with equal volumes of the RPMI media as the inner assay wells. The assay plates were kept in at a 37° C. static incubator overnight.

Cells from the overnight incubation were pelleted by centrifugation. One hundred uL of the assay supernatants were transferred from each of the assay wells into a new 96-well PCR tube plates and frozen at −80° C. for cytokine release analyses at a later time (see Example 7). The cell pellets were washed with FACS buffer, 2% FBS in DPBS, once and centrifuged prior to supernatant removal. The cell pellets in the assay plates were subjected to Annexin V, an early apoptosis marker, followed by detection and analysis. The Annexin V detection was performed using a commercially available FACS kit by BioLegend, Inc, following manufacturer recommended conditions and procedures.

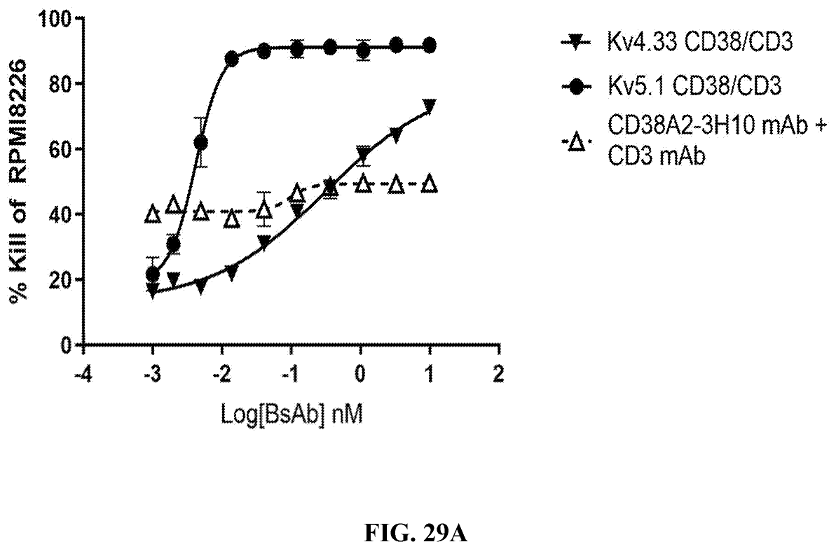

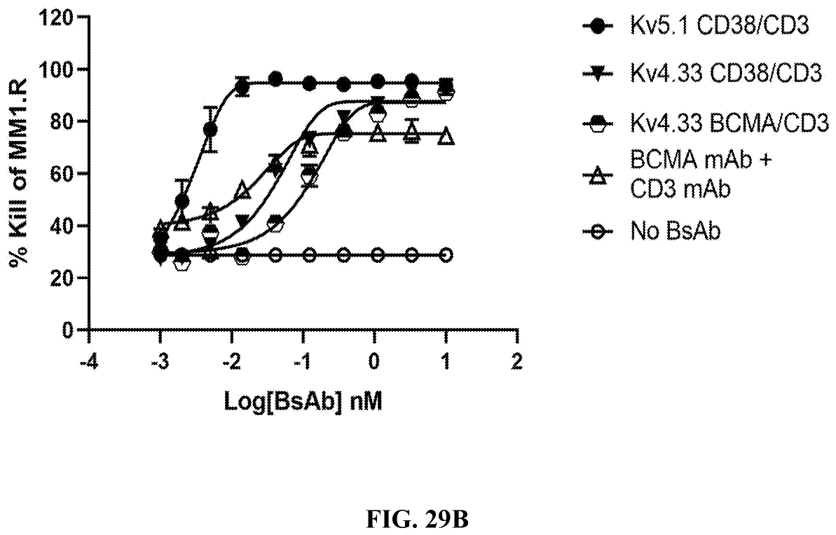

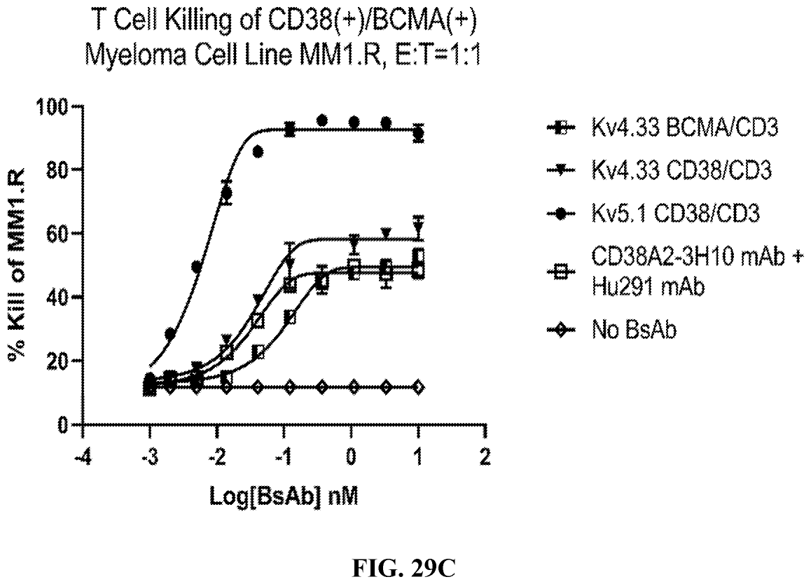

The assay plates were then subjected to FACS analysis on the Intellicyte iQue Screener (Sartorius) instrument with BL-1 channel for GFP and RL-1 channel Annexin V detection. Due to the constitutive expression of GFP, the RPMI GFP/Fluc target cells can be distinguished from the T cells on the FSC-A (forward scattering—area) vs. BL1-H (height) scatter plot, with the BL1-H high population being the target cells, the BL1-H low the effector cells. The RPMI cells were further gated on the RL1-H (Annexin V) vs. BL1-H scatter plot, with the RL1-H high population being the Annexin V high expression subset. The % Kill of the target cells is defined as the percentage of the Annexin V high expressing subset over the total cell counts of the RPMI in each assay condition. The % Kill of RMPI is then plotted against the bispecific antibody or mAb, parental mix or secondary antibody concentration (FIGS. 29A and B). The 4-parameter non-linear regression (4-PL) fitted dose-dependent curves of these % Kill vs Log[Concentration] plots produce an IC50 for each of the bispecific antibody or parental mAb mix control (see Table 6 below). The IC50 is defined as the concentration of the biological agent needed to produce 50% of the maximal observable effect for the defined experimental condition. The 4-PL fitted dose-dependent killing curves of the CD38/CD3 bispecific antibody as well as the parental monoclonal antibody mix control are presented in FIG. 29A and the IC50 of the corresponding molecules are listed in Table 6 below.

TABLE 6

TAA-dependent cytotoxicity
towards RPMI8226 cells

| | | |
|---|---|---|
| T cell | CD38/CD3 bispecific | Control |
| redirecting | antibodies | CD38 mAb and |

| bispecific Abs | Kv4.33 | Kv5.1 | CD3 mAb |
|---|---|---|---|
| IC50 pM | 316.2 | 4.2 | 95.8 |
| R\hat{ }2 | 0.990 | 0.990 | 0.822 |
| Baseline Kill % | 13.1 | 19.4 | 40.8 |
| Max Kill % | 80.9 | 91.2 | 49.5 |

The ability of bispecific antibodies to mediate killing of target cell expressing BCMA was conducted in a manner similar to the in vitro methods described above, with the following modifications: MM1.R was used as the target cell, three chain non-tandem (Kv4.33) BCMA/CD3 bispecific antibody was used, and the E/T ratio was 10:1. Similar to the RPMI-GFP/Fluc transgenic expression of GFP/luciferase marker, MM1.R cells (ATCC) were genetically modified to express GFP and luciferase for fluorescence and luminescence detection using the retroviral vector pMYs-IRES. The 4-PL non-linear fitted dose-dependent killing curves of the BCMA/CD3 bispecific antibody and CD38/CD3 TAA control, as well as the parental mAb mix control, are presented in FIGS. 29B and C, and the IC50 of the corresponding molecule listed in Table 7 below. The two independent runs were accompanied with different 2-parental mAb mix controls, either with anti-BCMA/anti-CD3 (C) or anti-CD38/anti-CD3 (D) combination.

TABLE 7

| | TAA-dependent cytotoxicity | | | |
|---|---|---|---|---|
| | towards RPMI8226 cells | | | |
| T cell | CD38/CD3 bispecific | | BCMA/CD3 | Control |
| redirecting | antibodies | | Bsp Abs | BCMA mAb and |
| bispecific Abs | Kv4.33 | Kv5.1 | Kv4.33 | CD3 mAb |
| IC50 pM | 40.5 | 3.0 | 134.4 | 19.3 |
| R\hat{ }2 | 0.994 | 0.980 | 0.975 | 0.977 |
| Baseline Kill % | 22.5 | 29.0 | 29.0 | 38.1 |
| Max Kill % | 92.8 | 95.2 | 90.0 | 76.3 |

The data in FIGS. 29A-C indicate that the non-tandem bispecific antibody (Kv4.33) is less potent at re-directing T cell-mediated cytotoxicity. Also observed is an antigen-dependent potency difference in Kv4.33 CD38/CD3 and Kv4.33 BCMA/CD3 bispecific antibodies, with the latter having a relatively lower efficacy in this effect.

### Example 10: Donor Response Profiling of CD38-Targeting Bispecific or mAb-Mediated In Vitro Tumor Cell Cytotoxicity

CD38 Expression Level Assessment of Tumor Cell Lines by Flow Cytometry.

Six known CD38(+) tumor cell lines, RPMI 8226 (ATCC, CCL-155), (ATCC, CRL-2975), IM-9(ATCC, CCL-159), Raji (ATCC, CCL-86), Daudi (ATCC, CCL-213) and NCI-H929 (ATCC CRL-9068). RPMI 8226, MM.1R, IM-9, and Raji cell lines, were labeled by transduction with pMYs-IRES, a retrovirus-derived vector with GFP and firefly luciferase genes and subsequent selection for a stable cell line of endogenous GFP and luciferase expression for fluorescence as well as luminescence detection.

Each of the 6 cell lines were cultured to sub-confluent in cell culture flasks the day prior to CD38 antigen staining. Cells with greater to 90% viability were titrated to 1E+6 per mL density and seeded in a V-bottom 96-well plate at 50,000 cells per well. FACS buffer, 2% fetal bovine serum (FBS) in DPBS, was added to wash the cells and supernatants aspired after gentle centrifugation at 500×g for 3 min at room temperature. The cell pellets were resuspended in FACS buffer with anti-CD38 APC-conjugate (Biolegend) at manufacturer recommended concentration along with designated unstained controls. The cells were incubated at room temperature in the dark for 20 min. FACS buffer was added to wash the cells and supernatants aspired after similar gentle centrifugation as described earlier. The cell pellets were resuspended in FACS buffer prior to flow cytometry analysis.

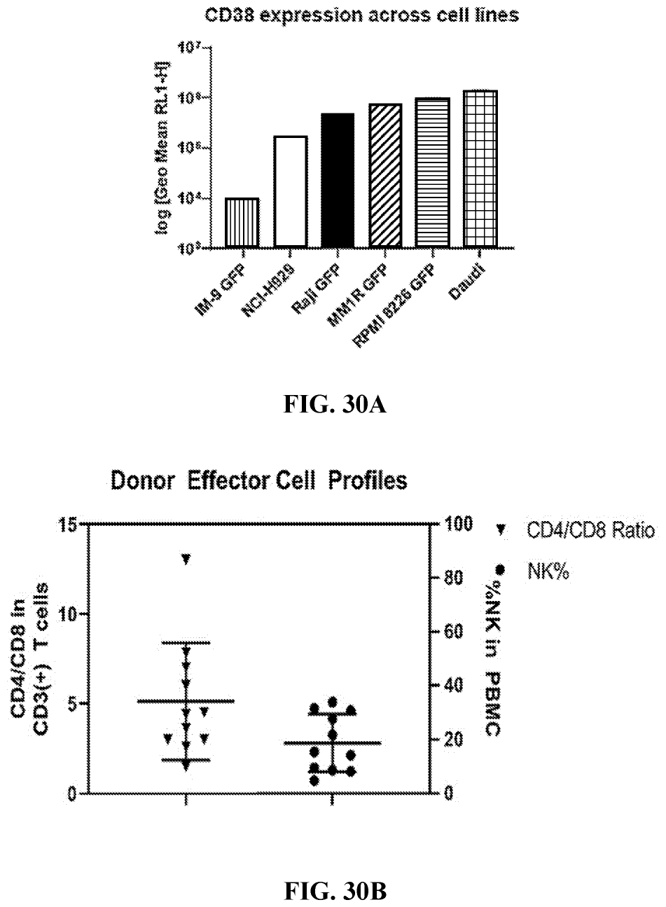

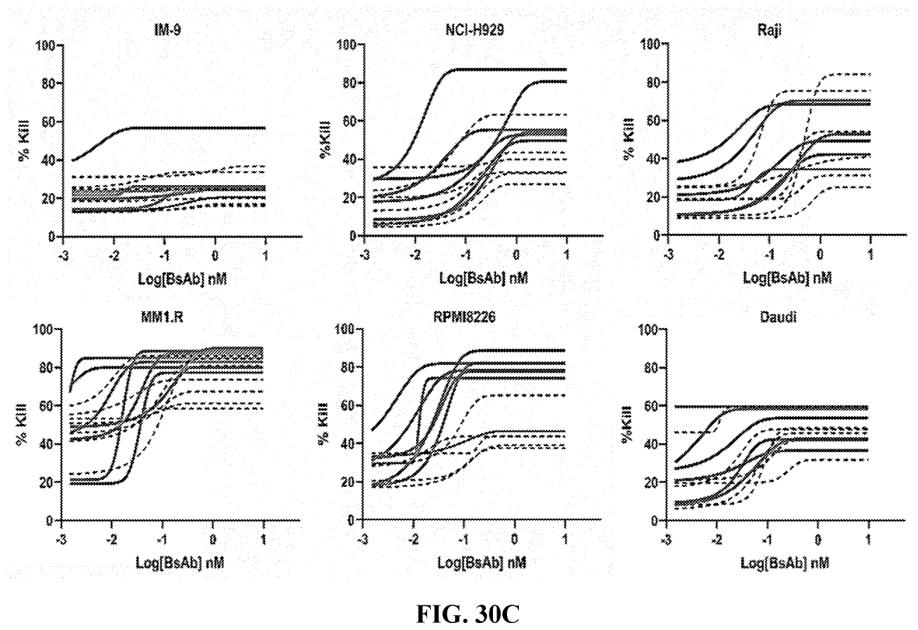

Flow cytometer (Intellicyte IQue Screener, Sartorius) was set up with BL-1 channel for GFP and RL-1 channel for CD38-APC detection. The CD38-APC unstained samples served as compensation reference for APC channels. Cells were then identified using the FSC-H (forward scattering fluorescence height) vs SSC-H (side scattering fluorescence-height) scatter plot of all events. The cell population was then plotted with FCS-H vs FCS-A (area) to identify the singlets. The singlets were further plotted with fluorescence channel BL1-H vs RL1-H and the geocentric mean of the RL1-H peak for each tumor cell line was exported. The geocentric mean on RL1-H peak of each stained cell line was plotted as representation of CD38 expression level using GraphPad Prism software (FIG. 30A).

### Example 11: CD38/CD3 Bispecific or CD38 mAb-Mediated Tumor Cell Killing by Previously Unstimulated Human PBMCs

The efficiencies of CD38/CD3 bispecific antibody to redirect primary human T cells to kill CD38-expressing tumor cell lines were assessed by subjecting equal amount of freshly isolated human PBMCs and each CD38(+) tumor cell lines, at a fixed E/T (effector cells/target cells) ratio, to a concentration gradient of CD38/CD3 bispecific antibody in 96-well plates overnight at 37 C. To compare the potency of redirected T cell cytotoxicity (RTCC) mediated by CD38/CD3 bispecific antibody with that of antibody-dependent cell-mediated cytotoxicity (ADCC), an NK cell mediated CD38-targeting commercial mAb drug Darzalex-dependent tumor cell killing phenomenon, the same concentration gradient of Darzalex was also used treat similar PBMCs/CD38(+) tumor cells mixture in parallel. Apoptosis marker presence on the tumor cells were quantified the next day using a flow cytometry method.

Freshly isolated human PBMCs were kept in RPMI media, supplemented with 10% FBS and 10 ng/mL IL-7 at 2-5E+6 cells/mL at 37° C. static incubator overnight. Prior to the cytotoxicity assay setup, an assay of cytotoxic versus helper T cell ratio were conducted by quantifying the percentage of CD4(+) and CD8(+) subpopulations, respectively, in total CD3(+) T cell counts using a compensated three-color flowcytometry method. A parallel assay of NK cell content in isolated PBMCs by a compensated two-color flow cytometry method staining CD3(+) for T cell and CD56 (+) for NK was also performed. The CD4(+)/CD8(+) ratio, as well as the CD56(+) in percentage of the total PBMC of each donor are presented in FIG. 30B.

The freshly isolated PBMCs as well as the GFP expressing and CD38 antigen level characterized tumor cells of viability >90% on the day of the assay setup were each washed with RPMI media once and density adjusted to achieve an E/T ratio of 10:1 in each assay, except for the Raji cell line using a 40:1 ratio, with approximately 15,000 target cells per data point. A serial 1:3 or 1:4 dilution of Kv5.1 anti-CD38/CD3 bispecific antibody molecule or anti-CD38 mAb Darzalex, was prepared in RPMI media and added to the E/T cell mixtures in each 96-well plates. A series of target cell and T cell only wells were also setup as the secondary antigen only and no-bispecific antibody controls. Tumor cells only and PBMCs only wells were also included to assess the intrinsic apoptosis baseline of each cell type. To address the edge effect of plate-based assays, only the inner rows and columns of the 96-well plates were used for setting the assays. The border rows and columns were filled with equal volumes of the RPMI media as the inner assay wells. The assay plates were than kept in at a 37° C. static incubator overnight.

Cells from the overnight incubation were pelleted by centrifugation. The cell pellets were washed with FACS buffer once and the centrifuged prior to supernatant removal. The cell pellets in each assay plate were then subjected to Annexin V, an early apoptosis marker, detection and analysis. The Annexin V detection was performed using a commercially available FACS kit by BioLegend, Inc, following manufacturer recommended conditions and procedures.

The assay plates were then subjected to FACS analysis on the Intellicyte IQue Screener (Sartorius) instrument with BL-1 channel for GFP and RL-1 channel Annexin V detection. Due to the constitutive expression of GFP, the tumor target cells can be distinguished from the effector cells on the FSC-A (forward scattering—area) vs BL1-H scatter plot, with the BL1-H high population being the target cells, the BL1-H low the effector cells. The GFP(+) tumor cells were further gated on the RL1-H (Annexin V) vs BL1-H scatter plot, with the RL1-H high population being the Annexin V high expressing subset. The % Kill of the target cells is defined as the percentage of the Annexin V high expressing subset over the total cell counts of the GFP high tumor cell population in each assay condition. The % Kill of each assay well was plotted against the Kv5.1 CD38/CD3 bispecific antibody or anti-CD38 mAb Darzalex concentration gradient.

The 5-parameter non-linear regression (5-PL) fitted dose-dependent response model was used to fit these % Kill vs Log[Concentration] plots in GraphPad Prism (FIG. 38C). PMBCs from multiple donors were profiled against each of the six CD38(+) tumor cells in duplicates. Each tumor cell lines were screened with PBMCs from at least 6 random healthy donors from the blood bank for respective RTCC by anti-CD38/CD3 bispecific antibody or ADCC by anti-CD38 mAb Darzalex. The percent tumor killing responses in either bispecific antibody or mAb gradient by each donor were plotted together with line style coded for bispecific antibody or mAb molecule only without distinguishing individual donors. In FIG. 38C, the dose-dependent killing of each CD38(+) tumor cell line by cytotoxic T cells or NK cells from each tested donor were plotted in the same panel. In all panels, in-tandem 3-chain Kv5.1 CD38/CD3 bispecific antibody responses are represented by grey curves (solid lines) and those of Darzalex's by dash-lines. The data shown in FIG. 30C indicates that Kv5.1 CD38/CD3 bispecific antibody exhibits greater potency in inducing CD38(+) tumor target cell death mediated by cytotoxic T cells than Darzalex by NK cells.

### Example 12: In Vivo Tumor Killing in a Mouse Model

Tumoricidal activity of the CD38/CD3 in-tandem (Kv5.1) and non-tandem (Kv4.33) bispecific antibodies will be tested in a CD38(+) multiple-myeloma (MM) or other CD38(+) solid tumor xenograft mouse model. Eight week old female NSG mice will be used for the study. CD38(+) MM or other solid tumor cell lines from ATCC will be virally transfected with a polycistronic expression cassette for luciferase and GFP genes. A single cell clone with stable polycistronic luciferase and GFP expression is selected from the CD38(+) tumor cell line transfectant. The CD38(+) tumor-FLuc cells are suspended in PBS or other appropriate formulation buffer and injected intravenously into the tail vein of each NSG mouse for tumor uptake and xenograft establishment either weeks or days prior to bispecific antibody treatment for evaluating antitumor efficacy for existing/established tumors. Animals with very small or very large tumor burden will be excluded based on the bioluminescence from IVIS imaging. The animals selected in study will be randomized in different groups. Alternatively, CD38(+) tumor-Fluc cell suspension can be intravenously injected to the NSG mice the same day of bispecific antibody treatment to assess its prophylactic effect against aggressive metastatic tumors.

On Day zero, approximately 1×10\hat{ }6 to 1×10\hat{ }7 previously unstimulated human PBMC from a single healthy donor is injected into each tumor-bearing NSG through tail vein in the tumor eradication model. On the same day, a treatment of PBS, control anti-CD38 mAb, bispecific CD38/CD3 Kv5.1, or bispecific CD38/CD3 Kv4.33, is administered via the opposite tail vein or intraperitoneally in the same volume of PBS/formulation buffer after tumor inoculation as in the tumor eradication model. In the prophylactic model, the MM-Fluc cell suspension is mixed with the previously unstimulated hPBMC and injected intravenously through the tail vein on one side. The bispecific antibody and control treatment agents are similarly administered through the opposite tail vein or intraperitoneally the same day of tumor cells/PBMC injection as described earlier. A single dose of the bispecific antibody and control treatments are administered, or multiple doses are administered weekly and can be continued up to 4 weeks until tumor burden reaches regulation criteria for the individual(s) animals to be euthanized.

Tumor growth in the mice will be monitored by measuring total photon flux on the dorsal side of each mouse weekly after tumor cell inoculation and continued weekly during treatment course. The monitoring can continue up to 4 or 5 weeks after the last treatment. The images will be taken about 10 to 20 minutes after 150 mg/kg luciferin intraperitoneal administration. The body weight of all mice will be monitored and recorded at the same time that tumor bioluminescence is measured. Blood samples are collected from each animal via tail vein prior to as well as the day after each treatment. After the administration of the last dose, blood samples are collected weekly thereafter. The blood samples will be analyzed via flow cytometry for tumor abundance and cell death markers, as well effector cell biomarkers such as activation, cytotoxicity, proliferation, activation-induced cell death, and receptor occupancy by bispecific antibody. Serum extracted from the blood samples are also analyzed for cytokine release, free bispecific antibody, and potential bispecific antibody fragments.

The anti-tumor efficacy of the bispecific antibodies Kv5.1 and Kv4.33 CD38/CD3 is evaluated primarily as the extent of survival extension in the bispecific-treated group vs control mAb, control bispecific, or PBMC-only treated groups. The biomarker profiles, such as T cell activation, T cell proliferation, T cell differentiation, T cell death as a response to drug induction and activation, in the effector cell compartment of the murine blood samples from treatment and control groups can also demonstrate the effectiveness of the bispecific antibodies Kv5.1 and Kv4.33 CD38/CD3 in redirecting human T cells against CD38(+) tumor cells in a dose and antigen (CD38)-specific manner.

TABLE 8
(Appendix to Examples 1-3):

| | ka | kd | KD | Rmax | Chi² |
|---|---|---|---|---|---|
| Molecules | (1/Ms) | (1/s) | (M) | (RU) | (RU²) |
| Kv4.33: EGFR/PDL1 | 4.12E+05 | 8.12E−03 | 1.97E−08 | 107 | 0.130 |
| Mobile: EGFR-his | | | | | |
| Kv5.1: EGFR/PDL1 | 2.42E+06 | 6.99E−03 | 2.90E−09 | 112 | 1.877 |
| Mobile: EGFR-his | | | | | |
| Control anti-EGFR Ab | 2.17E+06 | 1.46E−03 | 6.72E−10 | 342 | 5.985 |
| Mobile: EGFR-his | | | | | |
| Anti-EGFR mAb (D2GA1) | 5.09E+05 | 6.71E−03 | 1.32E−08 | 168 | 0.583 |
| Mobile: EGFR-his | | | | | |
| Kv4.33: EGFR/PDLl | 2.49E+06 | 2.55E−03 | 1.03E−09 | 44 | 0.232 |
| Mobile: PD-L1-his | | | | | |
| Kv5.1: EGFR/PDL1 | 1.92E+06 | 3.32E−03 | 1.73E−09 | 42 | 1.577 |
| Mobile: PD-L1-his | | | | | |
| Anti-PD-L1 mAb | 3.99E+06 | 2.94E−03 | 7.38E−10 | 134 | 2.470 |
| (H6b1LEM) | | | | | |
| Mobile: PD-L1-his | | | | | |
| Kv4.33: CD38/CD3 | 1.76E+06 | 2.33E−03 | 1.33E−09 | 62 | 1.097 |
| Mobile: CD38 | | | | | |
| Kv5.1: CD38/CD3 | 2.31E+06 | 2.04E−03 | 8.83E−10 | 92 | 1.753 |
| Mobile: CD38 | | | | | |
| Anti-CD38 (3H10m1) | 1.57E+06 | 1.60E−03 | 1.02E−09 | 169 | 4.554 |
| Mobile: CD38 | | | | | |
| Kv6.1: CD38/CD3 | 1.50E+06 | 2.29E−03 | 1.52E−09 | 52 | 0.390 |
| Mobile: CD38 | | | | | |
| Kv6.2: CD38/CD3 | 9.19E+05 | 2.29E−03 | 2.49E−09 | 77 | 0.303 |
| Mobile: CD38 | | | | | |
| Kv4.33: BCMA/CD3 | 7.62E+05 | 3.64E−02 | 4.77E−08 | 20 | 0.835 |
| Mobile: BCMA | | | | | |
| Anti-BCMA mAb (2C5) | 1.02E+06 | 2.75E−02 | 2.68E−08 | 44 | 0.406 |
| Mobile: BCMA | | | | | |
| EGFR | 2.1E+05 | 7.80E−03 | 3.71E−08 | 79 | 0.338 |
| Mobile: Kv4.33: | | | | | |
| EGFR/PDL1 | | | | | |
| EGFR | 2.03E+05 | 7.39E−03 | 3.64E−08 | 80 | 0.350 |
| Mobile: | | | | | |
| Kv5.1: EGFR/PDL1 | | | | | |
| PD-L1 | 4.46E+05 | 3.07E−03 | 6.89E−09 | 104 | 0.405 |
| Mobile: | | | | | |
| Kv4.33: EGFR/PDL1 | | | | | |
| PD-L1 | 1.60E+05 | 4.59E−03 | 2.87E−08 | 154 | 0.651 |
| Mobile: Kv5.1: | | | | | |
| EGFR/PDL1 | | | | | |
| Kv5.1: EGFR/PDL1 | 4.20E+06 | 2.43E−02 | 5.78E−09 | 9 | 2.476 |
| Mobile: PD-L1 | | | | | |
| Kv5.1: EGFR/PDL1 | 3.00E+06 | 1.52E−02 | 5.09E−09 | 47 | 1.774 |
| Mobile: EGFR | | | | | |

1. An activatable multi-specific antigen binding protein complex, comprising
a) a first polypeptide chain comprising (i) a first half Fab heavy region, (ii) a first linker, (iii) a second half Fab heavy region, and (iv) a first half Fc region, wherein the first half Fab heavy region comprises a first-variable region and first-constant region from a first Fab heavy chain, and wherein the second half Fab heavy region comprises a second-variable region and second-constant region from a second Fab heavy chain; and a second polypeptide chain comprising (i) a first half Fab light region, (ii) a second linker, (iii) a second half Fab light region, and (iv) a second half Fc region, wherein the first half Fab light region comprises a first-variable region and first-constant region from a first Fab light chain, and wherein the second half Fab light region comprises a second-variable region and second-constant region from a second Fab light chain, wherein the second linker is cleavable, and wherein the first and second polypeptide chains associate with each other to form the multi-specific antigen binding protein complex having a first full Fab domain that is capable of binding a first epitope, a second full Fab domain that is capable of binding a second epitope that differs from the first epitope, a full Fc domain that is capable of binding an Fc receptor, and a first and second linker, wherein the first full Fab domain and the second full Fab domain are different and are arranged in tandem; or
b) a first polypeptide chain comprising (i) a first half Fab heavy region, (ii) a first half Fc region, (iii) a first linker, and (iv) a second half Fab heavy region, wherein the first half Fab heavy region comprises a first-variable region and first-constant region from a first Fab heavy chain, and wherein the second half Fab heavy region comprises a second-variable region and second-constant region from a second Fab heavy chain; and a second polypeptide chain comprising (i) a first half Fab light region, (ii) a second half Fc region, (iii) a second linker, and (iv) a second half Fab light region, wherein the first half Fab light region comprises a first-variable region and first-constant region from a first Fab light chain, and wherein the second half Fab light region comprises a second-variable region and second-constant region from a second Fab light chain, wherein the second linker is cleavable, and wherein the first and second polypeptide chains associate with each other to form the multi-specific antigen binding protein complex having a first full Fab domain that is capable of binding a first epitope, a second full Fab domain that is capable of binding a second epitope that differs from the first epitope, a full Fc domain that is capable of binding an Fc receptor, and a first and second linker, wherein the first full Fab domain and the second full Fab domain are different and are arranged in a non-tandem manner.

2. The protein complex of claim 1, wherein: the first full Fab domain exhibits binding to the first epitope; the second full Fab domain exhibits a first level of binding to the second epitope when the second linker is un-cleaved; the second full Fab domain exhibits a second level of binding to the second epitope when the second linker is cleaved, the second level being increased relative to the first level; and upon cleavage of the second linker the first full Fab domain and the second full Fab domain are capable of binding to the first and second target epitopes, respectively, at the same time.

3. The protein complex of claim 1, wherein the second linker is cleavable with a matrix metalloprotease (MMP), and wherein the matrix metalloprotease is MMP1, MMP2, MMP3, MMP8, MMP9, MMP11, MMP13, MMP14, or MT1-MMP (membrane type 1 matrix metalloproteinase).

4. The protein complex of claim 1, wherein the second linker comprises the amino acid sequence of:
a)
(DP linker, SEQ ID NO: 11)
GGSGSGSGGSSGGGSGGGGS;
b)
(EG or EH linker, SEQ ID NOS: 12 or 44)
TSGSGGSGGSV;
c)
(EI or EU linker, SEQ ID NOS: 13 or 45)
TSGSGGSPLGMGGSGSV;
d)
(EJ or EV linker, SEQ ID NOS: 14 or 46)
TSGSGGSPLGVGGSGSV;
e)
(EK or EW linker, SEQ ID NOS: 15 or 47)
TSGSGGSPAALGGSGSV;
f)
(EL or EX linker, SEQ ID NOS: 16 or 48)
TSGSGGSPAGLGGSGSV;
g)
(EM or EY linker, SEQ ID NOS: 17 or 49)
TSGSGGSPLGMVGV;
h)
(EN or EZ linker, SEQ ID NOS: 18 or 50)
TSGSGGSPLGVVGV;
i)
(EO or FA linker, SEQ ID NOS: 19 or 51)
TSGSGGSPAALVGV;
j)
(EP or FB linker, SEQ ID NOS: 20 or 52)
TSGSGGSPAGLVGV;
k)
(EQ or FC linker, SEQ ID NOS: 21 or 53)
TSGSGGSPLGMVLV;
l)
(ER or FD linker, SEQ ID NOS: 22 or 54)
TSGSGGSPLGVVLV;
m)
(ES or FE linker, SEQ ID NOS: 23 or 55)
TSGSGGSPAALVLV;
or
n)
(ST or FF linker, SEQ ID NOS: 24 or 56)
TSGSGGSPAGLVLV.

5. The protein complex of claim 1, wherein the first polypeptide chain comprises a knob or hole mutation in the first half Fc region; the second polypeptide chain comprises a hole mutation in the second half Fc region if the first polypeptide chain comprises a knob mutation in the first half Fc region; and the second polypeptide chain comprises a knob mutation in the second half Fc region if the first polypeptide chain comprises a hole mutation in the first half Fc region.

6. The protein complex of claim 1, wherein
a) the first full Fab domain and the second full Fab domain are arranged in-tandem and: (i) the first polypeptide chain having the first half Fab heavy region comprises the amino acid sequences of SEQ ID NO: 1 and SEQ ID NO: 2, and the second half Fab heavy region comprises the amino acid sequences of SEQ ID NO: 4 and SEQ ID NO: 5, and (ii) the second polypeptide chain having the first half Fab light region comprises the amino acid sequences of SEQ ID NO: 9 and SEQ ID NO: 10, and the second half Fab light region comprises the amino acid sequences of SEQ ID NO: 25 and SEQ ID NO: 26; or
b) the first full Fab domain and the second full Fab domain are arranged in a non-tandem manner and: (i) the first polypeptide chain having the first half Fab heavy region comprises the amino acid sequences of SEQ ID NO: 30 and SEQ ID NO: 31, and the second half Fab heavy region comprises the amino acid sequences of SEQ ID NO: 36 and SEQ ID NO: 37, and (ii) the second polypeptide chain having the first half Fab light region comprises the amino acid sequences of SEQ ID NO: 38 and SEQ ID NO: 39, and the second half Fab light region comprises the amino acid sequences of SEQ ID NO: 57 and SEQ ID NO: 58.

7. A pharmaceutical composition comprising the multi-specific antigen binding protein complex of claim 1 and a pharmaceutically-acceptable excipient.

8. (canceled)

9. One or more nucleic acids encoding (i) the first polypeptide chain of the multi-specific antigen binding protein complex of claim 1 and (ii) the second polypeptide chain the multi-specific antigen binding protein complex of claim 1.

10. One or more expression vectors comprising the one or more nucleic acids of claim 9 operably linked to one or more promoters.

11. A host cell harboring the one or more expression vectors of claim 10.

12. A method for preparing a multi-specific antigen binding protein complex, comprising:
a) culturing a population of the host cell of claim 11 under conditions suitable for expressing the first and the second polypeptide chains and suitable for associating the first and second polypeptide chains with each other to form the multi-specific antigen binding protein complex; and
b) recovering the multi-specific antigen binding protein complex from the population of the host cell.

13. A method for treating a disease in a subject, comprising:
administering to the subject a therapeutically effective amount of the multi-specific antigen binding protein complex of claim 1.

14. The method of claim 13, wherein the disease comprises a cancer, comprising a prostate cancer, breast cancer, ovarian cancer, head and neck cancer, bladder cancer, skin cancer, colorectal cancer, anal cancer, rectum cancer, pancreatic cancer, lung cancer (including non-small cell lung and small cell lung cancers), leiomyoma cancer, brain cancer, glioma cancer, glioblastoma cancer, esophagus cancer, liver cancer, kidney cancer, stomach cancer, colon cancer, cervical cancer, uterine cancer, endometrial cancer, vulva cancer, larynx cancer, vaginal cancer, bone cancer, nasal cavity cancer, paranasal sinus cancer, nasopharynx cancer, oral cavity cancer, oropharynx cancer, larynx cancer, hypolarynx cancer, salivary gland cancer, ureter cancer, urethral cancer, penial cancer, or testicular cancer.

15. The method of claim 13, wherein the disease comprises a hematologic cancer, including a B chronic lymphocytic leukemia (B-CLL), B and T acute lymphocytic leukemia (ALL), acute myeloid leukemia (AML), chronic lymphocytic leukemia (CLL), chronic myelogenous leukemia (CIVIL), hairy cell leukemia (HCL), myeloproliferative disorder/neoplasm (MPDS), myelodysplasia syndrome, non-Hodgkin's lymphoma (NHL) including Burkitt's lymphoma (BL), Waldenstrom's Macroglobulinemia, mantle cell lymphoma, AIDS-related lymphoma, Hodgkin's Lymphoma (HL), T cell lymphoma (TCL), multiple myeloma (MM), plasma cell myeloma, plamocytoma, giant cell myeloma, heavy-chain myeloma, or light chain or Bence-Jones myeloma.

16. A method for binding a first and a second epitope, comprising:
a) contacting the first epitope with the multi-specific antigen binding protein complex of claim 1, wherein the second linker is in an un-cleaved state; and
b) binding the first epitope with the first Fab region, wherein the first Fab region binds the first epitope and the second Fab region exhibits a level of binding to the second epitope that is reduced relative to the level of binding to the second epitope exhibited when the second linker is in a cleaved state.

17. The method of claim 16 further comprising: cleaving the second linker to generate an activated multi-specific antigen binding protein complex wherein the second Fab region exhibits a level of binding to the second epitope that is increased relative to the level of binding to the second epitope exhibited when the second linker is in the un-cleaved state.

18. The method of claim 17 further comprising:
a) contacting the second target epitope with the activated multi-specific antigen binding protein complex; and
b) binding the second target epitope with the second Fab region.

19. The method of claim 16, wherein the first target epitope comprises a cell surface antigen expressed by a tumor or cancer cell, and wherein the second epitope comprises a cell surface antigen expressed by an effector T cell.

20. The method of claim 19, wherein the activated multi-specific antigen binding protein complex forms a cell synapse by binding the cell surface antigen expressed by the tumor or cancer cell and by binding the cell surface antigen expressed by the effector T cell.

21. The method of claim 20, wherein the effector T cell in the cell synapse kills the tumor or cancer cell by mediating cytotoxic cell killing.
